(19) United States
(12) Patent Application Publication
(10) Pub. No.: US 2021/0300898 A1
(43) Pub. Date: Sep. 30, 2021

(54) SMALL MOLECULES TARGETING MUTANT MAMMALIAN PROTEINS

(71) Applicant: Jnana Therapeutics, Inc., Boston, MA (US)

(72) Inventors: Giovanni Muncipinto, Cambridge, MA (US); Lyn H. Jones, Winchester, MA (US); Terry J. Rettenmaier, Braintree, MA (US)

(21) Appl. No.: 17/278,047

(22) PCT Filed: Sep. 20, 2019
(86) PCT No.: PCT/US2019/052196

§ 371 Date: Mar. 19, 2021

Related U.S. Application Data
(60) Provisional application No. 62/734,668, filed on Sep. 21, 2018.

(51) Int. Cl. C07D 401/12; C07D 215/20; C07D 401/06; C07D 413/12; C07D 405/12; C07D 405/14

(52) U.S. Cl. CPC C07D 401/12; C07D 215/20; C07D 405/14; C07D 413/12; C07D 405/12; C07D 401/06

(57) ABSTRACT

Disclosed are compounds, compositions, and methods useful for treating or preventing a disease or disorder associated with a mutation in a protein.

### RELATED APPLICATIONS

This application claims the benefit of priority to U.S. Provisional Patent Application No. 62/734,668, filed Sep. 21, 2018.

### BACKGROUND

Creatine transporter deficiency (CTD) has been reported to be the most common cerebral creatine deficiency syndrome (CCDS). Creatine transporter deficiency is an X-linked disorder caused by mutations in the SLC6A8 gene. The SLC6A8 gene, located on the short arm of the sex chromosome, provides instructions for making a protein that transports the compound creatine into cells. Creatine is needed for the body to store and use energy properly. People with CTD have intellectual disability, which can range from mild to severe, and delayed speech development. Some affected individuals develop behavioral disorders such as attention deficit hyperactivity disorder or autistic behaviors that affect communication and social interaction. They may also experience seizures. Children with CTD may experience slow growth and exhibit delayed development of motor skills such as sitting and walking. CTD is difficult to treat because the actual transporter responsible for transporting creatine to the brain and muscles is defective. There is no current standard of care.

### SUMMARY

One aspect of the invention provides compounds, compositions, and methods useful for treating or preventing a disease or disorder associated with a SLC6A8 mutation.

Accordingly, provided herein is a compound having the structure of Formula (I):

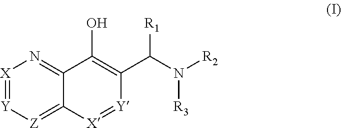

wherein

X, X′, Y, Y′, and Z are independently selected from N and C(R); provided that no more than two of X, X′, Y, Y′, and Z are N;

if Z and Y, or Y and X, or X′ and Y′, are C(R), then the two adjacent instances of R on any of them taken together may form a fused 3-8 membered ring;

R is independently selected from H, alkyl, alkenyl, alkynyl, cycloalkyl, heteroalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, haloalkyl, halocycloalkyl, halocycloheteroalkyl, —O-alkyl, —O-haloalkyl, —O-cycloalkyl, —N-alkyl, —N-haloalkyl, —N-cycloalkyl, —S-alkyl, —S-haloalkyl, —S-cycloalkyl, —O-heteroalkyl, —O— cycloheteroalkyl, —N-heteroalkyl, —N-cycloheteroalkyl, —S-heteroalkyl, —S-cycloheteroalkyl, —O-aryl, —N-aryl, —S-aryl, —O-heteroaryl, —N-heteroaryl, —S-heteroaryl, substituted or unsubstituted —O-alkylene-aryl, substituted or unsubstituted —N-alkylene-aryl, substituted or unsubstituted —S— alkylene-aryl, substituted or unsubstituted —O-alkylene-heteroaryl, substituted or unsubstituted —N-alkylene-heteroaryl, substituted or unsubstituted —S-alkylene-heteroaryl, halide, —CN, —NO₂, —S(O)Rₐ, —S(O)₂Rₐ, —C(O)Rₐ, —C(O)₂Rₐ, —C(O)NRₐR_{b}, OH, and C(O)NR′C(NR′)NRₐR_{b};

R′ is H, or alkyl;

Rₐ and R_{b} are independently H, alkyl, alkenyl, alkynyl, substituted or unsubstituted aryl, cycloalkyl, heteroalkyl, haloalkyl, cycloheteroalkyl, halocycloalkyl, halocycloheteroalkyl, substituted or unsubstituted heteroaryl, substituted or unsubstituted -alkylene-aryl, substituted or unsubstituted -alkylene-heteroaryl, alkylene-OR′, alkylene-NR′, alkylene-SR′, or Rₐ and R_{b} taken together with the nitrogen atom to which they are attached may form a 3-8 membered ring;

R₁ is H, alkyl, alkenyl, alkynyl, haloalkyl, heteroalkyl, heterohaloalkyl, cycloalkyl, halocycloalkyl, heterocycloalkyl, heterohalocycloalkyl, unsubstituted or substituted aryl, unsubstituted or substituted alkenyl-aryl, unsubstituted or substituted heteroaryl, unsubstituted or substituted alkenyl-heteroaryl, alkylene-OR′, alkylene-NR′, or alkylene-SR′; and

R₂, and R₃ are independently selected from H, alkyl, alkenyl, alkynyl, unsubstituted or substituted heteroaryl, unsubstituted or substituted aryl, haloalkyl, heteroalkyl, heterohaloalkyl, cycloalkyl, halocycloalkyl, heterocycloalkyl, heterohalocycloalkyl, alkylene-cycloalkyl, alkylene-aryl, alkylene-heteroaryl, alkylene-OR′, alkylene-NR′, alkylene-SR′, —S(O)Rₐ, —S(O)₂Rₐ, —C(O)Rₐ, —C(O)₂Rₐ, —C(O)NRₐR_{b}, —C(NRₐ)NRₐR_{b}, —N(Rₐ)C(NRₐ)NRₐR_{b}, and —C(O)NR′C(NR′)NRₐR_{b}; or R₂ and R₃ taken together may form a 4-8 membered ring.

Another aspect of the invention relates to methods of treating or preventing a disease or disorder associated with a SLC6A8 mutation, comprising administering to a subject in need thereof an effective amount of a compound of the invention.

In some embodiments, the invention relates to methods of increasing cellular trafficking of a creatine transporter, comprising administering to a subject in need thereof an effective amount of a compound of the invention.

In some embodiments, the invention relates to methods of correcting a defect in cellular creatine transporter function, comprising administering to a subject in need thereof an effective amount of a compound of the invention.

In certain embodiments, the subject is a mammal. In certain embodiments, the mammal is a human.

The invention provides several additional advantages. The prophylactic and therapeutic methods described herein are also effective for treating creatine transporter deficiency and associated symptoms. In some embodiments, the therapeutic method is effective in treating motor dysfunction, intellectual disability, language delay, speech delay, seizures, behaviors associated with autism and attention deficit hyperactivity disorder, fatigue, muscular hypotonia, low weight gain, and gastrointestinal and cardiac disorders.

In some embodiments, the therapeutic method is effective in treating inflammatory diseases. In some embodiments, the inflammatory disease is acute. In some embodiments, the inflammatory disease is chronic. In some embodiments, the inflammatory disease is selected from inflammatory bowel diseases (for example, ulcerative colitis or Crohn's disease), multiple sclerosis, psoriasis, arthritis, rheumatoid arthritis, osteoarthritis, juvenile arthritis, psoriatic arthritis, reactive arthritis, ankylosing spondylitis, cryopyrin associated periodic syndromes, Muckle-Wells syndrome, familial cold auto-inflammatory syndrome, neonatal-onset multisystem inflammatory disease, TNF receptor associated periodic syndrome, acute and chronic pancreatitis, atherosclerosis, gout, ankylosing spondylitis, fibrotic disorders (for example, hepatic fibrosis or idiopathic pulmonary fibrosis), nephropathy, sarcoidosis, scleroderma, anaphylaxis, diabetes (for example, diabetes mellitus type 1 or diabetes mellitus type 2), diabetic retinopathy, Still's disease, vasculitis, sarcoidosis, pulmonary inflammation, acute respiratory distress syndrome, wet and dry age-related macular degeneration, autoimmune hemolytic syndromes, autoimmune and inflammatory hepatitis, autoimmune neuropathy, autoimmune ovarian failure, autoimmune orchitis, autoimmune thrombocytopenia, silicone implant associated autoimmune disease, Sjogren's syndrome, familial Mediterranean fever, systemic lupus erythematosus, vasculitis syndromes (for example, temporal, Takayasu's and giant cell arteritis, Behçet's disease or Wegener's granulomatosis), vitiligo, secondary hematologic manifestation of autoimmune diseases (for example, anemias), drug-induced autoimmunity, Hashimoto's thyroiditis, hypophysitis, idiopathic thrombocytic pupura, metal-induced autoimmunity, myasthenia gravis, pemphigus, autoimmune deafness (for example, Meniere's disease), Goodpasture's syndrome, Graves' disease, HW-related autoimmune syndromes, Gullain-Barre disease, Addison's disease, anti-phospholipid syndrome, asthma, atopic dermatitis, Celiac disease, Cushing's syndrome, dermatomyositis, idiopathic adrenal adrenal atrophy, idiopathic thrombocytopenia, Kawasaki syndrome, Lambert-Eaton Syndrome, pernicious anemia, pollinosis, polyarteritis nodosa, primary biliary cirrhosis, primary sclerosing cholangitis, Raynaud's, Reiter's Syndrome, relapsing polychondritis, Schmidt's syndrome, thyrotoxidosis, sepsis, septic shock, endotoxic shock, exotoxin-induced toxic shock, gram negative sepsis, toxic shock syndrome, glomerulonephritis, peritonitis, interstitial cystitis, hyperoxia-induced inflammations, chronic obstructive pulmonary disease (COPD), vasculitis, graft vs. host reaction (for example, graft vs. host disease), allograft rejections (for example, acute allograft rejection or chronic allograft rejection), early transplantation rejection (for example, acute allograft rejection), reperfusion injury, pain (for example, acute pain, chronic pain, neuropathic pain, or fibromyalgia), chronic infections, meningitis, encephalitis, myocarditis, gingivitis, post surgical trauma, tissue injury, traumatic brain injury, enterocolitis, sinusitis, uveitis, ocular inflammation, optic neuritis, gastric ulcers, esophagitis, peritonitis, periodontitis, dermatomyositis, gastritis, myositis, polymyalgia, pneumonia and bronchitis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### BRIEF DESCRIPTION OF THE DRAWINGS

FIG. 1 is a table summarizing trafficking and correction data for exemplary compounds of the invention.

FIG. 2 is a table summarizing trafficking and correction data for exemplary compounds of the invention.

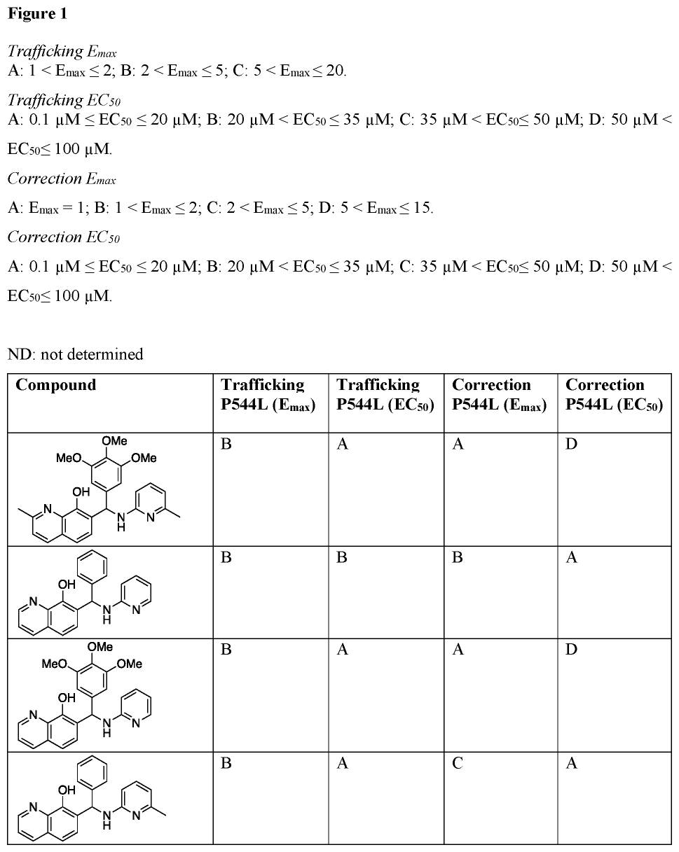

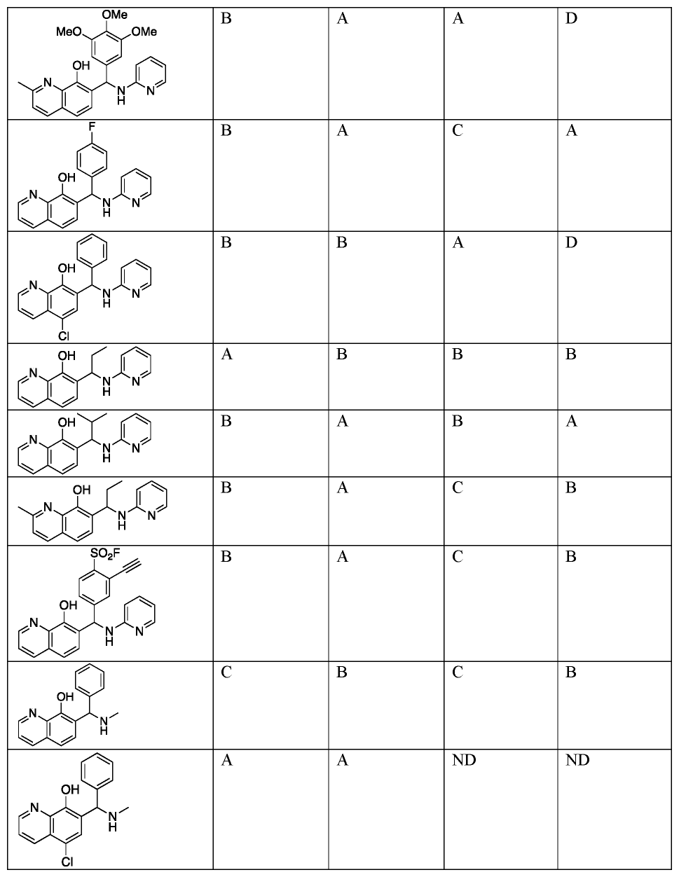

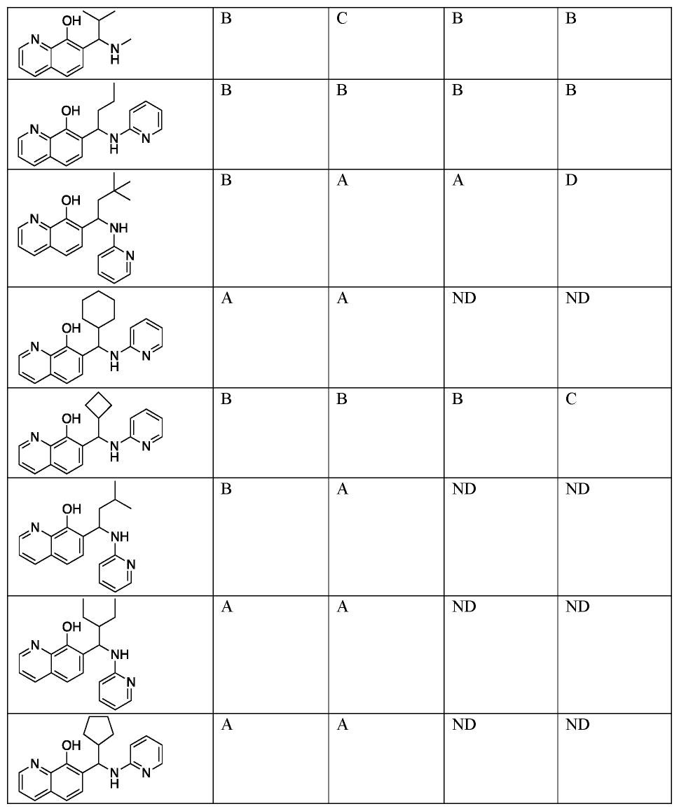

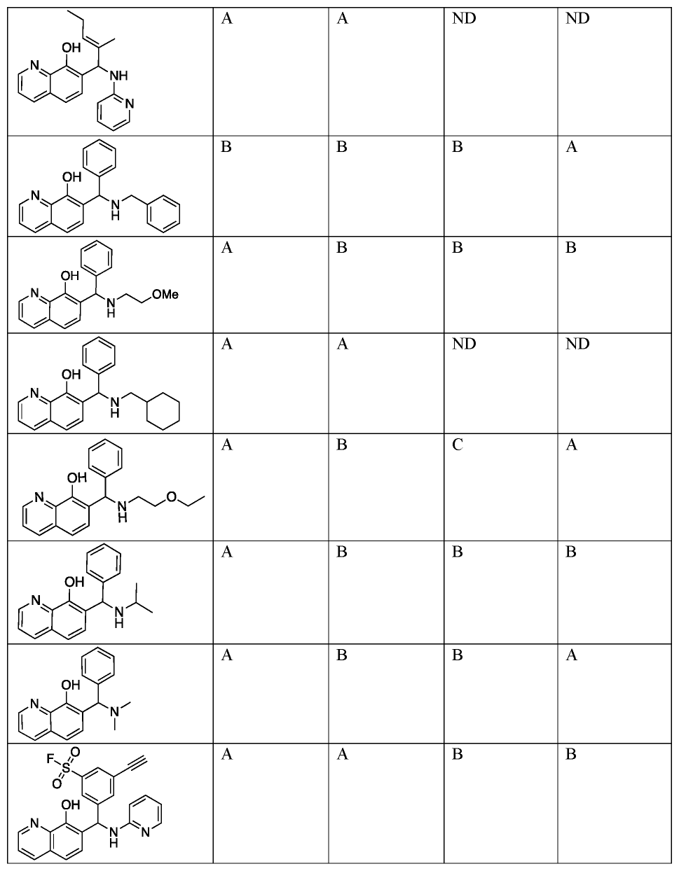

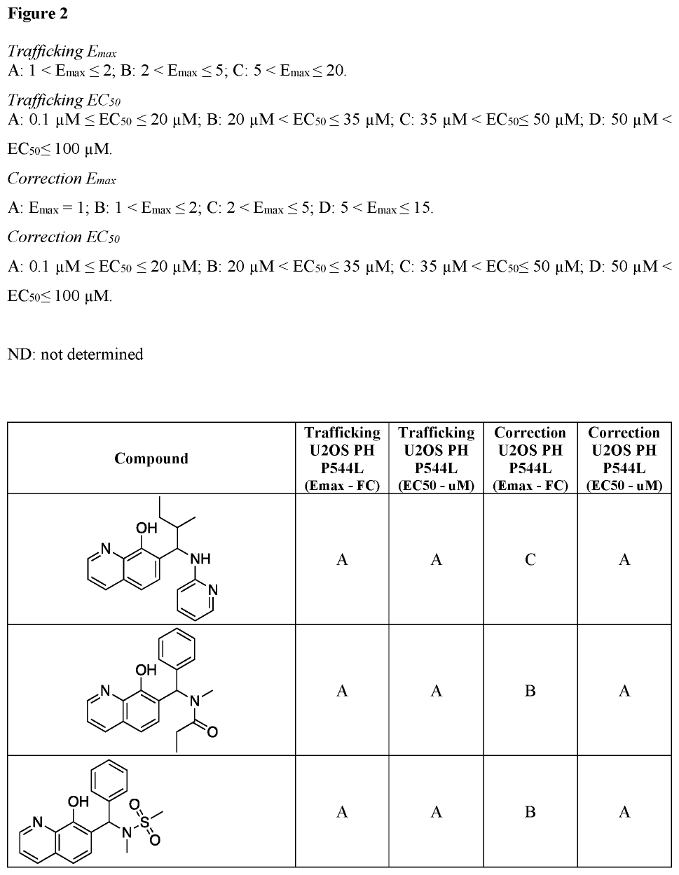

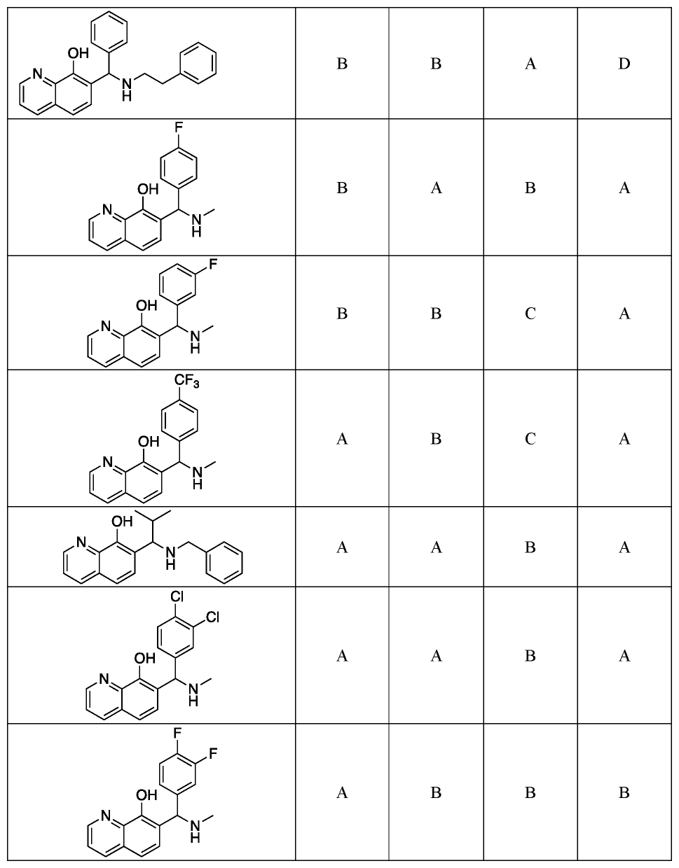

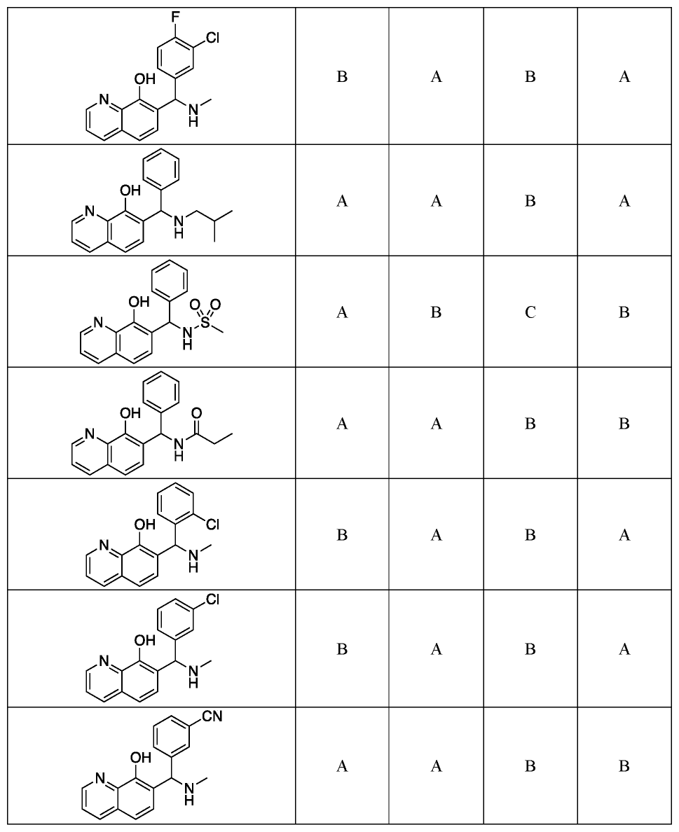

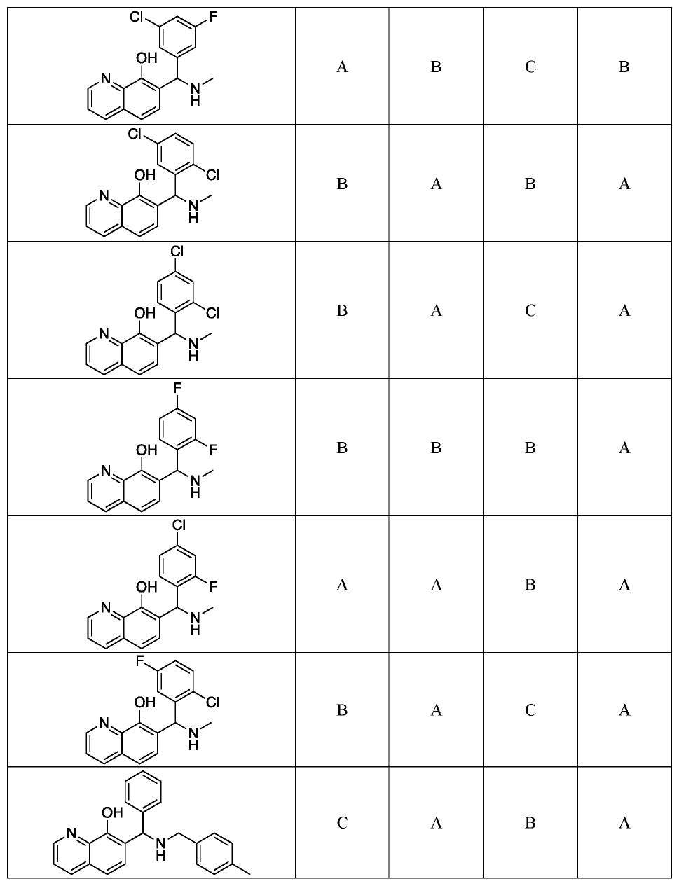

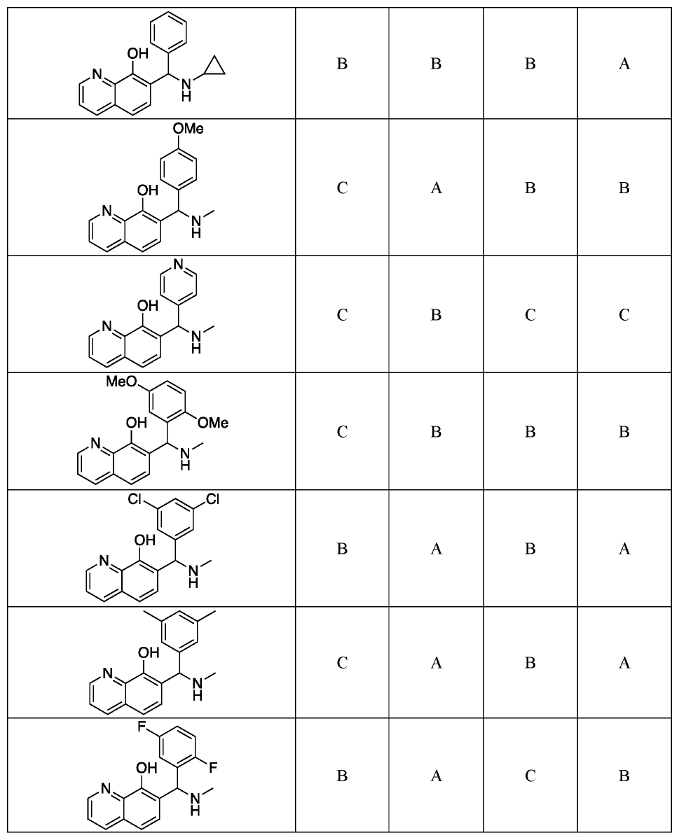

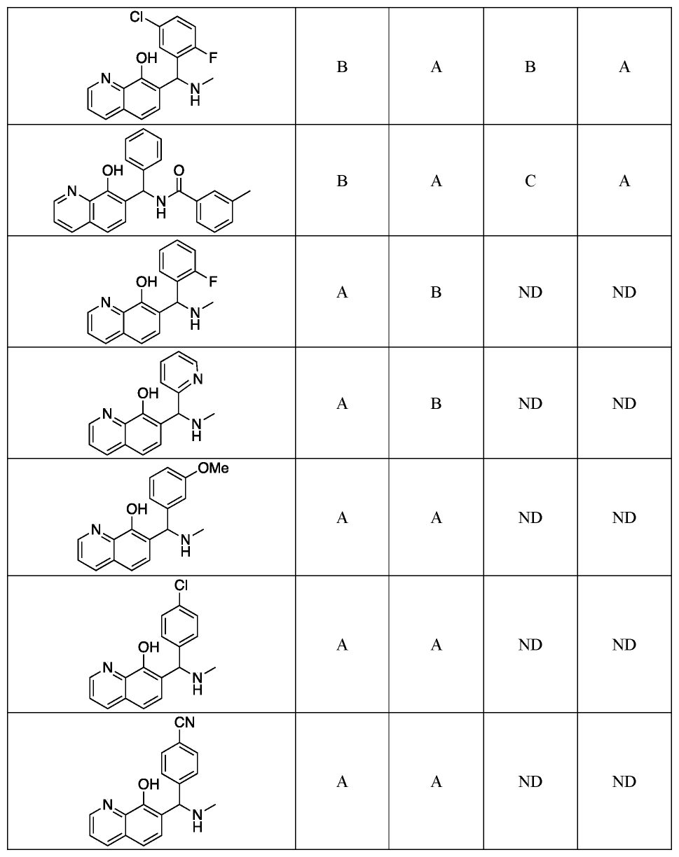

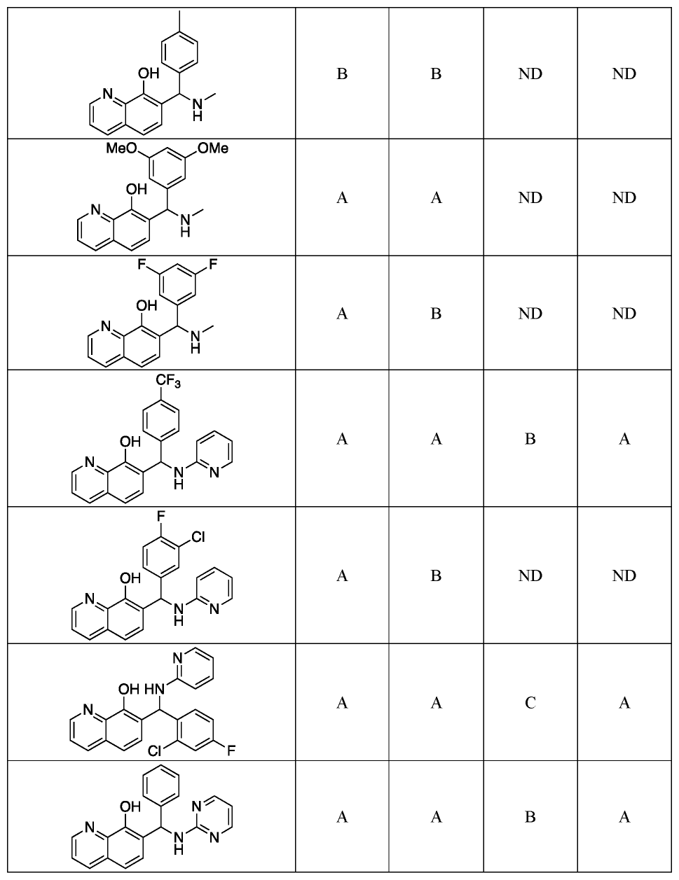

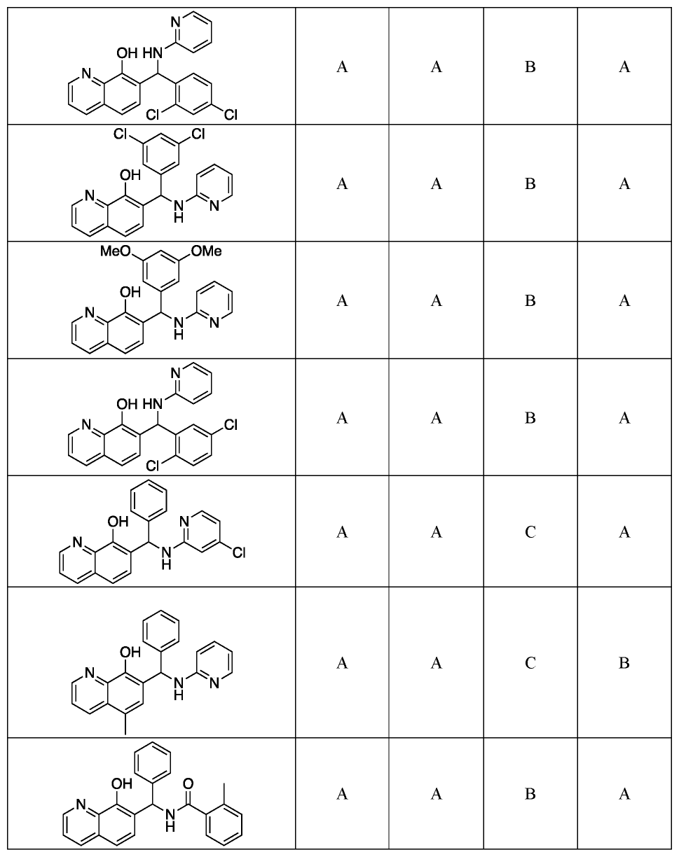

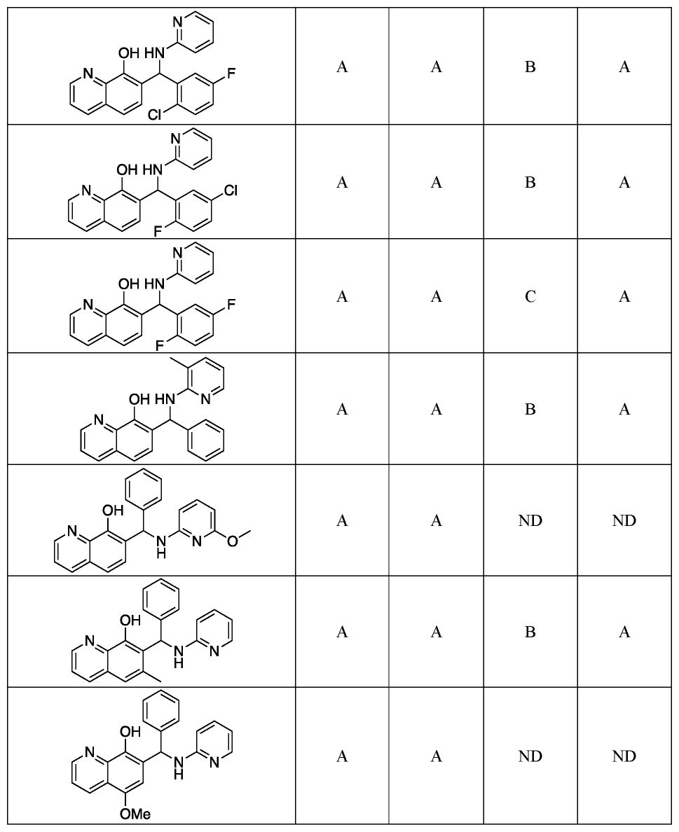

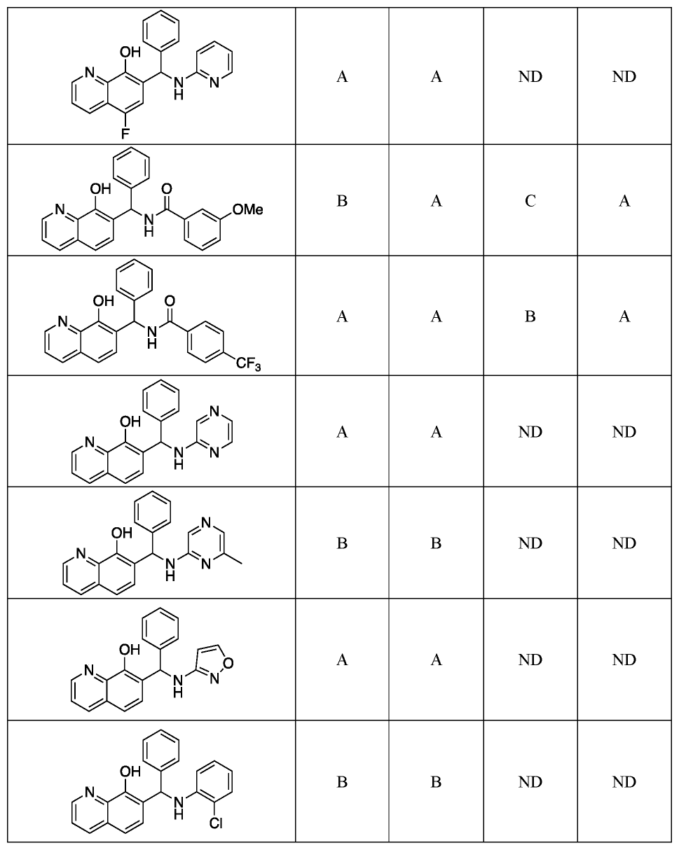

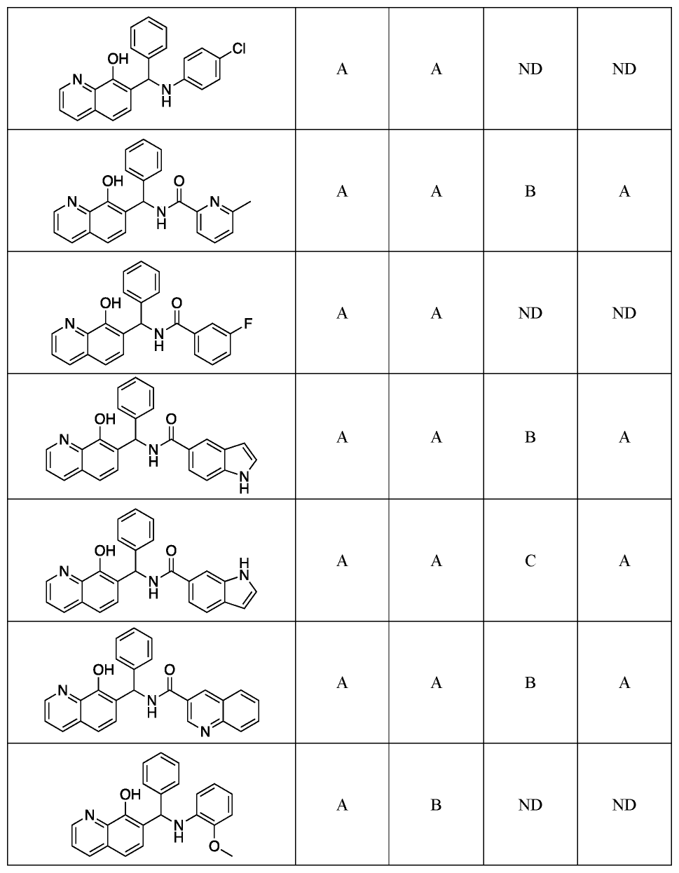

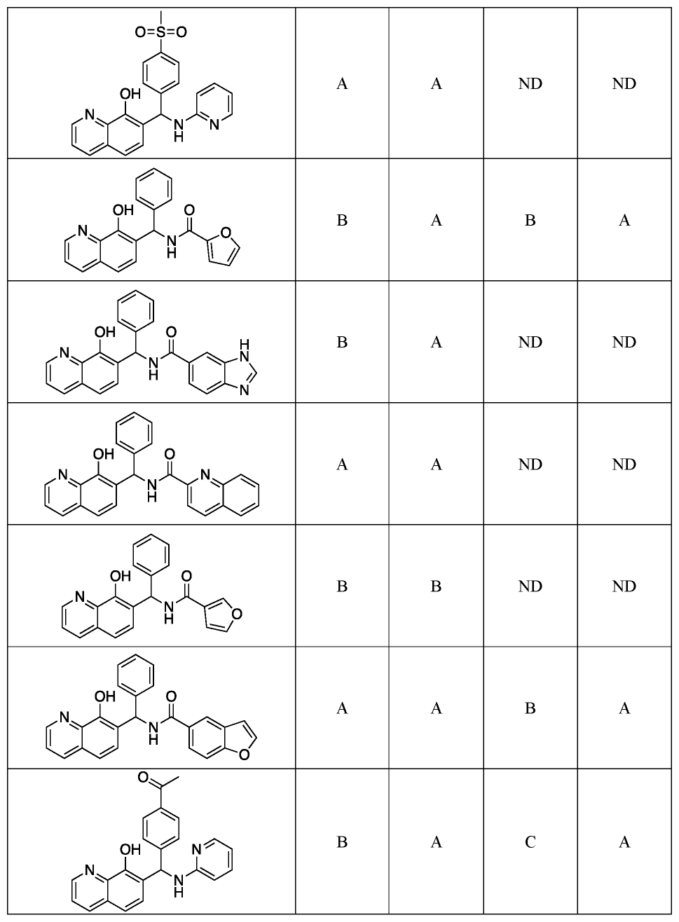

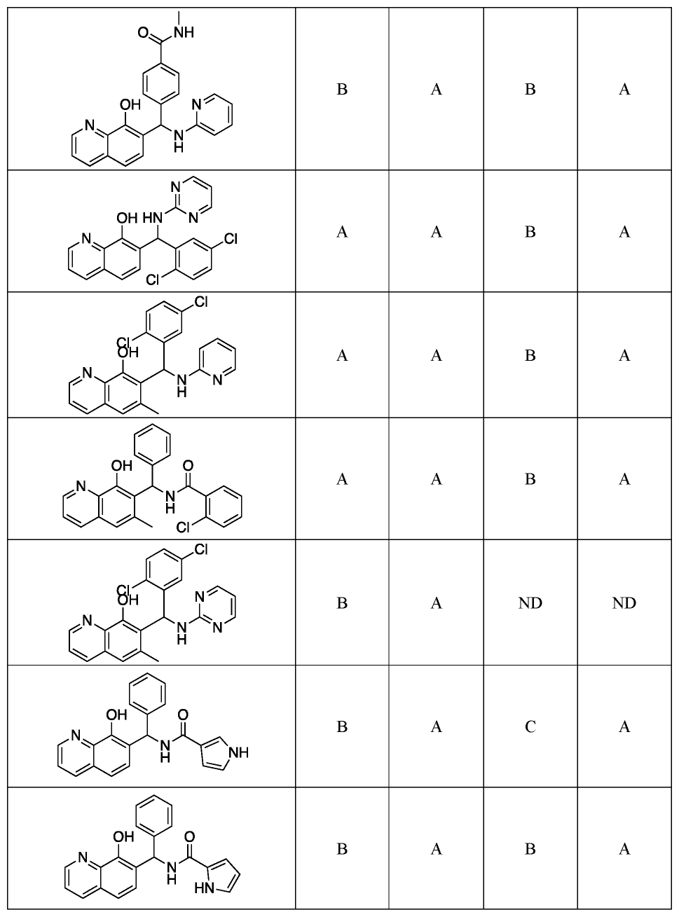

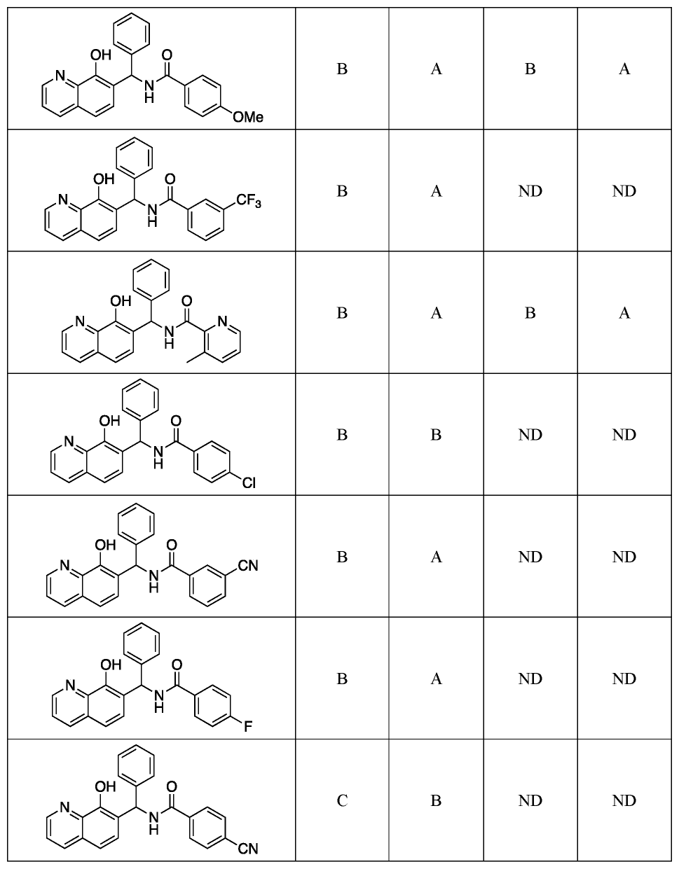

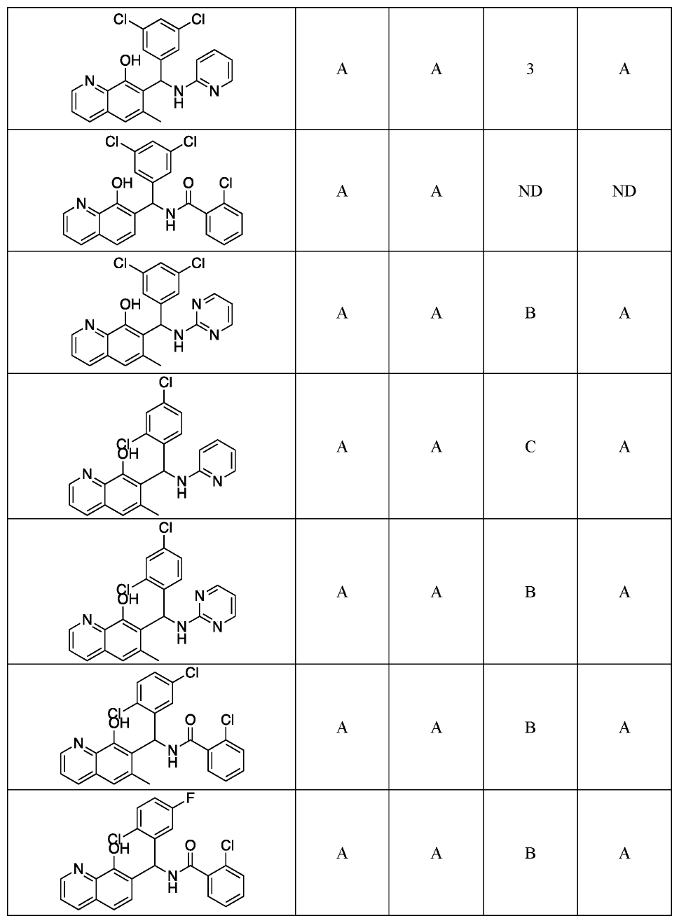

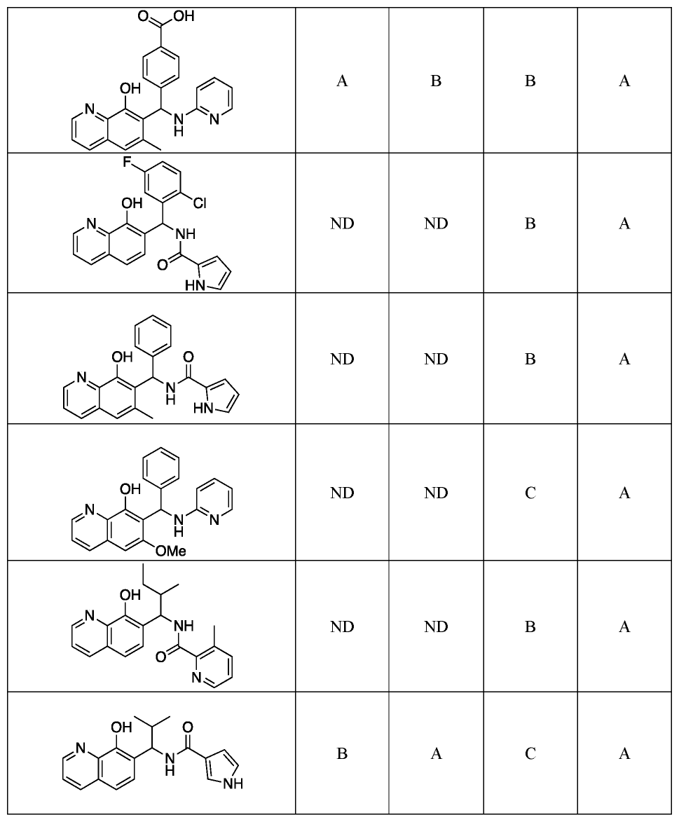

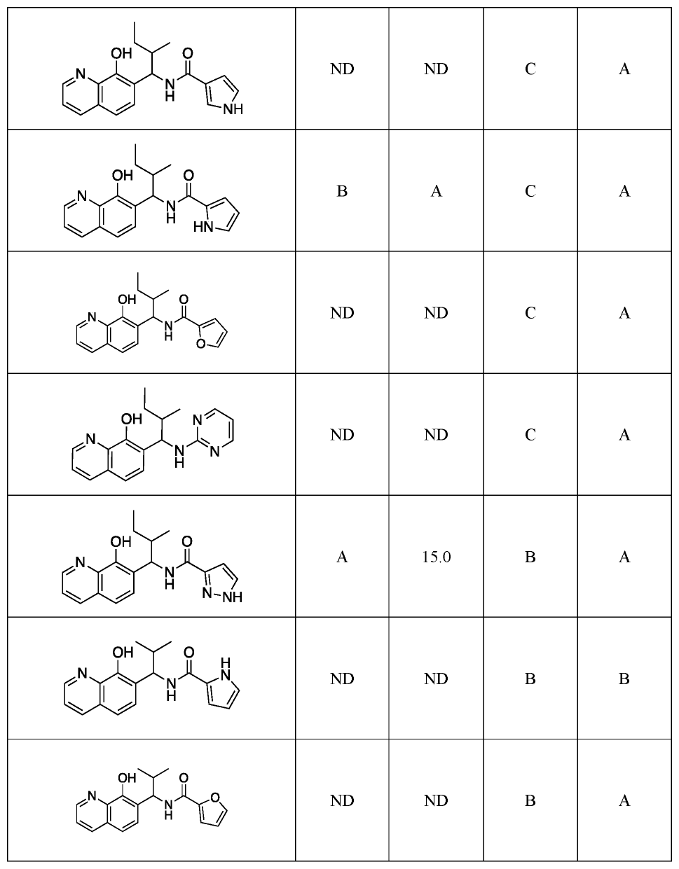

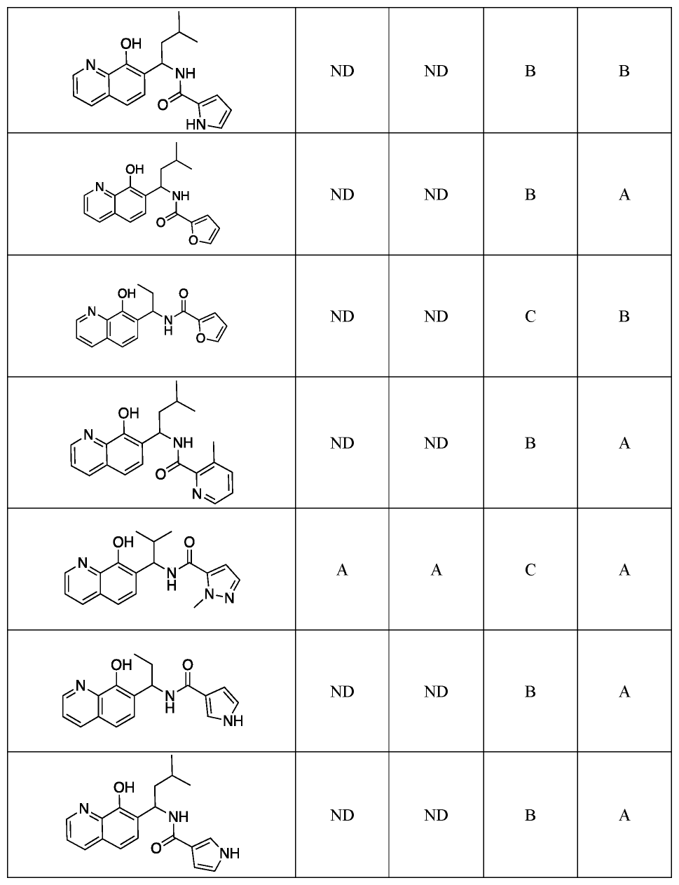

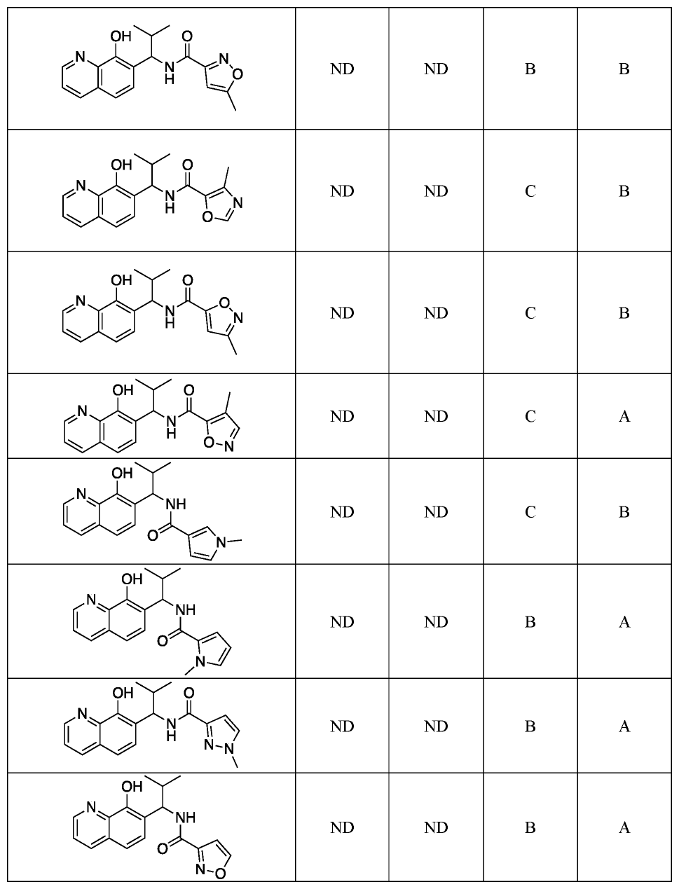

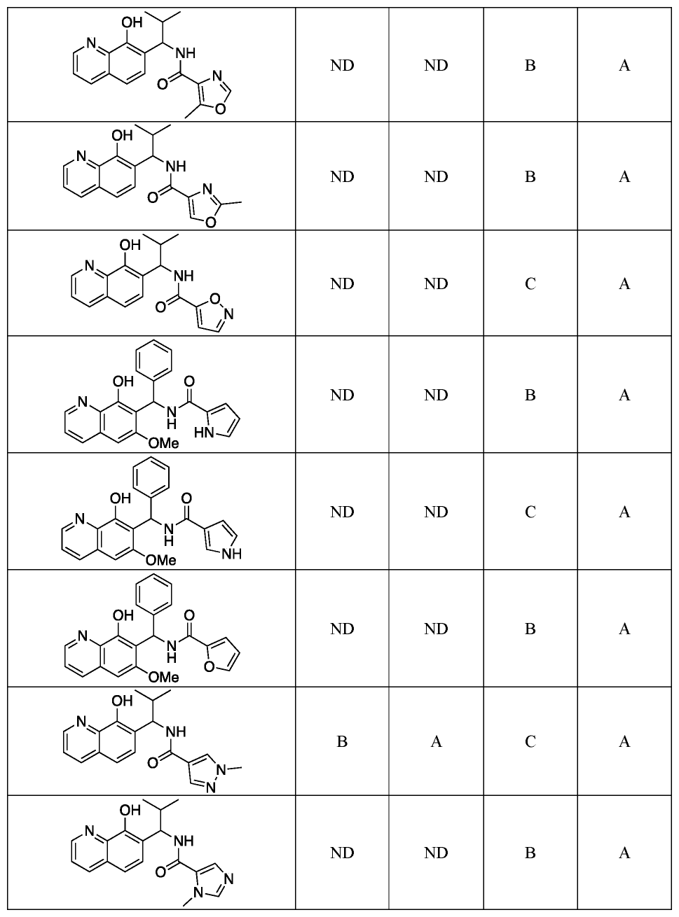

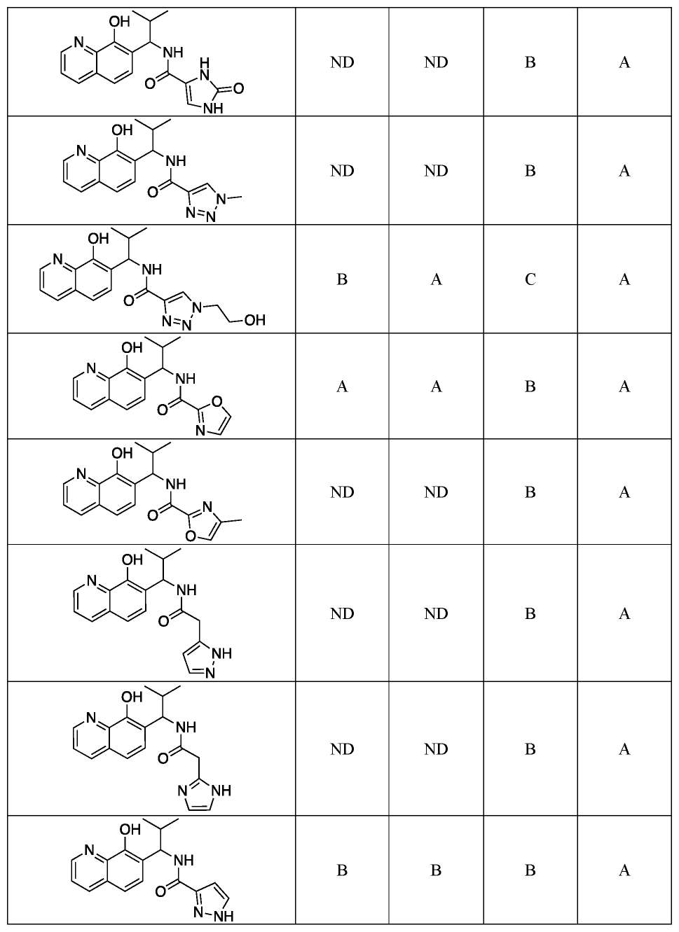

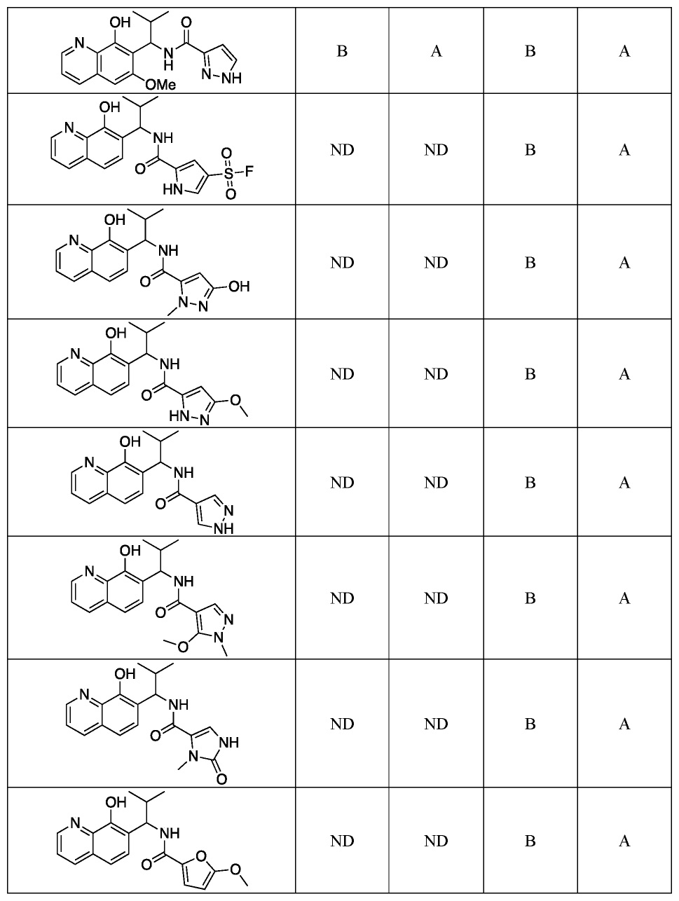

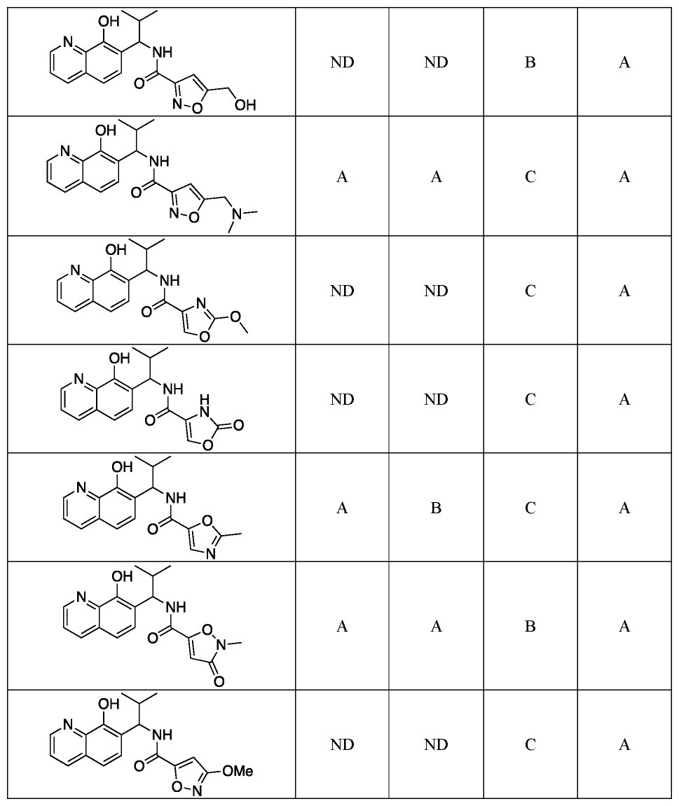

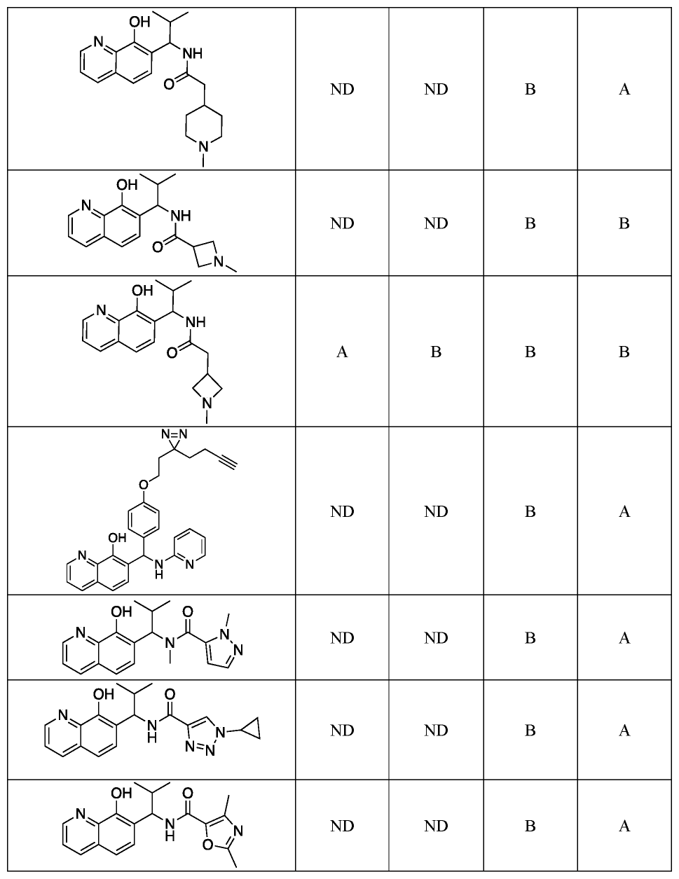

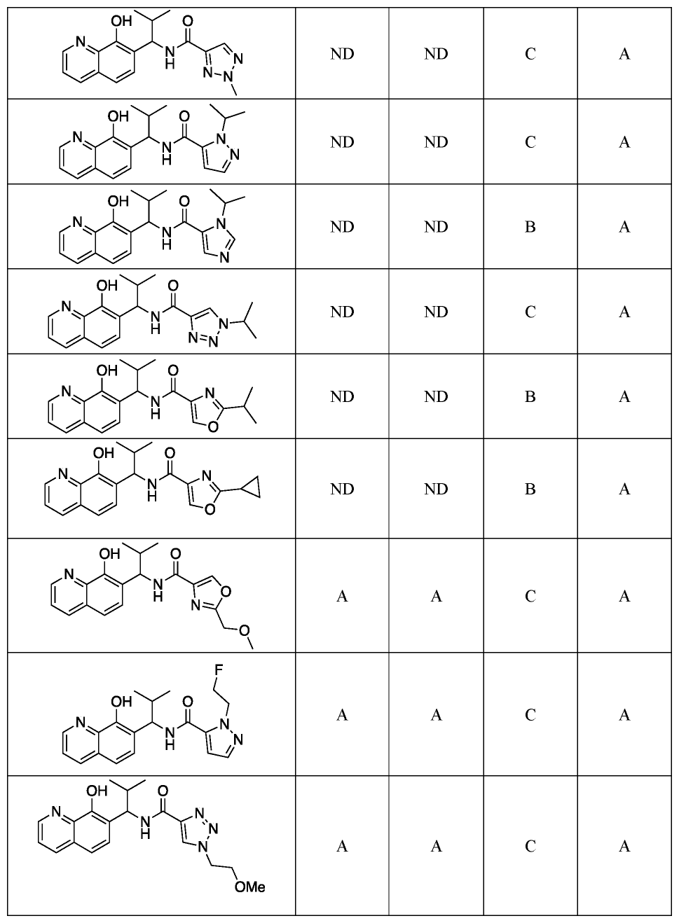

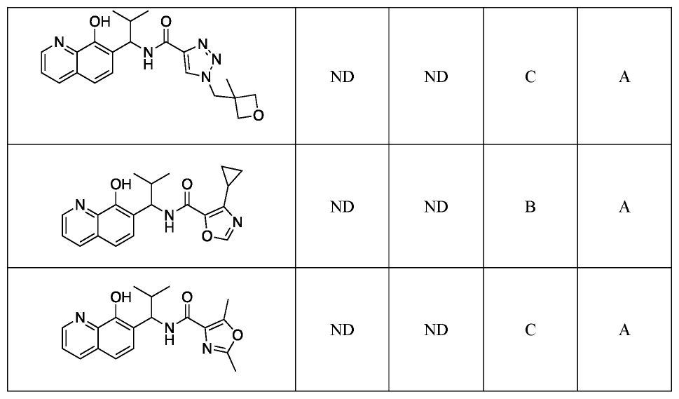

### DETAILED DESCRIPTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl groups may be substituted or unsubstituted.

As used herein, the term “alkylene” refers to an alkyl group having the specified number of carbons, for example from 2 to 12 carbon atoms, that contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Likewise, preferred cycloalkyls have from 3-10 carbon atoms in their ring structure, and more preferably have 3-6 carbons in the ring structure. Cycloalkyl groups may be substituted or unsubstituted.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “alkylthio” refers to an alkyl group, as defined above, having a sulfur moiety attached thereto. In certain embodiments, the “alkylthio” moiety is represented by one of —(S)-alkyl, —(S)-alkenyl, —(S)-alkynyl, and —(S)—(CH₂)ₘ—R¹, wherein m and R¹ are defined below. Representative alkylthio groups include methylthio, ethylthio, and the like. The terms “alkoxyl” or “alkoxy” as used herein refers to an alkyl group, as defined below, having an oxygen moiety attached thereto. Representative alkoxyl groups include methoxy, ethoxy, propoxy, tert-butoxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₁₀, where m and R₁₀ are described below.

The terms “amine” and “amino” are art-recognized and refer to both unsubstituted and substituted amines, e.g., a moiety that can be represented by the formulae:

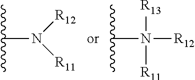

wherein R₁₁, R₁₂ and R₁₃ each independently represent a hydrogen, an alkyl, an alkenyl, —(CH₂)ₘ—R₁₀, or R₁₁ and R₁₂ taken together with the N atom to which they are attached complete a heterocycle having from 4 to 8 atoms in the ring structure; R₁₀ represents an alkenyl, aryl, cycloalkyl, a cycloalkenyl, a heterocyclyl, or a polycyclyl; and m is zero or an integer in the range of 1 to 8. In certain embodiments, only one of R₁₁ or R₁₂ can be a carbonyl, e.g., R₁₁, R₁₂, and the nitrogen together do not form an imide. In even more certain embodiments, R₁₁ and R₁₂ (and optionally R₁₃) each independently represent a hydrogen, an alkyl, an alkenyl, or —(CH₂)ₘ—R₁₀. Thus, the term “alkylamine” as used herein means an amine group, as defined above, having a substituted or unsubstituted alkyl attached thereto, i.e., at least one of R₁₁ and R₁₂ is an alkyl group. In certain embodiments, an amino group or an alkylamine is basic, meaning it has a conjugate acid with a pKₐ>7.00, i.e., the protonated forms of these functional groups have pKₐs relative to water above about 7.00.

The term “amide”, as used herein, refers to a group

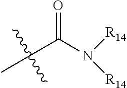

wherein each R₁₄ independently represent a hydrogen or hydrocarbyl group, or two R₁₄ are taken together with the N atom to which they are attached complete a heterocycle having from 4 to 8 atoms in the ring structure.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “carbonyl” is art-recognized and includes such moieties as can be represented by the formula:

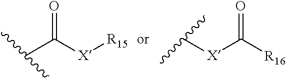

wherein X′ is a bond or represents an oxygen or a sulfur, and R₁₅ represents a hydrogen, an alkyl, an alkenyl, —(CH₂)ₘ—R₁₀ or a pharmaceutically acceptable salt, R₁₆ represents a hydrogen, an alkyl, an alkenyl or —(CH₂)ₘ—R₁₀, where m and R₁₀ are as defined above. Where X′ is an oxygen and R₁₅ or R₁₆ is not hydrogen, the formula represents an “ester.” Where X′ is an oxygen, and R₁₅ is as defined above, the moiety is referred to herein as a carboxyl group, and particularly when R₁₅ is a hydrogen, the formula represents a “carboxylic acid”. Where X′ is an oxygen, and R₁₆ is a hydrogen, the formula represents a “formate.” In general, where the oxygen atom of the above formula is replaced by a sulfur, the formula represents a “thiocarbonyl” group. Where X′ is a sulfur and R₁₅ or R₁₆ is not hydrogen, the formula represents a “thioester” group. Where X′ is a sulfur and R₁₅ is a hydrogen, the formula represents a “thiocarboxylic acid” group. Where X′ is a sulfur and R₁₆ is a hydrogen, the formula represents a “thioformate” group. On the other hand, where X′ is a bond, and R₁₅ is not hydrogen, the above formula represents a “ketone” group. Where X′ is a bond, and R₁₅ is a hydrogen, the above formula represents an “aldehyde” group.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “nitro” means —NO₂; the term “halogen” designates —F, —Cl, —Br, or —I; the term “sulfhydryl” means —SH; the term “hydroxyl” means —OH; the term “sulfonyl” means —SO₂—; the term “azido” means —N₃; the term “cyano” means —CN; the term “isocyanato” means —NCO; the term “thiocyanato” means —SCN; the term “isothiocyanato” means —NCS; and the term “cyanato” means —OCN.

The term “sulfamoyl” is art-recognized and includes a moiety that can be represented by the formula:

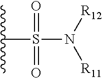

in which R₁₁ and R₁₂ are as defined above.

The term “sulfate” is art recognized and includes a moiety that can be represented by the formula:

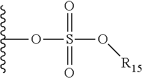

in which R₁₅ is as defined above.

The term “sulfonamide” is art recognized and includes a moiety that can be represented by the formula:

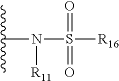

in which R₁₁ and R₁₆ are as defined above.

The term “sulfonate” is art-recognized and includes a moiety that can be represented by the formula:

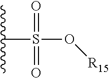

in which R₅₄ is an electron pair, hydrogen, alkyl, cycloalkyl, or aryl.

The terms “sulfoxido” or “sulfinyl”, as used herein, refers to a moiety that can be represented by the formula:

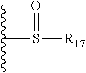

in which R₁₇ is selected from the group consisting of the group consisting of hydrogen, alkyl, alkenyl, alkynyl, cycloalkyl, heterocyclyl, aralkyl, or aryl.

The term “urea” is art-recognized and may be represented by the general formula

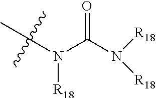

wherein each R₁₈ independently represents hydrogen or a hydrocarbyl, such as alkyl, or any occurrence of R₁₈ taken together with another and the intervening atom(s) complete a heterocycle having from 4 to 8 atoms in the ring structure.

As used herein, the definition of each expression, e.g., alkyl, m, n, etc., when it occurs more than once in any structure, is intended to be independent of its definition elsewhere in the same structure.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁-6 alkyl, C₃-6 cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

For purposes of this invention, the chemical elements are identified in accordance with the Periodic Table of the Elements, CAS version, Handbook of Chemistry and Physics, 67th Ed., 1986-87, inside cover.

### Compounds of the Invention

One aspect of the invention relates to compound of Formula (I):

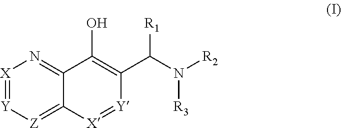

wherein

X, X′, Y, Y′, and Z are independently selected from N and C(R); provided that no more than two of X, X′, Y, Y′, and Z are N;

if Z and Y, or Y and X, or X′ and Y′, are C(R), then the two adjacent instances of R on any of them taken together may form a fused 3-8 membered ring;

R is independently selected from H, alkyl, alkenyl, alkynyl, cycloalkyl, heteroalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, haloalkyl, halocycloalkyl, halocycloheteroalkyl, —O-alkyl, —O-haloalkyl, —O-cycloalkyl, —N-alkyl, —N-haloalkyl, —N-cycloalkyl, —S-alkyl, —S-haloalkyl, —S-cycloalkyl, —O-heteroalkyl, —O— cycloheteroalkyl, —N-heteroalkyl, —N-cycloheteroalkyl, —S-heteroalkyl, —S-cycloheteroalkyl, —O-aryl, —N-aryl, —S-aryl, —O-heteroaryl, —N-heteroaryl, —S-heteroaryl, substituted or unsubstituted —O-alkylene-aryl, substituted or unsubstituted —N-alkylene-aryl, substituted or unsubstituted —S— alkylene-aryl, substituted or unsubstituted —O-alkylene-heteroaryl, substituted or unsubstituted —N-alkylene-heteroaryl, substituted or unsubstituted —S-alkylene-heteroaryl, halide, —CN, —NO₂, —S(O)Rₐ, —S(O)₂Rₐ, —C(O)Rₐ, —C(O)₂Rₐ, —C(O)NRₐR_{b}, OH, and C(O)NR′C(NR′)NRₐR_{b};

R′ is H, or alkyl;

Rₐ and R_{b} are independently H, alkyl, alkenyl, alkynyl, substituted or unsubstituted aryl, cycloalkyl, heteroalkyl, haloalkyl, cycloheteroalkyl, halocycloalkyl, halocycloheteroalkyl, substituted or unsubstituted heteroaryl, substituted or unsubstituted -alkylene-aryl, substituted or unsubstituted -alkylene-heteroaryl, alkylene-OR′, alkylene-NR′, alkylene-SR′, or Rₐ and R_{b} taken together with the nitrogen atom to which they are attached may form a 3-8 membered ring;

R₁ is H, alkyl, alkenyl, alkynyl, haloalkyl, heteroalkyl, heterohaloalkyl, cycloalkyl, halocycloalkyl, heterocycloalkyl, heterohalocycloalkyl, unsubstituted or substituted aryl, unsubstituted or substituted alkenyl-aryl, unsubstituted or substituted heteroaryl, unsubstituted or substituted alkenyl-heteroaryl, alkylene-OR′, alkylene-NR′, or alkylene-SR′; and

R₂, and R₃ are independently selected from H, alkyl, alkenyl, alkynyl, unsubstituted or substituted heteroaryl, unsubstituted or substituted aryl, haloalkyl, heteroalkyl, heterohaloalkyl, cycloalkyl, halocycloalkyl, heterocycloalkyl, heterohalocycloalkyl, alkylene-cycloalkyl, alkylene-aryl, alkylene-heteroaryl, alkylene-OR′, alkylene-NR′, alkylene-SR′, —S(O)Rₐ, —S(O)₂Rₐ, —C(O)Rₐ, —C(O)₂Rₐ, —C(O)NRₐR_{b}, —C(NRₐ)NRₐR_{b}, —N(Rₐ)C(NRₐ)NRₐR_{b}, and —C(O)NR′C(NR′)NRₐR_{b}; or R₂ and R₃ taken together may form a 4-8 membered ring.

In some embodiments, X is N. In some embodiments, X is C(R).

In some embodiments, X′ is N. In some embodiments, X′ is C(R).

In some embodiments, Y is N. In some embodiments, Y is C(R).

In some embodiments, Y′ is N. In some embodiments, Y′ is C(R).

In some embodiments, Z is N. In some embodiments, Z is C(R).

In some embodiments, X, Y, W, and Z are C(R). In some embodiments, X, X′, Y, Y′, W, and Z are C(R).

In some embodiments, Z and Y are C(R), and the two adjacent instances of R are taken together to form a fused 3-8 membered ring. In some embodiments, Y and X are C(R), and the two adjacent instances of R are taken together to form a fused 3-8 membered ring. In some embodiments, X′ and Y′ are C(R), and the two adjacent instances of R are taken together to form a fused 3-8 membered ring.

In some embodiments, R is H. In some embodiments, R is alkyl. In some embodiments, R is alkenyl. In some embodiments, R is alkynyl. In some embodiments, R is cycloalkyl. In some embodiments, R is heteroalkyl. In some embodiments, R is cycloheteroalkyl. In some embodiments, R is unsubstituted aryl. In some embodiments, R is substituted aryl. In some embodiments, R is unsubstituted -alkylene-aryl. In some embodiments, R is substituted -alkylene-aryl. In some embodiments, R is unsubstituted heteroaryl. In some embodiments, R is substituted heteroaryl. In some embodiments, R is unsubstituted -alkylene-heteroaryl. In some embodiments, R is substituted -alkylene-heteroaryl. In some embodiments, R is haloalkyl. In some embodiments, R is halocycloalkyl. In some embodiments, R is halocycloheteroalkyl. In some embodiments, R is —O-alkyl. In some embodiments, R is —O-haloalkyl. In some embodiments, R is —O-cycloalkyl. In some embodiments, R is —N-alkyl. In some embodiments, R is —N-haloalkyl. In some embodiments, R is —N-cycloalkyl. In some embodiments, R is —S-alkyl. In some embodiments, R is —S-haloalkyl. In some embodiments, R is —S-cycloalkyl. In some embodiments, R is —O-heteroalkyl. In some embodiments, R is —O-cycloheteroalkyl. In some embodiments, R is —N-heteroalkyl. In some embodiments, R is —N-cycloheteroalkyl. In some embodiments, R is —S-heteroalkyl. In some embodiments, R is —S-cycloheteroalkyl. In some embodiments, R is —O-aryl. In some embodiments, R is —N-aryl. In some embodiments, R is —S-aryl. In some embodiments, R is —O-heteroaryl. In some embodiments, R is —N-heteroaryl. In some embodiments, R is —S-heteroaryl. In some embodiments, R is substituted or unsubstituted —O-alkylene-aryl. In some embodiments, R is unsubstituted —N-alkylene-aryl. In some embodiments, R is substituted —N-alkylene-aryl. In some embodiments, R is unsubstituted —S— alkylene-aryl. In some embodiments, R is substituted —S-alkylene-aryl. In some embodiments, R is unsubstituted —O-alkylene-heteroaryl. In some embodiments, R is substituted —O-alkylene-heteroaryl. In some embodiments, R is unsubstituted —N-alkylene-heteroaryl. In some embodiments, R is substituted —N-alkylene-heteroaryl. In some embodiments, R is unsubstituted —S-alkylene-heteroaryl. In some embodiments, R is substituted —S-alkylene-heteroaryl. In some embodiments, R is halide. In some embodiments, R is Cl, F, or Br. In some embodiments, R is —CN. In some embodiments, R is —NO₂. In some embodiments, R is —S(O)Rₐ. In some embodiments, R is —S(O)₂Rₐ. In some embodiments, R is —C(O)Rₐ. In some embodiments, R is —C(O)₂Rₐ. In some embodiments, R is —C(O)NRₐR_{b}. In some embodiments, R is OH. In some embodiments, R is C(O)NR′C(NR′)NRₐR_{b}.

In some embodiments, R₁ is H. In some embodiments, R₁ is alkyl. In some embodiments, R₁ is methyl, ethyl, i-propyl, n-propyl, n-butyl, i-butyl, or t-butyl. In some embodiments, R₁ is alkenyl. In some embodiments, R₁ is alkynyl. In some embodiments, R₁ is haloalkyl. In some embodiments, R₁ is heteroalkyl. In some embodiments, R₁ is heterohaloalkyl. In some embodiments, R₁ is cycloalkyl. In some embodiments, cycloalkyl is cyclopropyl, cyclobutyl, cyclopentyl, or cyclohexyl. In some embodiments, R₁ is halocycloalkyl. In some embodiments, R₁ is heterocycloalkyl. In some embodiments, R₁ is heterohalocycloalkyl.

In some embodiments, R₁ is unsubstituted aryl. In some embodiments, R₁ is substituted aryl. In some embodiments ary is phenyl. In some embodiments, substituted aryl is substituted with one or more of —OMe, F, —CN, and —SO₂F. In some embodiments, R₁ is unsubstituted or substituted alkenyl-aryl. In some embodiments, R₁ is unsubstituted or substituted heteroaryl. In some embodiments, R₁ is unsubstituted or substituted alkenyl-heteroaryl.

In some embodiments, R₁ is alkylene-OR′. In some embodiments, R₁ is alkylene-NR′. In some embodiments, R₁ is alkylene-SR′.

In some embodiments, R₂ is H. In some embodiments, R₂ is alkyl. In some embodiments, R₂ is alkenyl. In some embodiments, R₂ is alkynyl. In some embodiments, R₂ is unsubstituted heteroaryl. In some embodiments, R₂ is substituted heteroaryl. In some embodiments, heteroaryl is pyridinyl. In some embodiments, R₂ is unsubstituted aryl. In some embodiments, R₂ is substituted aryl. In some embodiments, aryl is phenyl. In some embodiments, R₂ is haloalkyl. In some embodiments, R₂ is heteroalkyl. In some embodiments, R₂ is heterohaloalkyl. In some embodiments, R₂ is cycloalkyl. In some embodiments, R₂ is halocycloalkyl. In some embodiments, R₂ is heterocycloalkyl. In some embodiments, R₂ is heterohalocycloalkyl. In some embodiments, R₂ is alkylene-cycloalkyl. In some embodiments, R₂ is alkylene-aryl. In some embodiments, R₂ is alkylene-heteroaryl. In some embodiments, R₂ is alkylene-OR′. In some embodiments, R₂ is alkylene-NR′. In some embodiments, R₂ is alkylene-SR′ In some embodiments, R₂ is —S(O)Rₐ. In some embodiments, R₂ is —S(O)₂Rₐ. In some embodiments, R₂ is —C(O)Rₐ. In some embodiments, R₂ is —C(O)₂Rₐ. In some embodiments, R₂ is —C(O)NRₐR_{b}. In some embodiments, R₂ is —C(NRₐ)NRₐR_{b}. In some embodiments, R₂ is —N(Rₐ)C(NRₐ)NRₐR_{b}. In some embodiments, R₂ is —C(O).

In some embodiments, R₃ is H. In some embodiments, R₃ is alkyl. In some embodiments, R₃ is alkenyl. In some embodiments, R₃ is alkynyl. In some embodiments, R₃ is unsubstituted heteroaryl. In some embodiments, R₃ is substituted heteroaryl. In some embodiments, heteroaryl is pyridinyl. In some embodiments, R₃ is unsubstituted aryl. In some embodiments, R₃ is substituted aryl. In some embodiments, aryl is phenyl. In some embodiments, R₃ is haloalkyl. In some embodiments, R₃ is heteroalkyl. In some embodiments, R₃ is heterohaloalkyl. In some embodiments, R₃ is cycloalkyl. In some embodiments, R₃ is halocycloalkyl. In some embodiments, R₃ is heterocycloalkyl. In some embodiments, R₃ is heterohalocycloalkyl. In some embodiments, R₃ is alkylene-cycloalkyl. In some embodiments, R₃ is alkylene-aryl. In some embodiments, R₃ is alkylene-heteroaryl. In some embodiments, R₃ is alkylene-OR′. In some embodiments, R₃ is alkylene-NR′. In some embodiments, R₃ is alkylene-SR′ In some embodiments, R₃ is —S(O)Rₐ. In some embodiments, R₃ is —S(O)₂Rₐ. In some embodiments, R₃ is —C(O)Rₐ. In some embodiments, R₃ is —C(O)₂Rₐ. In some embodiments, R₃ is —C(O)NRₐR_{b}. In some embodiments, R₃ is —C(NRₐ)NRₐR_{b}. In some embodiments, R₃ is —N(Rₐ)C(NRₐ)NRₐR_{b}. In some embodiments, R₃ is —C(O).

In some embodiments, R′ is H. In some embodiments, R′ is alkyl. In some embodiments, R′ is methyl.

In some embodiments, Rₐ is H. In some embodiments, Rₐ is alkyl. In some embodiments, Rₐ is alkenyl. In some embodiments, Rₐ is alkynyl. In some embodiments, Rₐ is unsubstituted aryl. In some embodiments, Rₐ is substituted aryl. In some embodiments, Rₐ is cycloalkyl. In some embodiments, Rₐ is heteroalkyl. In some embodiments, Rₐ is haloalkyl. In some embodiments, Rₐ is cycloheteroalkyl. In some embodiments, Rₐ is halocycloalkyl. In some embodiments, Rₐ is halocycloheteroalkyl. In some embodiments, Rₐ is unsubstituted heteroaryl. In some embodiments, Rₐ is substituted heteroaryl. In some embodiments, Rₐ is unsubstituted -alkylene-aryl. In some embodiments, Rₐ is substituted -alkylene-aryl. In some embodiments, Rₐ is unsubstituted -alkylene-heteroaryl. In some embodiments, Rₐ is substituted -alkylene-heteroaryl. In some embodiments, Rₐ is alkylene-OR′. In some embodiments, Rₐ alkylene-NR′. In some embodiments, Rₐ alkylene-SR′.

In some embodiments, R_{b} is H. In some embodiments, R_{b} is alkyl. In some embodiments, R_{b} alkenyl. In some embodiments, R_{b} is alkynyl. In some embodiments, R_{b} is unsubstituted aryl. In some embodiments, R_{b} is substituted aryl. In some embodiments, R_{b} is cycloalkyl. In some embodiments, R_{b} is heteroalkyl. In some embodiments, R_{b} is haloalkyl. In some embodiments, R_{b} is cycloheteroalkyl. In some embodiments, R_{b} is halocycloalkyl. In some embodiments, R_{b} is halocycloheteroalkyl. In some embodiments, R_{b} is unsubstituted heteroaryl. In some embodiments, R_{b} is substituted heteroaryl. In some embodiments, R_{b} is unsubstituted -alkylene-aryl. In some embodiments, R_{b} is substituted -alkylene-aryl. In some embodiments, R_{b} is unsubstituted -alkylene-heteroaryl. In some embodiments, R_{b} is substituted -alkylene-heteroaryl. In some embodiments, R_{b} is alkylene-OR′. In some embodiments, R_{b} alkylene-NR′. In some embodiments, R_{b} alkylene-SR′.

In some embodiments, Rₐ and R_{b} taken together with the nitrogen atom to which they are attached may form a 3-8 membered ring.

In some embodiments, the compound is

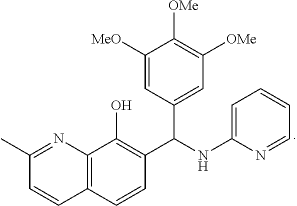 [SMILES: COc1cc(C(Nc2ccccn2)c2ccc3ccc(C)nc3c2O)cc(OC)c1OC]

In some embodiments, the compound is selected from:

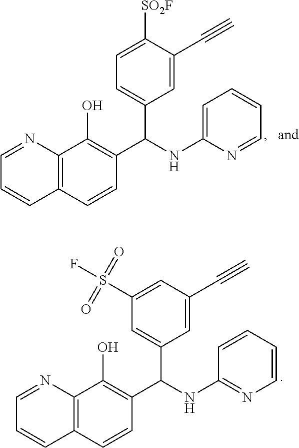 [SMILES: C#Cc1cc(C(Nc2ccccn2)c2ccc3cccnc3c2O)ccc1S(=O)(=O)F ; C#Cc1cc(C(Nc2ccccn2)c2ccc3cccnc3c2O)cc(S(=O)(=O)F)c1]

In some embodiments, the compound is selected from:

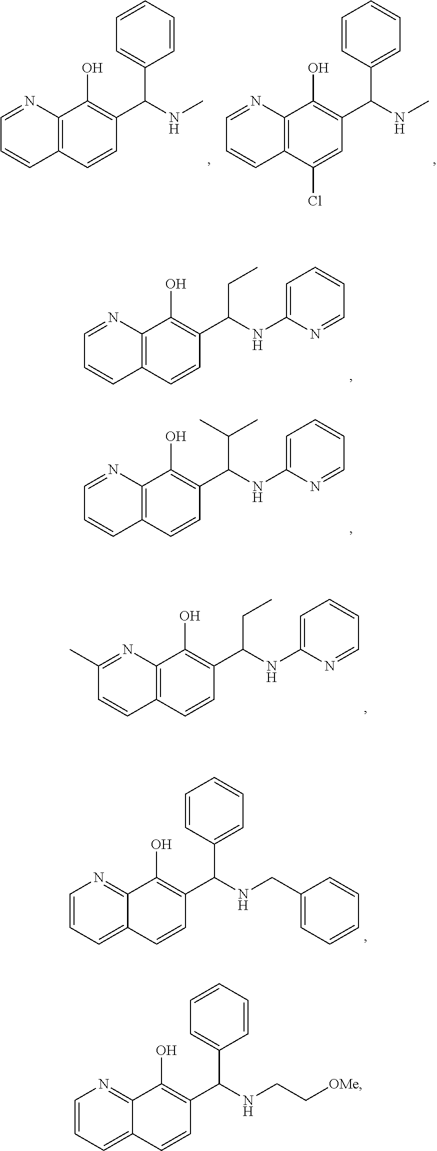

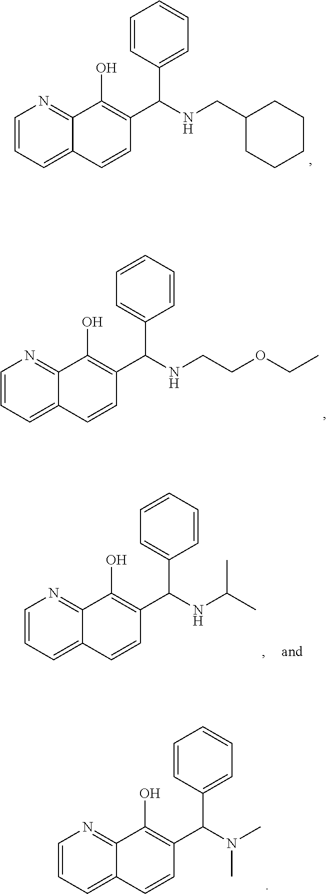 [SMILES: Oc1c(C(NCC2CCCCC2)c2ccccc2)ccc2cccnc12 ; CCOCCNC(c1ccccc1)c1ccc2cccnc2c1O ; CC(C)NC(c1ccccc1)c1ccc2cccnc2c1O ; CN(C)C(c1ccccc1)c1ccc2cccnc2c1O]

In some embodiments, the compound is selected from:

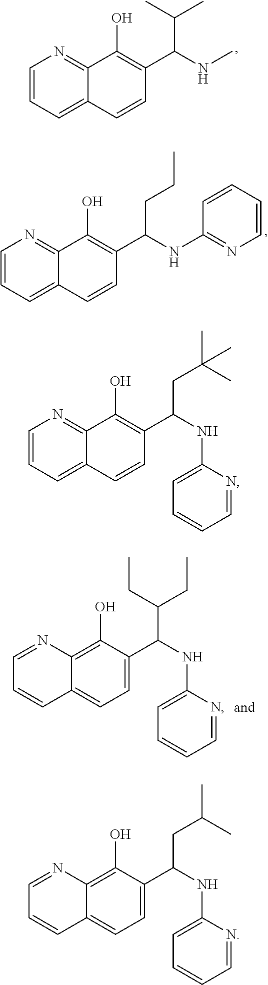

In some embodiments, the compound is selected from:

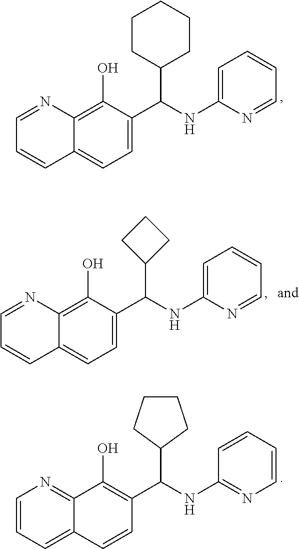 [SMILES: Oc1c(C(Nc2ccccn2)C2CCCCC2)ccc2cccnc12 ; Oc1c(C(Nc2ccccn2)C2CCC2)ccc2cccnc12 ; Oc1c(C(Nc2ccccn2)C2CCCC2)ccc2cccnc12]

In some embodiments, the compound is

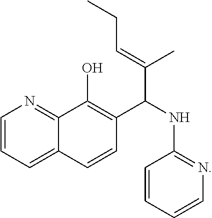 [SMILES: CC/C=C(\C)C(Nc1ccccn1)c1ccc2cccnc2c1O]

In some embodiments, the compound is selected from the following table:

| | |
|---|---|
| 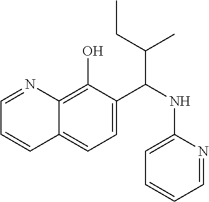 [SMILES: CCC(C)C(Nc1ccccn1)c1ccc2cccnc2c1O] | 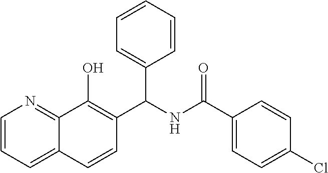 [SMILES: O=C(NC(c1ccccc1)c1ccc2cccnc2c1O)c1ccc(Cl)cc1] |
| 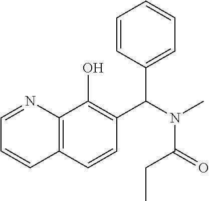 [SMILES: CCC(=O)N(C)C(c1ccccc1)c1ccc2cccnc2c1O] | 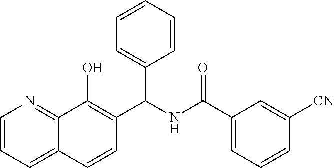 [SMILES: N#Cc1cccc(C(=O)NC(c2ccccc2)c2ccc3cccnc3c2O)c1] |
| 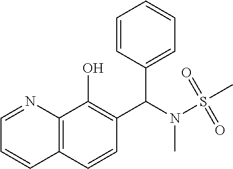 [SMILES: CN(C(c1ccccc1)c1ccc2cccnc2c1O)S(C)(=O)=O] | 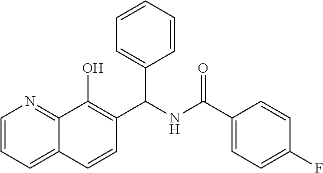 [SMILES: O=C(NC(c1ccccc1)c1ccc2cccnc2c1O)c1ccc(F)cc1] |
| 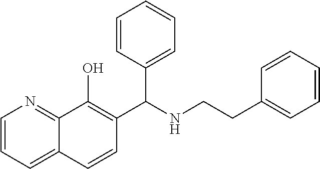 [SMILES: Oc1c(C(NCCc2ccccc2)c2ccccc2)ccc2cccnc12] | 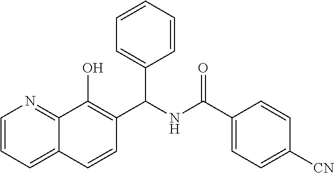 [SMILES: N#Cc1ccc(C(=O)NC(c2ccccc2)c2ccc3cccnc3c2O)cc1] |
| 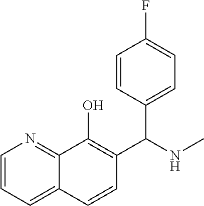 [SMILES: CNC(c1ccc(F)cc1)c1ccc2cccnc2c1O] | 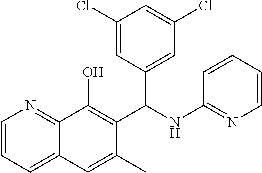 [SMILES: Cc1cc2cccnc2c(O)c1C(Nc1ccccn1)c1cc(Cl)cc(Cl)c1] |
| 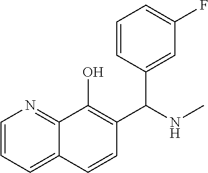 [SMILES: CNC(c1cccc(F)c1)c1ccc2cccnc2c1O] | 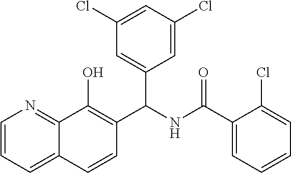 [SMILES: O=C(NC(c1cc(Cl)cc(Cl)c1)c1ccc2cccnc2c1O)c1ccccc1Cl] |
| 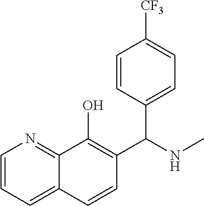 [SMILES: CNC(c1ccc(C(F)(F)F)cc1)c1ccc2cccnc2c1O] | 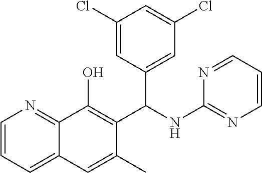 [SMILES: Cc1cc2cccnc2c(O)c1C(Nc1ncccn1)c1cc(Cl)cc(Cl)c1] |
| 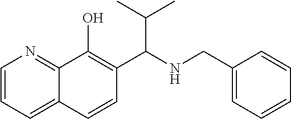 [SMILES: CC(C)C(NCc1ccccc1)c1ccc2cccnc2c1O] | 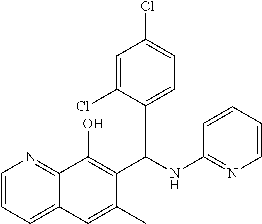 [SMILES: Cc1cc2cccnc2c(O)c1C(Nc1ccccn1)c1ccc(Cl)cc1Cl] |
| 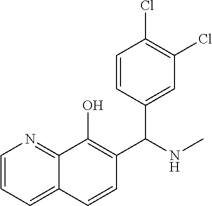 [SMILES: CNC(c1ccc(Cl)c(Cl)c1)c1ccc2cccnc2c1O] | 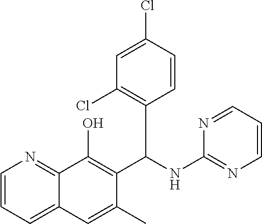 [SMILES: Cc1cc2cccnc2c(O)c1C(Nc1ncccn1)c1ccc(Cl)cc1Cl] |
| 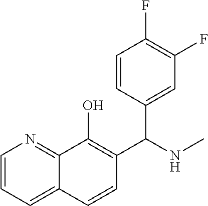 [SMILES: CNC(c1ccc(F)c(F)c1)c1ccc2cccnc2c1O] | 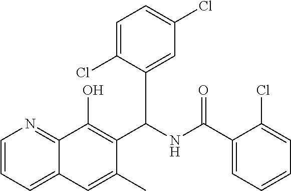 [SMILES: Cc1cc2cccnc2c(O)c1C(NC(=O)c1ccccc1Cl)c1cc(Cl)ccc1Cl] |
| 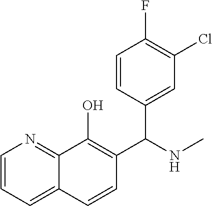 [SMILES: CNC(c1ccc(F)c(Cl)c1)c1ccc2cccnc2c1O] | 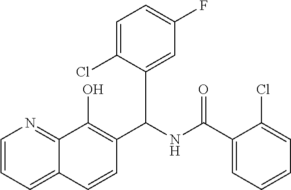 [SMILES: O=C(NC(c1cc(F)ccc1Cl)c1ccc2cccnc2c1O)c1ccccc1Cl] |
| 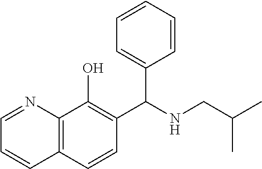 [SMILES: CC(C)CNC(c1ccccc1)c1ccc2cccnc2c1O] | 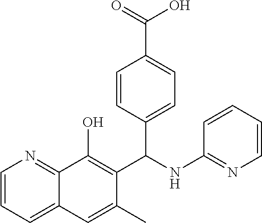 [SMILES: Cc1cc2cccnc2c(O)c1C(Nc1ccccn1)c1ccc(C(=O)O)cc1] |
| 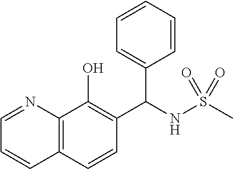 [SMILES: CS(=O)(=O)NC(c1ccccc1)c1ccc2cccnc2c1O] | 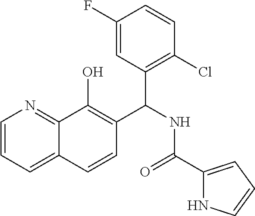 [SMILES: O=C(NC(c1cc(F)ccc1Cl)c1ccc2cccnc2c1O)c1ccc[nH]1] |
| 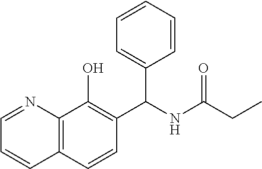 [SMILES: CCC(=O)NC(c1ccccc1)c1ccc2cccnc2c1O] | 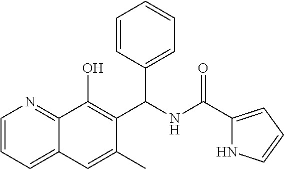 [SMILES: Cc1cc2cccnc2c(O)c1C(NC(=O)c1ccc[nH]1)c1ccccc1] |
| 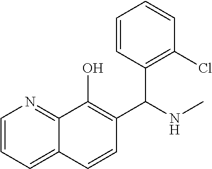 [SMILES: CNC(c1ccccc1Cl)c1ccc2cccnc2c1O] | 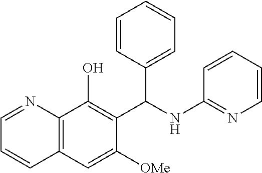 [SMILES: COc1cc2cccnc2c(O)c1C(Nc1ccccn1)c1ccccc1] |
| 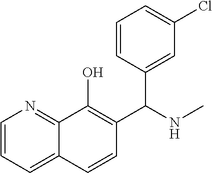 [SMILES: CNC(c1cccc(Cl)c1)c1ccc2cccnc2c1O] | 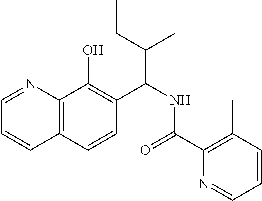 [SMILES: CCC(C)C(NC(=O)c1ncccc1C)c1ccc2cccnc2c1O] |
| 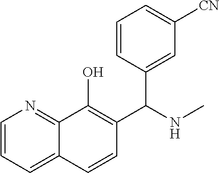 [SMILES: CNC(c1cccc(C#N)c1)c1ccc2cccnc2c1O] | 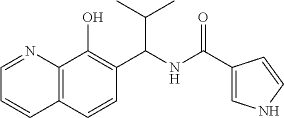 [SMILES: CC(C)C(NC(=O)c1cc[nH]c1)c1ccc2cccnc2c1O] |
| 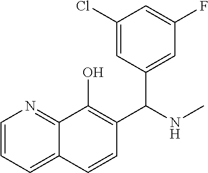 [SMILES: CNC(c1cc(F)cc(Cl)c1)c1ccc2cccnc2c1O] | 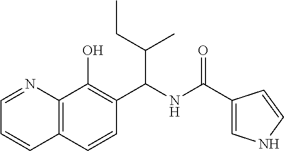 [SMILES: CCC(C)C(NC(=O)c1cc[nH]c1)c1ccc2cccnc2c1O] |
| 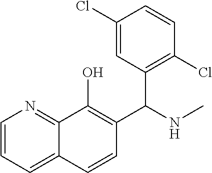 [SMILES: CNC(c1cc(Cl)ccc1Cl)c1ccc2cccnc2c1O] | 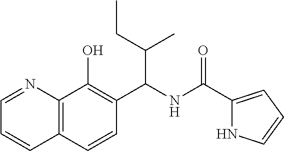 [SMILES: CCC(C)C(NC(=O)c1ccc[nH]1)c1ccc2cccnc2c1O] |
| 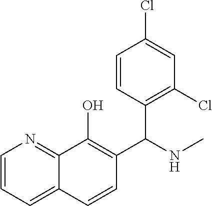 [SMILES: CNC(c1ccc(Cl)cc1Cl)c1ccc2cccnc2c1O] | 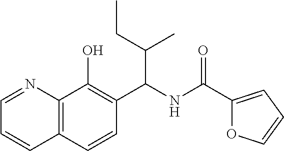 [SMILES: CCC(C)C(NC(=O)c1ccco1)c1ccc2cccnc2c1O] |
| 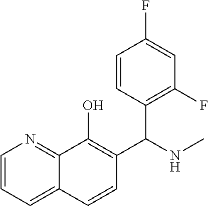 [SMILES: CNC(c1ccc(F)cc1F)c1ccc2cccnc2c1O] | 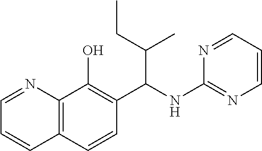 [SMILES: CCC(C)C(Nc1ncccn1)c1ccc2cccnc2c1O] |
| 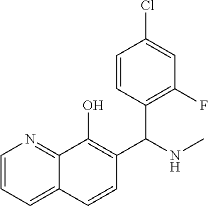 [SMILES: CNC(c1ccc(Cl)cc1F)c1ccc2cccnc2c1O] | 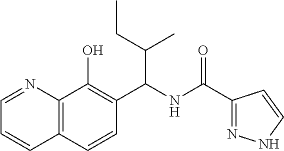 [SMILES: CCC(C)C(NC(=O)c1cc[nH]n1)c1ccc2cccnc2c1O] |
| 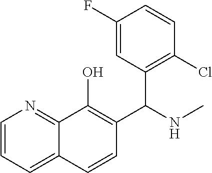 [SMILES: CNC(c1cc(F)ccc1Cl)c1ccc2cccnc2c1O] | 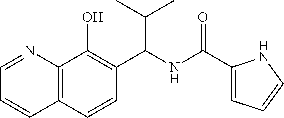 [SMILES: CC(C)C(NC(=O)c1ccc[nH]1)c1ccc2cccnc2c1O] |
| 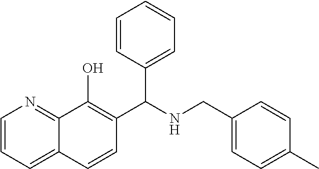 [SMILES: Cc1ccc(CNC(c2ccccc2)c2ccc3cccnc3c2O)cc1] | 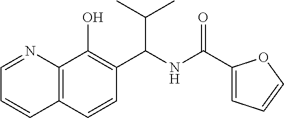 [SMILES: CC(C)C(NC(=O)c1ccco1)c1ccc2cccnc2c1O] |
| 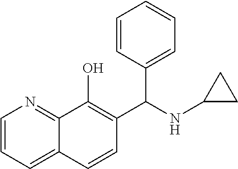 [SMILES: Oc1c(C(NC2CC2)c2ccccc2)ccc2cccnc12] | 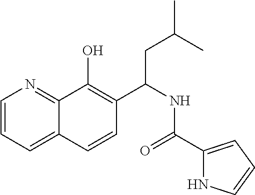 [SMILES: CC(C)CC(NC(=O)c1ccc[nH]1)c1ccc2cccnc2c1O] |
| 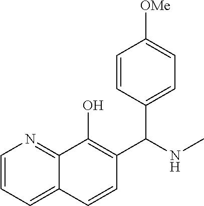 [SMILES: CNC(c1ccc(OC)cc1)c1ccc2cccnc2c1O] | 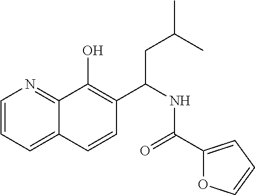 [SMILES: CC(C)CC(NC(=O)c1ccco1)c1ccc2cccnc2c1O] |
| 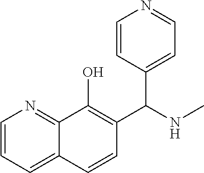 [SMILES: CNC(c1ccncc1)c1ccc2cccnc2c1O] | 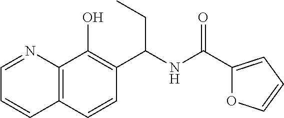 [SMILES: CCC(NC(=O)c1ccco1)c1ccc2cccnc2c1O] |
| 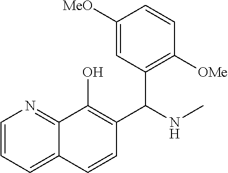 [SMILES: CNC(c1cc(OC)ccc1OC)c1ccc2cccnc2c1O] | 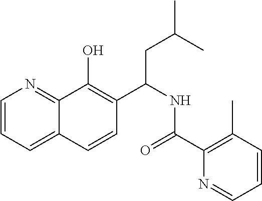 [SMILES: Cc1cccnc1C(=O)NC(CC(C)C)c1ccc2cccnc2c1O] |
| 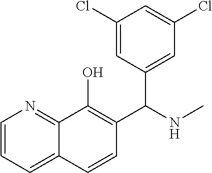 [SMILES: CNC(c1cc(Cl)cc(Cl)c1)c1ccc2cccnc2c1O] | 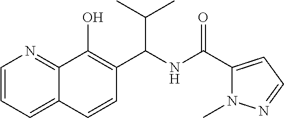 [SMILES: CC(C)C(NC(=O)c1ccnn1C)c1ccc2cccnc2c1O] |
| 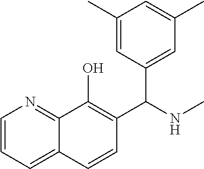 [SMILES: CNC(c1cc(C)cc(C)c1)c1ccc2cccnc2c1O] | 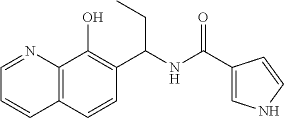 [SMILES: CCC(NC(=O)c1cc[nH]c1)c1ccc2cccnc2c1O] |
| 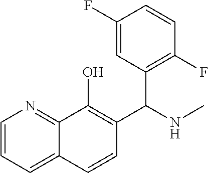 [SMILES: CNC(c1cc(F)ccc1F)c1ccc2cccnc2c1O] | 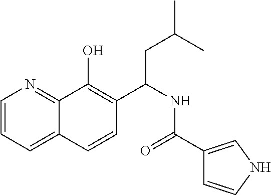 [SMILES: CC(C)CC(NC(=O)c1cc[nH]c1)c1ccc2cccnc2c1O] |
| 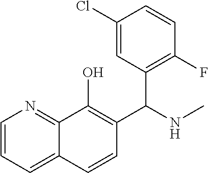 [SMILES: CNC(c1cc(Cl)ccc1F)c1ccc2cccnc2c1O] | 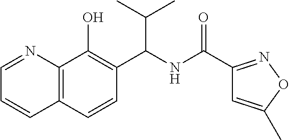 [SMILES: Cc1cc(C(=O)NC(c2ccc3cccnc3c2O)C(C)C)no1] |
| 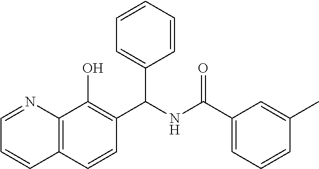 [SMILES: Cc1cccc(C(=O)NC(c2ccccc2)c2ccc3cccnc3c2O)c1] | 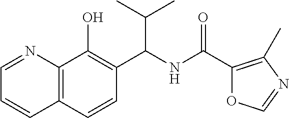 [SMILES: Cc1ncoc1C(=O)NC(c1ccc2cccnc2c1O)C(C)C] |
| 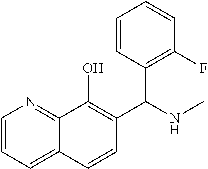 [SMILES: CNC(c1ccccc1F)c1ccc2cccnc2c1O] | 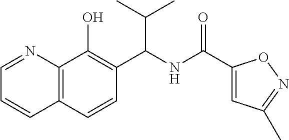 [SMILES: Cc1cc(C(=O)NC(c2ccc3cccnc3c2O)C(C)C)on1] |
| 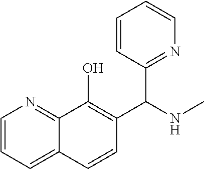 [SMILES: CNC(c1ccccn1)c1ccc2cccnc2c1O] | 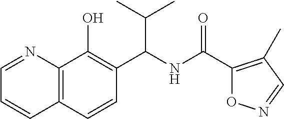 [SMILES: Cc1cnoc1C(=O)NC(c1ccc2cccnc2c1O)C(C)C] |
| 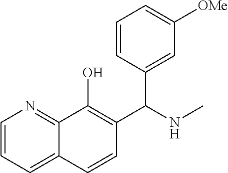 [SMILES: CNC(c1cccc(OC)c1)c1ccc2cccnc2c1O] | 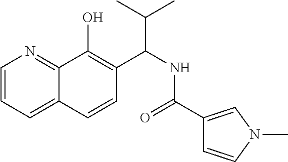 [SMILES: CC(C)C(NC(=O)c1ccn(C)c1)c1ccc2cccnc2c1O] |
| 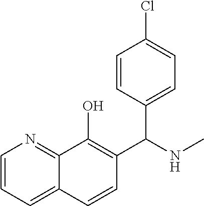 [SMILES: CNC(c1ccc(Cl)cc1)c1ccc2cccnc2c1O] | 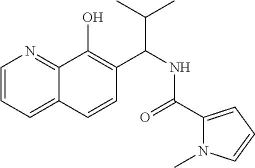 [SMILES: CC(C)C(NC(=O)c1cccn1C)c1ccc2cccnc2c1O] |
| 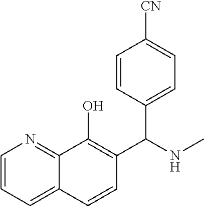 [SMILES: CNC(c1ccc(C#N)cc1)c1ccc2cccnc2c1O] | 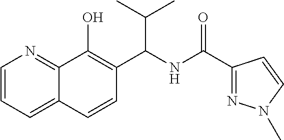 [SMILES: CC(C)C(NC(=O)c1ccn(C)n1)c1ccc2cccnc2c1O] |
| 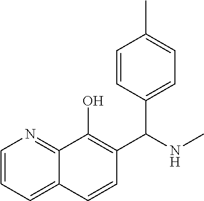 [SMILES: CNC(c1ccc(C)cc1)c1ccc2cccnc2c1O] | 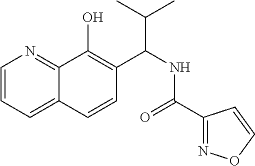 [SMILES: CC(C)C(NC(=O)c1ccon1)c1ccc2cccnc2c1O] |
| 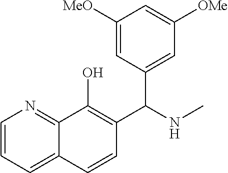 [SMILES: CNC(c1cc(OC)cc(OC)c1)c1ccc2cccnc2c1O] | 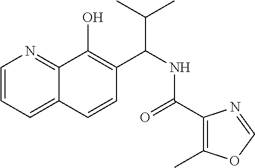 [SMILES: Cc1ocnc1C(=O)NC(c1ccc2cccnc2c1O)C(C)C] |
| 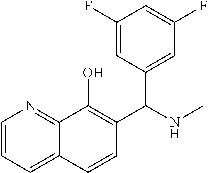 [SMILES: CNC(c1cc(F)cc(F)c1)c1ccc2cccnc2c1O] | 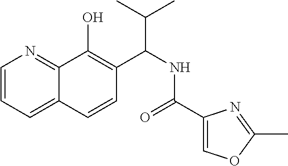 [SMILES: Cc1nc(C(=O)NC(c2ccc3cccnc3c2O)C(C)C)co1] |
| 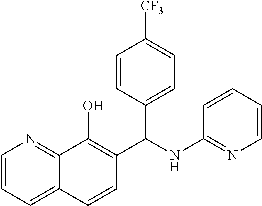 [SMILES: Oc1c(C(Nc2ccccn2)c2ccc(C(F)(F)F)cc2)ccc2cccnc12] | 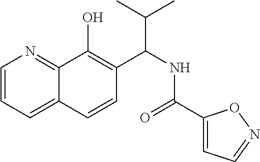 [SMILES: CC(C)C(NC(=O)c1ccno1)c1ccc2cccnc2c1O] |
| 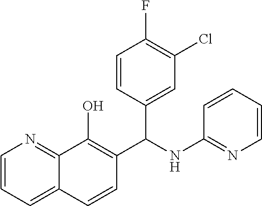 [SMILES: Oc1c(C(Nc2ccccn2)c2ccc(F)c(Cl)c2)ccc2cccnc12] | 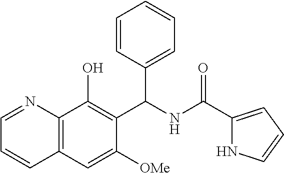 [SMILES: COc1cc2cccnc2c(O)c1C(NC(=O)c1ccc[nH]1)c1ccccc1] |
| 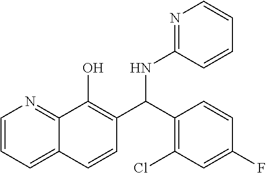 [SMILES: Oc1c(C(Nc2ccccn2)c2ccc(F)cc2Cl)ccc2cccnc12] | 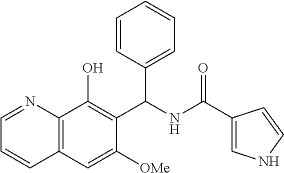 [SMILES: COc1cc2cccnc2c(O)c1C(NC(=O)c1cc[nH]c1)c1ccccc1] |
| 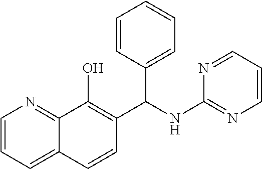 [SMILES: Oc1c(C(Nc2ncccn2)c2ccccc2)ccc2cccnc12] | 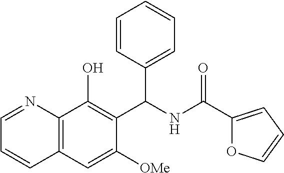 [SMILES: COc1cc2cccnc2c(O)c1C(NC(=O)c1ccco1)c1ccccc1] |
| 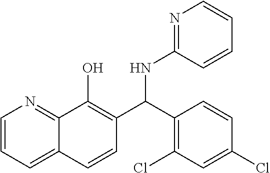 [SMILES: Oc1c(C(Nc2ccccn2)c2ccc(Cl)cc2Cl)ccc2cccnc12] | 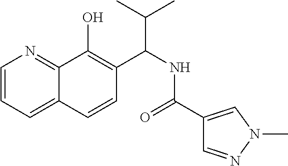 [SMILES: CC(C)C(NC(=O)c1cnn(C)c1)c1ccc2cccnc2c1O] |
| 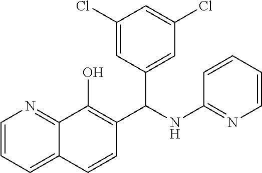 [SMILES: Oc1c(C(Nc2ccccn2)c2cc(Cl)cc(Cl)c2)ccc2cccnc12] | 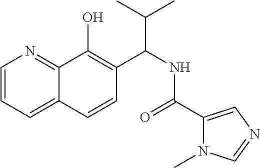 [SMILES: CC(C)C(NC(=O)c1cncn1C)c1ccc2cccnc2c1O] |
| 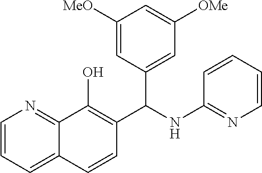 [SMILES: COc1cc(OC)cc(C(Nc2ccccn2)c2ccc3cccnc3c2O)c1] | 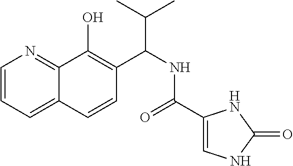 [SMILES: CC(C)C(NC(=O)c1c[nH]c(=O)[nH]1)c1ccc2cccnc2c1O] |
| 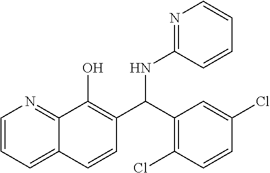 [SMILES: Oc1c(C(Nc2ccccn2)c2cc(Cl)ccc2Cl)ccc2cccnc12] | 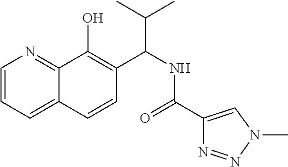 [SMILES: CC(C)C(NC(=O)c1cn(C)nn1)c1ccc2cccnc2c1O] |
| 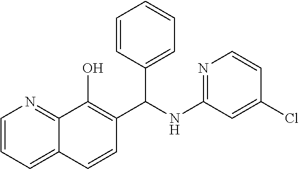 [SMILES: Oc1c(C(Nc2cc(Cl)ccn2)c2ccccc2)ccc2cccnc12] | 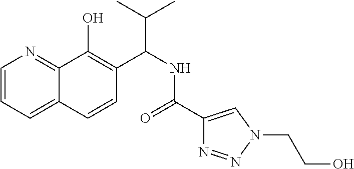 [SMILES: CC(C)C(NC(=O)c1cn(CCO)nn1)c1ccc2cccnc2c1O] |
| 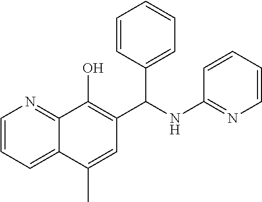 [SMILES: Cc1cc(C(Nc2ccccn2)c2ccccc2)c(O)c2ncccc12] | 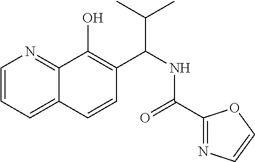 [SMILES: CC(C)C(NC(=O)c1ncco1)c1ccc2cccnc2c1O] |
| 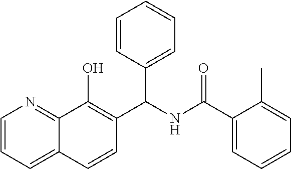 [SMILES: Cc1ccccc1C(=O)NC(c1ccccc1)c1ccc2cccnc2c1O] | 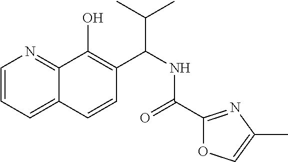 [SMILES: Cc1coc(C(=O)NC(c2ccc3cccnc3c2O)C(C)C)n1] |
| 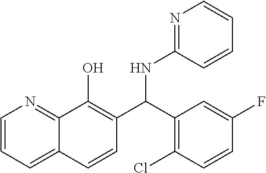 [SMILES: Oc1c(C(Nc2ccccn2)c2cc(F)ccc2Cl)ccc2cccnc12] | 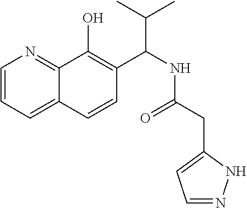 [SMILES: CC(C)C(NC(=O)Cc1ccn[nH]1)c1ccc2cccnc2c1O] |
| 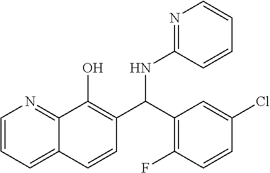 [SMILES: Oc1c(C(Nc2ccccn2)c2cc(Cl)ccc2F)ccc2cccnc12] | 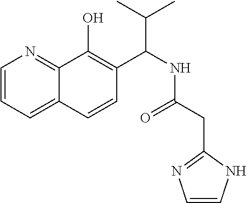 [SMILES: CC(C)C(NC(=O)Cc1ncc[nH]1)c1ccc2cccnc2c1O] |
| 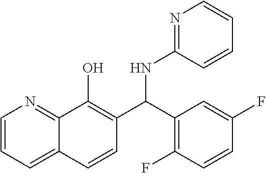 [SMILES: Oc1c(C(Nc2ccccn2)c2cc(F)ccc2F)ccc2cccnc12] | 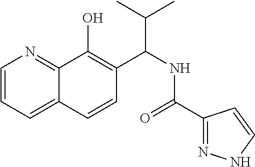 [SMILES: CC(C)C(NC(=O)c1cc[nH]n1)c1ccc2cccnc2c1O] |
| 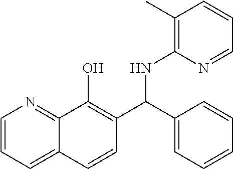 [SMILES: Cc1cccnc1NC(c1ccccc1)c1ccc2cccnc2c1O] | 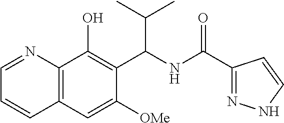 [SMILES: COc1cc2cccnc2c(O)c1C(NC(=O)c1cc[nH]n1)C(C)C] |
| 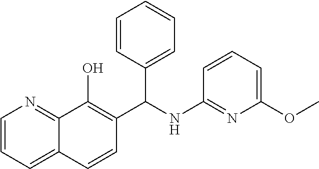 [SMILES: COc1cccc(NC(c2ccccc2)c2ccc3cccnc3c2O)n1] | 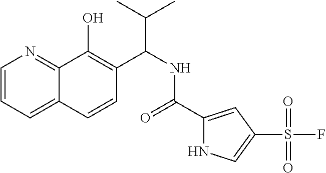 [SMILES: CC(C)C(NC(=O)c1cc(S(=O)(=O)F)c[nH]1)c1ccc2cccnc2c1O] |
| 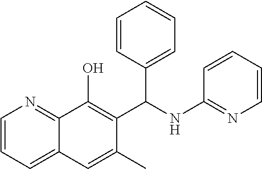 [SMILES: Cc1cc2cccnc2c(O)c1C(Nc1ccccn1)c1ccccc1] | 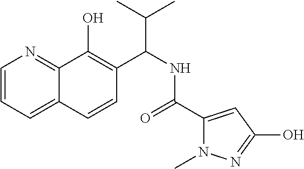 [SMILES: CC(C)C(NC(=O)c1cc(O)nn1C)c1ccc2cccnc2c1O] |
| 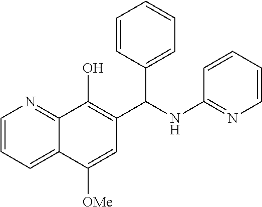 [SMILES: COc1cc(C(Nc2ccccn2)c2ccccc2)c(O)c2ncccc12] | 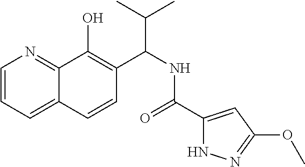 [SMILES: COc1cc(C(=O)NC(c2ccc3cccnc3c2O)C(C)C)[nH]n1] |
| 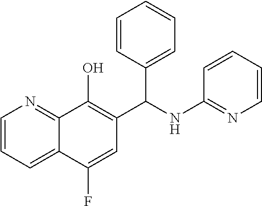 [SMILES: Oc1c(C(Nc2ccccn2)c2ccccc2)cc(F)c2cccnc12] | 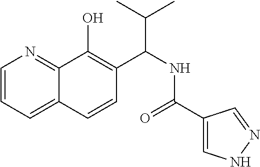 [SMILES: CC(C)C(NC(=O)c1cn[nH]c1)c1ccc2cccnc2c1O] |
| 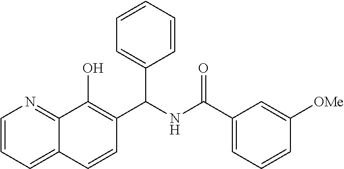 [SMILES: COc1cccc(C(=O)NC(c2ccccc2)c2ccc3cccnc3c2O)c1] | 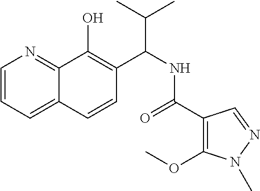 [SMILES: COc1c(C(=O)NC(c2ccc3cccnc3c2O)C(C)C)cnn1C] |
| 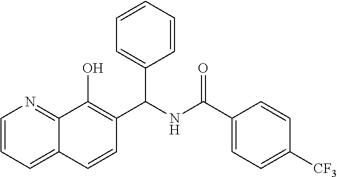 [SMILES: O=C(NC(c1ccccc1)c1ccc2cccnc2c1O)c1ccc(C(F)(F)F)cc1] | 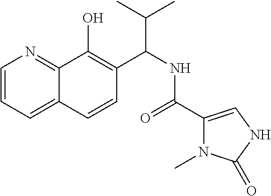 [SMILES: CC(C)C(NC(=O)c1c[nH]c(=O)n1C)c1ccc2cccnc2c1O] |
| 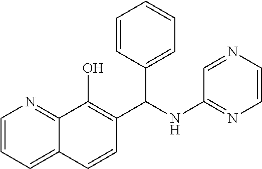 [SMILES: Oc1c(C(Nc2cnccn2)c2ccccc2)ccc2cccnc12] | 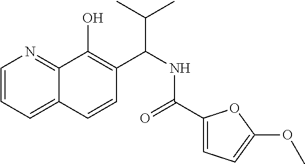 [SMILES: COc1ccc(C(=O)NC(c2ccc3cccnc3c2O)C(C)C)o1] |
| 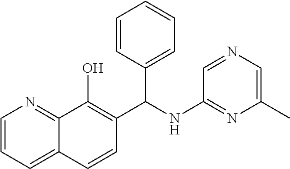 [SMILES: Cc1cncc(NC(c2ccccc2)c2ccc3cccnc3c2O)n1] | 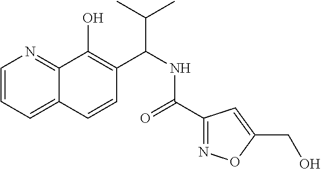 [SMILES: CC(C)C(NC(=O)c1cc(CO)on1)c1ccc2cccnc2c1O] |
| 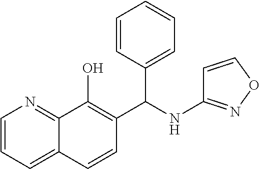 [SMILES: Oc1c(C(Nc2ccon2)c2ccccc2)ccc2cccnc12] | 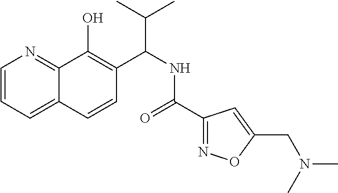 [SMILES: CC(C)C(NC(=O)c1cc(CN(C)C)on1)c1ccc2cccnc2c1O] |
| 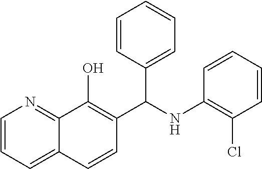 [SMILES: Oc1c(C(Nc2ccccc2Cl)c2ccccc2)ccc2cccnc12] | 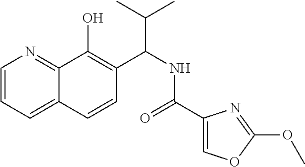 [SMILES: COc1nc(C(=O)NC(c2ccc3cccnc3c2O)C(C)C)co1] |
| 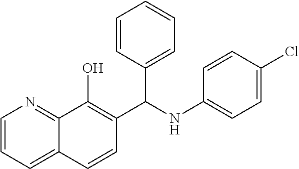 [SMILES: Oc1c(C(Nc2ccc(Cl)cc2)c2ccccc2)ccc2cccnc12] | 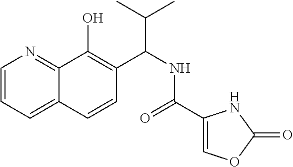 [SMILES: CC(C)C(NC(=O)c1coc(=O)[nH]1)c1ccc2cccnc2c1O] |
| 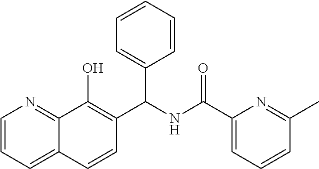 [SMILES: Cc1cccc(C(=O)NC(c2ccccc2)c2ccc3cccnc3c2O)n1] | 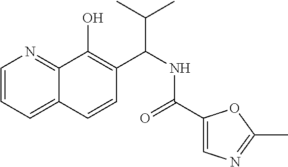 [SMILES: Cc1ncc(C(=O)NC(c2ccc3cccnc3c2O)C(C)C)o1] |
| 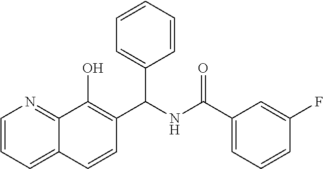 [SMILES: O=C(NC(c1ccccc1)c1ccc2cccnc2c1O)c1cccc(F)c1] | 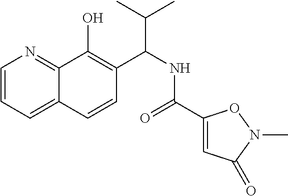 [SMILES: CC(C)C(NC(=O)c1cc(=O)n(C)o1)c1ccc2cccnc2c1O] |
| 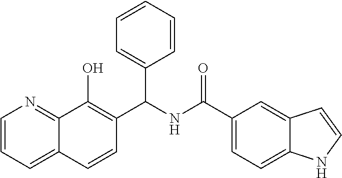 [SMILES: O=C(NC(c1ccccc1)c1ccc2cccnc2c1O)c1ccc2[nH]ccc2c1] | 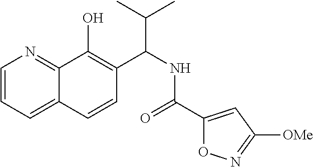 [SMILES: COc1cc(C(=O)NC(c2ccc3cccnc3c2O)C(C)C)on1] |
| 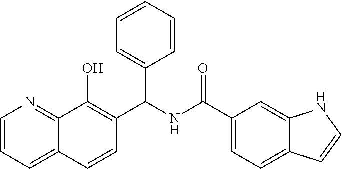 [SMILES: O=C(NC(c1ccccc1)c1ccc2cccnc2c1O)c1ccc2cc[nH]c2c1] | 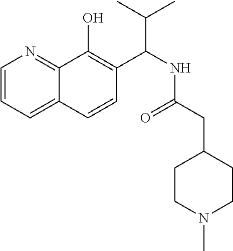 [SMILES: CC(C)C(NC(=O)CC1CCN(C)CC1)c1ccc2cccnc2c1O] |
| 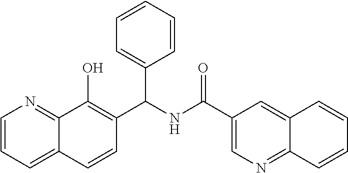 [SMILES: O=C(NC(c1ccccc1)c1ccc2cccnc2c1O)c1cnc2ccccc2c1] | 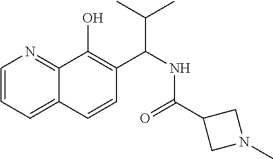 [SMILES: CC(C)C(NC(=O)C1CN(C)C1)c1ccc2cccnc2c1O] |
| 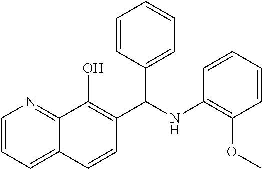 [SMILES: COc1ccccc1NC(c1ccccc1)c1ccc2cccnc2c1O] | 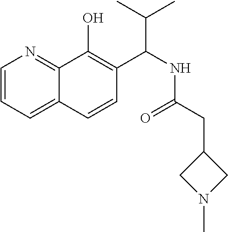 [SMILES: CC(C)C(NC(=O)CC1CN(C)C1)c1ccc2cccnc2c1O] |
| 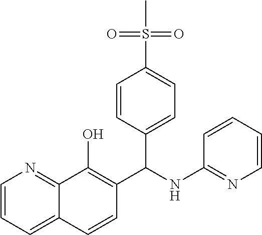 [SMILES: CS(=O)(=O)c1ccc(C(Nc2ccccn2)c2ccc3cccnc3c2O)cc1] | 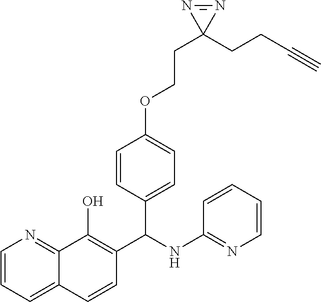 [SMILES: C#CCCC1(CCOc2ccc(C(Nc3ccccn3)c3ccc4cccnc4c3O)cc2)N=N1] |
| 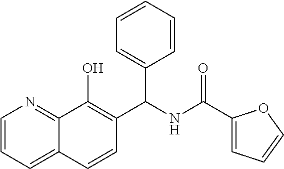 [SMILES: O=C(NC(c1ccccc1)c1ccc2cccnc2c1O)c1ccco1] | 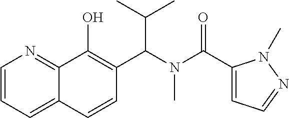 [SMILES: CC(C)C(c1ccc2cccnc2c1O)N(C)C(=O)c1ccnn1C] |
| 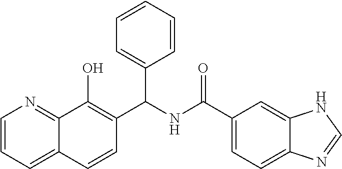 [SMILES: O=C(NC(c1ccccc1)c1ccc2cccnc2c1O)c1ccc2nc[nH]c2c1] | 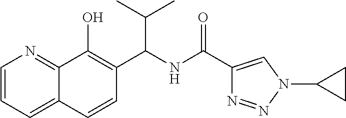 [SMILES: CC(C)C(NC(=O)c1cn(C2CC2)nn1)c1ccc2cccnc2c1O] |
| 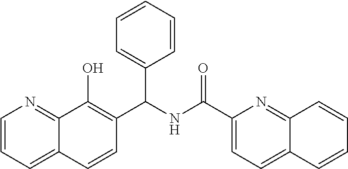 [SMILES: O=C(NC(c1ccccc1)c1ccc2cccnc2c1O)c1ccc2ccccc2n1] | 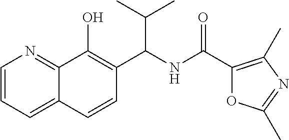 [SMILES: Cc1nc(C)c(C(=O)NC(c2ccc3cccnc3c2O)C(C)C)o1] |
| 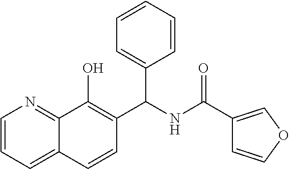 [SMILES: O=C(NC(c1ccccc1)c1ccc2cccnc2c1O)c1ccoc1] | 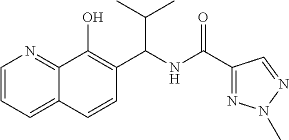 [SMILES: CC(C)C(NC(=O)c1cnn(C)n1)c1ccc2cccnc2c1O] |
| 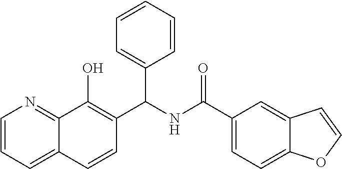 [SMILES: O=C(NC(c1ccccc1)c1ccc2cccnc2c1O)c1ccc2occc2c1] | 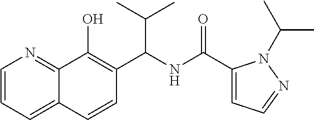 [SMILES: CC(C)C(NC(=O)c1ccnn1C(C)C)c1ccc2cccnc2c1O] |
| 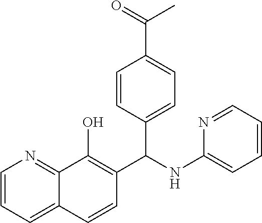 [SMILES: CC(=O)c1ccc(C(Nc2ccccn2)c2ccc3cccnc3c2O)cc1] | 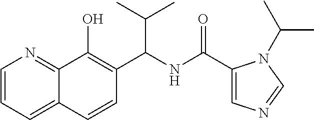 [SMILES: CC(C)C(NC(=O)c1cncn1C(C)C)c1ccc2cccnc2c1O] |
| 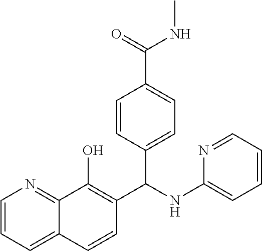 [SMILES: CNC(=O)c1ccc(C(Nc2ccccn2)c2ccc3cccnc3c2O)cc1] | 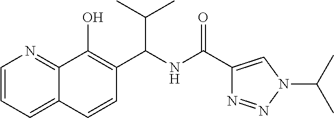 [SMILES: CC(C)C(NC(=O)c1cn(C(C)C)nn1)c1ccc2cccnc2c1O] |
| 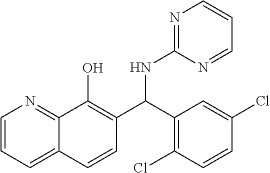 [SMILES: Oc1c(C(Nc2ncccn2)c2cc(Cl)ccc2Cl)ccc2cccnc12] | 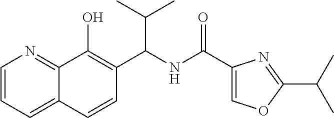 [SMILES: CC(C)c1nc(C(=O)NC(c2ccc3cccnc3c2O)C(C)C)co1] |
| 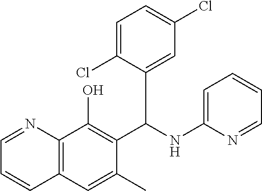 [SMILES: Cc1cc2cccnc2c(O)c1C(Nc1ccccn1)c1cc(Cl)ccc1Cl] | 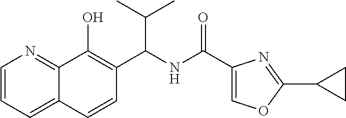 [SMILES: CC(C)C(NC(=O)c1coc(C2CC2)n1)c1ccc2cccnc2c1O] |
| 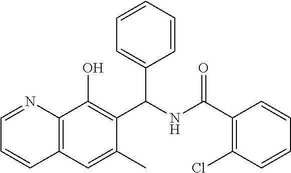 [SMILES: Cc1cc2cccnc2c(O)c1C(NC(=O)c1ccccc1Cl)c1ccccc1] | 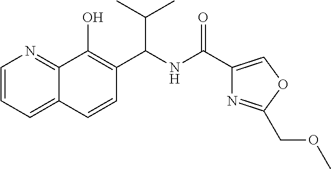 [SMILES: COCc1nc(C(=O)NC(c2ccc3cccnc3c2O)C(C)C)co1] |
| 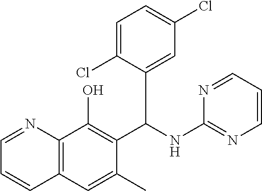 [SMILES: Cc1cc2cccnc2c(O)c1C(Nc1ncccn1)c1cc(Cl)ccc1Cl] | 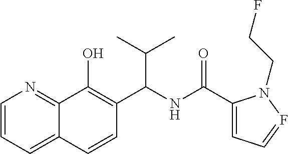 |
| 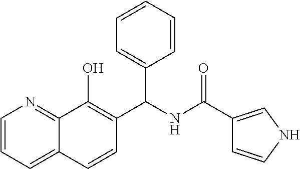 [SMILES: O=C(NC(c1ccccc1)c1ccc2cccnc2c1O)c1cc[nH]c1] | 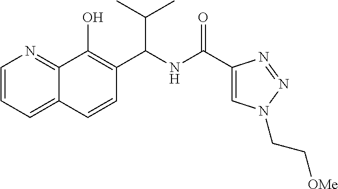 [SMILES: COCCn1cc(C(=O)NC(c2ccc3cccnc3c2O)C(C)C)nn1] |
| 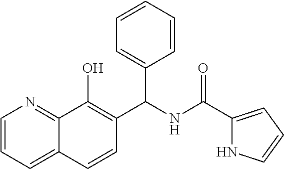 [SMILES: O=C(NC(c1ccccc1)c1ccc2cccnc2c1O)c1ccc[nH]1] | 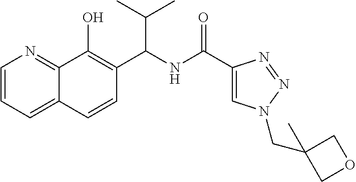 [SMILES: CC(C)C(NC(=O)c1cn(CC2(C)COC2)nn1)c1ccc2cccnc2c1O] |
| 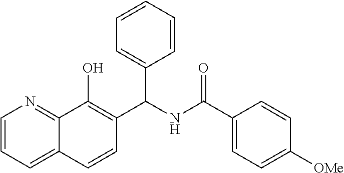 [SMILES: COc1ccc(C(=O)NC(c2ccccc2)c2ccc3cccnc3c2O)cc1] | 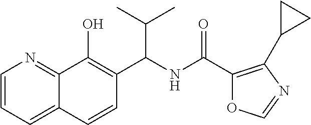 [SMILES: CC(C)C(NC(=O)c1ocnc1C1CC1)c1ccc2cccnc2c1O] |
| 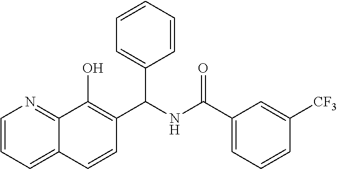 [SMILES: O=C(NC(c1ccccc1)c1ccc2cccnc2c1O)c1cccc(C(F)(F)F)c1] | 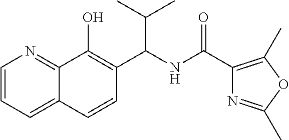 [SMILES: Cc1nc(C(=O)NC(c2ccc3cccnc3c2O)C(C)C)c(C)o1] |
| 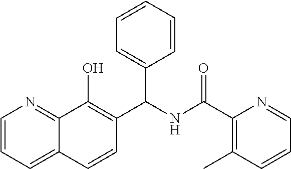 [SMILES: Cc1cccnc1C(=O)NC(c1ccccc1)c1ccc2cccnc2c1O] | |

In some embodiments, the compounds are atropisomers.

Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. Such compounds are useful, for example, as analytical tools, as probes in biological assays, or as therapeutic agents in accordance with the present invention. For example, in the case of variable R′, the (C₁-C₄)alkyl or the —O—(C₁-C₄)alkyl can be suitably deuterated (e.g., —CD₃, —OCD₃).

Any compound of the invention can also be radiolabed for the preparation of a radiopharmaceutical agent.

### Methods of Treatment

Creatine Transporter Deficiency (CTD)

CTD is an inborn error of creatine metabolism in which creatine is not properly transported to the brain and muscles due to defective creatine transporters. CTD is an X-linked disorder caused by mutations in the SLC6A8 gene. The SLC6A8 gene is located on the short arm of the sex chromosome, Xq28. Hemizygous males with CTD express speech and behavior abnormalities, intellectual disabilities, development delay, seizures, and autistic behavior. Heterozygous females with CTD generally express fewer, less severe symptoms. CTD is one of three different types of cerebral creatine deficiency (CCD). The other two types of CCD are guanidinoacetate methyltransferase (GAMT) deficiency and L-arginine: glycine amidinotransferase (AGAT) deficiency. Clinical presentation of CTD is similar to that of GAMT and AGAT deficiency. CTD was first identified in 2001 with the presence of a hemizygous nonsense mutation in the SLC6A8 gene in a male patient.

CTD is difficult to treat because the actual transporter responsible for transporting creatine to the brain and muscles is defective. Studies in which oral creatine monohydrate supplements were given to patients with CTD found that patients did not respond to treatment. However, similar studies conducted in which patients that had GAMT or AGAT deficiency were given oral creatine monohydrate supplements found that patient's clinical symptoms improved. Patients with CTD are unresponsive to oral creatine monohydrate supplements because regardless of the amount of creatine they ingest, the creatine transporter is still defective, and therefore creatine is incapable of being transported across the BBB.

Accordingly, in certain embodiments, the invention provides methods of disease or disorder associated with a SLC6A8 mutation, comprising administering to a mammal in need thereof a therapeutically effective amount of a compound or a pharmaceutical composition of the invention.

In some embodiments, the disease or disorder is creatine transporter deficiency. In some embodiments, the disease or disorder is motor dysfunction. In some embodiments, the disease or disorder is intellectual disability. In some embodiments, the disease or disorder is language delay or speech delay. In some embodiments, the disease or disorder is hypotonia. In some embodiments, the disease or disorder is seizures. In some embodiments, the disease or disorder is behaviours associated with autism and attention deficit hyperactivity disorder. In some embodiments, the disease or disorder is fatigue. In some embodiments, the disease or disorder is muscular hypotonia. In some embodiments, the disease or disorder is low weight gain. In some embodiments, the disease or disorder is gastrointestinal disorders. In some embodiments, the disease or disorder is cardiac disorders.

In certain embodiments, the invention provides methods of increasing cellular trafficking of a creatine transporter, comprising administering to a subject in need thereof an effective amount of a compound of the invention.

In some embodiments, the creatine transporter is SLC6A8. In some embodiments, the creatine transporter is a mutant creatine transporter.

In certain embodiments, the invention provides methods of correcting a defect in cellular creatine transporter function, comprising administering to a subject in need thereof an effective amount of a compound of the invention.

In some embodiments, the creatine transporter is SLC6A8. In some embodiments, the creatine transporter is a mutant creatine transporter. In some embodiments, the cellular concentration of creatine is increased.

In some embodiments, the invention relates to method of decreasing accumulation or the concentration of guanidinoacetic acid or a salt thereof in a cell, comprising administering to a mammal in need thereof a therapeutically effective amount of a compound that increases transport of guanidinoacetic acid or a salt thereof by a mutant creatine transporter.

In some embodiments, the creatine transporter is SLC6A8. In some embodiments, the creatine transporter is a mutant creatine transporter.

In some embodiments, the compound decreases intracellular accumulation of guanidinoacetic acid or a salt thereof. In some embodiments, the compound decreases the intracellular concentration of guanidinoacetic acid or a salt thereof.

In some embodiments, the invention relates to methods of increasing transport of guanidinoacetic acid or a salt thereof across the blood-brain barrier, comprising administering to a mammal in need thereof a therapeutically effective amount of a compound that increases transport of guanidinoacetic acid or a salt thereof by a mutant creatine transporter. In some embodiments, the mutant creatine transporter is mutant SLC6A8.

Pharmaceutical Compositions, Routes of Administration, and Dosing

In certain embodiments, the invention is directed to a pharmaceutical composition, comprising a compound of the invention and a pharmaceutically acceptable carrier. In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of ischemia-reperfusion injury.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Phannaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (al-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (μm), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt or cocrystal. When used in medicine the salts or cocrystals should be pharmaceutically acceptable, but non-pharmaceutically acceptable salts or cocrystals may conveniently be used to prepare pharmaceutically acceptable salts or cocrystals thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The invention is further described in the following examples, which do not limit the scope of the invention described in the claims.

### Example 1: PathHunter MEM-EA Pharmacotrafficking Assay for SLC6A8 CTD Mutants Cell Lines

### Preparation of Cells

U-2 OS MEM-EA cells were purchased from Eurofins (catalog #93-1101C₃). From these parental cells, stable cell lines expressing SLC6A8 CTD mutants were made using standard cell culture protocols, involving transfections of plasmids followed by antibiotic selection. These plasmids encoded CTD mutant SLC6A8 proteins with a C-terminal ProLink2 tag. U-2 OS MEM-EA cells and derived stable cell lines were grown in RPMI medium 1640 (Thermo Fisher Scientific, catalog #A10491-01) supplemented with 10% Fetal Bovine Serum (FBS), 200 ug/mL hygromycin B (Thermo Fisher Scientific, catalog #10687010), 100 mg/mL streptomycin, and 100 U/mL penicillin. Cells were grown at 37° C. in a humidified CO₂ incubator.

### Assay

U-2 OS MEM-EA cells stably expressing SLC6A8 CTD mutants were plated into white-walled 96-well plates (Corning, catalog #3903) at a density of 20,000 cells per well. For background subtraction, the parental U-2 OS MEM-EA cells were also plated. After 24 hrs, compounds were dispensed directly into the plated cells using the Tecan D300e Digital Dispenser. After an additional 24 hrs, the media with compound was again removed and white covers (Thermo Fisher Scientific, catalog #236272) were placed on the bottoms of the 96-well plates. Luminescence indicative of SLC6A8 CTD mutant cell surface localization was measured according to the manufacturer's protocol, using the PathHunter Detection kit (Eurofins catalog #93-0001L) and an EnVision plate reader (PerkinElmer, 2104 multilabel reader). Data were analyzed in Excel. Background signal from wells containing parental cells was subtracted, and then fold-changes were computed with respect to DMSO.

### Example 2: Corrector Assay for SLC6A8 CTD Mutant Cell Lines

### Preparation of Cells

A number of SLC6A8 CTD mutant cell lines were made in U-2 OS MEM-EA cells, 293T cells, HeLa cells, and CHO cells. All cells lines were generated as described above for U-2 OS MEM-EA cells, namely stable cell lines expressing SLC6A8 CTD mutants were made using standard cell culture protocols involving transfections of plasmids followed by antibiotic selection.

### Assay

Stable cell lines expressing CTD mutants were plated into 96-well plates (Corning, catalog #3595) at a density of 40,000 cells per well. After 24 hrs, compounds were dispensed directly into the plated cells using a Tecan D300e Digital Dispenser.

After an additional 24 hrs, the media with compound was removed. Cells were then incubated with a solution of 100 uM D3-creatine (SIGMA, 616249-1G) in media (without FBS) This solution was incubated with the cells for a 30 min incubation at 37° C. After the incubation, the media was removed, and the cells were washed once with 180 uL of phosphate buffered solution (PBS). To extract metabolites, water was added to the cells for 1 hour with vigorous shaking at 700 rpm. Cell extracts were analyzed on an ABSciex-4000 triple quad mass spectrometer coupled with a RapidFire sample desalting/injection system with a graphitic carbon desalting column and a basic buffer system in reverse phase. Abundances of D3-creatine were analyzed in Excel, and then fold-changes were computed with respect to DMSO.

### Example 3: General Procedures for the Synthesis of Representative Compounds of the Invention

### General Procedure A

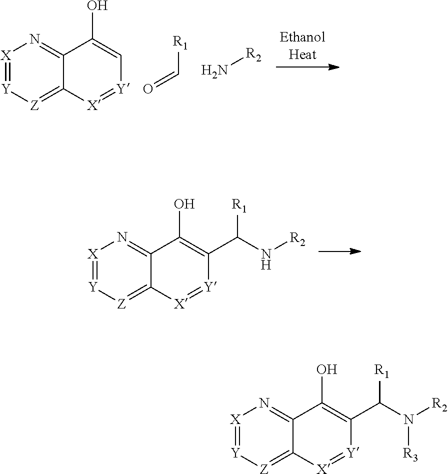

### Step 1

The appropriate aldehyde (1 equiv.) and amine (1.1 equiv.) were dissolved in Ethanol (0.3M) and the reaction was stirred at RT for 10-15 min before the appropriate quinoline (1 equiv.) was added and the reaction was stirred overnight at 60-80° C. In some case microwave-assisted synthesis was used. The reaction mixture was concentrated under vacuum and the crude was purified by prep HPLC.

### Step 2

Depending on the nature of R₃, different known reaction conditions were used to install R₃

### General Procedure B

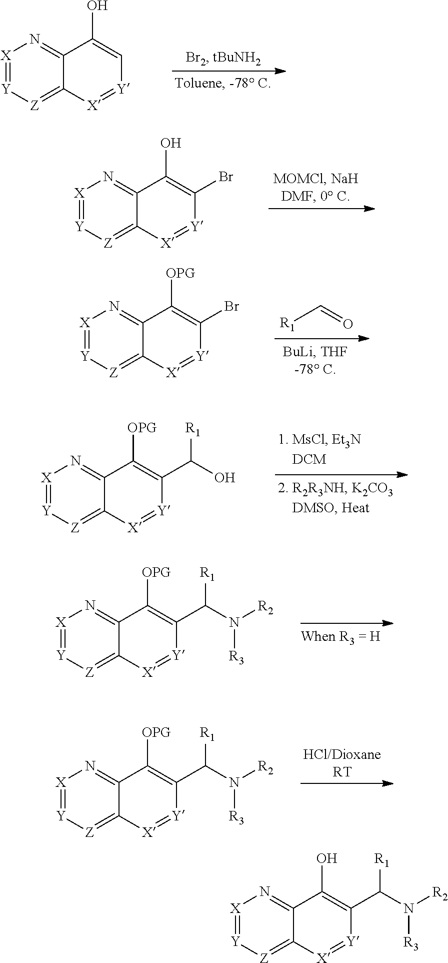

### Step 1

To a solution of t-BuNH₂ (2 equiv.) in toluene (0.1M) was added Br₂ (1 equiv.) dropwise at −78° C., and the mixture was stirred for 10 min. A solution of the appropriate quinoline (1 equiv.) in CHCl₃ (3M) was added dropwise to the above mixture at −78° C., and the resulting mixture was stirred at −78° C. for 1 hr. The reaction was quenched with saturated aq. NaHCO₃ solution and the aqueous layer was extracted with EtOAc (×3). The combined organic layers were washed with brine, dried over anhydrous Na₂SO₄, filtered and concentrated under vacuum. The residue was purified by flash chromatography.

### Step 2

To a solution of the above intermediate (1 equiv.) in anhydrous DMF (0.2M) was added NaH (1.2 eq, 60% in mineral oil) portion wise followed by dropwise addition of MOMCl (0.95 equiv.) at 0° C. The reaction mixture was stirred at 0° C. until full conversion. The mixture was then poured into ice-water and the aqueous layer extracted with EtOAc (×3). The combined organic layers were washed with water and brine, dried over anhydrous Na₂SO₄, filtered and concentrated under vacuum. The residue was purified by flash chromatography.

### Step 3

To a solution of the above intermediate (1 equiv.) in anhydrous THF (0.2M) was added n-BuLi (1.6 mol/L. 1.05 equiv.) dropwise at −78° C. under N₂ atmosphere. The reaction was stirred at this temperature for 30 min, then the appropriate aldehyde (1.5 equiv.) was added dropwise and the resulting mixture was stirred at −78° C. until full conversion. The reaction was quenched with saturated aq. NH₄Cl solution and the aqueous layer was extracted with EtOAc (×3). The combined organic layers were washed with brine, dried over anhydrous Na₂SO₄, filtered and concentrated under vacuum. The residue was purified by flash chromatography.

### Step 4

To a mixture of the above intermediate (1 equiv.) and TEA (3 equiv.) in DCM (0.1M) was added MSCl (1.2 equiv.) dropwise at 0° C. and the resulting mixture was stirred at RT until full conversion. The reaction was quenched with saturated aq. NH₄Cl solution and the aqueous layer was extracted with EtOAc (×2). The combined organic layers were washed with brine, dried over anhydrous Na₂SO₄, filtered and concentrated under vacuum. The crude was used for next step without any further purification.

### Step 5

To a solution of the mesylate intermediate (1 equiv.) in DMSO, K₂CO₃ (1.5 equiv.) was added followed by the appropriate amine (2 equiv.) and the resulting mixture was stirred at 40-50° C. until full conversion. The mixture was diluted with DCM and washed with water and brine. The organic layer was then dried over anhydrous Na₂SO₄, filtered and concentrated under vacuum. The residue was purified by prep HPLC.

### Step 6

Depending on the nature of R₃, different known reaction conditions were used to install R₃. When R₃═H Step 6 is skipped and the deprotection was performed using the conditions in Step 7.

### Step 7

To a solution of the above intermediate (1 equiv.) in 1,4-dioxane (0.1M) was added 4N HCl/1,4-dioxane (20 equiv.), and the mixture was stirred at RT until full deprotection. The mixture was concentrated under vacuum and the crude was purified by prep HPLC.

### General Procedure C

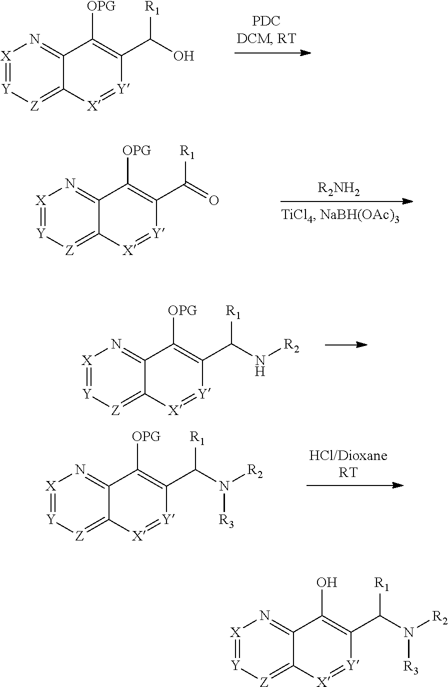

### Step 1

To a solution of the appropriate quinoline (1 eq, synthesized as in step 3 of general procedure B) in DCM (0.1M), PDC (1.2 equiv.) was added and reaction stirred at RT until full conversion. The mixture was diluted with DCM and washed with water and brine. The organic layer was then dried over anhydrous Na₂SO₄, filtered and concentrated under vacuum. The residue was purified by flash chromatography.

### Step 2

To a solution of the above intermediate (1 equiv.) in DCM (0.1M) TiCl₄ (1M in DCM, 1.2 equiv.) was added followed by the appropriate amine (1.5 eq). The resulting mixture was stirred at RT for few hours, then NaBH(OAc)₃ (2 equiv.) was added followed by methanol (1/3 volume of DCM). The reaction was then stirred at RT until full conversion. The reaction was quenched with saturated aq. NaHCO₃ solution and the aqueous layer was extracted with EtOAc (×3). The combined organic layers were washed with brine, dried over anhydrous Na₂SO₄, filtered and concentrated under vacuum. The residue was purified by flash chromatography or prep HPLC.

### Step 3

Depending on the nature of R₃, different known reaction conditions were used to install R₃. When R₃═H Step 3 is skipped and the deprotection was performed using the conditions in Step 4.

### Step 4

To a solution of the above intermediate (1 equiv.) in 1,4-dioxane (0.1M) was added 4N HCl/1,4-dioxane (20 equiv.), and the mixture was stirred at RT until full deprotection. The mixture was concentrated under vacuum and the crude was purified by prep HPLC.

### General Procedure D

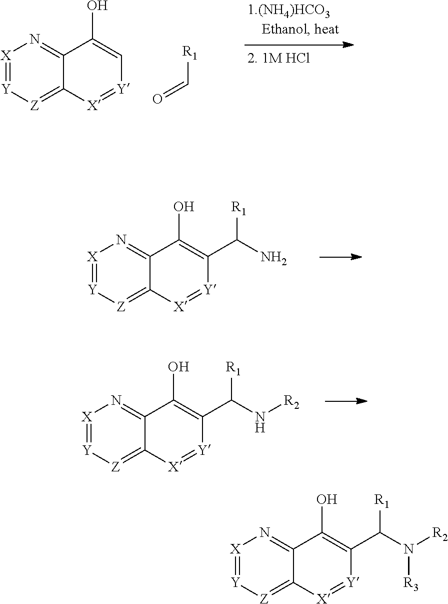

### Step 1-2

The appropriate quinoline (1 equiv.) and the appropriate aldehyde (2 equiv.) were dissolved in Ethanol (0.3M), then ammonium bicarbonate (3 equiv.) was added and reaction stirred overnight at 80° C. The reaction mixture was concentrated under vacuum and the residue was dissolved in 1M HCl (0.3M) and the resulting mixture was stirred at 80° C. for 1 hour and the crude product was purified by prep HPLC.

### Steps 2 & 3

Depending on the nature of R₂ and R₃, different known reaction conditions were used to install R₂ and R₃

### General Procedure E

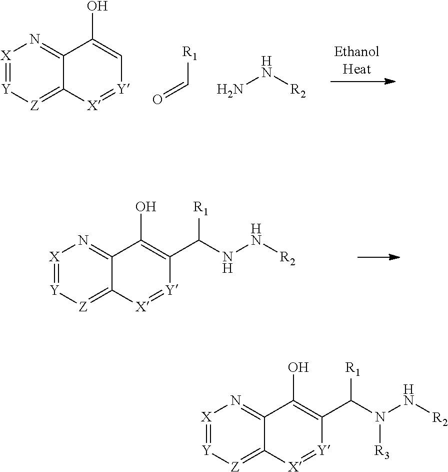

### Step 1

The appropriate aldehyde (1 equiv.) and hydrazine (1.1 equiv.) were dissolved in Ethanol (0.3M) and the reaction was stirred at RT for 10-15 min before the appropriate quinoline (1 equiv.) was added and the reaction was stirred overnight at 60-80° C. In some case microwave-assisted synthesis was used. The reaction mixture was concentrated under vacuum and the crude was purified by prep HPLC.

### Step 2

Depending on the nature of R₃, different known reaction conditions were used to install R₃

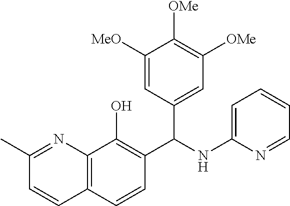 [SMILES: COc1cc(C(Nc2ccccn2)c2ccc3ccc(C)nc3c2O)cc(OC)c1OC]

This compound was synthesized using General Procedure A (LC/MS m/z 432.33 [M+H⁺])

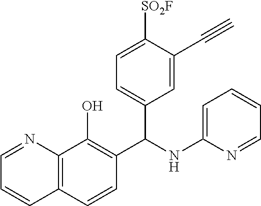 [SMILES: C#Cc1cc(C(Nc2ccccn2)c2ccc3cccnc3c2O)ccc1S(=O)(=O)F]

This compound was synthesized using General Procedure A (LC/MS m/z 434.37 [M+H⁺])

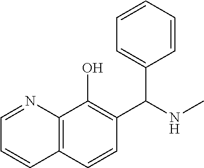 [SMILES: CNC(c1ccccc1)c1ccc2cccnc2c1O]

This compound was synthesized using General Procedure A (LC/MS m/z 265.09 [M+H⁺])

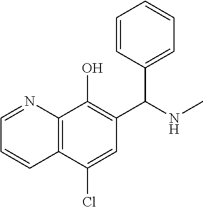 [SMILES: CNC(c1ccccc1)c1cc(Cl)c2cccnc2c1O]

This compound was synthesized using General Procedure A (LC/MS m/z 299.40 [M+H⁺])

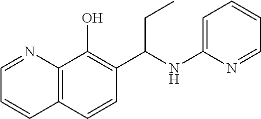 [SMILES: CCC(Nc1ccccn1)c1ccc2cccnc2c1O]

This compound was synthesized using General Procedure A (LC/MS m/z 280.34 [M+H⁺])

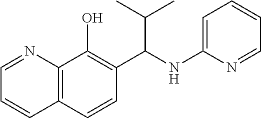 [SMILES: CC(C)C(Nc1ccccn1)c1ccc2cccnc2c1O]

This compound was synthesized using General Procedure A (LC/MS m/z 294.30 [M+H⁺])

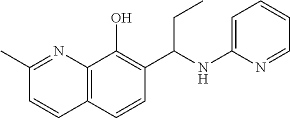 [SMILES: CCC(Nc1ccccn1)c1ccc2ccc(C)nc2c1O]

This compound was synthesized using General Procedure A (LC/MS m/z 294.33 [M+H⁺])

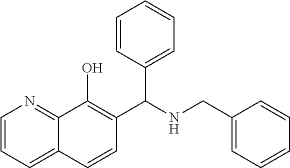 [SMILES: Oc1c(C(NCc2ccccc2)c2ccccc2)ccc2cccnc12]

This compound was synthesized using General Procedure B (LC/MS m/z 341.44 [M+H⁺])

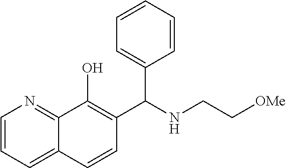 [SMILES: COCCNC(c1ccccc1)c1ccc2cccnc2c1O]

This compound was synthesized using General Procedure A (LC/MS m/z 309.43 [M+H⁺])

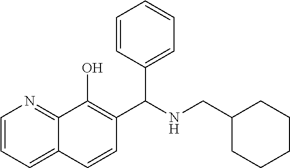 [SMILES: Oc1c(C(NCC2CCCCC2)c2ccccc2)ccc2cccnc12]

This compound was synthesized using General Procedure A (LC/MS m/z 347.47 [M+H⁺])

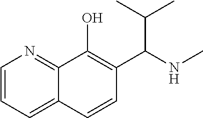 [SMILES: CNC(c1ccc2cccnc2c1O)C(C)C]

This compound was synthesized using General Procedure A (LC/MS m/z 231.12 [M+H⁺])

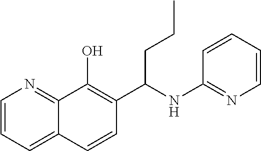 [SMILES: CCCC(Nc1ccccn1)c1ccc2cccnc2c1O]

This compound was synthesized using General Procedure A (LC/MS m/z 294.37 [M+H⁺])

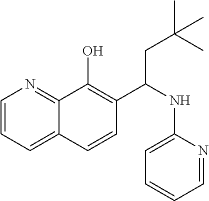 [SMILES: CC(C)(C)CC(Nc1ccccn1)c1ccc2cccnc2c1O]

This compound was synthesized using General Procedure A (LC/MS m/z 322.42 [M+H⁺])

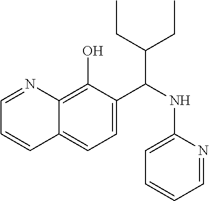 [SMILES: CCC(CC)C(Nc1ccccn1)c1ccc2cccnc2c1O]

This compound was synthesized using General Procedure A (LC/MS m/z 322.49 [M+H⁺])

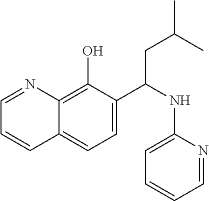 [SMILES: CC(C)CC(Nc1ccccn1)c1ccc2cccnc2c1O]

This compound was synthesized using General Procedure A (LC/MS m/z 308.39 [M+H⁺])

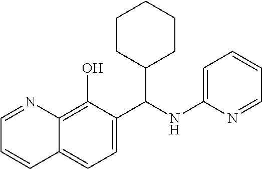 [SMILES: Oc1c(C(Nc2ccccn2)C2CCCCC2)ccc2cccnc12]

This compound was synthesized using General Procedure A (LC/MS m/z 334.41 [M+H⁺])

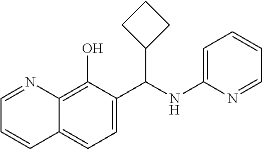 [SMILES: Oc1c(C(Nc2ccccn2)C2CCC2)ccc2cccnc12]

This compound was synthesized using General Procedure A (LC/MS m/z 306.39 [M+H⁺])

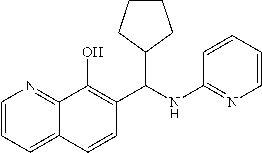 [SMILES: Oc1c(C(Nc2ccccn2)C2CCCC2)ccc2cccnc12]

This compound was synthesized using General Procedure A (LC/MS m/z 320.46 [M+H⁺])

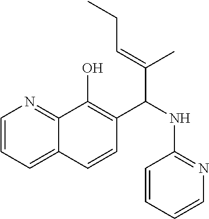 [SMILES: CC/C=C(\C)C(Nc1ccccn1)c1ccc2cccnc2c1O]

This compound was synthesized using General Procedure A (LC/MS m/z 320.46 [M+H⁺])

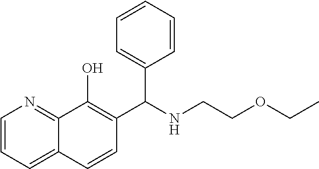 [SMILES: CCOCCNC(c1ccccc1)c1ccc2cccnc2c1O]

This compound was synthesized using General Procedure A (LC/MS m/z 323.45 [M+H⁺])

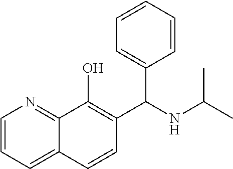 [SMILES: CC(C)NC(c1ccccc1)c1ccc2cccnc2c1O]

This compound was synthesized using General Procedure B (LC/MS m/z 293.40 [M+H⁺])

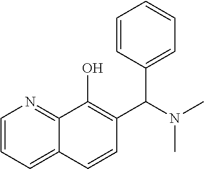 [SMILES: CN(C)C(c1ccccc1)c1ccc2cccnc2c1O]

This compound was synthesized using General Procedure B (LC/MS m/z 279.38 [M+H⁺])

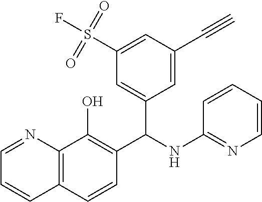 [SMILES: C#Cc1cc(C(Nc2ccccn2)c2ccc3cccnc3c2O)cc(S(=O)(=O)F)c1]

This compound was synthesized using General Procedure A (LC/MS m/z 434.33 [M+H⁺])

### General Procedure 1

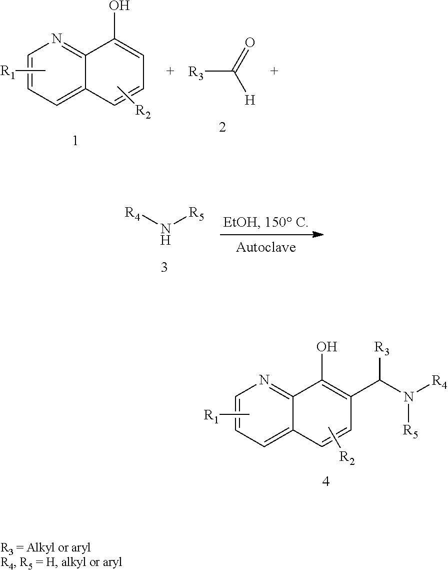

A solution of the appropriate 8-hydroxyquinoline 1 (1 eq.), alkyl or aryl aldehyde 2 (1.2 eq.) and alkyl or aryl amine 3 (1.2 eq.) in EtOH (0.1M) was transferred to an autoclave reactor, which was then sealed and heated to 150° C. for 12 hours. The reaction mixture was concentrated under vacuum and the residue was purified by reverse-phase flash column chromatography, prep-TLC or prep-HPLC to give the desired compound 4.

### Example: Synthesis of Compound 5

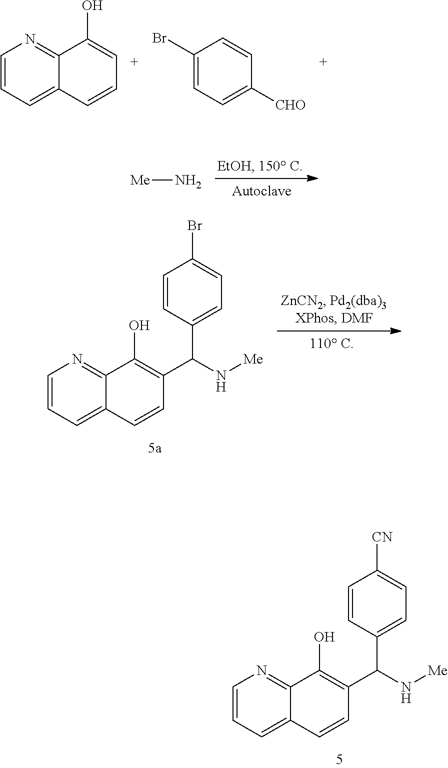 [SMILES: Oc1cccc2cccnc12 ; O=Cc1ccc(Br)cc1 ; CN ; CNC(c1ccc(Br)cc1)c1ccc2cccnc2c1O ; CNC(c1ccc(C#N)cc1)c1ccc2cccnc2c1O]

### Step 1

A solution of 8-hydroxyquinoline (290 mg, 2.0 mmol), methyl amine (2.0M in EtOH, 1.2 mL) and 4-bromobenzaldehyde (442 mg, 2.4 mmol) in EtOH (20 mL) was transferred to an autoclave reactor, which was then sealed and heated to 150° C. for 12 hours. The reaction mixture was concentrated under vacuum and the residue was purified by reverse-phase flash column chromatography, followed by prep-HPLC to give 5a (25 mg, yield: 3.6%) as a white solid.

### Step 2

ZnCN₂ (16 mg, 0.14 mmol), Pd₂(dba)₃ (8 mg, 0.01 mmol) and XPhos (6 mg, 0.01 mmol) were added under N₂ atmosphere to a solution of 5a (25 mg, 0.07 mmol) in DMF (2 mL), and the mixture was heated to 110° C. for 8 hours. The reaction was cooled to room temperature, concentrated under vacuum and the residue was purified by prep-HPLC to give 5 (4 mg, yield: 20%) as a white solid.

### General Procedure 2

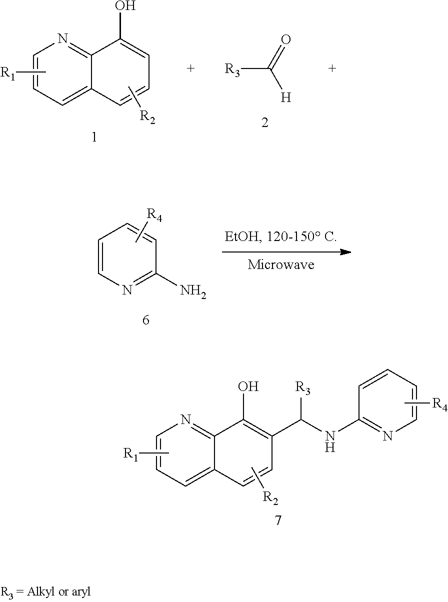

A solution of the appropriate 8-hydroxyquinoline 1 (1 eq.), alkyl or aryl aldehyde 2 (1.2 eq.) and 2-aminopyridine 6 (1.2 eq.) in EtOH (0.1M) was transferred to a microwave reaction vial, which was then sealed and stirred under microwave condition at 120-150° C. (150° C. for alkyl aldehyde) for 2 to 6 hours. The reaction mixture was then cooled to room temperature and concentrated under vacuum. The residue was purified by reverse-phase flash column chromatography, prep-TLC or prep-HPLC to give compound 7.

### Example: Synthesis of Compound 8

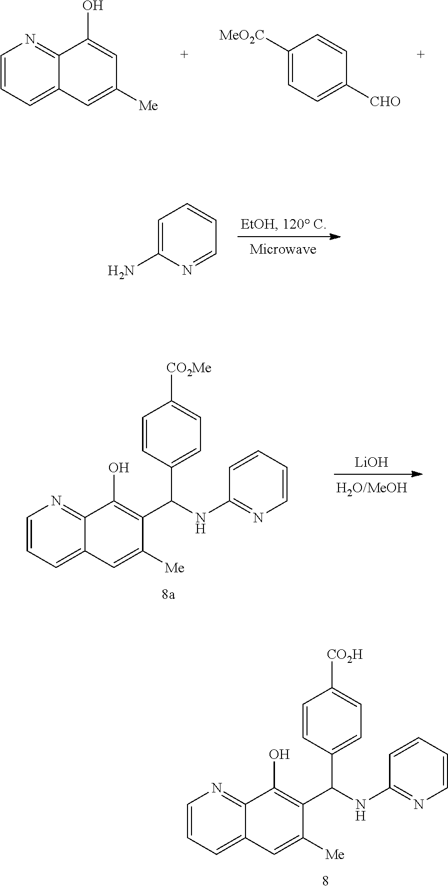 [SMILES: Cc1cc(O)c2ncccc2c1 ; COC(=O)c1ccc(C=O)cc1 ; Nc1ccccn1 ; COC(=O)c1ccc(C(Nc2ccccn2)c2c(C)cc3cccnc3c2O)cc1 ; "LiOH" ; Cc1cc2cccnc2c(O)c1C(Nc1ccccn1)c1ccc(C(=O)O)cc1]

### Step 1

A solution of 6-methyl-8-hydroxyquinoline (160 mg, 1 mmol), methyl 4-formylbenzoate (197 mg, 1.2 mmol) and 2-aminopyridine (110 mg, 1.2 mmol) in EtOH (10 mL) was transferred to a microwave reaction vial, which was then sealed and stirred under microwave condition at 120° C. for 2 hours. The reaction mixture was then cooled to room temperature and concentrated under vacuum. The residue was purified by reverse-phase flash column chromatography, followed by prep-TLC to give 8a (40 mg, yield: 10%) as a light yellow solid.

### Step 2

8a (40 mg, 0.1 mmol) was added to a solution of lithium hydroxide monohydrate (10 mg, 0.24 mmol) in methanol (2 mL) and H₂O (2 mL), and the resulting mixture was stirred overnight at room temperature. The mixture was then acidified with aq. HCl (1N) solution to pH=4 and concentrated under vacuum. The residue was purified by prep-HPLC to give 8 (12.6 mg, yield: 33%) as a yellow solid.

### General Procedure 3

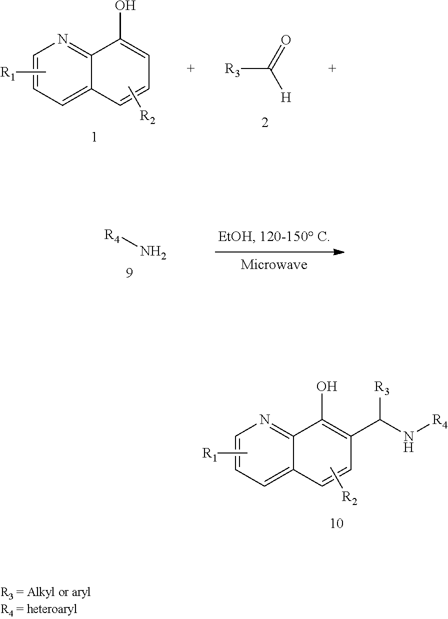

A solution of the appropriate 8-hydroxyquinoline 1 (1 eq.), alkyl or aryl aldehyde 2 (1.2 eq.) and heteroaryl amine 9 (1.2 eq.) in EtOH (0.1M) was transferred to a microwave reaction vial, which was then sealed and stirred under microwave condition at 120° C. (150° C. for alkyl aldehydes) for 2 to 6 hours. The reaction mixture was then cooled to room temperature and concentrated under vacuum. The residue was purified by reverse-phase flash column chromatography, prep-TLC or prep-HPLC to give compound 10.

### Example: Synthesis of Compound 11

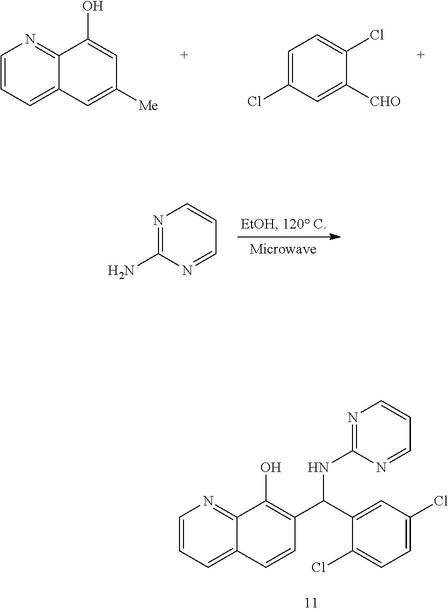 [SMILES: Cc1cc(O)c2ncccc2c1 ; O=Cc1cc(Cl)ccc1Cl ; Nc1ncccn1 ; Oc1c(C(Nc2ncccn2)c2cc(Cl)ccc2Cl)ccc2cccnc12]

A solution of 6-methyl-8-hydroxyquinoline (160 mg, 1 mmol), 2,5-dichlorobenzaldehyde (210 mg, 1.2 mmol) and 2-aminopyrimidine (110 mg, 1.2 mmol) in EtOH (10 mL) was transferred to a microwave reaction vial, which was then sealed and heated to 120° C. under microwave conditions for 4 hours. The reaction mixture was cooled to room temperature and concentrated under vacuum. The residue was purified by reverse-phase flash column chromatography, followed by prep-TLC to give the desired compound 11 (5.9 mg, yield: 1.5%) as a yellow solid.

### General Procedure 4

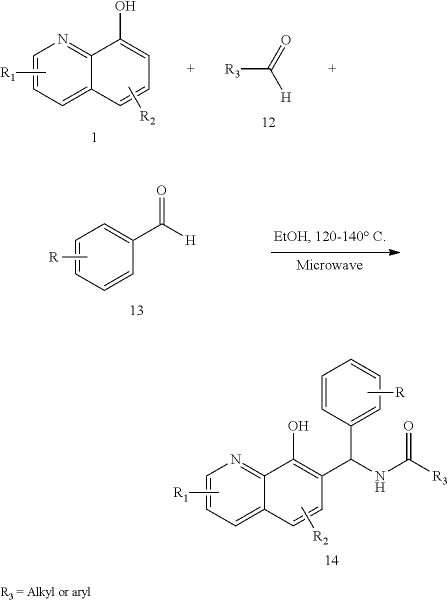

A solution of the appropriate 8-hydroxyquinoline 1 (1 eq.), alkyl or aryl amide 12 (1.2 eq.) and aryl aldehyde 13 (1.2 eq.) in EtOH (0.1M) was transferred to a microwave reaction vial, which was then sealed and stirred under microwave condition at 120-140° C. for 2 to 12 hours (alkyl amides required longer reaction time). The reaction mixture was then cooled to room temperature and concentrated under vacuum. The reside was purified by reverse-phase flash column chromatography, prep-TLC or prep-HPLC to give compound 14.

### Example: Synthesis of Compound 15

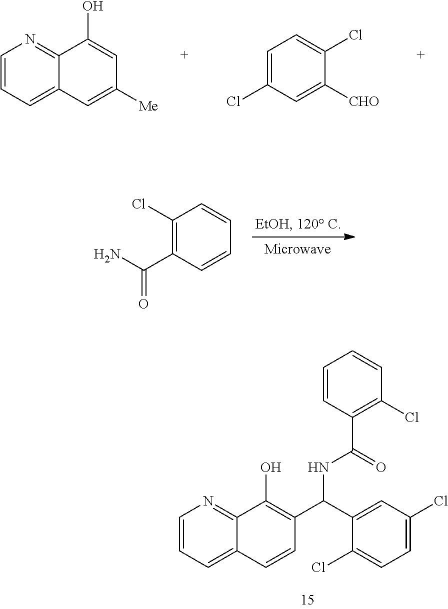 [SMILES: Cc1cc(O)c2ncccc2c1 ; O=Cc1cc(Cl)ccc1Cl ; NC(=O)c1ccccc1Cl ; O=C(NC(c1cc(Cl)ccc1Cl)c1ccc2cccnc2c1O)c1ccccc1Cl]

A solution of 6-methyl-8-hydroxyquinoline (160 mg, 1 mmol), 2,5-dichlorobenzaldehyde (210 mg, 1.2 mmol) and 2-chlorobenzamide (186 mg, 1.2 mmol) in EtOH (10 mL) was transferred to a microwave reaction vial, which was then sealed and stirred under microwave condition at 120° C. for 2 hours. The reaction mixture was then cooled to room temperature and concentrated under vacuum. The reside was purified by reverse-phase flash column chromatography followed by prep-HPLC to give 15 (4.9 mg, yield: 1.5%) as a white solid.

### General Procedure 5

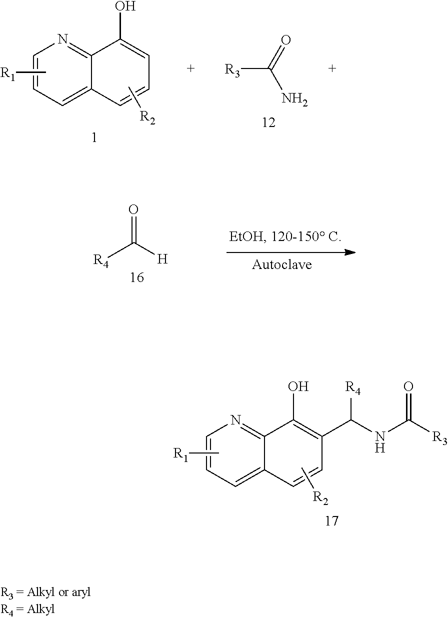

A solution of the appropriate 8-hydroxyquinoline 1 (1 eq.), alkyl or aryl amide 12 (1.2 eq.), and alkyl aldehyde 16 (1.2 eq.) in EtOH (0.1M) was transferred to an autoclave reactor, which was then sealed and heated to 120-150° C. for 12 to 24 hours. The reaction mixture was concentrated under vacuum and the residue was purified by reverse-phase flash column chromatography, prep-TLC or prep-HPLC to give compound 17.

### Example: Synthesis of Compound 18

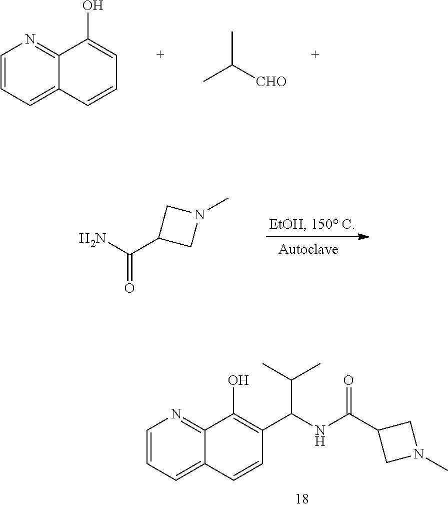

A solution of 8-hydroxyquinoline (145 mg, 1 mmol), isobutyraldehyde (85 mg, 1.2 mmol) and 1-methylazetidine-3-carboxamide (140 mg, 1.2 mmol) in EtOH (10 mL) was transferred to an autoclave reactor, which was then sealed and heated to 150° C. for 12 hours. The reaction mixture was concentrated under vacuum and the residue was purified by reverse-phase flash column chromatography, followed by prep-HPLC to give 18 (11 mg, yield: 3.5%) as a white solid.

### General Procedure 6

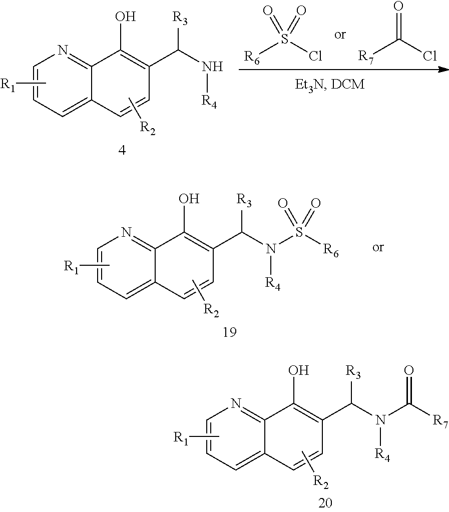

### Compound 4 was Prepared According to General Procedure 1

The appropriate compound 4 (1 eq.) was dissolved in DCM (0.05M), then Et₃N (2 eq.) was added followed by the appropriate sulfonyl chloride or acyl chloride (1.2 eq.) at 0° C. The reaction was slowly warmed to room temperature and stirred for 15-30 minutes. The mixture was concentrated under vacuum and the residue was purified by prep-HPLC to give compound 19 or 20.

### Example: Synthesis of Compound 21

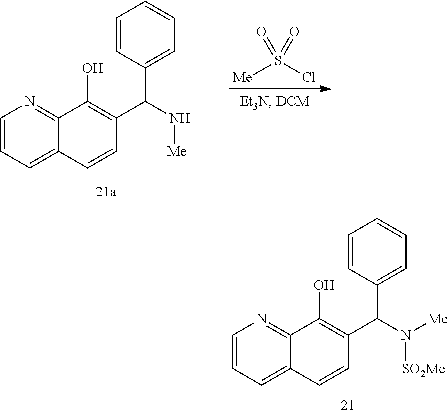 [SMILES: CNC(c1ccccc1)c1ccc2cccnc2c1O ; CS(=O)(=O)Cl ; CN(C(c1ccccc1)c1ccc2cccnc2c1O)S(C)(=O)=O]

21a (6.0 mg, 0.023 mmol) was dissolved in DCM (0.5 mL), then Et₃N (6.33 uL, 0.046 mmol) was added followed by methanesulfonyl chloride (5 uL, 0.028 mmol) at 0° C. The reaction was slowly warmed to room temperature and stirred for 30 minutes. The mixture was concentrated under vacuum and the residue was purified by prep-HPLC to give 21 as a white solid.

| | General | |
|---|---|---|
| Compound | procedure | Experimental data |
| 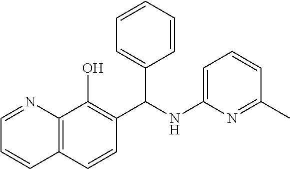 [SMILES: Cc1cccc(NC(c2ccccc2)c2ccc3cccnc3c2O)n1] | 2 | 7-(((6-methylpyridin-2- yl)amino)(phenyl)methyl)quinolin-8-ol LC-MS: m/z 342 (M + H)⁺. 1H NMR (400 MHz, DMSO-d6) δ 9.91 (brs, 1H), 8.78 (dd, J = 4.2, 1.7 Hz, 1H), 8.25-8.17 (m, 1H), 7.58 (d, J = 8.6 Hz, 1H), 7.46 (dd, J = 8.3, 4.2 Hz, 1H), 7.35-7.27 (m, 3H), 7.24-7.15 (m, 4H), 7.13- 7.07 (m, 1H), 6.75 (d, J = 8.8 Hz, 1H), 6.36 (d, J = 8.3 Hz, 1H), 6.27 (d, J = 7.1 Hz, 1H), 2.14 (s, 3H). |
| 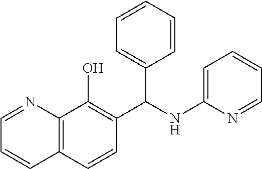 [SMILES: Oc1c(C(Nc2ccccn2)c2ccccc2)ccc2cccnc12] | 2 | 7-(phenyl(pyridin-2-ylamino)methyl)quinolin-8-ol LC-MS: m/z 328 (M + H)⁺. 1H NMR (400 MHz, DMSO-d6) δ 9.98 (brs, 1H), 8.85 (dd, J = 4.1, 1.8 Hz, 1H), 8.29 (dd, J = 8.4, 1.8 Hz, 1H), 7.91 (d, J = 5.0 Hz, 1H), 7.62 (d, J = 8.4 Hz, 1H), 7.53 (dd, J = 8.3, 4.3 Hz, 1H), 7.45-7.33 (m, 5H), 7.31-7.27 (m, 2H), 7.21-7.17 (m, 1H), 6.86 (d, J = 8.4 Hz, 1H), 6.68 (d, J = 8.5 Hz, 1H), 6.48-6.45 (m, 1H). |
| 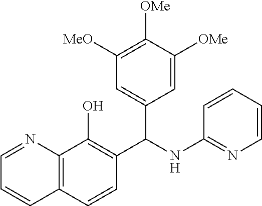 [SMILES: COc1cc(C(Nc2ccccn2)c2ccc3cccnc3c2O)cc(OC)c1OC] | 2 | 7-((pyridin-2-ylamino)(3,4,5- trimethoxyphenyl)methyl)quinolin-8-ol LC-MS: m/z 418 (M + H)⁺. 1H NMR (400 MHz, DMSO-d₆) δ 9.94 (brs, 1H), 8.85 (dd, J = 4.2, 1.8 Hz, 1H), 8.36-8.23 (m, 1H), 7.92 (d, J = 4.8 Hz, 1H), 7.64 (d, J = 8.5 Hz, 1H), 7.53 (dd, J = 8.3, 4.2 Hz, 1H), 7.42-7.28 (m, 3H), 6.77-6.73 (m, 3H), 6.67 (d, J = 8.5 Hz, 1H), 6.49- 6.46 (m, 1H), 3.70 (d, J = 1.7 Hz, 6H), 3.60 (d, J = 1.7 Hz, 3H). - No references, but can buy it |
| 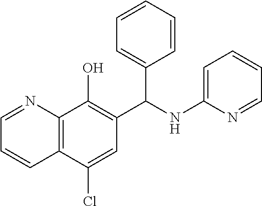 [SMILES: Oc1c(C(Nc2ccccn2)c2ccccc2)cc(Cl)c2cccnc12] | 2 | 5-chloro-7-(phenyl(pyridin-2- ylamino)methyl)quinolin-8-ol LC-MS: m/z 362 (M + H)⁺. 1H NMR (400 MHz, DMSO-d6) δ 10.29 (brs, 1H), 8.95 (dd, J = 4.2, 1.5 Hz, 1H), 8.46 (dd, J = 8.6, 1.6 Hz, 1H), 7.92 (dd, J = 5.1, 1.8 Hz, 1H), 7.78 (s, 1H), 7.71 (dd, J = 8.6, 4.2 Hz, 1H), 7.47 (d, J = 8.7 Hz, 1H), 7.44-7.27 (m, 5H), 7.23-7.20 (m, 1H), 6.87 (d, J = 8.5 Hz, 1H), 6.69 (d, J = 8.4 Hz, 1H), 6.49 (dd, J = 7.0, 5.1 Hz, 1H). |
| 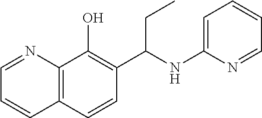 [SMILES: CCC(Nc1ccccn1)c1ccc2cccnc2c1O] | A | 7-(1-(pyridin-2-ylamino)propyl)quinolin-8-ol LC-MS: m/z 280 (M + H)⁺. 1H NMR (400 MHz, DMSO-d6) δ 9.77 (brs, 1H), 8.77 (dd, J = 4.2, 1.6 Hz, 1H), 8.18 (dd, J = 8.3, 1.6 Hz, 1H), 7.79 (dd, J = 5.1, 1.8 Hz, 1H), 7.48 (d, J = 8.5 Hz, 1H), 7.44 (dd, J = 8.3, 4.2 Hz, 1H), 7.30- 7.20 (m, 2H), 6.92 (d, J = 8.3 Hz, 1H), 6.42 (d, J = 8.4 Hz, 1H), 6.32 (dd, J = 7.0, 5.1 Hz, 1H), 5.23- |
| | | 5.18 (m, 1H), 1.80-1.72 (m, 2H), 0.87 (t, J = 7.3 |
| | | Hz, 3H). |
| 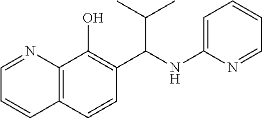 [SMILES: CC(C)C(Nc1ccccn1)c1ccc2cccnc2c1O] | A | 7-(2-methyl-1-(pyridin-2-ylamino)propyl)quinolin- 8-ol LC-MS: m/z 294 (M + H)⁺. 1H NMR (400 MHz, DMSO-d6) δ 9.75 (brs, 1H), 8.76 (dd, J = 4.2, 1.6 Hz, 1H), 8.19 (dd, J = 8.3, 1.6 Hz, 1H), 7.79 (dd, J = 5.2, 1.8 Hz, 1H), 7.49 (d, J = 8.5 Hz, 1H), 7.44 (dd, J = 8.3, 4.2 Hz, 1H), 7.28 (d, J = 8.5 Hz, 1H), 7.22 (ddd, J = 8.8, 7.0, 2.0 Hz, |
| | | 1H), 6.85 (d, J = 9.0 Hz, 1H), 6.46 (d, J = 8.5 Hz, |
| | | 1H), 6.31 (dd, J = 7.0, 5.0 Hz, 1H), 5.13 (t, J = 8.4 |
| | | Hz, 1H), 2.11 (dq, J = 13.7, 6.8 Hz, 1H), 0.95 (d, J = |
| | | 6.7 Hz, 3H), 0.76 (d, J = 6.7 Hz, 3H). |
| 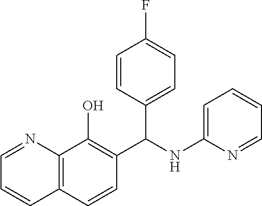 [SMILES: Oc1c(C(Nc2ccccn2)c2ccc(F)cc2)ccc2cccnc12] | 2 | 7-((4-fluorophenyl)(pyridin-2- ylamino)methyl)quinolin-8-ol LC-MS: m/z 346 (M + H)⁺. 1H NMR (400 MHz, DMSO-d6) δ 9.98 (brs, 1H), 8.85 (dd, J = 4.3, 1.6 Hz, 1H), 8.29 (dd, J = 8.4, 1.6 Hz, 1H), 7.91 (dd, J = 5.2, 1.8 Hz, 1H), 7.61 (d, J = 8.5 Hz, 1H), 7.53 (dd, J = 8.3, 4.2 Hz, 1H), 7.41- 7.35 (m, 5H), 7.15-7.08 (m, 2H), 6.85 (d, J = 8.4 Hz, 1H), 6.67 (d, J = 8.4 Hz, 1H), 6.47 (dd, J = 7.0, 5.1 Hz, 1H). |
| 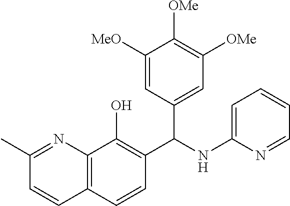 [SMILES: COc1cc(C(Nc2ccccn2)c2ccc3ccc(C)nc3c2O)cc(OC)c1OC] | A | 2-methyl-7-((pyridin-2-ylamino)(3,4,5- trimethoxyphenyl)methyl)quinolin-8-ol LC-MS: m/z 432 (M + H)⁺. 1H NMR (400 MHz, DMSO-d6) δ 9.46 (brs, 1H), 8.15 (d, J = 8.4 Hz, 1H), 7.92 (dd, J = 5.3, 1.9 Hz, 1H), 7.56 (d, J = 8.5 Hz, 1H), 7.42-7.30 (m, 4H), 6.77-6.73 (m, 3H), 6.66 (d, J = 8.5 Hz, 1H), 6.47 (dd, J = 6.8, 5.3 Hz, 1H), 3.69 (s, 6H), 3.59 (s, 3H), 2.69 (s, 3H). |
| 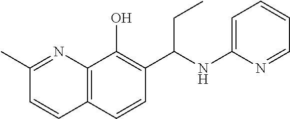 [SMILES: CCC(Nc1ccccn1)c1ccc2ccc(C)nc2c1O] | A | 2-methyl-7-(1-(pyridin-2-ylamino)propyl)quinolin- 8-ol LC-MS: m/z 294 (M + H)⁺. 1H NMR (400 MHz, DMSO-d6) δ 9.39 (brs, 1H), 8.13 (d, J = 8.4 Hz, 1H), 7.87 (dd, J = 5.1, 1.8 Hz, 1H), 7.47 (d, J = 8.5 Hz, 1H), 7.38 (d, J = 8.4 Hz, 1H), 7.34-7.27 (m, 2H), 6.98 (d, J = 8.2 Hz, 1H), 6.48 (d, J = 8.5 Hz, 1H), 6.39 (dd, J = 7.0, 5.1 Hz, |
| | | 1H), 5.24 (q, J = 7.6 Hz, 1H), 2.69 (s, 3H), 1.92- |
| | | 1.75 (m, 2H), 0.93 (t, J = 7.3 Hz, 3H). |
| 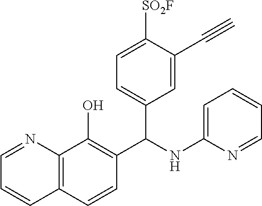 [SMILES: C#Cc1cc(C(Nc2ccccn2)c2ccc3cccnc3c2O)ccc1S(=O)(=O)F] | A | 2-ethynyl-4-((8-hydroxyquinolin-7-yl)(pyridin-2- ylamino)methyl)benzenesulfonyl fluoride LC-MS: m/z 434 (M + H)⁺. 1H NMR (400 MHz, DMSO-d6) δ 8.88 (dd, J = 4.2, 1.6 Hz, 1H), 8.37-8.28 (m, 1H), 8.11 (d, J = 8.4 Hz, 1H), 7.98-7.90 (m, 1H), 7.83 (d, J = 1.8 Hz, 1H), 7.70 (d, J = 8.4 Hz, 1H), 7.58-7.56 (m, 3H), 7.45-7.38 (m, 2H), 6.95 (d, J = 7.9 Hz, 1H), 6.74 (d, J = 8.3 Hz, 1H), 6.58-6.47 (m, 1H), 4.92 (s, 1H). |
| 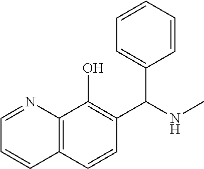 [SMILES: CNC(c1ccccc1)c1ccc2cccnc2c1O] | A | 7-((methylamino)(phenyl)methyl)quinolin-8-ol LC-MS: m/z 265 (M + H)⁺. 1H NMR (400 MHz, DMSO-d6) δ 8.82 (d, J = 4.1 Hz, 1H), 8.25 (d, J = 8.5 Hz, 1H), 7.56 (d, J = 8.6 Hz, 1H), 7.53-7.42 (m, 3H), 7.38-7.25 (m, 3H), 7.21-7.18 (m, 1H), 5.25 (s, 1H), 2.31 (s, 3H). |
| 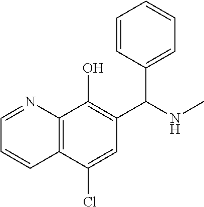 [SMILES: CNC(c1ccccc1)c1cc(Cl)c2cccnc2c1O] | A | 5-chloro-7- ((methylamino)(phenyl)methyl)quinolin-8-ol LC-MS: m/z 299 (M + H)⁺. 1H NMR (400 MHz, DMSO-d6) δ 8.85 (dd, J = 4.2, 1.6 Hz, 1H), 8.39-8.32 (m, 2H), 7.66 (s, 1H), 7.60 (dd, J = 8.5, 4.2 Hz, 1H), 7.41-7.36 (m, 2H), 7.23 (dd, J = 7.5, 7.5 Hz, 2H), 7.15-7.11 (m, 1H), 5.15 (s, 1H), 2.22 (s, 3H). |
| 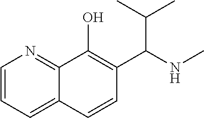 [SMILES: CNC(c1ccc2cccnc2c1O)C(C)C] | A | 7-(2-methyl-1-(methylamino)propyl)quinolin-8-ol LC-MS: m/z 231 (M + H)⁺. 1H NMR (400 MHz, DMSO-d₆) δ 8.83 (dd, J = 4.2, 1.6 Hz, 1H), 8.32-8.24 (m, 2H), 7.52 (dd, J = 8.3, 4.1 Hz, 1H), 7.46-7.34 (m, 2H), 3.93 (d, J = 6.7 Hz, 1H), 2.22 (s, 3H), 2.12-2.05 (m, 1H), 0.99 (d, J = 6.7 Hz, 3H), 0.80 (d, J = 6.8 Hz, 3H). |
| 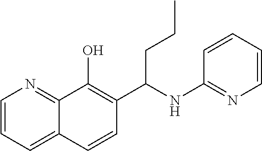 [SMILES: CCCC(Nc1ccccn1)c1ccc2cccnc2c1O] | A | 7-(1-(pyridin-2-ylamino)butyl)quinolin-8-ol LC-MS: m/z 294 (M + H)⁺. 1H NMR (400 MHz, DMSO-d6) δ (ppm): 9.90 (br s, 1H), 8.85-8.83 (m, 1H), 8.27-8.24 (m, 1H), 7.57 (d, J = 8.0 Hz, 1H), 7.36 (d, J = 8.0 Hz , 1H), 6.99 (d, J = 8.0 Hz, 1H), 6.50 (d, J = 8.0 Hz, 1H), 6.41- 6.38 (m, 2H), 5.41-5.35 (m, 1H), 1.81-1.77 (m, 2H), 1.53-1.26 (m, 2H), 0.92 (t, J = 8.0 Hz, 3H). |
| 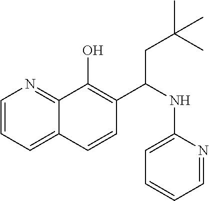 [SMILES: CC(C)(C)CC(Nc1ccccn1)c1ccc2cccnc2c1O] | A | 7-(3,3-dimethyl-1-(pyridin-2- ylamino)butyl)quinolin-8-ol LC-MS: m/z 322 (M + H)⁺. 1H NMR (400 MHz, DMSO-d6) δ (ppm): 10.00 (br s, 1H), 8.84-8.82 (m, 1H), 8.26 (d, J = 8.0 Hz, 1H), 7.63 (d, J = 8.0 Hz, 1H), 7.51 (t, J = 4.0 Hz, 1H), 7.36 (d, J = 8.0 Hz, 1H), 7.35-7.25 (m, 2H), 6.94 (d, J = 8.0 Hz, 1H), 6.51 (d, J = 8.0 Hz, 1H), 6.40-6.37 (m, 1H), 5.57-5.53 (m, 1H), 1.86-1.83 (m, 1H), 1.67-1.62 (m, 1H), 0.98 (s, 9H). |
| 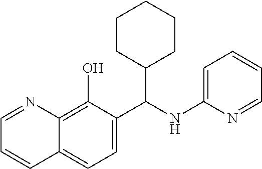 [SMILES: Oc1c(C(Nc2ccccn2)C2CCCCC2)ccc2cccnc12] | A | 7-(cyclohexyl(pyridin-2-ylamino)methyl)quinolin- 8-ol LC-MS: m/z 334 (M + H)⁺. 1H NMR (400 MHz, DMSO-d6) δ (ppm): 9.81 (br s, 1H), 8.84-8.83 (m, 1H), 8.27-8.24 (m, 1H), 7.87 (d, J = 4.0 Hz, 1H), 7.57-7.49 (m, 2H), 7.36 (d, J = 8.0 Hz, 1H), 7.35-7.25 (m, 2H), 7.30 (d, J = 8.0 Hz, 1H), 6.53 (d, J = 8.0 Hz, 1H), 6.39-6.36 (m, 1H), 5.28 (t, J = 8.0 Hz, 1H), 2.19-1.60 (m, 5H), 1.24-1.10 (m, 6H). |
| 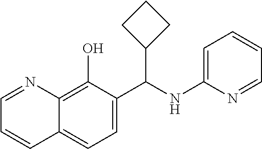 [SMILES: Oc1c(C(Nc2ccccn2)C2CCC2)ccc2cccnc12] | A | 7-(cyclobutyl(pyridin-2-ylamino)methyl)quinolin- 8-ol LC-MS: m/z 306 (M + H)⁺. 1H NMR (400 MHz, DMSO-d6) δ (ppm): 9.93 (br s, 1H), 8.85-8.83 (m, 1H), 8.27 (t, J = 4.0 Hz, 1H), 7.80 (d, J = 4.0 Hz, 1H), 7.58 (d, J = 8.0 Hz, 1H), 7.53 (t, J = 4.0 Hz, 1H), 7.49-7.35 (m, 2H), 7.10 (s, 1H), 6.53(d, J = 8.0 Hz, 1H), 6.43 (t, J = 4.0 Hz, 1H), 5.47 (t, J = 8.0 Hz, 1H), 1.76-1.61 (m, 3H), 0.95-0.92 (m, 6H). |
| 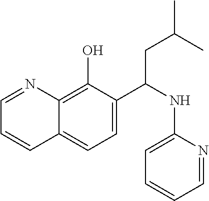 | A | 7-(2-methyl-1-(pyridin-2-ylamino)propyl)quinolin- 8-ol LC-MS: m/z 294 (M + H)⁺. 1H NMR (400 MHz, DMSO-d6) δ (ppm): 9.93 (br s, 1H), 8.85-8.83 (m, 1H), 8.27 (t, J = 4.0 Hz, 1H), 7.80 (d, J = 4.0 Hz, 1H), 7.58 (d, J = 8.0 Hz, 1H), 7.53 (t, J = 4.0 Hz, 1H), 7.49-7.35 (m, 2H), 7.10 (s, 1H), 6.53(d, J = 8.0 Hz, 1H), 6.43 (t, J = 4.0 Hz, 1H), 5.47 (t, J = 4.0 Hz, 1H), 1.76-1.61 (m, 3H), 0.95-0.92 (m, 6H). |
| 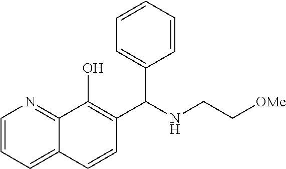 [SMILES: COCCNC(c1ccccc1)c1ccc2cccnc2c1O] | A | 7-(((2- methoxyethyl)amino)(phenyl)methyl)quinolin-8-ol LC-MS: m/z 309 (M + H)⁺. 1H NMR (400 MHz, DMSO-d6) δ (ppm): 8.83 (d, J = 2.8 Hz, 1H), 8.27 (t, J = 1.2 Hz, 1H), 7.60 (d, J = 8.0 Hz, 1H), 7.53-7.46 (m, 3H), 7.38-7.19 (m, 4H), 5.40 (s, 1H), 3.48-3.45 (m, 2H), 3.24 (s, 3H), 2.68-2.65 (m, 2H). |
| 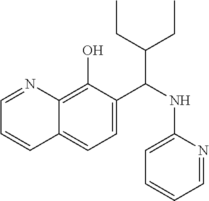 [SMILES: CCC(CC)C(Nc1ccccn1)c1ccc2cccnc2c1O] | A | 7-(2-ethyl-1-(pyridin-2-ylamino)butyl)quinolin-8- ol LC-MS: m/z 322 (M + H)⁺. 1H NMR (400 MHz, DMSO-d6) δ (ppm): 10.08 (br s, 1H), 8.84 (t, J = 1.6 Hz, 1H), 8.26 (t, J = 1.6 Hz, 1H), 7.86(d, J = 4.0 Hz, 1H), 7.59-7.49 (m, 2H), 7.36-7.28 (m, 2H), 6.85 (d, J = 9.2 Hz, 1H), 6.58 (d, J = 8.4 Hz, 1H), 6.39-6.36 (m, 1H), 5.49 (t, J = 8.0 Hz, 1H), 1.89-1.85 (m, 1H), 1.56-1.49 (m, 2H), 1.30-1.21 (m, 2H), 0.84-0.79 (m, 6H). |
| 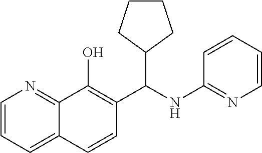 [SMILES: Oc1c(C(Nc2ccccn2)C2CCCC2)ccc2cccnc12] | A | 7-(cyclopentyl(pyridin-2-ylamino)methyl)quinolin- 8-ol LC-MS: m/z 320 (M + H)⁺. 1H NMR (400 MHz, DMSO-d6) δ (ppm): 9.18 (s, 1H), 8.90-8.89 (m, 1H), 8.36 (t, J = 8.0 Hz, 1H), 8.00 (d, J = 4.0 Hz, 1H), 7.90-7.80 (m, 1H), 7.60 (d, J = 8.0 Hz, 2H), 7.48 (d, J = 8.0 Hz, 1H), 6.99 (d, J = 8.0 Hz 1H), 6.83 (t, J = 8.0 Hz, 1H), 5.06 (s, 1H), 2.67-2.61 (m, 1H), 1.95-1.91 (m, 1H), 1.68- 1.23 (m, 7H). |
| 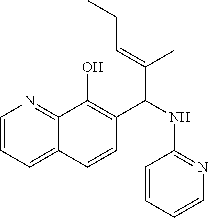 [SMILES: CC/C=C(\C)C(Nc1ccccn1)c1ccc2cccnc2c1O] | A | (Z)-7-(2-methyl-1-(pyridin-2-ylamino)pent-2-en-1- yl)quinolin-8-ol LC-MS: m/z 320 (M + H)⁺. 1H NMR (400 MHz, CD3OD) δ (ppm): 8.86-8.85 (m, 1H), 8.30 (t, J = 4.0 Hz, 1H), 7.93-7.85 (m, 2H), 7.60-7.42 (m, 3H), 7.13 (d, J = 8.0 Hz, 1H), 6.94 (t, J = 8.0 Hz, 1H), 5.82 (s, 1H), 5.61 (s, 1H), 2.30-2.10 (m, 2H), 1.68 (s, 3H), 1.0 (t, J = 8.0 Hz, 3H). |
| 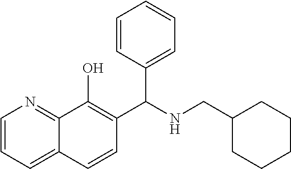 [SMILES: Oc1c(C(NCC2CCCCC2)c2ccccc2)ccc2cccnc12] | A | 7- (((cyclohexylmethyl)amino)(phenyl)methyl)quinolin- 8-ol LC-MS: m/z 347 (M + H)⁺. 1H NMR (400 MHz, CD3OD) δ (ppm): 9.03 (d, J = 4.4 Hz, 1H), 8.75 (d, J = 8.4 Hz, 1H), 7.88 (d, J = 4.8 Hz, 1H), 7.86-7.75 (m, 2H), 7.67 (d, J = 7.6 Hz, 2H), 7.51-7.44 (m, 3H), 7.30-7.19 (m, 5H), 6.18 (s, 1H), 3.30-3.25 (m, 2H), 3.14-3.10 (m, 2H). |
| 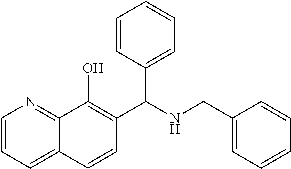 [SMILES: Oc1c(C(NCc2ccccc2)c2ccccc2)ccc2cccnc12] | B | 7-((benzylamino)(phenyl)methyl)quinolin-8-ol LC-MS: m/z 341 (M + H)⁺. 1H NMR (400 MHz, CD3OD) δ (ppm): 9.07-9.05 (m, 1H), 8.85-8.82 (m, 1H), 7.95-7.91 (m, 1H), 7.86-7.80 (m, 2H), 7.66 (d, J = 7.2 Hz, 2H), 7.52- 7.44 (m, 9H), 6.15 (s, 1H), 4.30 (d, J = 3.2 Hz, 2H). |
| 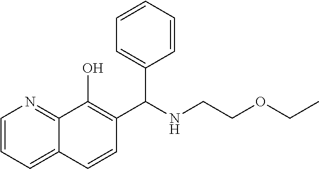 [SMILES: CCOCCNC(c1ccccc1)c1ccc2cccnc2c1O] | A | 7-(((2- ethoxyethyl)amino)(phenyl)methyl)quinolin-8-ol LC-MS: m/z 323 (M + H)⁺. 1H NMR (400 MHz, CD3OD) δ (ppm): 8.78 (d, J = 3.2 Hz, 1H), 8.20 (d, J = 7.6 Hz, 1H), 7.49-7.44 (m, 3H), 7.34-7.22 (m, 5H), 5.34 (s, 1H), 3.64-3.60 (m, 2H), 3.54-3.48 (m, 2H), 2.90-2.81 (m, 2H), 1.22-1.17 (m, 3H). |
| 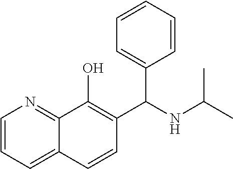 [SMILES: CC(C)NC(c1ccccc1)c1ccc2cccnc2c1O] | B | 7-((isopropylamino)(phenyl)methyl)quinolin-8-ol LC-MS: m/z 293 (M + H)⁺. 1H NMR (400 MHz, DMSO-d6) δ (ppm): 8.82 (d, J = 3.2 Hz, 1H), 8.31 (s, 1H), 8.25 (d, J = 8.0 Hz, 1H), 7.55-7.47 (m, 4H), 7.34-7.17 (m, 4H), 5.5 (s, 1H), 2.70-2.63 (m, 1H), 1.10-1.06 (m, 6H). |
| 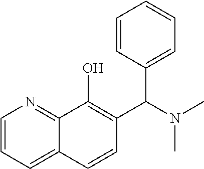 [SMILES: CN(C)C(c1ccccc1)c1ccc2cccnc2c1O] | B | 7-((dimethylamino)(phenyl)methyl)quinolin-8-ol LC-MS: m/z 279 (M + H)⁺. 1H NMR (400 MHz, CD3OD) δ (ppm): 9.01-9.00 (m, 1H), 8.65 (d, J = 8.4 Hz, 1H), 7.84-7.70 (m, 5H), 7.51-7.42 (m, 3H), 5.94 (s, 1H), 3.65 (s, 1H), 3.31-3.30 (s, 6H). |
| 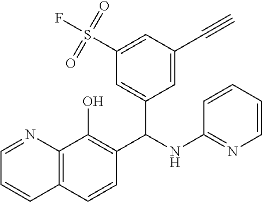 [SMILES: C#Cc1cc(C(Nc2ccccn2)c2ccc3cccnc3c2O)cc(S(=O)(=O)F)c1] | A | 3-ethynyl-5-((8-hydroxyquinolin-7-yl)(pyridin-2- ylamino)methyl)benzenesulfonyl fluoride LC-MS: m/z 434 (M + H)⁺. 1H NMR (400 MHz, DMSO-d6) δ 8.80 (d, J = 4.6 Hz, 1H), 8.25 (d, J = 8.4 Hz, 1H), 7.99 (s, 2H), 7.88 (s, 2H), 7.57-7.54 (m, 2H), 7.52-7.48 (m, 1H), 7.39-7.35 (m, 3H), 6.90 (d, J = 8.2 Hz, 1H), 6.67 (d, J = 8.4 Hz, 1H), 6.52-6.43 (m, 1H), 4.48 (s, 1H). |
| 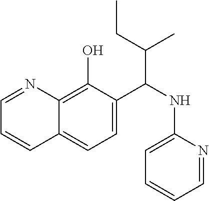 [SMILES: CCC(C)C(Nc1ccccn1)c1ccc2cccnc2c1O] | 2 | 7-(2-methyl-1-(pyridin-2-ylamino)butyl)quinolin- 8-ol LC-MS: m/z 308 (M + H)⁺. 1H NMR (400 MHz, DMSO-d6) δ (ppm): 10.00 (br s, 1H), 8.84 (d, J = 4.0 Hz, 1H), 8.27 (d, J = 8.4 Hz, 1H), 7.86(s, 1H), 7.59-7.49 (m, 2H), 7.36-7.28 (m, 2H), 6.93-6.84 (m, 1H), 6.59-6.52 (m, 1H), 6.40 (t, J = 4.8 Hz, 1H), 5.43-5.25 (m, 1H), 2.02- 1.95 (m, 2H), 1.70-1.30 (m, 2H), 0.96-0.75 (m, 6H). |
| 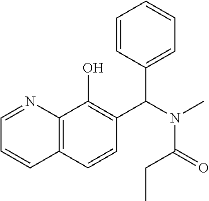 [SMILES: CCC(=O)N(C)C(c1ccccc1)c1ccc2cccnc2c1O] | 6 | N-((8-hydroxyquinolin-7-yl)(phenyl)methyl)-N- methylpropionamide LC-MS: m/z 321 (M + H)⁺. 1H NMR (400 MHz, DMSO-d6) mixture of rotamers δ 8.88 (dd, J = 10.6, 4.1 Hz, 1H), 8.36- 8.33 (m, 1H), 7.62-7.56 (m, 1H), 7.47-7.23 (m, 5H), 7.19-7.02 (m, 3H), 2.80 (s, 2H), 2.64 (s, 1H), 2.48-2.37 (m, 2H), 1.01 (dt, J = 14.5, 7.3 Hz, 3H). |
| 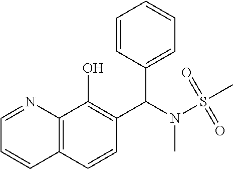 [SMILES: CN(C(c1ccccc1)c1ccc2cccnc2c1O)S(C)(=O)=O] | 6 | N-((8-hydroxyquinolin-7-yl)(phenyl)methyl)-N- methylmethanesulfonamide LC-MS: m/z 343 (M + H)⁺. 1H NMR (400 MHz, DMSO-d6) δ 8.90 (dd, J = 4.2, 1.6 Hz, 1H), 8.43 (s, 1H), 8.36 (d, J = 8.3 Hz, 1H), 7.61 (dd, J = 8.3, 4.2 Hz, 1H), 7.43-7.26 (m, 5H), 7.20 (d, J = 7.6 Hz, 2H), 7.14 (d, J = 8.6 Hz, 1H), 6.81 (s, 1H), 3.01 (s, 3H), 2.69 (s, 3H). |
| 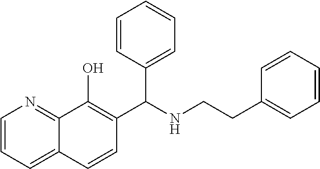 [SMILES: Oc1c(C(NCCc2ccccc2)c2ccccc2)ccc2cccnc12] | 1 | 7-((phenethylamino)(phenyl)methyl)quinolin-8-ol LC-MS: m/z 355 (M + H)⁺. 1H NMR (400 MHz, CD3OD) δ (ppm): 9.03 (d, J = 4.4 Hz, 1H), 8.75 (d, J = 8.4 Hz, 1H), 7.88-7.75 (m, 3H), 7.67 (d, J = 7.6 Hz, 2H), 7.51-7.44 (m, 3H), 7.30-7.19 (m, 5H), 6.18 (s, 1H), 3.30-3.25 (m, 2H), 3.14-3.10 (m, 2H). |
| 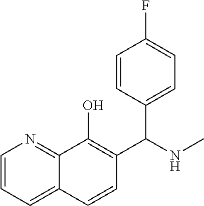 [SMILES: CNC(c1ccc(F)cc1)c1ccc2cccnc2c1O] | 1 | 7-((4-fluorophenyl)(methylamino)methyl)quinolin- 8-ol LC-MS: m/z 283 (M + H)⁺. 1H NMR (400 MHz, DMSO-d6) δ 8.82 (dd, J = 4.2, 1.7 Hz, 1H), 8.30-8.20 (m, 1H), 7.57 (d, J = 8.6 Hz, 1H), 7.52-7.46 (m, 3H), 7.36 (d, J = 8.5 Hz, 1H), 7.14-7.09 (m, 2H), 5.24 (s, 1H), 2.28 (s, 3H). |
| 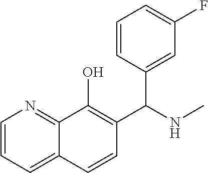 [SMILES: CNC(c1cccc(F)c1)c1ccc2cccnc2c1O] | 1 | 7-((3-fluorophenyl)(methylamino)methyl)quinolin- 8-ol LC-MS: m/z 283 (M + H)⁺. 1H NMR (400 MHz, CD3OD) δ (ppm): 9.21 (d, J = 4.8 Hz, 1H), 9.00 (d, J = 8.4 Hz, 1H), 8.05-8.01 (m, 1H), 7.91 (s, 2H), 7.54-7.42 (m, 3H), 7.22 (t, J = 8.4 Hz, 1H), 6.19 (s, 1H), 2.80 (s, 3H). |
| 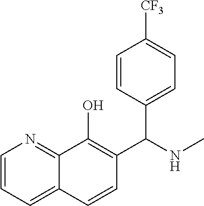 [SMILES: CNC(c1ccc(C(F)(F)F)cc1)c1ccc2cccnc2c1O] | 1 | 7-((methylamino)(4- (trifluoromethyl)phenyl)methyl)quinolin-8-ol LC-MS: m/z 333 (M + H)⁺. 1H NMR (400 MHz, CD3OD) δ (ppm): 8.81 (s, 1H), 8.25 (d, J = 8.4 Hz, 1H), 7.72-7.62 (m, 4H), 7.51-7.35 (m, 3H), 5.52 (s, 1H), 2.56 (s, 3H). |
| 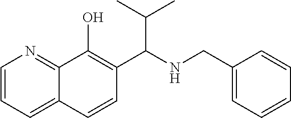 [SMILES: CC(C)C(NCc1ccccc1)c1ccc2cccnc2c1O] | 1 | 7-(1-(benzylamino)-2-methylpropyl)quinolin-8-ol LC-MS: m/z 307 (M + H)⁺. 1H NMR (400 MHz, CD3OD) δ (ppm): 8.78 (d, J = 4.0 Hz, 1H), 8.24 (d, J = 8.4 Hz, 1H), 7.48-7.45 (m, 1H), 7.33-7.19 (m, 7H), 3.83-3.77 (m, 2H), 3.59 (d, J = 13.2 Hz, 1H), 2.14-2.08 (m, 2H), 1.00 (d, J = 6.8 Hz, 3H), 0.85(d, J = 6.8 Hz, 3H). |
| 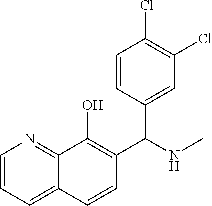 [SMILES: CNC(c1ccc(Cl)c(Cl)c1)c1ccc2cccnc2c1O] | 1 | 7-((3,4- dichlorophenyl)(methylamino)methyl)quinolin-8- ol LC-MS: m/z 333, 335 (M + H)⁺. 1H NMR (400 MHz, CD3OD) δ (ppm): 8.79 (d, J = 4.0 Hz, 1H), 8.22 (d, J = 8.4 Hz, 1H), 7.66 (s, 1H), 7.49-7.34 (m, 5H), 5.23 (s, 1H), 2.43 (s, 3H). |
| 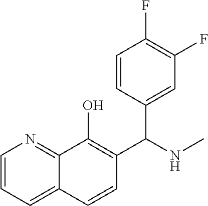 [SMILES: CNC(c1ccc(F)c(F)c1)c1ccc2cccnc2c1O] | 1 | 7-((3,4- difluorophenyl)(methylamino)methyl)quinolin-8-ol LC-MS: m/z 301 (M + H)⁺. 1H NMR (400 MHz, CD3OD) δ (ppm): 8.79 (d, J = 4.0 Hz, 1H), 8.22 (d, J = 8.4 Hz, 1H), 7.49-7.46 (m, 1H), 7.43-7.33 (m, 3H), 7.27-7.17 (m, 2H), 5.22 (s, 1H), 2.44 (s, 3H). |
| 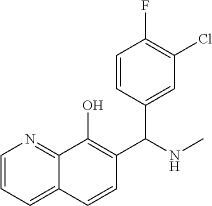 [SMILES: CNC(c1ccc(F)c(Cl)c1)c1ccc2cccnc2c1O] | 1 | 7-((3-chloro-4- fluorophenyl)(methylamino)methyl)quinolin-8-ol LC-MS: m/z 317 (M + H)⁺. 1H NMR (400 MHz, CD3OD) δ (ppm): 8.79 (d, J = 4.4 Hz, 1H), 8.22 (d, J = 8.4 Hz, 1H), 7.61(d, J = 6.8 Hz, 1H), 7.49-7.33 (m, 4H), 7.19 (t, J = 9.2 Hz, 1H), 5.22 (s, 1H), 2.43 (s, 3H). |
| 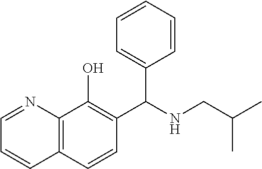 [SMILES: CC(C)CNC(c1ccccc1)c1ccc2cccnc2c1O] | 1 | 7-((isobutylamino)(phenyl)methyl)quinolin-8-ol LC-MS: m/z 307 (M + H)⁺. 1H NMR (400 MHz, DMSO-d6) δ 8.81 (dd, J = 4.1, 1.6 Hz, 1H), 8.24 (dd, J = 8.2, 1.7 Hz, 1H), 7.58-7.43 (m, 5H), 7.38-7.25 (m, 4H), 7.21- 7.17 (m, 1H), 5.32 (s, 1H), 2.36 (dd, J = 11.3, 6.4 Hz, 1H), 2.27 (dd, J = 11.3, 7.1 Hz, 1H), 1.77 (dt, J = 13.5, 6.7 Hz, 1H), 0.89 (dd, J = 8.5, 6.6 Hz, 6H). |
| 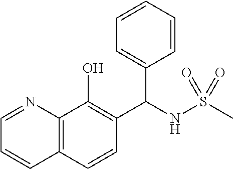 [SMILES: CS(=O)(=O)NC(c1ccccc1)c1ccc2cccnc2c1O] | 6 | N-((8-hydroxyquinolin-7- yl)(phenyl)methyl)methanesulfonamide LC-MS: m/z 329 (M + H)⁺. 1H NMR (400 MHz, DMSO-d6) δ 8.80 (dd, J = 4.2, 1.7 Hz, 1H), 8.24 (dd, J = 8.4, 1.7 Hz, 1H), 7.58 (d, J = 8.6 Hz, 1H), 7.49 (dd, J = 8.3, 4.2 Hz, 1H), 7.41-7.21 (m, 6H), 7.17-7.14 (m, 1H), 6.18 (s, 1H), 2.61 (s, 3H). |
| 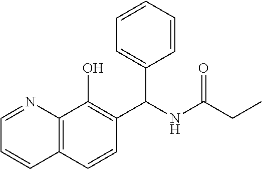 [SMILES: CCC(=O)NC(c1ccccc1)c1ccc2cccnc2c1O] | 6 | N-((8-hydroxyquinolin-7- yl)(phenyl)methyl)propionamide LC-MS: m/z 307 (M + H)⁺. 1H NMR (400 MHz, DMSO-d6) δ 9.95 (s, 1H), 8.89-8.82 (m, 1H), 8.70 (d, J = 8.8 Hz, 1H), 8.34 8.26 (m, 1H), 7.57-7.54 (m, 2H), 7.43 (d, J = 8.6 Hz, 1H), 7.32-7.19 (m, 5H), 6.72 (d, J = 8.7 Hz, 1H), 2.24 (qd, J = 7.5, 4.5 Hz, 2H), 1.02 (t, J = 7.6 Hz, 3H). |
| 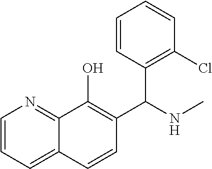 [SMILES: CNC(c1ccccc1Cl)c1ccc2cccnc2c1O] | 1 | 7-((2-chlorophenyl)(methylamino)methyl)quinolin- 8-ol LC-MS: m/z 299 (M + H)⁺. 1H NMR (400 MHz, CD3OD) δ (ppm): 9.06 (d, J = 4.8 Hz, 1H), 8.78 (d, J = 8.4 Hz, 1H), 7.92-7.86 (m, 2H), 7.72(d, J = 8.4 Hz, 1H), 7.59-7.46 (m, 4H), 6.42 (s, 1H), 2.83 (s, 3H). |
| 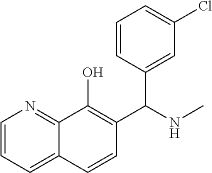 [SMILES: CNC(c1cccc(Cl)c1)c1ccc2cccnc2c1O] | 1 | 7-((3-chlorophenyl)(methylamino)methyl)quinolin- 8-ol LC-MS: m/z 299 (M + H)⁺. 1H NMR (400 MHz, CD3OD) δ (ppm): 8.91 (d, J = 4.0 Hz, 1H), 8.31 (d, J = 8.4 Hz, 1H), 7.7 0 (s, 1H), 7.61-7.56 (m, 2H), 7.49-7.44 (m, 4H), 5.85 (s, 1H), 2.76 (s, 3H). |
| 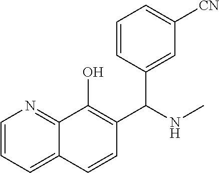 [SMILES: CNC(c1cccc(C#N)c1)c1ccc2cccnc2c1O] | 1 | 3-((8-hydroxyquinolin-7- yl)(methylamino)methyl)benzonitrile LC-MS: m/z 290 (M + H)⁺. 1H NMR (400 MHz, CD3OD) δ (ppm): 8.87 (d, J = 3.2 Hz, 1H), 8.28 (d, J = 8.4 Hz, 1H), 7.98 (s, 1H), 7.90 (d, J = 7.6 Hz, 1H), 7.73 (d, J = 7.6 Hz, 1H), 7.61-7.47 (m, 4H), 5.70 (s, 1H), 2.66 (s, 3H). |
| 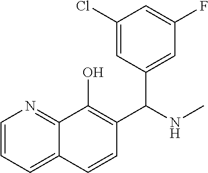 [SMILES: CNC(c1cc(F)cc(Cl)c1)c1ccc2cccnc2c1O] | 1 | 7-((3-chloro-5- fluorophenyl)(methylamino)methyl)quinolin-8-ol LC-MS: m/z 317 (M + H)⁺. 1H NMR (400 MHz, CD3OD) δ (ppm): 8.79 (s, 1H), 8.23 (d, J = 7.2 Hz, 1H), 7.47-7.28 (m, 6H), 5.27 (s, 1H), 2.45 (s, 3H). |
| 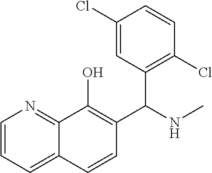 [SMILES: CNC(c1cc(Cl)ccc1Cl)c1ccc2cccnc2c1O] | 1 | 7-((2,5- dichlorophenyl)(methylamino)methyl)quinolin-8- ol LC-MS: m/z 333, 335 (M + H)⁺. 1H NMR (400 MHz, CD3OD) δ (ppm): 9.10 (d, J = 4.0 Hz, 1H), 8.96-9.86 (m, 1H), 7.97 (s, 2H), 7.79 (d, J = 8.4 Hz, 1H), 7.66 (d, J = 8.4 Hz, 1H), 7.56-7.50 (m, 2H), 6.40 (s, 1H), 2.84 (s, 3H). |
| 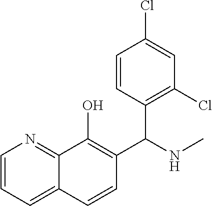 [SMILES: CNC(c1ccc(Cl)cc1Cl)c1ccc2cccnc2c1O] | 1 | 7-((2,4- dichlorophenyl)(methylamino)methyl)quinolin-8- ol LC-MS: m/z 333, 335 (M + H)⁺. 1H NMR (400 MHz, CD3OD) δ (ppm): 9.07 (d, J = 4.8 Hz, 1H), 8.82 (d, J = 8.0 Hz, 1H), 7.94 (d, J = 3.2 Hz, 1H), 7.87 (d, J = 8.4 Hz, 1H), 7.76 (d, J = 8.8 Hz, 1H), 7.65-7.58 (m, 3H), 6.39 (s, 1H), 2.83 (s, 3H). |
| 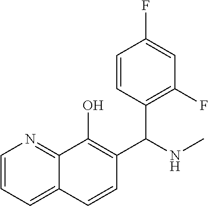 [SMILES: CNC(c1ccc(F)cc1F)c1ccc2cccnc2c1O] | 1 | 7-((2,4- difluorophenyl)(methylamino)methyl)quinolin-8-ol LC-MS: m/z 301 (M + H)⁺. 1H NMR (400 MHz, CD3OD) δ (ppm): 9.01 (d, J = 4.8 Hz, 1H), 8.64 (d, J = 8.4 Hz, 1H), 7.82-7.66 (m, 4H), 7.18-7.11 (m, 2H), 6.23 (s, 1H), 2.80 (s, 3H). |
| 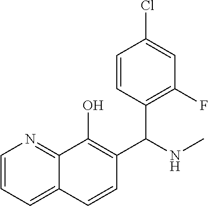 [SMILES: CNC(c1ccc(Cl)cc1F)c1ccc2cccnc2c1O] | 1 | 7-((4-chloro-2- fluorophenyl)(methylamino)methyl)quinolin-8-ol LC-MS: m/z 317 (M + H)⁺. 1H NMR (400 MHz, CD3OD) δ (ppm): 8.92 (d, J = 4.4 Hz, 1H), 8.36 (d, J = 8.0 Hz, 1H), 7.76-7.72 (m, 1H), 7.63-7.60 (m, 1H), 7.52 (s, 2H), 7.39 (t, J = 8.4 Hz, 2H), 6.14 (s, 1H), 2.79 (s, 3H). |
| 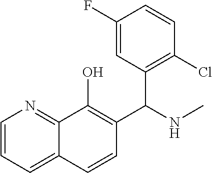 [SMILES: CNC(c1cc(F)ccc1Cl)c1ccc2cccnc2c1O] | 1 | 7-((2-chloro-5- fluorophenyl)(methylamino)methyl)quinolin-8-ol LC-MS: m/z 317 (M + H)⁺. 1H NMR (400 MHz, CD3OD) δ (ppm): 8.80 (s, 1H), 8.23 (d, J = 8.0 Hz, 1H), 7.48-7.23 (m 5H), 7.07 (t, J = 7.2 Hz, 1H), 5.65 (s, 1H), 2.50 (s, 3H). |
| 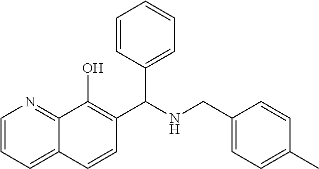 | 1 | 7-(((4- methylbenzyl)amino)(phenyl)methyl)quinolin-8-ol LC-MS: m/z 341 (M + H)⁺. 1H NMR (400 MHz, CD3OD) δ (ppm): 8.78 (s, 1H), 8.21 (s, 1H), 7.50-7.42 (m, 3H), 7.32-7.21 (m, 7H), 7.15 (d, J = 7.2 Hz, 2H), 5.25 (s, 1H), 3.85- 3.75 (m, 2H), 2.31 (s, 3H). |
| 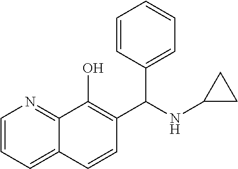 [SMILES: Oc1c(C(NC2CC2)c2ccccc2)ccc2cccnc12] | 1 | 7-((cyclopropylamino)(phenyl)methyl)quinol-8- ol LC-MS: m/z 291 (M + H)⁺. 1H NMR (400 MHz, CD3OD) δ (ppm): 8.78 (s, 1H), 8.20 (d, J = 8.0 Hz, 1H), 7.52-7.44 (m, 3H), 7.37-7.23 (m, 5H), 5.50 (s, 1H), 2.25 (d, J = 3.6 Hz, 1H), 0.59-0.48 (m, 4H). |
| 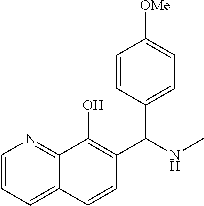 [SMILES: CNC(c1ccc(OC)cc1)c1ccc2cccnc2c1O] | 1 | 7-((4- methoxyphenyl)(methylamino)methyl)quinolin-8- ol LC-MS: m/z 295 (M + H)⁺. 1H NMR (400 MHz, DMSO-d6) δ 8.81 (dd, J = 4.2, 1.8 Hz, 1H), 8.24 (dd, J = 8.4, 1.7 Hz, 1H), 7.55-7.45 (m, 2H), 7.36-7.32 (m, 3H), 6.89- 6.80 (m, 2H), 5.15 (s, 1H), 3.69 (s, 3H), 2.29 (s, 3H). |
| 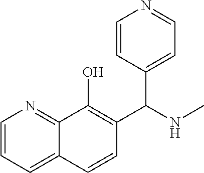 | 1 | 7-((methylamino)(pyridin-4-yl)methyl)quinolin-8- ol LC-MS: m/z 295 (M + H)⁺. 1H NMR (400 MHz, DMSO-d6) δ 8.84 (dd, J = 4.2, 1.7 Hz, 1H), 8.51-8.43 (m, 2H), 8.28 (dd, J = 8.3, 1.7 Hz, 1H), 8.20 (s, 1H), 7.60 (d, J = 8.5 Hz, 1H), 7.53 (dd, J = 8.3, 4.2 Hz, 1H), 7.48-7.43 (m, 2H), 7.39 (d, J = 8.5 Hz, 1H), 5.29 (s, 1H), 2.29 (s, 3H). |
| 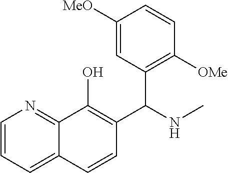 | 1 | 7-((2,5- dimethoxyphenyl)(methylamino)methyl)quinolin- 8-ol LC-MS: m/z 295 (M + H)⁺. 1H NMR (400 MHz, DMSO-d6) δ 8.74 (dd, J = 4.1, 1.8 Hz, 1H), 8.16 (dd, J = 8.2, 1.8 Hz, 1H), 7.42 (dd, J = 8.2, 4.1 Hz, 1H), 7.22-7.17 (m, 2H), 6.90-6.80 (m, 2H), 6.73 (dd, J = 8.8, 3.0 Hz, 1H), 5.35 (s, 1H), 3.65 (s, 3H), 3.58 (s, 3H), 2.25 (s, 3H). |
| 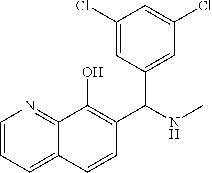 [SMILES: CNC(c1cc(Cl)cc(Cl)c1)c1ccc2cccnc2c1O] | 1 | 7-((3,5- dichlorophenyl)(methylamino)methyl)quinolin-8- ol LC-MS: m/z 333, 335 (M + H)⁺. 1H NMR (400 MHz, CD3OD) δ (ppm): 9.07 (d, J = 4.4 Hz, 1H), 8.80 (d, J = 8.4 Hz, 1H), 7.92-7.89 (m, 1H), 7.83-7.76 (m, 2H), 7.67 (s, 2H), 7.56 (s, 1H), 6.07(s, 1H), 2.79 (s, 3H). |
| 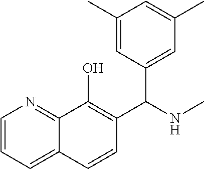 [SMILES: CNC(c1cc(C)cc(C)c1)c1ccc2cccnc2c1O] | 1 | 7-((3,5- dimethylphenyl)(methylamino)methyl)quinolin-8- ol LC-MS: m/z 293 (M + H)⁺. 1H NMR (400 MHz, CD3OD) δ (ppm): 9.06 (d, J = 4.8 Hz, 1H), 8.84 (d, J = 8.4 Hz, 1H), 7.94-7.91 (m, 1H), 7.82-7.75 (m, 2H), 7.21 (s, 2H), 7.09 (s, 1H), 5.97 (s, 1H), 2.75 (s, 3H), 2.32 (s, 3H), 2.31 (s, 3H) |
| 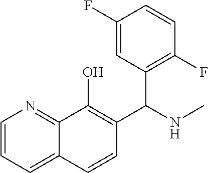 [SMILES: CNC(c1cc(F)ccc1F)c1ccc2cccnc2c1O] | 1 | 7-((2,5- difluorophenyl)(methylamino)methyl)quinolin-8-ol LC-MS: m/z 301 (M + H)⁺. 1H NMR (400 MHz, DMSO-d6) δ (ppm): 10.42 (br s, 2H), 8.96 (d, J = 4.0 Hz, 1H), 8.48 (d, J = 8.0 Hz, 1H), 7.96 (t, J = 5.6 Hz, 1H), 7.88 (d, J = 8.8 Hz, 1H), 7.71-7.68 (m, 1H), 7.57 (d, J = 8.8 Hz, 1H), 7.35-7.32 (m, 2H), 6.23 (s, 1H), 2.59 (s, 3H). |
| 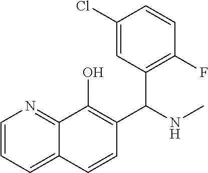 [SMILES: CNC(c1cc(Cl)ccc1F)c1ccc2cccnc2c1O] | 1 | 7-((5-chloro-2- fluorophenyl)(methylamino)methyl)quinolin-8-ol LC-MS: m/z 317 (M + H)⁺. 1H NMR (400 MHz, CD3OD) δ (ppm): 8.79 (s, 1H), 8.23 (d, J = 8.0 Hz, 1H), 7.57 (d, J = 4.8 Hz, 1H), 7.49 (d, J = 3.6 Hz, 1H), 7.36-7.28 (m, 3H), 7.13 (t, J = 9.2 Hz, 1H), 5.55(s, 1H) 2.03 (s, 3H). |
| 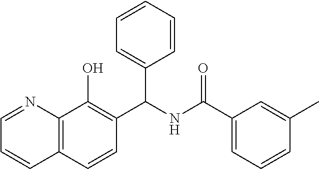 [SMILES: Cc1cccc(C(=O)NC(c2ccccc2)c2ccc3cccnc3c2O)c1] | 4 | N((8-hydroxyquinolin-7-yl)(phenyl)methyl)-3- methylbenzamide LC-MS: m/z 369 (M + H)⁺. 1H NMR (400 MHz, DMSO-d6) δ 10.06 (brs, 1H), 9.20 (d, J = 8.7 Hz, 1H), 8.87 (dd, J = 4.2, 1.8 Hz, 1H), 8.32 (dd, J = 8.3, 1.9 Hz, 1H), 7.79-7.65 (m, 3H), 7.56 (dd, J = 8.4, 4.1 Hz, 1H), 7.44 (d, J = 8.6 Hz, 1H), 7.40-7.29 (m, 6H), 7.29-7.18 (m, 1H), 6.99 (d, J = 8.6 Hz, 1H), 2.36 (s, 3H). |
| 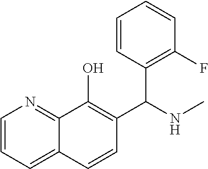 [SMILES: CNC(c1ccccc1F)c1ccc2cccnc2c1O] | 1 | 7-((2-fluorophenyl)(methylamino)methyl)quinolin- 8-ol LC-MS: m/z 283 (M + H)⁺. 1H NMR (400 MHz, CD3OD) δ (ppm): 9.12 (d, J = 4.8 Hz, 1H), 8.98 (d, J = 8.4 Hz, 1H), 8.04-8.01 (m, 1H), 7.90-7.84 (m, 2H), 7.77 (t, J = 8.0 Hz, 1H), 7.574-7.49 (m, 1H), 7.38-7.24 (m, 2H), 6.40 (s, 1H), 2.82 (s, 3H). |
| 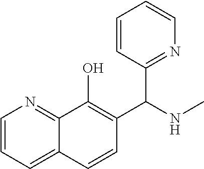 [SMILES: CNC(c1ccccn1)c1ccc2cccnc2c1O] | 1 | 7-((methylamino)(pyridin-2-yl)methyl)quinolin-8- ol LC-MS: m/z 266 (M + H)⁺. 1H NMR (400 MHz, CD3OD) δ (ppm): 9.20-9.15 (m, 2H), 8.82 (d, J = 4.8 Hz, 1H), 8.18-8.15 (m, 1H), 8.01 (t, J = 8.0 Hz, 1H), 7.92 (s, 2H), 7.69 (d, J = 7.6 Hz, 1H), 7.60 (t, J = 6.0 Hz, 1H), 6.31 (s, 1H), 2.74 (s, 3H). |
| 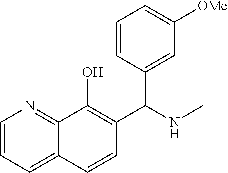 [SMILES: CNC(c1cccc(OC)c1)c1ccc2cccnc2c1O] | 1 | 7-((3- methoxyphenyl)(methylamino)methyl)quinolin-8- ol LC-MS: m/z 295 (M + H)⁺. 1H NMR (400 MHz, CD3OD) δ (ppm): 8.92 (d, J = 4.4 Hz, 1H), 8.36 (d, J = 8.4 Hz, 1H), 7.63-7.61 (m, 1H), 7.50 (s, 2H), 7.40 (t, J = 8.0 Hz, 1H), 7.22-7.16 (m, 2H), 6.99 (d, J = 8.0 Hz, 1H), 5.84 (s, 1H), 3.81 (s, 3H), 2.75 (s, 3H). |
| 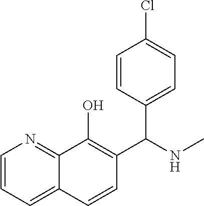 [SMILES: CNC(c1ccc(Cl)cc1)c1ccc2cccnc2c1O] | 1 | 7-((4-chlorophenyl)(methylamino)methyl)quinolin- 8-ol LC-MS: m/z 299 (M + H)⁺. 1H NMR (400 MHz, CD3OD) δ (ppm): 8.88 (d, J = 3.2 Hz, 1H), 8.28 (d, J = 8.4 Hz, 1H), 7.61-7.54 (m, 3H), 7.46-7.42 (m, 4H), 5.75 (s, 1H), 2.71 (s, 3H) |
| 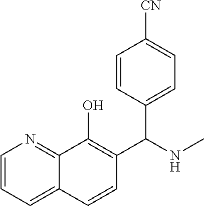 [SMILES: CNC(c1ccc(C#N)cc1)c1ccc2cccnc2c1O] | 1 | 4-((8-hydroxyquinolin-7- yl)(methylamino)methyl)benzonitrile LC-MS: m/z 290 (M + H)⁺. 1H NMR (400 MHz, CD3OD) δ (ppm): 9.09 (d, J = 4.4 Hz, 1H), 8.89 (d, J = 8.0 Hz, 1H), 7.98 (t, J = 6.8 Hz, 1H), 7.87-7.84 (m, 6H), 6.25 (s, 1H), 2.80 (s, 3H) |
| 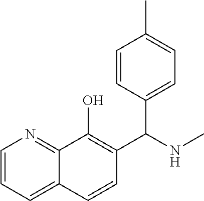 [SMILES: CNC(c1ccc(C)cc1)c1ccc2cccnc2c1O] | 1 | 7-((methylamino)(p-tolyl)methyl)quinolin-8-ol LC-MS: m/z 279 (M + H)⁺. 1H NMR (400 MHz, CD3OD) δ (ppm): 9.07 (d, J = 5.2 Hz, 1H), 8.86 (d, J = 8.4 Hz, 1H), 7.95 (t, J = 6.8 Hz, 1H), 7.83-7.78 (m, 2H), 7.50 (d, J = 7.2 Hz, 2H), 7.31 (d, J = 8.0 Hz, 2H), 6.02 (s, 1H), 2.76 (s, 3H), 2.35 (s, 3H) |
| 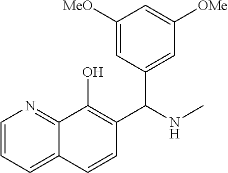 [SMILES: CNC(c1cc(OC)cc(OC)c1)c1ccc2cccnc2c1O] | 1 | 7-((3,5- dimethoxyphenyl)(methylamino)methyl)quinolin- 8-ol LC-MS: m/z 325 (M + H)⁺. 1H NMR (400 MHz, CD3OD) δ (ppm): 9.09 (d, J = 4.8 Hz, 1H), 8.89 (d, J = 8.4 Hz, 1H), 7.98-7.95 (m, 1H), 7.84 (s, 2H), 6.80 (s, 2H), 6.54 (s, 1H), 6.02 (s, 1H), 3.80 (s, 6H), 2.78 (s, 3H) |
| 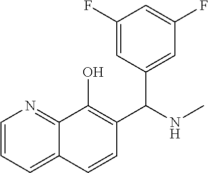 [SMILES: CNC(c1cc(F)cc(F)c1)c1ccc2cccnc2c1O] | 1 | 7-((3,5- difluorophenyl)(methylamino)methyl)quinolin-8-ol LC-MS: m/z 301 (M + H)⁺. 1H NMR (400 MHz, CD3OD) δ (ppm): 9.11 (d, J = 4.4 Hz, 1H), 8.93 (d, J = 8.4 Hz, 1H), 8.01-7.98 (m, 1H), 7.90-7.85 (m, 2H), 7.33 (d, J = 7.2 Hz, 2H), 7.11-7.07 (m, 1H), 6.17 (s, 1H), 2.80 (s, 3H) |
| 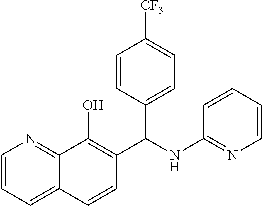 [SMILES: Oc1c(C(Nc2ccccn2)c2ccc(C(F)(F)F)cc2)ccc2cccnc12] | 2 | 7-((pyridin-2-ylamino)(4- (trifluoromethyl)phenyl)methyl)quinolin-8-ol LC-MS: m/z 396 (M + H)⁺. 1H NMR (400 MHz, DMSO-d6) δ (ppm): 10.04 (br s, 1H), 8.87 (t, J = 2.0 Hz, 1H), 8.31 (d, J = 8.0 Hz, 1H), 7.93 (d, J = 4.8 Hz, 1H), 7.67 (d, J = 6.4 Hz, 2H), 7.59-7.53 (m, 4H), 7.48-7.38 (m, 3H), 6.96 (d, J = 8.4 Hz, 1H), 6.72 (d, J = 8.4 Hz, 1H), 6.51 (t, J = 6.0 Hz, 1H) |
| 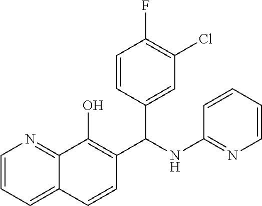 [SMILES: Oc1c(C(Nc2ccccn2)c2ccc(F)c(Cl)c2)ccc2cccnc12] | 2 | 7-((3-chloro-4-fluorophenyl)(pyridin-2- ylamino)methyl)quinolin-8-ol LC-MS: m/z 380 (M + H)⁺. 1H NMR (400 MHz, DMSO-d6) δ (ppm): 10.05 (br s, 1H), 8.86 (t, J = 2.0 Hz, 1H), 8.31 (d, J = 8.4 Hz, 1H), 7.93 (d, J = 4.8 Hz, 1H), 7.61-7.33 (m, 8H), 6.86 (d, J = 8.0 Hz, 1H), 6.70 (d, J = 8.4 Hz, 1H), 6.51 (t, 5.6 Hz, 1H) |
| 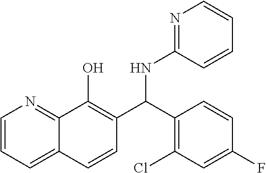 [SMILES: Oc1c(C(Nc2ccccn2)c2ccc(F)cc2Cl)ccc2cccnc12] | 2 | 7-((2-chloro-4-fluorophenyl)(pyridin-2- ylamino)methyl)quinolin-8-ol LC-MS: m/z 380 (M + H)⁺. 1H NMR (400 MHz, DMSO-d6) δ (ppm): 9.95 (br s, 1H), 8.84 (d, J = 3.6 Hz, 1H), 8.46 (d, J = 8.4 Hz, 1H), 8.01-7.98 (m, 2H), 7.68-7.65 (m, 1H), 7.51- 7.22 (m, 5H), 7.16 (t, J = 8.0 Hz, 1H), 6.98 (t, J = 6.4 Hz, 1H), 6.80 (d, J = 4.0 Hz, 1H) |
| 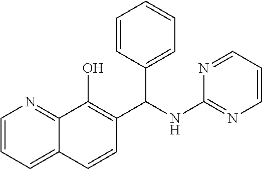 [SMILES: Oc1c(C(Nc2ncccn2)c2ccccc2)ccc2cccnc12] | 3 | 7-(phenyl(pyrimidin-2-ylamino)methyl)quinolin-8- ol LC-MS: m/z 329 (M + H)⁺. 1H NMR (400 MHz, DMSO-d6) δ (ppm): 9.92 (br s, 1H), 8.84 (s, 1H), 8.33-8.28 (m, 3H), 8.08 (d, J = 8.8 Hz, 1H), 7.75 (d, J = 8.0 Hz, 1H), 7.55-7.52 (m, 1H), 7.40-7.18 (m, 6H), 6.99 (d, J = 9.2 Hz, 1H), 6.60 (t, 4.4 Hz, 1H) |
| 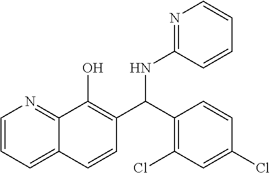 [SMILES: Oc1c(C(Nc2ccccn2)c2ccc(Cl)cc2Cl)ccc2cccnc12] | 2 | 7-((2,4-dichlorophenyl)(pyridin-2- ylamino)methyl)quinolin-8-ol LC-MS: m/z 396, 398 (M + H)⁺. 1H NMR (400 MHz, DMSO-d6) δ (ppm): 9.43 (br s, 1H), 8.93 (d, J = 4.0 Hz, 1H), 8.43 (d, J = 8.4 Hz, 1H), 8.01-7.95 (m, 2H), 7.66-7.47 (m, 4H), 7.38 (s, 1H), 7.25 (d, J = 8.4 Hz, 1H), 7.17-7.17 (m, 1H), 6.99 (t, 6.4 Hz, 1H), 6.74 (d, J = 7.6 Hz, 1H) |
| 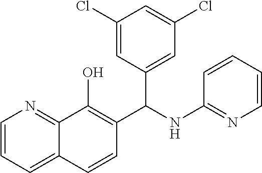 [SMILES: Oc1c(C(Nc2ccccn2)c2cc(Cl)cc(Cl)c2)ccc2cccnc12] | 2 | 7-((3,5-dichlorophenyl)(pyridin-2- ylamino)methyl)quinolin-8-ol LC-MS: m/z 396, 398 (M + H)⁺. 1H NMR (400 MHz, DMSO-d6) δ (ppm): 9.69 (s, 1H), 8.95 (d, J = 3.8 Hz, 1H), 8.48 (d, J = 8.3 Hz, 1H), 8.09-7.96 (m, 2H), 7.69 (dd, J = 7.8, 4.1 Hz, 1H), 7.60 (s, 1H), 7.54 (d, J = 11.2 Hz, 4H), 7.27 (d, J = 8.8 Hz, 1H), 6.97 (t, J = 6.5 Hz, 1H), 6.82 (d, J = 7.3 Hz, 1H). |
| 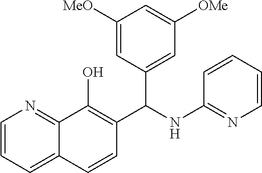 [SMILES: COc1cc(OC)cc(C(Nc2ccccn2)c2ccc3cccnc3c2O)c1] | 2 | 7-((3,5-dimethoxyphenyl)(pyridin-2- ylamino)methyl)quinolin-8-ol LC-MS: m/z 388 (M + H)+. 1H NMR (400 MHz, DMSO-d6) δ (ppm): 9.78 (s, 1H), 8.99 (d, J = 4.1 Hz, 1H), 8.61 (d, J = 8.1 Hz, 1H), 8.08-8.06 (s, 1H), 8.00 (t, J = 8.0 Hz, 1H), 7.82-7.71 (m, 1H), 7.60 (s, 2H), 7.29 (d, J = 7.5 Hz, 1H), 6.95 (t, J = 6.5 Hz, 1H), 6.80 (d, J = 6.5 Hz, 1H), 6.69 (s, 2H), 6.47 (s, 1H), 3.73 (s, 6H). |
| 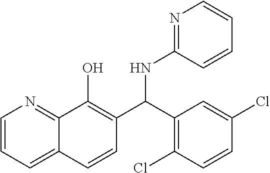 [SMILES: Oc1c(C(Nc2ccccn2)c2cc(Cl)ccc2Cl)ccc2cccnc12] | 2 | 7-((2,5-dichlorophenyl)(pyridin-2- ylamino)methyl)quinolin-8-ol LC-MS: m/z 396, 398 (M + H)⁺. 1H NMR (400 MHz, DMSO-d6) δ (ppm): 9.26 (br s, 1H), 8.94-8.85 (m, 1H), 8.39 (d, J = 8.2 Hz, 1H), 8.05-7.85 (m, 2H), 7.69-7.56 (m, 2H), 7.53-7.42 (m, 2H), 7.38 (s, 1H), 7.23 (d, J = 8.3 Hz, 1H), 7.12 (d, J = 7.0 Hz, 1H), 6.98-6.87 (m, 1H), 6.74 (d, J = 7.0 Hz, 1H) |
| 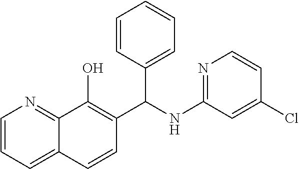 [SMILES: Oc1c(C(Nc2cc(Cl)ccn2)c2ccccc2)ccc2cccnc12] | 2 | 7-(((4-chloropyridin-2- yl)amino)(phenyl)methyl)quinolin-8-ol LC-MS: m/z 362 (M + H)⁺. 1H NMR (400 MHz, DMSO-d6) δ (ppm): 9.96 (br s, 1H), 8.85 (s, 1H), 8.29 (d, J = 8.1 Hz, 1H), 7.89 (d, J = 5.3 Hz, 1H), 7.69 (d, J = 8.1 Hz, 1H), 7.60- 7.49 (m, 2H), 7.40 (d, J = 8.4 Hz, 1H), 7.37-7.25 (m, 4H), 7.24-7.15 (m, 1H), 6.85 (d, J = 8.0 Hz, 1H), 6.76 (s, 1H), 6.55 (d, J = 5.0 Hz, 1H). |
| 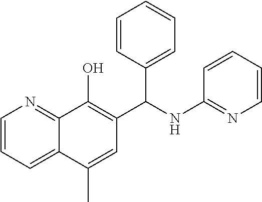 [SMILES: Cc1cc(C(Nc2ccccn2)c2ccccc2)c(O)c2ncccc12] | 2 | 5-methyl-7-(phenyl(pyridin-2- ylamino)methyl)quinolin-8-ol LC-MS: m/z 342 (M + H)⁺. 1H NMR (400 MHz, CD3OD) δ (ppm): 8.86 (s, 1H), 8.43 (d, J = 8.6 Hz, 1H), 7.92 (t, J = 8.0 Hz, 1H), 7.87 (d, J = 5.9 Hz, 1H), 7.59 (dd, J = 7.7, 3.8 Hz, 1H), 7.48-7.38 (m, 4H), 7.35 (d, J = 6.8 Hz, 1H), 7.20-7.09 (m, 2H), 6.92 (t, J = 6.6 Hz, 1H), 6.61 (s, 1H), 2.54 (s, 3H). |
| 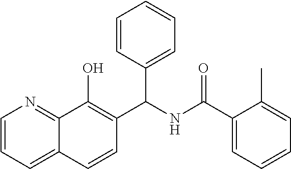 [SMILES: Cc1ccccc1C(=O)NC(c1ccccc1)c1ccc2cccnc2c1O] | 4 | N-((8-hydroxyquinolin-7-yl)(phenyl)methyl)-2- methylbenzamide LC-MS: m/z 369 (M + H)⁺. 1H NMR (400 MHz, DMSO-d6) δ (ppm): 10.03 (br s, 1H), 9.21 (d, J = 9.0 Hz, 1H), 8.87 (dd, J = 4.1, 1.5 Hz, 1H), 8.32 (dd, J = 8.3, 1.5 Hz, 1H), 7.67 (d, J = 8.6 Hz, 1H), 7.56 (dd, J = 8.3, 4.2 Hz, 1H), 7.43 (d, J = 8.6 Hz, 1H), 7.39-7.28 (m, 6H), 7.27-7.18 (m, 3H), 6.97 (d, J = 9.0 Hz, 1H), 2.28 (s, 3H). |
| 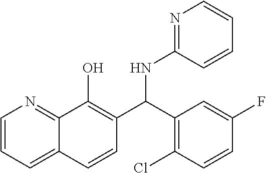 [SMILES: Oc1c(C(Nc2ccccn2)c2cc(F)ccc2Cl)ccc2cccnc12] | 2 | 7-((2-chloro-5-fluorophenyl)(pyridin-2- ylamino)methyl)quinolin-8-ol LC-MS: m/z 380 (M + H)⁺. 1H NMR (400 MHz, DMSO-d6) δ (ppm): 9.62 (s, 1H), 8.95 (d, J = 3.7 Hz, 1H), 8.50 (d, J = 7.4 Hz, 1H), 8.11-7.95 (m, 2H), 7.74-7.66 (m, 1H), 7.65- 7.57 (m, 1H), 7.51 (d, J = 8.6 Hz, 1H), 7.40-7.12 (m, 4H), 6.99 (t, J = 6.6 Hz, 1H), 6.75 (d, J = 6.7 Hz, 1H). |
| 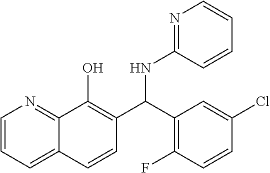 [SMILES: Oc1c(C(Nc2ccccn2)c2cc(Cl)ccc2F)ccc2cccnc12] | 2 | 7-((5-chloro-2-fluorophenyl)(pyridin-2- ylamino)methyl)quinolin-8-ol LC-MS: m/z 380 (M + H)⁺. 1H NMR (400 MHz, DMSO-d6) δ (ppm): 9.36 (s, 1H), 8.91 (d, J = 2.8 Hz, 1H), 8.39 (d, J = 8.3 Hz, 1H), 8.06-7.88 (m, 2H), 7.63 (dd, J = 8.1, 4.1 Hz, 1H), 7.54-7.41 (m, 3H), 7.40-7.28 (m, 2H), 7.20 (d, J = 8.4 Hz, 1H), 6.94 (t, J = 6.2 Hz, 1H), 6.82 (d, J = 6.7 Hz, 1H). |
| 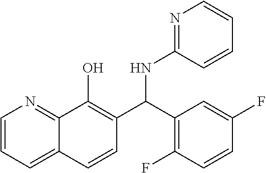 [SMILES: Oc1c(C(Nc2ccccn2)c2cc(F)ccc2F)ccc2cccnc12] | 2 | 7-((2,5-difluorophenyl)(pyridin-2- ylamino)methyl)quinolin-8-ol LC-MS: m/z 364 (M + H)⁺. 1H NMR (400 MHz, DMSO-d6) δ (ppm): 9.61 (s, 1H), 8.95 (d, J = 3.9 Hz, 1H), 8.49 (d, J = 8.2 Hz, 1H), 8.10-7.91 (m, 2H), 7.75-7.64 (m, 1H), 7.52 (d, J = 8.5 Hz, 1H), 7.46-7.21 (m, 5H), 6.98 (t, J = 6.4 Hz, 1H), 6.86 (d, J = 7.0 Hz, 1H). |
| 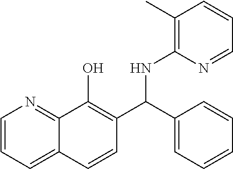 [SMILES: Cc1cccnc1NC(c1ccccc1)c1ccc2cccnc2c1O] | 2 | 7-(((3-methylpyridin-2- yl)amino)(phenyl)methyl)quinolin-8-ol LC-MS: m/z 342 (M + H)⁺. 1H NMR (400 MHz, DMSO-d6) δ (ppm): 10.02 (br s, 1H), 8.84 (d, J = 1.6 Hz, 1H), 8.29 (d, J = 8.2 Hz, 1H), 7.82 (d, J = 4.6 Hz, 1H), 7.72 (d, J = 8.5 Hz, 1H), 7.53 (dd, J = 7.7, 3.7 Hz, 1H), 7.42-7.33 (m, 3H), 7.28-7.25 (m, 3H), 7.19-7.15 (m, 1H), 7.04 (d, J = 8.4 Hz, 1H), 6.47 (t, J = 5.7 Hz, 1H), 6.39 (d, J = 7.6 Hz, 1H), 2.20 (s, 3H). |
| 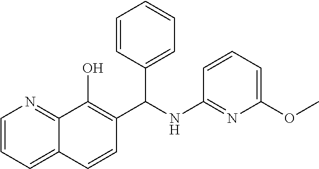 [SMILES: COc1cccc(NC(c2ccccc2)c2ccc3cccnc3c2O)n1] | 2 | 7-(((6-methoxypyridin-2- yl)amino)(phenyl)methyl)quinolin-8-ol LC-MS: m/z 358 (M + H)⁺. 1H NMR (400 MHz, DMSO-d6) δ (ppm): 9.91 (s, 1H), 8.84 (dd, J = 4.2, 1.6 Hz, 1H), 8.33-8.21 (m, 1H), 7.64 (d, J = 8.5 Hz, 1H), 7.53 (dd, J = 8.3, 4.2 Hz, 1H), 7.44-7.34 (m, 4H), 7.33-7.24 (m, 3H), 7.20 (t, J = 7.3 Hz, 1H), 6.80 (d, J = 8.1 Hz, 1H), 6.21 (d, J = 7.9 Hz, 1H), 5.84 (d, J = 7.7 Hz, 1H), 3.61 (s, 3H). |
| 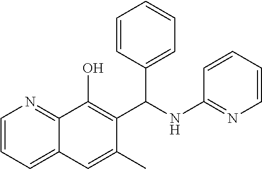 [SMILES: Cc1cc2cccnc2c(O)c1C(Nc1ccccn1)c1ccccc1] | 2 | 6-methyl-7-(phenyl(pyridin-2- ylamino)methyl)quinolin-8-ol LC-MS: m/z 342 (M + H)⁺. 1H NMR (400 MHz, DMSO-d6) δ 9.84 (s, 1H), 8.70 (dd, J = 4.1, 1.6 Hz, 1H), 8.15 (dd, J = 8.3, 1.6 Hz, 1H), 7.89 (dd, J = 5.0, 1.8 Hz, 1H), 7.44 (dd, J = 8.3, 4.2 Hz, 1H), 7.33-7.29 (m, 1H), 7.19- 7.18 (m, 5H), 7.13-7.08 (m, 1H), 6.94 (s, 2H), 6.71 (d, J = 8.4 Hz, 1H), 6.42 (dd, J = 6.9, 5.1 Hz, 1H), 2.38 (s, 3H) |
| 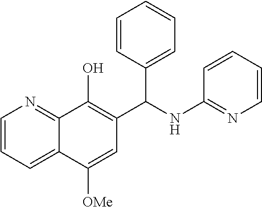 [SMILES: COc1cc(C(Nc2ccccn2)c2ccccc2)c(O)c2ncccc12] | 2 | 5-methoxy-7-(phenyl(pyridin-2- ylamino)methyl)quinolin-8-ol LC-MS: m/z 358 (M + H)⁺. 1H NMR (400 MHz, DMSO-d6) δ 9.37 (brs, 1H), 8.87 (dd, J = 4.2, 1.7 Hz, 1H), 8.44 (dd, J = 8.4, 1.7 Hz, 1H), 7.94 (dd, J = 5.1, 1.9 Hz, 1H), 7.52 (dd, J = 8.5, 4.2 Hz, 1H), 7.47-7.33 (m, 4H), 7.31- 7.27 (m, 2H), 7.23-7.12 (m, 2H), 6.91 (d, J = 9.0 Hz, 1H), 6.68 (d, J = 8.4 Hz, 1H), 6.52-6.44 (m, 1H), 3.87 (s, 3H). |
| 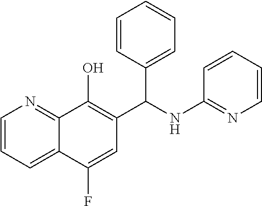 [SMILES: Oc1c(C(Nc2ccccn2)c2ccccc2)cc(F)c2cccnc12] | 2 | 5-fluoro-7-(phenyl(pyridin-2- ylamino)methyl)quinolin-8-ol LC-MS: m/z 346 (M + H)⁺. 1H NMR (400 MHz, DMSO-d6) δ 9.97 (brs, 1H), 8.94 (dd, J = 4.2, 1.6 Hz, 1H), 8.40 (dd, J = 8.4, 1.7 Hz, 1H), 7.95-7.91 (m, 1H), 7.64 (dd, J = 8.5, 4.2 Hz, 1H), 7.53-7.25 (m, 7H), 7.25-7.15 (m, 1H), 6.88 (d, J = 8.6 Hz, 1H), 6.68 (d, J = 8.4 Hz, 1H), 6.49 (dd, J = 6.9, 5.1 Hz, 1H). |
| 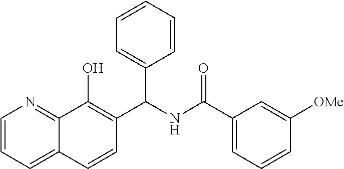 [SMILES: COc1cccc(C(=O)NC(c2ccccc2)c2ccc3cccnc3c2O)c1] | 4 | N-((8-hydroxyquinolin-7-yl)(phenyl)methyl)-3- methoxybenzamide LC-MS: m/z 385 (M + H)⁺. 1H NMR (400 MHz, DMSO-d6) δ (ppm): 10.01 (br s, 1H), 9.22 (d, J = 8.7 Hz, 1H), 8.87 (dd, J = 4.2, 1.6 Hz, 1H), 8.32 (dd, J = 8.3, 1.6 Hz, 1H), 7.69 (d, J = 8.6 Hz, 1H), 7.61-7.50 (m, 2H), 7.48- 7.36 (m, 3H), 7.34-7.28 (m, 4H), 7.27-7.20 (m, 1H), 7.11 (dd, J = 7.9, 2.2 Hz, 1H), 6.99 (d, J = 8.7 Hz, 1H), 3.80 (s, 3H). |
| 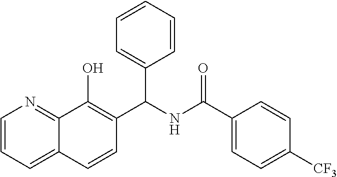 [SMILES: O=C(NC(c1ccccc1)c1ccc2cccnc2c1O)c1ccc(C(F)(F)F)cc1] | 4 | N-((8-hydroxyquinolin-7-yl)(phenyl)methyl)-4- (trifluoromethyl)benzamide LC-MS: m/z 423 (M + H)⁺. 1H NMR (400 MHz, DMSO-d6) δ (ppm): 10.08 (br s, 1H), 9.48 (d, J = 8.6 Hz, 1H), 8.87 (dd, J = 4.2, 1.6 Hz, 1H), 8.32 (dd, J = 8.4, 1.6 Hz, 1H), 8.12 (d, J = 8.1 Hz, 2H), 7.86 (d, J = 8.3 Hz, 2H), 7.66 (d, J = 8.6 Hz, 1H), 7.57 (dd, J = 8.2, 4.2 Hz, 1H), 7.44 (d, J = 8.6 Hz, 1H), 7.34-7.33 (m, 4H), 7.29-7.20 (m, 1H), 7.00 (d, J = 8.6 Hz, 1H). |
| 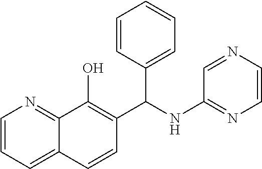 [SMILES: Oc1c(C(Nc2cnccn2)c2ccccc2)ccc2cccnc12] | 3 | 7-(phenyl(pyrazin-2-ylamino)methyl)quinolin-8-ol LC-MS: m/z 329 (M + H)⁺. 1H NMR (400 MHz, DMSO) δ 10.01 (br s, 1H), 8.90-8.82 (m, 1H), 8.34-8.25 (m, 1H), 8.14 (d, J = 1.4 Hz, 1H), 7.95 (d, J = 8.1 Hz, 1H), 7.88 (dd, J = 2.8, 1.4 Hz, 1H), 7.66 (d, J = 2.8 Hz, 1H), 7.59- 7.50 (m, 2H), 7.45-7.38 (m, 1H), 7.37-7.27 (m, 4H), 7.25-7.16 (m, 1H), 6.80 (d, J = 8.1 Hz, 1H). |
| 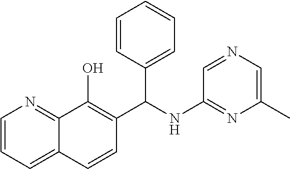 [SMILES: Cc1cncc(NC(c2ccccc2)c2ccc3cccnc3c2O)n1] | 3 | 7-(((6-methylpyrazin-2- yl)amino)(phenyl)methyl)quinolin-8-ol LC-MS: m/z 343 (M + H)⁺. 1H NMR (400 MHz, CD3OD) δ(ppm): 8.80 (dd, J = 4.2, 1.5 Hz, 1H), 8.21 (dd, J = 8.3, 1.5 Hz, 1H), 7.78 (s, 1H), 7.54 (s, 1H), 7.51-7.43 (m, 2H), 7.40 (d, J = 7.3 Hz, 2H), 7.35 (d, J = 8.6 Hz, 1H), 7.32- 7.25 (m, 2H), 7.25-7.18 (m, 1H), 6.79 (s, 1H), 2.27 (s, 3H) |
| 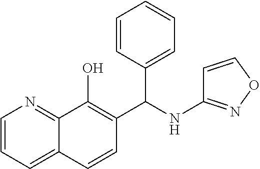 [SMILES: Oc1c(C(Nc2ccon2)c2ccccc2)ccc2cccnc12] | 3 | 7-((isoxazol-3-ylamino)(phenyl)methyl)quinolin-8- ol LC-MS: m/z 318 (M + H)⁺. 1H NMR (400 MHz, DMSO-d6) δ (ppm): 10.00 (br s, 1H), 8.89-8.81 (m, 1H), 8.34 (d, J = 1.6 Hz, 1H), 8.31-8.24 (m, 1H), 7.60 (d, J = 8.5 Hz, 1H), 7.54 (dd, J = 8.3, 4.2 Hz, 1H), 7.44-7.34 (m, 3H), 7.30 (t, J = 7.6 Hz, 2H), 7.24-7.13 (m, 2H), 6.28 (d, J = 8.2 Hz, 1H), 6.03 (d, J = 1.6 Hz, 1H). |
| 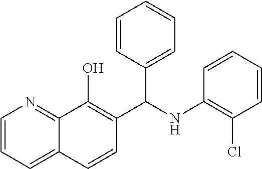 [SMILES: Oc1c(C(Nc2ccccc2Cl)c2ccccc2)ccc2cccnc12] | 1 | 7-(((2- chlorophenyl)amino)(phenyl)methyl)quinolin-8-ol LC-MS: m/z 361 (M + H)⁺. 1H NMR (400 MHz, DMSO-d6) δ (ppm): 10.23 (br s, 1H), 8.92-8.83 (m, 1H), 8.34-8.24 (m, 1H), 7.65 (d, J = 8.5 Hz, 1H), 7.59-7.53 (m, 1H), 7.47- 7.40 (m, 3H), 7.37-7.20 (m, 4H), 7.11-6.95 (m, 1H), 6.71-6.64 (m, 1H), 6.61 (td, J = 7.7, 1.4 Hz, 1H), 6.21 (d, J = 7.3 Hz, 1H), 5.76 (d, J = 7.3 Hz, 1H). |
| 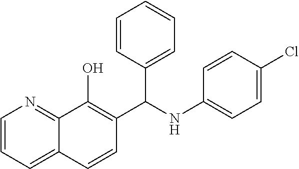 [SMILES: Oc1c(C(Nc2ccc(Cl)cc2)c2ccccc2)ccc2cccnc12] | 1 | 7-(((4- chlorophenyl)amino)(phenyl)methyl)quinolin-8-ol LC-MS: m/z 361 (M + H)⁺. 1H NMR (400 MHz, DMSO-d6) δ (ppm): 10.05 (br s, 1H), 8.89-8.81 (m, 1H), 8.31-8.24 (m, 1H), 7.61-7.50 (m, 2H), 7.41-7.37 (m, 3H), 7.32 (t, J = 7.5 Hz, 2H), 7.23-7.21 (m, 1H), 7.03 (d, J = 8.8 Hz, 2H), 6.70 (d, J = 7.1 Hz, 1H), 6.63 (d, J = 8.8 Hz, 2H), 6.11 (d, J = 7.0 Hz, 1H). |
| 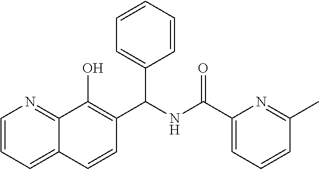 [SMILES: Cc1cccc(C(=O)NC(c2ccccc2)c2ccc3cccnc3c2O)n1] | 4 | N-((8-hydroxyquinolin-7-yl)(phenyl)methyl)-6- methylpicolinamide LC-MS: m/z 370 (M + H)⁺. 1H NMR (400 MHz, DMSO-d6) δ (ppm): 10.20 (br s, 1H), 9.60 (d, J = 8.7 Hz, 1H), 8.87 (d, J = 3.7 Hz, 1H), 8.34 (d, J = 8.3 Hz, 1H), 7.93-7.84 (m, 2H), 7.68 (d, J = 8.4 Hz, 1H), 7.57 (dd, J = 8.1, 4.0 Hz, 1H), 7.53-7.42 (m, 2H), 7.38-7.28 (m, 4H), 7.23 (t, J = 6.9 Hz, 1H), 6.72 (d, J = 9.1 Hz, 1H), 2.58 (s, 3H). |
| 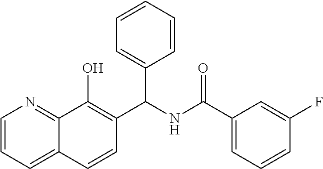 [SMILES: O=C(NC(c1ccccc1)c1ccc2cccnc2c1O)c1cccc(F)c1] | 4 | 3-fluoro-N-((8-hydroxyquinolin-7- yl)(phenyl)methyl)benzamide LC-MS: m/z 373 (M + H)⁺. 1H NMR (400 MHz, DMSO-d6) δ (ppm): 10.04 (s, 1H), 9.32 (d, J = 8.5 Hz, 1H), 8.87 (dd, J = 4.1, 1.5 Hz, 1H), 8.32 (dd, J = 8.3, 1.4 Hz, 1H), 7.82- 7.72 (m, 2H), 7.66 (d, J = 8.6 Hz, 1H), 7.59-7.49 (m, 2H), 7.46-7.37 (m, 2H), 7.36-7.29 (m, 4H), 7.28-7.21 (m, 1H), 6.98 (d, J = 8.5 Hz, 1H). |
| 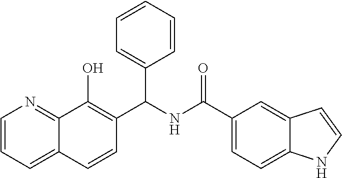 [SMILES: O=C(NC(c1ccccc1)c1ccc2cccnc2c1O)c1ccc2[nH]ccc2c1] | 4 | N-((8-hydroxyquinolin-7-yl)(phenyl)methyl)-1H- indole-5-carboxamide LC-MS: m/z 394 (M + H)⁺. 1H NMR (400 MHz, DMSO-d6) δ (ppm): 11.31 (s, 1H), 9.99 (br s, 1H), 9.05 (d, J = 8.8 Hz, 1H), 8.87 (dd, J = 4.2, 1.5 Hz, 1H), 8.32 (dd, J = 8.3, 1.5 Hz, 1H), 8.25 (s, 1H), 7.77 (d, J = 8.6 Hz, 1H), 7.70 (dd, J = 8.6, 1.6 Hz, 1H), 7.56 (dd, J = 8.3, 4.2 Hz, 1H), 7.47-7.40 (m, 3H), 7.37-7.27 (m, 4H), 7.24 (d, J = 6.8 Hz, 1H), 7.03 (d, J = 8.9 Hz, 1H), 6.54 (s, 1H). |
| 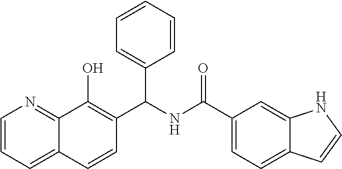 [SMILES: O=C(NC(c1ccccc1)c1ccc2cccnc2c1O)c1ccc2cc[nH]c2c1] | 4 | N-((8-hydroxyquinolin-7-yl)(phenyl)methyl)-1H- indole-6-carboxamide LC-MS: m/z 394 (M + H)⁺. 1H NMR (400 MHz, DMSO-d6) δ (ppm): 11.36 (s, 1H), 10.02 (br s, 1H), 9.12 (d, J = 9.1 Hz, 1H), 8.92-8.83 (m, 1H), 8.32 (d, J = 6.8 Hz, 1H), 8.03 (s, 1H), 7.77 (d, J = 8.6 Hz, 1H), 7.64-7.53 (m, 3H), 7.52-7.48 (m, 1H), 7.44 (d, J = 8.6 Hz, 1H), 7.37-7.27 (m, 4H), 7.24 (d, J = 6.8 Hz, 1H), 7.04 (d, J = 8.7 Hz, 1H), 6.49 (s, 1H). |
| 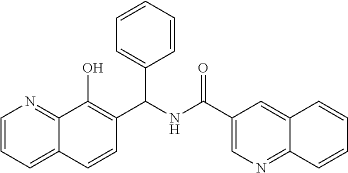 [SMILES: O=C(NC(c1ccccc1)c1ccc2cccnc2c1O)c1cnc2ccccc2c1] | 4 | N-((8-hydroxyquinolin-7- yl)(phenyl)methyl)quinoline-3-carboxamide LC-MS: m/z 406 (M + H)⁺. 1H NMR (400 MHz, DMSO-d6) δ (ppm): 10.03 (br s, 1H), 9.58 (t, J = 13.3 Hz, 1H), 9.32 (d, J = 2.1 Hz, 1H), 8.95 (d, J = 1.8 Hz, 1H), 8.91-8.84 (m, 1H), 8.33 (d, J = 8.3 Hz, 1H), 8.15-8.06 (m, 2H), 7.87 (t, J = 7.7 Hz, 1H), 7.74-7.66 (m, 2H), 7.57 (dd, J = 8.3, 4.2 Hz, 1H), 7.46 (d, J = 8.6 Hz, 1H), 7.42-7.31 (m, 4H), 7.26 (t, J = 6.7 Hz, 1H), 7.05 (d, J = 8.4 Hz, 1H). |
| 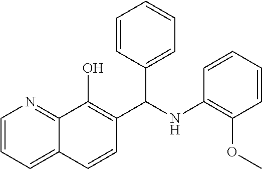 [SMILES: COc1ccccc1NC(c1ccccc1)c1ccc2cccnc2c1O] | 1 | 7-(((2- methoxyphenyl)amino)(phenyl)methyl)quinolin-8- ol LC-MS: m/z 357 (M + H)⁺. 1H NMR (400 MHz, DMSO-d6) δ (ppm): 10.05 (br s, 1H), 8.85 (dd, J = 4.1, 1.5 Hz, 1H), 8.29 (dd, J = 8.3, 1.5 Hz, 1H), 7.60 (d, J = 8.5 Hz, 1H), 7.54 (dd, J = 8.3, 4.2 Hz, 1H), 7.45-7.37 (m, 3H), 7.32 (t, J = 7.5 Hz, 2H), 7.23 (t, J = 7.3 Hz, 1H), 6.84 (dd, J = 7.9, 1.1 Hz, 1H), 6.64 (dd, J = 7.6, 6.5 Hz, 1H), 6.58-6.49 (m, 1H), 6.47-6.38 (m, 1H), 6.11 (d, |
| | | J = 6.5 Hz, 1H), 5.27 (d, J = 6.6 Hz, 1H), 3.81 (s, |
| | | 3H). |
| 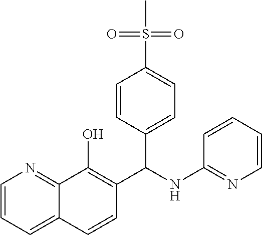 [SMILES: CS(=O)(=O)c1ccc(C(Nc2ccccn2)c2ccc3cccnc3c2O)cc1] | 2 | 7-((4-(methylsulfonyl)phenyl)(pyridin-2- ylamino)methyl)quinolin-8-ol LC-MS: m/z 406 (M + H)⁺. 1H NMR (400 MHz, DMSO-d6) δ (ppm): 9.31 (s, 1H), 8.97-8.87 (m, 1H), 8.37 (dd, J = 8.4, 1.5 Hz, 1H), 8.03 (d, J = 5.8 Hz, 1H), 7.97-7.90 (m, 3H), 7.75-7.65 (m, 2H), 7.66-7.53 (m, 2H), 7.51-7.36 (m, 2H), 7.16 (d, J = 8.9 Hz, 1H), 6.95-6.88 (m, 1H), 6.82 (t, J = 7.5 Hz, 1H), 3.16 (s, 3H). |
| 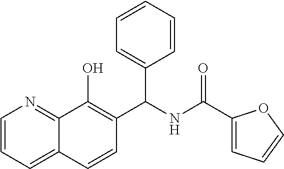 [SMILES: O=C(NC(c1ccccc1)c1ccc2cccnc2c1O)c1ccco1] | 4 | N-((8-hydroxyquinolin-7-yl)(phenyl)methyl)furan- 2-carboxamide LC-MS: m/z 345 (M + H)⁺. 1H NMR (400 MHz, DMSO-d6) δ (ppm): 10.02 (br s, 1H), 9.10 (d, J = 8.9 Hz, 1H), 8.87 (dd, J = 4.1, 1.4 Hz, 1H), 8.32 (dd, J = 8.3, 1.3 Hz, 1H), 7.86 (d, J = 0.9 Hz, 1H), 7.67 (d, J = 8.5 Hz, 1H), 7.56 (dd, J = 8.3, 4.2 Hz, 1H), 7.43 (d, J = 8.6 Hz, 1H), 7.36-7.20 (m, 6H), 6.90 (d, J = 8.9 Hz, 1H), 6.64 (dd, J = 3.4, 1.7 Hz, 1H). |
| 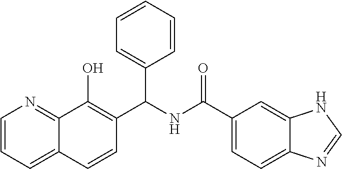 [SMILES: O=C(NC(c1ccccc1)c1ccc2cccnc2c1O)c1ccc2nc[nH]c2c1] | 4 | N-((8-hydroxyquinolin-7-yl)(phenyl)methyl)-1H- benzo[d]imidazole-6-carboxamide LC-MS: m/z 395 (M + H)⁺. 1H NMR (400 MHz, DMSO-d6) δ (ppm): 12.65 (s, 1H), 10.04 (br s, 1H), 9.21 (d, J = 8.6 Hz, 1H), 8.87 (dd, J = 4.1, 1.5 Hz, 1H), 8.37-8.29 (m, 2H), 8.16 (s, 1H), 7.82 (d, J = 7.9 Hz, 1H), 7.76 (d, J = 8.5 Hz, 1H), 7.59-7.54 (m, 1H), 7.44 (d, J = 8.6 Hz, 1H), 7.39-7.29 (m, 4H), 7.24 (t, J = 6.7 Hz, 1H), 7.04 (d, J = 8.7 Hz, 1H). |
| 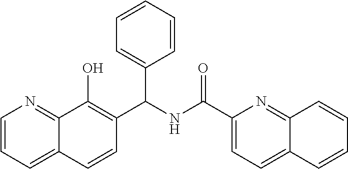 [SMILES: O=C(NC(c1ccccc1)c1ccc2cccnc2c1O)c1ccc2ccccc2n1] | 4 | N-((8-hydroxyquinolin-7- yl)(phenyl)methyl)quinoline-2-carboxamide LC-MS: m/z 406 (M + H)⁺. 1H NMR (400 MHz, DMSO-d6) δ (ppm): 9.92 (d, J = 9.1 Hz, 1H), 8.89 (dd, J = 4.2, 1.5 Hz, 1H), 8.60 (d, J = 8.5 Hz, 1H), 8.35 (dd, J = 8.4, 1.5 Hz, 1H), 8.20 (dd, J = 10.9, 8.6 Hz, 2H), 8.10 (d, J = 8.1 Hz, 1H), 7.88 (dd, J = 11.2, 4.2 Hz, 1H), 7.79- 7.69 (m, 2H), 7.58 (dd, J = 8.3, 4.2 Hz, 1H), 7.47 (d, J = 8.5 Hz, 1H), 7.41 (d, J = 7.5 Hz, 2H), 7.33 (t, J = 7.6 Hz, 2H), 7.25 (t, J = 7.3 Hz, 1H), 6.82 (d, |
| | | J = 9.1 Hz, 1H). |
| 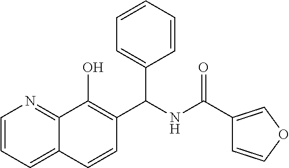 [SMILES: O=C(NC(c1ccccc1)c1ccc2cccnc2c1O)c1ccoc1] | 4 | N-((8-hydroxyquinolin-7-yl)(phenyl)methyl)furan- 3-carboxamide LC-MS: m/z 345 (M + H)⁺. 1H NMR (400 MHz, DMSO-d6) δ (ppm): 9.98 (br s, 1H), 8.92 (d, J = 8.6 Hz, 1H), 8.87 (d, J = 2.8 Hz, 1H), 8.38-8.28 (m, 2H), 7.74 (s, 1H), 7.62 (d, J = 8.5 Hz, 1H), 7.57 (dd, J = 8.2, 4.2 Hz, 1H), 7.45 (d, J = 8.5 Hz, 1H), 7.37-7.21 (m, 5H), 7.01-6.87 (m, 2H). |
| 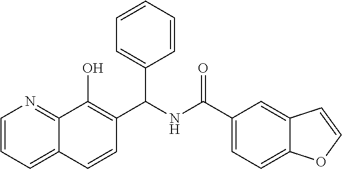 [SMILES: O=C(NC(c1ccccc1)c1ccc2cccnc2c1O)c1ccc2occc2c1] | 4 | N-((8-hydroxyquinolin-7- yl)(phenyl)methyl)benzofuran-5-carboxamide LC-MS: m/z 395 (M + H)⁺. 1H NMR (400 MHz, DMSO-d6) δ (ppm): 10.03 (br s, 1H), 9.26 (d, J = 8.7 Hz, 1H), 8.88 (dd, J = 4.2, 1.5 Hz, 1H), 8.36-8.28 (m, 2H), 8.09 (d, J = 2.2 Hz, 1H), 7.92 (dd, J = 8.7, 1.7 Hz, 1H), 7.73 (d, J = 8.6 Hz, 1H), 7.68 (d, J = 8.7 Hz, 1H), 7.57 (dd, J = 8.3, 4.2 Hz, 1H), 7.45 (d, J = 8.6 Hz, 1H), 7.37- 7.30 (m, 4H), 7.27-7.21 (m, 1H), 7.07 (dd, J = 5.9, 5.2 Hz, 1H), 7.03 (d, J = 8.7 Hz, 1H). |
| 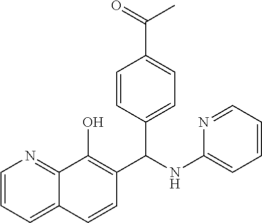 [SMILES: CC(=O)c1ccc(C(Nc2ccccn2)c2ccc3cccnc3c2O)cc1] | 2 | 1-(4-((8-hydroxyquinolin-7-yl)(pyridin-2- ylamino)methyl)phenyl)ethan-1-one LC-MS: m/z 370 (M + H)⁺. 1H NMR (400 MHz, DMSO-d6) δ (ppm): 10.03 (brs, 1H), 8.86 (d, J = 3.2 Hz, 1H), 8.30 (dd, J = 8.4, 1.2 Hz, 1H), 7.93-7.88 (m, 3H), 7.59-7.53 (m, 4H), 7.51-7.38 (m, 3H), 6.93 (d, J = 8.4 Hz, 1H), 6.71 (d, J = 8.4 Hz, 1H), 6.51-6.48 (m, 1H), 2.53 (s, 3H) |
| 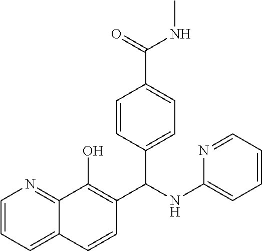 [SMILES: CNC(=O)c1ccc(C(Nc2ccccn2)c2ccc3cccnc3c2O)cc1] | 2 | 4-((8-hydroxyquinolin-7-yl)(pyridin-2- ylamino)methyl)-N-methylbenzamide LC-MS: m/z 385 (M + H)⁺. 1H NMR (400 MHz, DMSO-d6) δ (ppm): 9.88 (br s, 1H), 8.85 (dd, J = 4.2, 1.5 Hz, 1H), 8.38-8.26 (m, 2H), 7.91 (d, J = 3.6 Hz, 1H), 7.73 (d, J = 8.3 Hz, 2H), 7.59 (d, J = 8.5 Hz, 1H), 7.54 (dd, J = 8.3, 4.2 Hz, 1H), 7.45-7.30 (m, 5H), 6.90 (d, J = 8.3 Hz, 1H), 6.69 (d, J = 8.4 Hz, 1H), 6.52-6.43 (m, 1H), 2.74 (d, J = 4.5 Hz, 3H). |
| 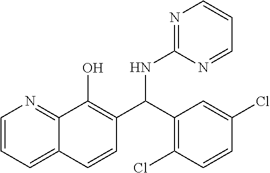 [SMILES: Oc1c(C(Nc2ncccn2)c2cc(Cl)ccc2Cl)ccc2cccnc12] | 3 | 7-((2,5-dichlorophenyl)(pyrimidin-2- ylamino)methyl)quinolin-8-ol LC-MS: m/z 397 (M + H)⁺. 1H NMR (400 MHz, DMSO-d6) δ (ppm): 10.03 (br s, 1H), 8.86 (dd, J = 4.1, 1.4 Hz, 1H), 8.31 (dd, J = 10.1, 3.1 Hz, 3H), 7.99 (d, J = 8.4 Hz, 1H), 7.56 (dd, J = 8.3, 4.2 Hz, 1H), 7.47 (t, J = 7.0 Hz, 1H), 7.43-7.30 (m, 4H), 7.06 (d, J = 8.4 Hz, 1H), 6.62 (t, J = 4.8 Hz, 1H). |
| 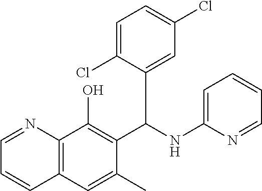 [SMILES: Cc1cc2cccnc2c(O)c1C(Nc1ccccn1)c1cc(Cl)ccc1Cl] | 2 | 7-((2,5-dichlorophenyl)(pyridin-2- ylamino)methyl)-6-methylquinolin-8-ol LC-MS: m/z 410 (M + H)⁺. 1H NMR (400 MHz, DMSO-d6) δ 8.74 (dd, J = 4.2, 1.6 Hz, 1H), 8.21 (dd, J = 8.4, 1.6 Hz, 1H), 7.94 (d, J = 5.2 Hz, 1H), 7.62 (d, J = 2.5 Hz, 1H), 7.51 (dd, J = 8.2, 4.2 Hz, 1H), 7.45-7.20 (m, 5H), 6.83 (d, J = 7.5 Hz, 1H), 6.78 (d, J = 8.4 Hz, 1H), 6.52-6.49 (m, 1H), 2.46 (s, 3H). |
| 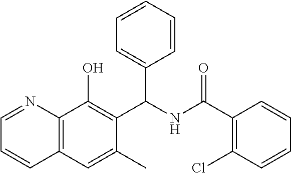 [SMILES: Cc1cc2cccnc2c(O)c1C(NC(=O)c1ccccc1Cl)c1ccccc1] | 4 | 2-chloro-N-((8-hydroxy-6-methylquinolin-7- yl)(phenyl)methyl)benzamide LC-MS: m/z 403 (M + H)⁺. 1H NMR (400 MHz, DMSO-d6) δ 8.94 (d, J = 8.4 Hz, 1H), 8.80 (dd, J = 4.2, 1.6 Hz, 1H), 8.25 (dd, J = 8.4, 1.6 Hz, 1H), 7.58-7.36 (m, 5H), 7.31- 7.29 (m, 5H), 7.26-7.19 (m, 1H), 6.96 (d, J = 8.3 Hz, 1H), 2.48 (s, 3H). |
| 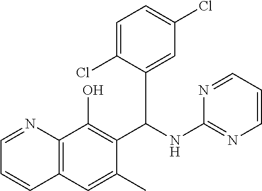 [SMILES: Cc1cc2cccnc2c(O)c1C(Nc1ncccn1)c1cc(Cl)ccc1Cl] | 3 | 7-((2,5-dichlorophenyl)(pyrimidin-2- ylamino)methyl)-6-methylquinolin-8-ol LC-MS: m/z 411 (M + H)⁺. 1H NMR (400 MHz, DMSO-d6) δ 8.76 (d, J = 4.2 Hz, 1H), 8.33 (d, J = 4.8 Hz, 2H), 8.22 (d, J = 7.9 Hz, 1H), 7.60 (d, J = 8.3 Hz, 1H), 7.53-7.51 (m, 2H), 7.42 (d, J = 8.4 Hz, 1H), 7.37-7.32 (m, 1H), 7.26 (s, 1H), 6.88 (d, J = 8.4 Hz, 1H), 6.66 (dd, J = 4.8, 4.8 Hz, 1H), 2.45 (s, 3H). |
| 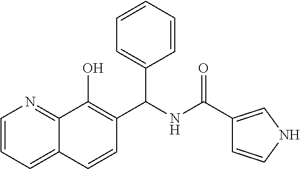 [SMILES: O=C(NC(c1ccccc1)c1ccc2cccnc2c1O)c1cc[nH]c1] | 4 | N-((8-hydroxyquinolin-7-yl)(phenyl)methyl)-1H- pyrrole-3-carboxamide LC-MS: m/z 344 (M + H)⁺. 1H NMR (400 MHz, DMSO-d6) δ (ppm): 11.17 (s, 1H), 9.93 (br s, 1H), 8.87 (d, J = 2.9 Hz, 1H), 8.46 (d, J = 9.0 Hz, 1H), 8.32 (d, J = 8.0 Hz, 1H), 7.71 (d, J = 8.5 Hz, 1H), 7.56 (dd, J = 8.2, 4.1 Hz, 1H), 7.49-7.39 (m, 2H), 7.34-7.18 (m, 5H), 6.95 (d, J = 9.0 Hz, 1H), 6.76 (d, J = 2.1 Hz, 1H), 6.60 (s, 1H). |
| 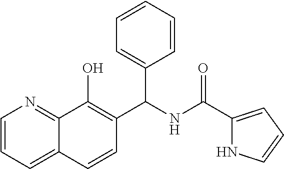 [SMILES: O=C(NC(c1ccccc1)c1ccc2cccnc2c1O)c1ccc[nH]1] | 4 | N-((8-hydroxyquinolin-7-yl)(phenyl)methyl)-1H- pyrrole-2-carboxamide LC-MS: m/z 344 (M + H)⁺. 1H NMR (400 MHz, DMSO-d6) δ (ppm): 11.47 (s, 1H), 9.98 (br s, 1H), 8.86 (dd, J = 4.1, 1.3 Hz, 1H), 8.68 (d, J = 8.9 Hz, 1H), 8.32 (dd, J = 8.3, 1.3 Hz, 1H), 7.67 (d, J = 8.6 Hz, 1H), 7.56 (dd, J = 8.3, 4.2 Hz, 1H), 7.44 (d, J = 8.6 Hz, 1H), 7.34-7.20 (m, 5H), 6.99 (s, 1H), 6.95 (d, J = 8.9 Hz, 1H), 6.88 (s, 1H), 6.10 (d, J = 3.3 Hz, 1H). |
| 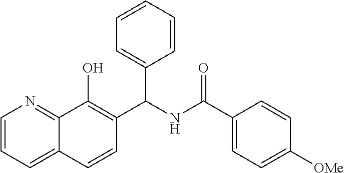 [SMILES: COc1ccc(C(=O)NC(c2ccccc2)c2ccc3cccnc3c2O)cc1] | 4 | N-((8-hydroxyquinolin-7-yl)(phenyl)methyl)-4- methoxybenzamide LC-MS: m/z 385 (M + H)⁺. 1H NMR (400 MHz, DMSO-d6) δ (ppm): 9.99 (br s, 1H), 9.06 (d, J = 9.0 Hz, 1H), 8.87 (d, J = 2.9 Hz, 1H), 8.32 (d, J = 7.2 Hz, 1H), 7.93 (d, J = 8.8 Hz, 2H), 7.70 (d, J = 8.5 Hz, 1H), 7.56 (dd, J = 8.3, 4.2 Hz, 1H), 7.43 (d, J = 8.6 Hz, 1H), 7.33-7.27 (m, 4H), 7.26-7.19 (m, 1H), 7.03-6.96 (m, 3H), 3.81 (s, 3H). |
| 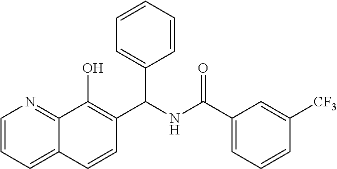 [SMILES: O=C(NC(c1ccccc1)c1ccc2cccnc2c1O)c1cccc(C(F)(F)F)c1] | 4 | N-((8-hydroxyquinolin-7-yl)(phenyl)methyl)-3- (trifluoromethyl)benzamide LC-MS: m/z 423 (M + H)⁺. 1H NMR (400 MHz, DMSO-d6) δ (ppm): 10.05 (br s, 1H), 9.51 (d, J = 8.4 Hz, 1H), 8.90-8.84 (m, 1H), 8.36-8.28 (m, 2H), 8.25 (d, J = 7.8 Hz, 1H), 7.92 (d, J = 7.8 Hz, 1H), 7.73 (t, J = 7.8 Hz, 1H), 7.63 (d, J = 8.6 Hz, 1H), 7.57 (dd, J = 8.3, 4.2 Hz, 1H), 7.44 (d, J = 8.6 Hz, 1H), 7.36-7.29 (m, 4H), 7.28-7.20 (m, 1H), 7.00 (d, J = 8.4 Hz, 1H). |
| 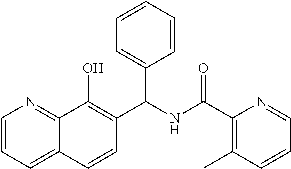 [SMILES: Cc1cccnc1C(=O)NC(c1ccccc1)c1ccc2cccnc2c1O] | 4 | N-((8-hydroxyquinolin-7-yl)(phenyl)methyl)-3- methylpicolinamide LC-MS: m/z 370 (M + H)⁺. 1H NMR (400 MHz, DMSO-d6) δ (ppm): 10.20 (br s, 1H), 9.50 (d, J = 9.1 Hz, 1H), 8.87 (dd, J = 4.1, 1.4 Hz, 1H), 8.48 (d, J = 4.1 Hz, 1H), 8.34 (dd, J = 8.3, 1.3 Hz, 1H), 7.77 (d, J = 7.7 Hz, 1H), 7.69 (d, J = 8.5 Hz, 1H), 7.57 (dd, J = 8.3, 4.2 Hz, 1H), 7.51-7.42 (m, 2H), 7.37 (d, J = 7.4 Hz, 2H), 7.32 (t, J = 7.6 Hz, 2H), 7.24 (t, J = 7.1 Hz, 1H), 6.81 (d, J = 9.1 Hz, 1H), 2.52 (s, 3H). |
| 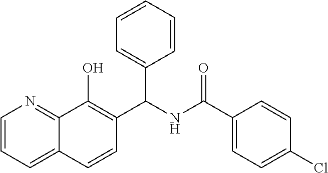 [SMILES: O=C(NC(c1ccccc1)c1ccc2cccnc2c1O)c1ccc(Cl)cc1] | 4 | 4-chloro-N-((8-hydroxyquinolin-7- yl)(phenyl)methyl)benzamide LC-MS: m/z 389 (M + H)⁺. 1H NMR (400 MHz, DMSO-d6) δ (ppm): 10.05 (br s, 1H), 9.31 (d, J = 8.5 Hz, 1H), 8.87 (d, J = 4.0 Hz, 1H), 8.32 (d, J = 8.2 Hz, 1H), 7.96 (d, J = 8.5 Hz, 2H), 7.66 (d, J = 8.6 Hz, 1H), 7.59-7.51 (m, 3H), 7.43 (d, J = 8.6 Hz, 1H), 7.37-7.29 (m, 4H), 7.28-7.18 (m, 1H), 6.98 (d, J = 8.6 Hz, 1H). |
| 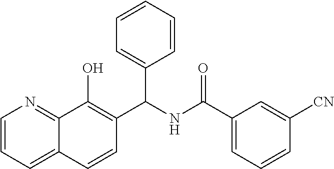 [SMILES: N#Cc1cccc(C(=O)NC(c2ccccc2)c2ccc3cccnc3c2O)c1] | 4 | 3-cyano-N-((8-hydroxyquinolin-7- yl)(phenyl)methyl)benzamide LC-MS: m/z 380 (M + H)⁺. 1H NMR (400 MHz, DMSO-d6) δ (ppm): 10.04 (br s, 1H), 9.43 (d, J = 8.4 Hz, 1H), 8.87 (dd, J = 4.1, 1.5 Hz, 1H), 8.42 (s, 1H), 8.32 (dd, J = 8.3, 1.4 Hz, 1H), 8.22 (d, J = 8.1 Hz, 1H), 8.02 (d, J = 7.7 Hz, 1H), 7.70 (t, J = 7.8 Hz, 1H), 7.63 (d, J = 8.6 Hz, 1H), 7.57 (dd, J = 8.3, 4.2 Hz, 1H), 7.44 (d, J = 8.7 Hz, 1H), 7.34-7.30 (m, 4H), 7.29-7.20 (m, 1H), 6.97 (d, J = 8.3 Hz, 1H). |
| 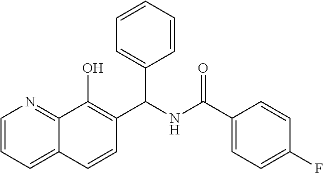 [SMILES: O=C(NC(c1ccccc1)c1ccc2cccnc2c1O)c1ccc(F)cc1] | 4 | 4-fluoro-N-((8-hydroxyquinolin-7- yl)(phenyl)methyl)benzamide LC-MS: m/z 373 (M + H)⁺. 1H NMR (400 MHz, DMSO-d6) δ (ppm): 10.01 (br s, 1H), 9.25 (d, J = 8.6 Hz, 1H), 8.90-8.84 (m, 1H), 8.32 (d, J = 8.4 Hz, 1H), 8.06-7.96 (m, 2H), 7.67 (d, J = 8.6 Hz, 1H), 7.56 (dd, J = 8.3, 4.2 Hz, 1H), 7.43 (d, J = 8.6 Hz, 1H), 7.35-7.27 (m, 6H), 7.26-7.18 (m, 1H), 6.98 (d, J = 8.6 Hz, 1H). |
| 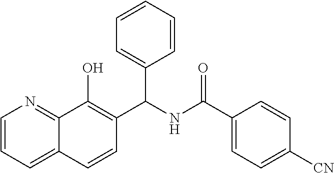 [SMILES: N#Cc1ccc(C(=O)NC(c2ccccc2)c2ccc3cccnc3c2O)cc1] | 4 | 4-cyano-N-((8-hydroxyquinolin-7- yl)(phenyl)methyl)benzamide LC-MS: m/z 380 (M + H)⁺. 1H NMR (400 MHz, DMSO-d6) δ (ppm): 10.05 (br s, 1H), 9.50 (d, J = 8.5 Hz, 1H), 8.87 (dd, J = 4.1, 1.4 Hz, 1H), 8.32 (dd, J = 8.3, 1.3 Hz, 1H), 8.08 (d, J = 8.4 Hz, 2H), 7.97 (d, J = 8.4 Hz, 2H), 7.64 (d, J = 8.6 Hz, 1H), 7.57 (dd, J = 8.3, 4.2 Hz, 1H), 7.43 (d, J = 8.6 Hz, 1H), 7.37-7.30 (m, 4H), 7.28-7.21 (m, 1H), 6.98 (d, J = 8.5 Hz, 1H). |
| 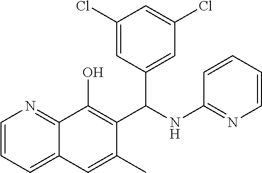 [SMILES: Cc1cc2cccnc2c(O)c1C(Nc1ccccn1)c1cc(Cl)cc(Cl)c1] | 2 | 7-((3,5-dichlorophenyl)(pyridin-2- ylamino)methyl)-6-methylquinolin-8-ol LC-MS: m/z 410 (M + H)⁺. 1H NMR (400 MHz, DMSO-d6) δ (ppm): 9.95 (br s, 1H), 8.78 (d, J = 3.5 Hz, 1H), 8.23 (d, J = 8.1 Hz, 1H), 7.99 (d, J = 4.4 Hz, 1H), 7.53 (dd, J = 8.2, 4.1 Hz, 1H), 7.45-7.39 (m, 2H), 7.29 (s, 1H), 7.23-7.17 (m, 2H), 7.13 (d, J = 8.0 Hz, 1H), 7.00 (d, J = 7.8 Hz, 1H), 6.84 (d, J = 8.4 Hz, 1H), 6.60-6.49 (m, 1H), 2.54 (s, 3H). |
| 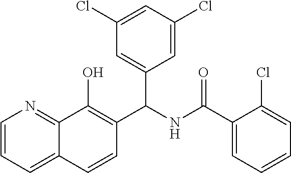 [SMILES: O=C(NC(c1cc(Cl)cc(Cl)c1)c1ccc2cccnc2c1O)c1ccccc1Cl] | 4 | 2-chloro-N-((3,5-dichlorophenyl)(8- hydroxyquinolin-7-yl)methyl)benzamide LC-MS: m/z 457 (M + H)⁺. 1H NMR (400 MHz, DMSO-d6) δ (ppm): 10.03- 9.85 (m, 1H), 8.55 (s, 1H), 8.35 (d, J = 7.8 Hz, 1H), 7.68-7.59 (m, 3H), 7.54-7.41 (m, 6H), 7.35 (t, J = 7.5 Hz, 1H), 6.94 (d, J = 8.4 Hz, 1H), 6.72 (t, J = 8.0 Hz, 1H). |
| 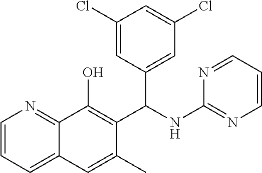 [SMILES: Cc1cc2cccnc2c(O)c1C(Nc1ncccn1)c1cc(Cl)cc(Cl)c1] | 3 | 7-((3,5-dichlorophenyl)(pyrimidin-2- ylamino)methyl)-6-methylquinolin-8-ol LC-MS: m/z 411 (M + H)⁺. 1H NMR (400 MHz, CDCl3) δ (ppm): 8.62 (dd, J = 4.2, 1.4 Hz, 1H), 8.23 (d, J = 4.8 Hz, 2H), 7.99 (dd, J = 8.3, 1.4 Hz, 1H), 7.34 (dd, J = 8.3, 4.2 Hz, 1H), 7.20 (s, 1H), 7.14 (dd, J = 4.2, 2.3 Hz, 2H), 6.91 (s, 2H), 6.49 (t, J = 4.8 Hz, 1H), 2.63 (s, 3H). |
| 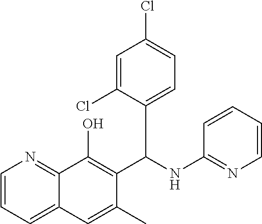 [SMILES: Cc1cc2cccnc2c(O)c1C(Nc1ccccn1)c1ccc(Cl)cc1Cl] | 2 | 7-((2,4-dichlorophenyl)(pyridin-2- ylamino)methyl)-6-methylquinolin-8-ol LC-MS: m/z 410 (M + H)⁺. 1H NMR (400 MHz, DMSO) δ 9.67 (br s, 1H), 8.80 (d, J = 3.6 Hz, 1H), 8.27 (d, J = 7.9 Hz, 1H), 7.98 (d, J = 4.5 Hz, 1H), 7.66 (d, J = 8.5 Hz, 1H), 7.57 (dd, J = 8.2, 4.2 Hz, 1H), 7.53 (d, J = 1.9 Hz, 1H), 7.44 (t, J = 7.9 Hz, 2H), 7.30 (s, 1H), 7.25 (d, J = 7.4 Hz, 1H), 6.89 (d, J = 7.4 Hz, 1H), 6.85-6.76 (m, 1H), 6.59-6.48 (m, 1H), 2.51 (s, 3H). |
| 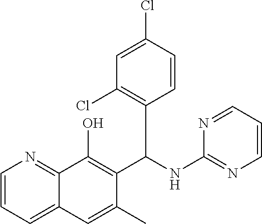 [SMILES: Cc1cc2cccnc2c(O)c1C(Nc1ncccn1)c1ccc(Cl)cc1Cl] | 3 | 7-((2,4-dichlorophenyl)(pyrimidin-2- ylamino)methyl)-6-methylquinolin-8-ol LC-MS: m/z 411 (M + H)⁺. 1H NMR (400 MHz, DMSO-d6) δ (ppm): 9.88 (br s, 1H), 8.77 (d, J = 3.4 Hz, 1H), 8.31 (d, J = 4.8 Hz, 2H), 8.22 (d, J = 7.9 Hz, 1H), 7.56-7.44 (m, 4H), 7.40-7.33 (m, 1H), 7.23 (s, 1H), 6.90 (d, J = 8.3 Hz, 1H), 6.64 (t, J = 4.8 Hz, 1H), 2.45 (s, 3H). |
| 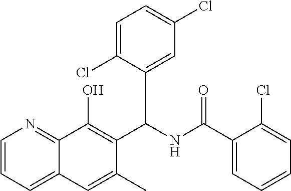 [SMILES: Cc1cc2cccnc2c(O)c1C(NC(=O)c1ccccc1Cl)c1cc(Cl)ccc1Cl] | 4 | 2-chloro-N-((2,5-dichlorophenyl)(8-hydroxy-6- methylquinolin-7-yl)methyl)benzamide LC-MS: m/z 471, 473 (M + H)⁺. 1H NMR (400 MHz, CD3OD) δ (ppm): 8.73 (d, J = 7.2 Hz, 1H), 8.17 (d, J = 8.4 Hz, 1H), 7.66 (s, 1H), 7.51-7.35 (m, 6H) 7.30-7.26 (m, 2H), 6.95 (s, 1H), 2.62 (s, 3H) |
| 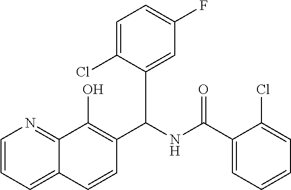 [SMILES: O=C(NC(c1cc(F)ccc1Cl)c1ccc2cccnc2c1O)c1ccccc1Cl] | 4 | 2-chloro-N-((2-chloro-5-fluorophenyl)(8- hydroxyquinolin-7-yl)methyl)benzamide LC-MS: m/z 441, 443 (M + H)⁺. 1H NMR (400 MHz, CDCl3) δ (ppm): 8.72 (d, J = 4.0 Hz, 1H), 8.34 (d, J = 8.0 Hz, 1H), 7.72-7.65 (m, 3H), 7.53 (dd, J = 8.0 Hz, 4.4 Hz, 1H) 7.40- 7.20 (m, 6H), 6.93 (d, J = 8.0 Hz, 1H), 6.88 (dd, J = 8.0 Hz, 2.4 Hz, 1H) |
| 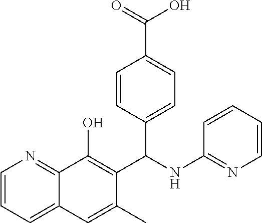 [SMILES: Cc1cc2cccnc2c(O)c1C(Nc1ccccn1)c1ccc(C(=O)O)cc1] | 2 | 4-((8-hydroxy-6-methylquinolin-7-yl)(pyridin-2- ylamino)methyl)benzoic acid LC-MS: m/z 386 (M + H)⁺. 1H NMR (400 MHz, DMSO-d6) δ (ppm): 12.84 (br s, 1H), 9.99 (br s, 1H), 8.85 (d, J = 4.0 Hz, 1H), 8.30 (d, J = 8.0 Hz, 1H), 8.04 (d, J = 4.4 Hz, 1H), 7.92 (d, J = 8.0 Hz, 2H) 7.60 (dd, J = 8.4 Hz, 4.4 Hz, 1H), 7.47 (t, J = 7.6 Hz, 1H), 7.43 (d, J = 8.0 Hz, 2H), 7.34 (s, 1H), 7.17-7.11 (m, 2H), 6.88 (d, J = 8.4 Hz, 1H), 6.58 (t, J = 6.0 Hz, 1H), 2.58 (s, 3H) |
| 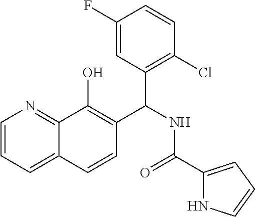 [SMILES: O=C(NC(c1cc(F)ccc1Cl)c1ccc2cccnc2c1O)c1ccc[nH]1] | 4 | N-((2-chloro-5-fluorophenyl)(8-hydroxyquinolin- 7-yl)methyl)-1H-pyrrole-2-carboxamide LC-MS: m/z 396 (M + H)⁺. 1H NMR (400 MHz, CD3OD) δ (ppm): 8.66 (s, 1H), 8.27 (d, J = 7.6 Hz, 1H), 7.53-7.51 (m, 1H), 7.43-7.37 (m, 3H), 7.10-7.07 (m, 1H), 7.04-6.83 (m, 4H), 6.12 (s, 1H) |
| 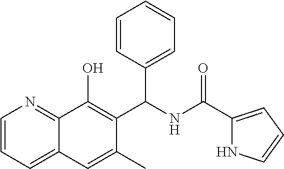 [SMILES: Cc1cc2cccnc2c(O)c1C(NC(=O)c1ccc[nH]1)c1ccccc1] | 4 | N-((8-hydroxy-6-methylquinolin-7- yl)(phenyl)methyl)-1H-pyrrole-2-carboxamide LC-MS: m/z 358 (M + H)⁺. 1H NMR (400 MHz, DMSO-d6) δ (ppm): 11.63 (br s, 1H), 8.80 (dd, J = 4.4 Hz, 1.6 Hz, 1H), 8.40 (dd, J = 8.4 Hz, 3.6 Hz, 1H), 8.25 (dd, J = 8.4 Hz, 1.6 Hz, 1H), 7.54 (dd, J = 8.4 Hz, 4.0 Hz, 1H), 7.32-7.24 (m, 3H), 7.23-7.20 (m, 3H), 6.90 (dd, J = 4.0 Hz, 1.2 Hz, 1H), 6.85 (d, J = 8.8 Hz, 1H), 6.75 (s, 1H), 6.12 (dd, J = 3.6 Hz, 2.0 Hz, 1H), 2.54 (s, 3H) |
| 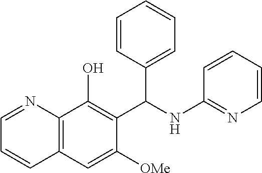 [SMILES: COc1cc2cccnc2c(O)c1C(Nc1ccccn1)c1ccccc1] | 2 | 6-methoxy-7-(phenyl(pyridin-2- ylamino)methyl)quinolin-8-ol LC-MS: m/z 358 (M + H)⁺. 1H NMR (400 MHz, DMSO-d6) δ (ppm): 10.02 (br s, 1H), 8.70 (dd, J = 4.0 Hz, 1.2 Hz, 1H), 8.21 (dd, J = 8.4 Hz, 1.2 Hz, 1H), 7.97 (dd, J = 4.8 Hz, 1.2 Hz, 1H), 7.51 (dd, J = 8.4 Hz, 4.4 Hz, 1H), 7.42-7.33 (m, 3H), 7.25-7.13 (m, 4H), 6.93-6.88 (m, 2H), 6.75 (d, J = 8.4 Hz, 1H), 6.51 (dd, J = 6.4 Hz, 1.2 Hz, 1H), 3.89 (s, 3H) |
| 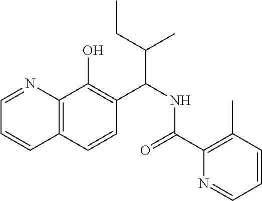 [SMILES: CCC(C)C(NC(=O)c1ncccc1C)c1ccc2cccnc2c1O] | 5 | N-(1-(8-hydroxyquinolin-7-yl)-2-methylbutyl)-3- methylpicolinamide LC-MS: m/z 350 (M + H)⁺. 1H NMR (400 MHz,CD3OD) δ (ppm): mixture of stereoisomers (dd, J = 4.4 Hz, 1.2 Hz, 1H), 8.43 (d, J = 8.4 Hz, 1.2 Hz, 1H), 8.23 (dd, J = 8.4 Hz, 0.8 Hz, 1H), 7.72-7.66 (m, 1H), 7.50-7.45 (m, 2H), 7.41-7.33 (m, 2H), 5.40 (d, J = 8.0 Hz, 0.5H), 5.29 (d, J = 7.6 Hz, 0.5H), 2.56 and 2.55 (two set of s, total 3H), 2.27-2.23 (m, 1H), 1.81-1.72 (m, 0.5H), 1.48-1.30 (m, 1H), 1.21-1.16 (m, 0.5H), 1.07 (d, J = 6.8 Hz, 1.5H), 0.98 (t, J = 7.2 Hz, 1.5H), 0.92 (t, J = 7.2 Hz, 1.5H), 0.84 (d, J = 6.8 Hz, 1.5H) |
| 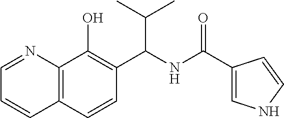 [SMILES: CC(C)C(NC(=O)c1cc[nH]c1)c1ccc2cccnc2c1O] | 5 | N-(1-(8-hydroxyquinolin-7-yl)-2-methylpropyl)- 1H-pyrrole-3-carboxamide LC-MS: m/z 310 (M + H)⁺. 1H NMR (400 MHz, DMSO-d6) δ (ppm): 11.12 (br s, 1H), 9.75 (br s, 1H), 8.85 (dd, J = 4.0 Hz, 1.2 Hz, 1H), 8.29 (dd, J = 8.4 Hz, 1.6 Hz, 1H), 7.87 (d, J = 9.2 Hz, 1H), 7.67 (d, J = 8.4 Hz, 1H), 7.53 (dd, J = 8.4 Hz, 4.4 Hz, 1H), 7.41-7.34 (m, 2H), |
| | | 6.74 (dd, J = 4.4 Hz, 2.4 Hz, 1H), 6.51 (dd, J = 4.0 |
| | | Hz, 2.4 Hz, 1H), 5.36 (t, J = 9.2 Hz, 1H), 2.22- |
| | | 2.13 (m, 1H), 1.02 (d, J = 6.8 Hz, 3H), 0.78 (d, J = |
| | | 6.8 Hz, 3H) |
| 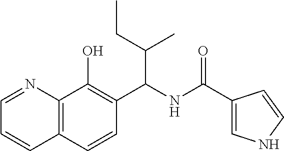 [SMILES: CCC(C)C(NC(=O)c1cc[nH]c1)c1ccc2cccnc2c1O] | 5 | N-(1-(8-hydroxyquinolin-7-yl)-2-methylbutyl)-1H- pyrrole-3-carboxamide LC-MS: m/z 324 (M + H)⁺. 1H NMR (400 MHz,CD3OD) δ (ppm): mixture of stereoisomers. 8.79 (dd, J = 4.0 Hz, 1.6 Hz, 1H), 8.23 (d, J = 8.4 Hz, 1.6 Hz, 1H), 7.52 (d, J = 8.4 Hz, 1H), 7.47 (dd, J = 8.4 Hz, 4.0 Hz, 1H), 7.39- 7.33 (m, 2H), 6.74 (dd, J = 4.8 Hz, 2.4 Hz, 1H), 6.56 (dt, J = 4.8 Hz, 2.0 Hz, 1H), 5.39-5.28 (m, 1H), 2.23-2.16 (m, 1H), 1.82-1.76 (m, 0.5H), 1.38- |
| | | 1.29 (m, 1H), 1.16-1.09 (m, 0.5H), 1.08 (d, J = 6.8 |
| | | Hz, 1.5H), 0.98 (t, J = 7.2 Hz, 1.5H), 0.88 (t, J = |
| | | 7.2 Hz, 1.5H), 0.80 (d, J = 6.8 Hz, 1.5H) |
| 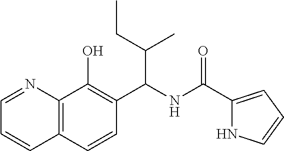 [SMILES: CCC(C)C(NC(=O)c1ccc[nH]1)c1ccc2cccnc2c1O] | 5 | N-(1-(8-hydroxyquinolin-7-yl)-2-methylbutyl)-1H- pyrrole-2-carboxamide LC-MS: m/z 324 (M + H)⁺. 1H NMR (400 MHz,CD3OD) δ (ppm): mixture of stereoisomers. 8.79 (dd, J = 4.0 Hz, 1.2 Hz, 1H), 8.22 (d, J = 8.4 Hz, 1.2 Hz, 1H), 7.52 (d, J = 8.4 Hz, 1H), 7.48 (dd, J = 8.4 Hz, 4.4 Hz, 1H), 7.37 (d, J = 8.4 Hz, 1H), 6.89-6.83 (m, 2H), 6.16 (dd, J = 6.0 Hz, 2.4 Hz, 1H), 5.42-5.30 (m, 1H), 2.20-2.16 (m, 1H), 1.83-1.75 (m, 0.5H), 1.40-1.31 (m, 1H), |
| | | 1.18-1.08 (m, 0.5H), 1.05 (d, J = 6.8 Hz, 1.5H), |
| | | 0.98 (t, J = 7.2 Hz, 1.5H), 0.92 (t, J = 7.2 Hz, |
| | | 1.5H), 0.83 (d, J = 6.8 Hz, 1.5H) |
| 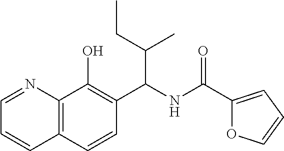 [SMILES: CCC(C)C(NC(=O)c1ccco1)c1ccc2cccnc2c1O] | 5 | N-(1-(8-hydroxyquinolin-7-yl)-2- methylbutyl)furan-2-carboxamide LC-MS: m/z 325 (M + H)⁺. 1H NMR (400 MHz, DMSO-d6) δ (ppm): mixture of stereoisomers. 9.81 (br s, 1H), 8.85 (dd, J = 4.0 Hz, 1.6 Hz, 1H), 8.51 (d, J = 9.2 Hz, 0.5H), 8.43 (d, J = 9.2 Hz, 0.5H), 8.29 (dd, J = 8.0 Hz, 1.2 Hz, 1H), 7.82 (s, 1H), 7.68 (dd, J = 8.4 Hz, 0.8 Hz, 1H), 7.53 (dd, J = 8.4 Hz, 4.0 Hz, 1H), 7.40 (d, J = 8.4 Hz, 1H), 7.13 (dd, J = 11.6 Hz, 3.6 Hz, 1H), |
| | | 6.63-6.59 (m, 1H), 5.47 (t, J = 9.2 Hz, 0.5H), 5.41 |
| | | (t, J = 9.2 Hz, 0.5H), 2.08-2.00 (m, 1H), 1.73-1.58 |
| | | (m, 0.5H), 1.33-1.17 (m, 1H), 1.12-1.02 (m, 0.5H), |
| | | 0.98 (d, J = 6.8 Hz, 1.5H), 0.90 (t, J = 7.2 Hz, |
| | | 1.5H), 0.81 (t, J = 7.2 Hz, 1.5H), 0.71 (d, J = 6.8 |
| | | Hz, 1.5H) |
| 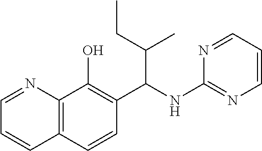 [SMILES: CCC(C)C(Nc1ncccn1)c1ccc2cccnc2c1O] | 3 | 7-(2-methyl-1-(pyrimidin-2- ylamino)butyl)quinolin-8-ol LC-MS: m/z 309 (M + H)⁺. 1H NMR (400 MHz, CD3OD) δ (ppm): mixture of stereoisomers. 8.79 (d, J = 3.2 Hz 1H), 8.29-8.17 (m, 3H), 7.49 (d, J = 8.4 Hz, 1H), 7.45 (dd, J = 8.0 Hz, 4.0 Hz, 1H), 7.33 (d, J = 8.4 Hz, 1H), 6.54 (dd, J = 8.4 Hz, 4.0 Hz, 1H), 5.40 (d, J = 8.0 Hz, 0.67H), 5.27 (d, J = 7.6 Hz, 0.33H), 2.24-2.18 (m, 1H), 1.83-1.77 (m, 0.33H), 1.43-1.10 (m, 1.67H), |
| | | 1.04 (d, J = 6.8 Hz, 2H), 0.95 (t, J = 7.2 Hz, 1H), |
| | | 0.90 (t, J = 7.2 Hz, 2H), 0.82 (d, J = 6.8 Hz, 1H) |
| 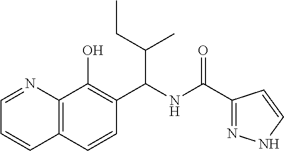 [SMILES: CCC(C)C(NC(=O)c1cc[nH]n1)c1ccc2cccnc2c1O] | 5 | N-(1-(8-hydroxyquinolin-7-yl)-3-methylbutyl)-1H- pyrazole-3-carboxamide LC-MS: m/z 325 (M + H)⁺. 1H NMR (400 MHz, DMSO-d6) δ (ppm): mixture of stereoisomers. 13.24 (br s, 1H), 9.70 (br s, 1H), 8.85 (dd, J = 4.4 Hz, 1.6 Hz 1H), 8.38-8.30 (m, 2H), 7.80 (br s, 1H), 7.63-7.56 (m, 1H), 7.53 (dd, J = 8.4 Hz, 4.0 Hz, 1H), 7.39 (d, J = 8.4 Hz, 1H), 6.61 (br s, 1H), 5.37-5.30 (m, 1H), 2.10-2.02 (m, 1H), 1.69-1.63 (m, 0.5H), 1.41-1.26 (m, 1H), 1.22- |
| | | 1.02 (m, 0.5H), 0.95 (d, J = 6.8 Hz, 1.5H), 0.91 (d, |
| | | J = 6.8 Hz, 1.5H), 0.76 (d, J = 6.8 Hz, 1.5H), 0.68 |
| | | (d, J = 6.8 Hz, 1.5H) |
| 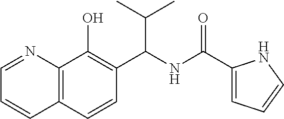 [SMILES: CC(C)C(NC(=O)c1ccc[nH]1)c1ccc2cccnc2c1O] | 5 | N-(1-(8-hydroxyquinolin-7-yl)-2-methylpropyl)- 1H-pyrrole-2-carboxamide LC-MS: m/z 310 (M + H)⁺. 1H NMR (400 MHz, DMSO-d6) δ (ppm): 11.42 (br s, 1H), 9.72 (br s, 1H), 8.85 (dd, J = 4.4 Hz, 1.6 Hz 1H), 8.30 (dd, J = 8.4 H, 1.6 Hz, 1H), 8.07 (d, J = 9.6 Hz, 1H), 7.66 (d, J = 8.8 Hz, 1H), 7.53 (dd, J = 8.4 Hz, 4.4 Hz, 1H), 7.40 (d, J = 8.8 Hz, 1H), |
| | | 6.90 (dd, J = 2.4 Hz, 1.6 Hz, 1H), 6.84 (dd, J = 3.6 |
| | | Hz, 2.4 Hz, 1H), 6.09 (dd, J = 3.6 Hz, 2.4 Hz, 1H), |
| | | 5.40 (d, J = 9.2 Hz, 1H), 2.24-2.12 (m, 1H), 1.02 |
| | | (d, J = 6.8 Hz, 3H), 0.79 (d, J = 6.8 Hz, 3H) |
| 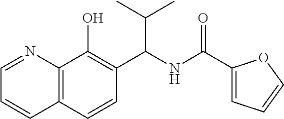 [SMILES: CC(C)C(NC(=O)c1ccco1)c1ccc2cccnc2c1O] | 5 | N-(1-(8-hydroxyquinolin-7-yl)-2- methylpropyl)furan-2-carboxamide LC-MS: m/z 311 (M + H)⁺. 1H NMR (400 MHz, CD3OD) δ (ppm): 8.81 (d, J = 4.4 Hz, 1.6 Hz 1H), 8.23 (dd, J = 8.4 H, 1.6 Hz, 1H), 7.66 (dd, J = 1.6 Hz, 0.8 Hz, 1H), 7.50 (d, J = 8.4 Hz, 1H), 7.47 (dd, J = 8.4 Hz, 4.0 Hz, 1H), 7.37 (d, J = 8.4 Hz, 1H), 7.10 (dd, J = 3.6 Hz, 0.8 Hz, |
| | | 1H), 6.57 (dd, J = 3.6 Hz, 1.6 Hz, 1H), 5.21 (d, J = |
| | | 9.2 Hz, 1H), 2.44-2.36 (m, 1H), 1.12 (d, J = 6.4 Hz, |
| | | 3H), 0.86 (d, J = 6.8 Hz, 3H) |
| 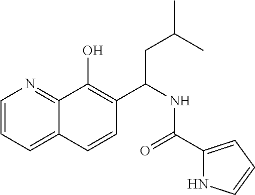 [SMILES: CC(C)CC(NC(=O)c1ccc[nH]1)c1ccc2cccnc2c1O] | 5 | N-(1-(8-hydroxyquinolin-7-yl)-3-methylbutyl)-1H- pyrrole-2-carboxamide LC-MS: m/z 324 (M + H)⁺. 1H NMR (400 MHz, CD3OD) δ (ppm): 8.79 (d, J = 3.2 Hz 1H), 8.21 (d, J = 7.6 Hz, 1H), 7.54 (d, J = 8.8 Hz, 1H), 7.46 (dd, J = 8.0 Hz, 4.0 Hz, 1H), 7.37 (d, J = 8.4 Hz, 1H), 6.90 (t, J = 2.0 Hz, 2H), 6.17 (t, J = 3.2 Hz, 1H), 5.69 (dd, J = 9.2 Hz, 6.0 Hz, 1H), 1.96-1.87 (m, 1H), 1.83-1.76 (m, 1H), 1.74- 1.65 (m, 1H), 1.01 (d, J = 6.4 Hz, 6H) |
| 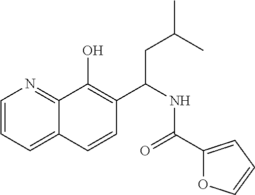 [SMILES: CC(C)CC(NC(=O)c1ccco1)c1ccc2cccnc2c1O] | 5 | N-(1-(8-hydroxyquinolin-7-yl)-3- methylbutyl)furan-2-carboxamide LC-MS: m/z 325 (M + H)⁺. 1H NMR (400 MHz, CD3OD) δ (ppm): mixture of two rotamers, ation is ca. 5:1. 8.82 (major) and 8.53 (minor) (d, J = 3.6 Hz, 1H), 8.32-8.22 (m, 1H), 7.70-7.50 (m, 1H), 7.26 and 7.07 (two set of d, J = 8.4 Hz, total 1H), 7.04 (s, 1H), 6.55 (s, 1H), 5.91 and 5.62 (two set of t, J = 7.2 Hz, total 1H), 2.02-1.94 (m, 1H), 1.90-1.74 (m, 1H), 1.68-1.58 (m, 1H), 1.04 and 1.02 (major, two set of d, J = 6.8 Hz, 5H), 0.62 and 0.39 (minor, two set of d, J = |
| | | 6.4 Hz, 1H) |
| 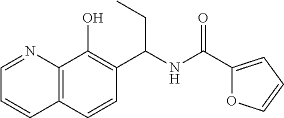 [SMILES: CCC(NC(=O)c1ccco1)c1ccc2cccnc2c1O] | 5 | N-(1-(8-hydroxyquinolin-7-yl)propyl)furan-2- carboxamide LC-MS: m/z 297 (M + H)⁺. 1H NMR (400 MHz, CD3OD) δ (ppm): 8.83 (d, J = 3.2 Hz, 1H), 8.27 (d, J = 7.6 Hz, 1H), 7.58 (s, 1H), 7.49 (dd, J = 8.4 Hz, 3.2 Hz, 2H), 7.25 (d, J = 8.4 Hz, 1H), 7.10 (d, J = 3.6 Hz, 1H), 6.55 (dd, J = 3.2 Hz, 1.6 Hz, 1H), 5.37 (t, J = 7.2 Hz, 1H), 2.09- |
| | | 2.01 (m, 2H), 1.00 (t, J = 7.2 Hz, 3H) |
| 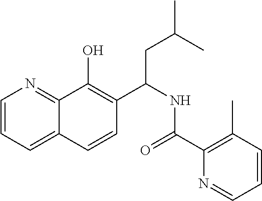 [SMILES: Cc1cccnc1C(=O)NC(CC(C)C)c1ccc2cccnc2c1O] | 5 | N-(1-(8-hydroxyquinolin-7-yl)-3-methylbutyl)-3- methylpicolinamide LC-MS: m/z 350 (M + H)⁺. 1H NMR (400 MHz, CD3OD) δ (ppm): 8.81 (dd, J = 4.0 Hz, 1.2 Hz, 1H), 8.44 (d, J = 4.0 Hz, 1H), 8.22 (dd, J = 8.4 Hz, 1.6 Hz, 1H), 7.70 (d, J = 7.6 Hz, 1H), 7.54 (d, J = 8.4 Hz, 1H), 7.47 (dd, J = 8.4 Hz, 4.4 Hz, 1H), 7.42-7.36 (m, 1H), 5.67 (dd, J = 8.8 Hz, 2.4 Hz, 1H), 2.54 (s, 3H), 1.97-1.83 (m, 2H), 1.71-1.63 (m, 2H), 1.03 (d, J = 6.4 Hz, 3H), 1.02 (d, J = 6.4 Hz, 3H) |
| 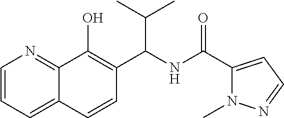 [SMILES: CC(C)C(NC(=O)c1ccnn1C)c1ccc2cccnc2c1O] | 5 | N-(1-(8-hydroxyquinolin-7-yl)-2-methylpropyl)-1- methyl-1H-pyrazole-5-carboxamide LC-MS: m/z 325 (M + H)⁺. 1H NMR (400 MHz, CD3OD) δ (ppm): 9.01 (dd, J = 5.2 Hz, 1.6 Hz 1H), 8.94 (dd, J = 8.4 Hz, 0.8 Hz, 1H), 7.95 (dd, J = 8.4 Hz, 1.2 Hz, 1H), 7.83 (d, J = 8.8 Hz, 1H), 7.78 (d, J = 8.8 Hz, 1H), 7.47 (d, J = 2.0 Hz, 1H), 6.89 (d. J = 2.0 Hz, 1H), 5.18 |
| | | (d, J = 10.4 Hz, 1H), 4.03 (s, 3H), 2.53-2.45 (m, |
| | | 1H), 1.23 (d, J = 6.4 Hz, 3H), 0.84 (d, J = 6.4 Hz, |
| | | 3H) |
| 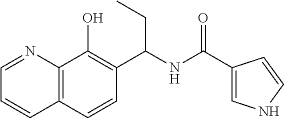 [SMILES: CCC(NC(=O)c1cc[nH]c1)c1ccc2cccnc2c1O] | 5 | N-(1-(8-hydroxyquinolin-7-yl)propyl)-1H-pyrrole- 3-carboxamide LC-MS: m/z 296 (M + H)⁺. 1H NMR (400 MHz, CD3OD) δ (ppm): 8.82 (dd, J = 4.0 Hz, 1.2 Hz, 1H), 8.32 (d, J = 7.6 Hz, 1H), 7.57 (d, J = 8.4 Hz, 1H), 7.53 (dd, J = 8.4 Hz, , 4.4 Hz, 1H), 7.42 (d, J = 8.4 Hz, 1H), 7.38 (dd, J = 4.4 Hz, 2.8 Hz, 1H), 6.75 (dd, J = 4.4 Hz, 1.6 Hz, |
| | | 1H), 6.59 (dd, J = 2.8 Hz, 1.6 Hz, 1H), 5.44 (t, J = |
| | | 7.6 Hz, 1H), 2.03 (penta, J = 7.2 Hz, 2H), 1.02 (t, J = |
| | | 7.2 Hz, 3H) |
| 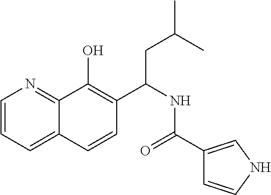 [SMILES: CC(C)CC(NC(=O)c1cc[nH]c1)c1ccc2cccnc2c1O] | 5 | N-(1-(8-hydroxyquinolin-7-yl)-3-methylbutyl)-1H- pyrrole-3-carboxamide LC-MS: m/z 324 (M + H)⁺. 1H NMR (400 MHz, CD3OD) δ (ppm): two rotamer, ratio is 1:1, 8.87 (d, J = 3.2 Hz) and 8.58 (d, J = 4.0 Hz) (total 1H), 8.29 and 8.22 (two set of d, J = 8.0 Hz, total 1H), 7.59-7.52 (m, 1H), 7.47- 7.43 (m, 1H), 7.39-7.32 (m, 1H), 7.34 and 7.07 (two set of d, J = 8.4 Hz, total 1H), 6.73-6.57 (m, total 1H), 5.88 and 5.67 (two set of t, J = 8.0 Hz, 1H), 1.99-1.91 (m) and 1.68-1.22 (two set of m, total 1H), 1.84-1.63 (m, 2H), 1.00 (d, J = 6.8 Hz, |
| | | 3H), 0.58 and 0.42 (two set of d, J = 6.8 Hz, 3H) |
| 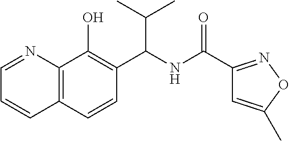 [SMILES: Cc1cc(C(=O)NC(c2ccc3cccnc3c2O)C(C)C)no1] | 5 | N-(1-(8-hydroxyquinolin-7-yl)-2-methylpropyl)-5- methylisoxazole-3-carboxamide LC-MS: m/z 326 (M + H)⁺. 1H NMR (400 MHz, DMSO-d6) δ (ppm): 9.80 (br s, 1H), 8.92 (d, J = 10.0 Hz, 1H), 8.86 (d, J = 2.8 Hz, 1H), 8.30 (d, J = 7.6 Hz, 1H), 7.68 (d, J = 8.8 Hz, 1H), 7.55 (dd, J = 8.0 Hz, 4.0 Hz, 1H), 7.40 (d, J = 8.8 Hz, 1H), 6.51 (s, 1H), 5.34 (d, J = 9.6 Hz, 1H), 2.45 (s, 3H), 2.27-2.22 (m, 1H), 1.04 (d, J = |
| | | 6.4 Hz, 3H), 0.76 (d, J = 6.4 Hz, 3H) |
| 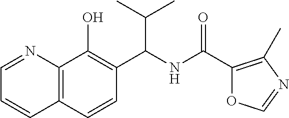 [SMILES: Cc1ncoc1C(=O)NC(c1ccc2cccnc2c1O)C(C)C] | 5 | N-(1-(8-hydroxyquinolin-7-yl)-2-methylpropyl)-4- methyloxazole-5-carboxamide LC-MS: m/z 326 (M + H)⁺. 1H NMR (400 MHz, CD3OD) δ (ppm): 9.01 (dd, J = 4.4 Hz, 1.2 Hz, 1H), 8.98 (dd, J = 8.4 Hz, 1.2 Hz, 1H), 8.21 (s, 1H), 7.97 (dd, J = 8.4 Hz, 4.4 Hz, 1H), 7.87 (d, J = 8.4 Hz, 1H), 7.79 (d, J = 8.8 Hz, 1H), 5.16 (d, J = 10.4 Hz, 1H), 2.58-2.51 (m, 1H), |
| | | 2.41 (s, 3H), 1.22 (d, J = 6.4 Hz, 3H), 0.84 (d, J = |
| | | 6.8 Hz, 3H) |
| 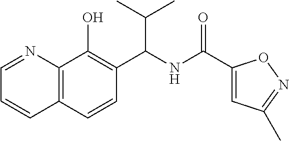 [SMILES: Cc1cc(C(=O)NC(c2ccc3cccnc3c2O)C(C)C)on1] | 5 | N-(1-(8-hydroxyquinolin-7-yl)-2-methylpropyl)-3- methylisoxazole-5-carboxamide LC-MS: m/z 326 (M + H)⁺. 1H NMR (400 MHz, CDCl3) δ (ppm): 8.72 (d, J = 3.6 Hz, 1H), 8.07 (d, J = 8.4 Hz, 1H), 7.97 (d, J = 9.2 Hz, 1H), 7.36 (dd, J = 8.4 Hz, , 4.4 Hz, 1H), 7.32 (d, J = 8.4 Hz, 1H), 7.24 (d, J = 8.4 Hz, 1H), 6.63 (s, 1H), 5.02 (t, J = 9.2 Hz, 1H), 2.41-2.35 (m, 1H), 2.10 (s, 3H), 1.04 (d, J = 6.4 Hz, 3H), 0.81 (d, |
| | | J = 6.8 Hz, 3H) |
| 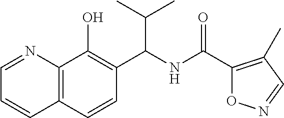 [SMILES: Cc1cnoc1C(=O)NC(c1ccc2cccnc2c1O)C(C)C] | 5 | N-(1-(8-hydroxyquinolin-7-yl)-2-methylpropyl)-4- methylisoxazole-5-carboxamide LC-MS: m/z 326 (M + H)⁺. 1H NMR (400 MHz, CDCl3) δ (ppm): 8.71 (d, J = 3.2 Hz, 1H), 8.09-8.06 (m, 2H), 7.92 (dd, J = 8.4 Hz, 1.2 Hz, 1H), 7.36 (dd, J = 8.4 Hz, , 4.0 Hz, 1H), 7.33 (d, J = 8.4 1H), 7.24 (d, J = 8.4 Hz, 1H), 5.04 (t, J = 9.2 Hz, 1H), 2.43-2.35 (m, 1H), 2.25 (s, |
| | | 3H), 1.03 (d, J = 6.8 Hz, 3H), 0.82 (d, J = 6.8 Hz, |
| | | 3H) |
| 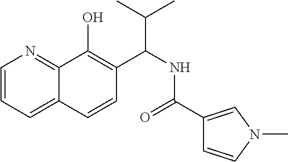 [SMILES: CC(C)C(NC(=O)c1ccn(C)c1)c1ccc2cccnc2c1O] | 5 | N-(1-(8-hydroxyquinolin-7-yl)-2-methylpropyl)-1- methyl-1H-pyrrole-3-carboxamide LC-MS: m/z 324 (M + H)⁺. 1H NMR (400 MHz, CD3OD) δ (ppm): 8.79 (d, J = 3.6 Hz, 1H), 8.22 (d, J = 8.4 Hz, 1H), 7.52-7.44 (m, 2H), 7.35 (d, J = 8.4 Hz, 1H), 7.24 (s, 1H), 6.64 (s, 1H), 6.51 (s, 1H), 5.19 (d, J = 9.6 Hz, 1H), 3.66 (s, 3H), 2.40-2.34 (m, 1H), 1.11 (d, J = 6.4 Hz, 3H), 0.84 (d, J = 6.8 Hz, 3H) |
| 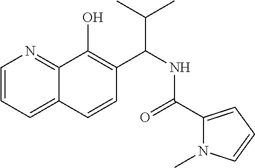 [SMILES: CC(C)C(NC(=O)c1cccn1C)c1ccc2cccnc2c1O] | 5 | N-(1-(8-hydroxyquinolin-7-yl)-2-methylpropyl)-1- methyl-1H-pyrrole-2-carboxamide LC-MS: m/z 324 (M + H)⁺. 1H NMR (400 MHz, CD3OD) δ (ppm): 8.99 (d, J = 4.4 Hz, 1.6 Hz, 1H), 8.96 (d, 8.4 Hz, 1H), 7.95 (dd, J = 8.0 Hz, 4.0 Hz, 1H), 7.85 (d, J = 8.8 Hz, 1H), 7.78 (d, J = 8.8 Hz, 1H), 6.68 (dd, J = 4.0 Hz, 2.4 Hz, 1H), 6.84 (d, J = 2.0 Hz, 1H), 6.06 (dd, J = 4.0 Hz, 1.2 Hz, 1H), 5.04 (d J = 10.4 Hz, 1H), 3.83 (s, 3H), 2.63-2.52 (m, 1H), 1.20 (d, J = 6.8 Hz, 3H), 0.82 (d, J = 6.8 Hz, 3H) |
| 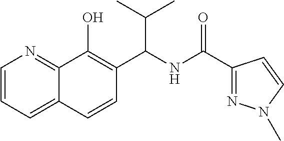 [SMILES: CC(C)C(NC(=O)c1ccn(C)n1)c1ccc2cccnc2c1O] | 5 | N-(1-(8-hydroxyquinolin-7-yl)-2-methylpropyl)-1- methyl-1H-pyrazole-3-carboxamide LC-MS: m/z 325 (M + H)⁺. 1H NMR (400 MHz, CD3OD) δ (ppm): 8.81 (dd, J = 4.4 Hz, 1.6 Hz, 1H), 8.25 (dd, J = 8.4 Hz, 1.6 Hz, 1H), 7.60 (d, J = 2.4 Hz, 1H), 7.51-7.46 (m, 2H), 7.38 (d, J = 8.8 Hz, 1H), 6.68 (d, J = 2.0 Hz, 1H), 5.17 (d J = 8.8 Hz, 1H), 3.95 (s, 3H), 2.47-2.40 (m, 1H), 1.10 (d, J = 6.8 Hz, 3H), 0.87 (d, J = 6.8 Hz, |
| | | 3H) |
| 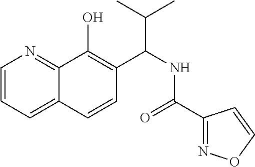 [SMILES: CC(C)C(NC(=O)c1ccon1)c1ccc2cccnc2c1O] | 5 | N-(1-(8-hydroxyquinolin-7-yl)-2- methylpropyl)isoxazole-3-carboxamide LC-MS: m/z 312 (M + H)⁺. 1H NMR (400 MHz, CDCl3) δ (ppm): mixture of rotamer (ratio is ca. 2:1) 8.83-8.72 (m, 2H), 8.39- 8.36 (m, 1H), 7.95-7.89 (m, 1H), 7.76-7.62 (m, 2H), 7.55 (d, J = 8.4 Hz, 1H), 6.71 (minor) and 6.69 (major) (two set of d, J = 1.6 Hz, 1H), 5.13- 5.02 (two set of t, J = 9.2 Hz, 1H), 2.62-2.45 (m, 1H), 1.02 (d, J = 6.8 Hz, 3H), 0.81 (minor) and 0.78 (major) (two set of d, J = 6.8 Hz, 3H) |
| 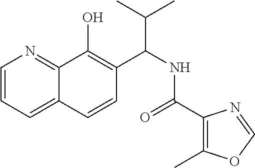 [SMILES: Cc1ocnc1C(=O)NC(c1ccc2cccnc2c1O)C(C)C] | 5 | N-(1-(8-hydroxyquinolin-7-yl)-2-methylpropyl)-5- methyloxazole-4-carboxamide LC-MS: m/z 326 (M + H)⁺. 1H NMR (400 MHz, DMSO-d6) δ (ppm): 9.89 (br s, 1H), 8.86 (dd, J = 4.0 Hz, 1.2 Hz, 1H), 8.40 (d, J = 10.0 Hz, 1H), 8.34 (s, 1H), 8.31 (dd, J = 8.4 Hz, 1.2 Hz, 1H), 7.59 (d, J = 8.8 Hz, 1H), 7.55 (dd, J = 8.4 Hz, 4.0 Hz, 1H), 7.39 (d, J = 8.4 Hz, 1H), 5.17 (t, J = 9.6 Hz, 1H), 2.53 (s, 3H), 2.36-2.28 (m, 1H), 1.01 (d, J = 6.8 Hz, 3H), 0.77 (d, J = 6.4 Hz, 3H) |
| 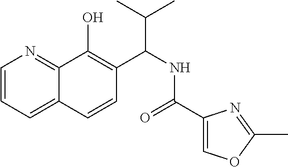 [SMILES: Cc1nc(C(=O)NC(c2ccc3cccnc3c2O)C(C)C)co1] | 5 | N-(1-(8-hydroxyquinolin-7-yl)-2-methylpropyl)-2- methyloxazole-4-carboxamide LC-MS: m/z 326 (M + H)⁺. 1H NMR (400 MHz, CD3OD) δ (ppm): 8.81 (dd, J = 4.0 Hz, 1.2 Hz, 1H), 8.24-8.20 (m, 2H), 7.49- 7.44 (m, 2H), 7.37 (d, J = 8.8 Hz, 1H), 5.15 (d, J = 9.2 Hz, 1H), 2.48 (s, 3H), 2.52-2.40 (m, 1H), 1.09 (d, J = 6.4 Hz, 3H), 0.86 (d, J = 6.4 Hz, 3H) |
| 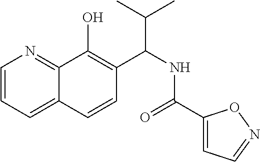 [SMILES: CC(C)C(NC(=O)c1ccno1)c1ccc2cccnc2c1O] | 5 | N-(1-(8-hydroxyquinolin-7-yl)-2- methylpropyl)isoxazole-5-carboxamide LC-MS: m/z 312 (M + H)⁺. 1H NMR (400 MHz, DMSO-d6) δ (ppm): 9.75 (br s, 1H), 9.16 (d, J = 8.8 Hz, 1H), 8.85 (d, J = 2.0 Hz, 1H), 8.73 (d, J = 1.2 Hz, 1H), 8.29 (d, J = 8.4 Hz, 1.2 Hz, 1H), 7.68 (d, J = 8.4 Hz, 1H), 7.55 (dd, J = 8.4 Hz, 4.4 Hz, 1H), 7.41 (d, J = 8.4 Hz, 1H), 7.09 (d, J = 1.2 Hz, 1H), 5.39 (t, J = 9.6 Hz, 1H), 2.29- 2.16 (m, 1H), 1.05 (d, J = 6.8 Hz, 3H), 0.77 (d, J = |
| | | 6.4 Hz, 3H) |
| 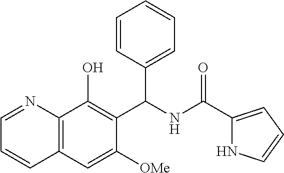 [SMILES: COc1cc2cccnc2c(O)c1C(NC(=O)c1ccc[nH]1)c1ccccc1] | 4 | N-((8-hydroxy-6-methoxyquinolin-7- yl)(phenyl)methyl)-1H-pyrrole-2-carboxamide LC-MS: m/z 374 (M + H)⁺. 1H NMR (400 MHz, CD3OD) δ (ppm): 8.67 (dd, J = 4.4 Hz, 1.6 Hz, 1H), 8.18 (d, J = 8.4 Hz, 1H), 7.46 (dd, J = 8.0 Hz, 4.0 Hz, 1H), 7.37-7.33 (m, 2H), 7.27-7.22 (m, 2H), 7.21-7.15 (m, 2H), 6.95- 6.90 (m, 2H), 6.79 (dd, J = 4.0 Hz, 1.6 Hz, 1H), 6.20 (dd, J = 3.6 Hz, 2.8 Hz, 1H), 3.98 (s, 3H) |
| 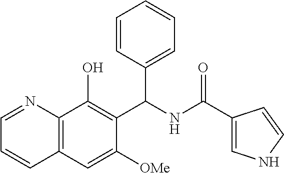 [SMILES: COc1cc2cccnc2c(O)c1C(NC(=O)c1cc[nH]c1)c1ccccc1] | 4 | N-((8-hydroxy-6-methoxyquinolin-7- yl)(phenyl)methyl)-1H-pyrrole-3-carboxamide LC-MS: m/z 374 (M + H)⁺. 1H NMR (400 MHz, DMSO-d6) δ (ppm): 11.28 (br s, 1H), 8.73 (dd, J = 4.4 Hz, 1.2 Hz, 1H), 8.71 (d, J = 8.4 Hz, 1.2 Hz, 1H), 8.17 (d, J = 8.4 Hz, 1H), 7.57 (dd, J = 8.4 Hz, 4.4 Hz, 1H), 7.37-7.25 (m, 5H), 7.24-7.21 (m, 1H), 7.16 (d, J = 8.4 Hz, 1H), 7.00 (s, 1H), 6.82 (d, J = 2.4 Hz, 1H), 6.42 (d, J = 2.0 Hz, 1H), 3.96 (s, 3H) |
| 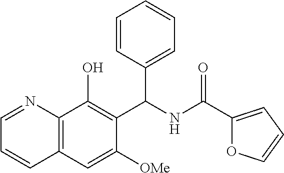 [SMILES: COc1cc2cccnc2c(O)c1C(NC(=O)c1ccco1)c1ccccc1] | 4 | N-((8-hydroxy-6-methoxyquinolin-7- yl)(phenyl)methyl)furan-2-carboxamide LC-MS: m/z 375 (M + H)⁺. 1H NMR (400 MHz, DMSO-d6) δ (ppm): 8.74 (dd, J = 4.4 Hz, 1.2 Hz, 1H), 8.63 (d, J = 9.6 Hz, 1H), 8.29 (dd, J = 8.4 Hz, 1.6 Hz, 1H), 7.90 (s, 1H), 7.58 (dd, J = 8.4 Hz, 4.4 Hz, 1H), 7.31-7.28 (m, 4H), 7.26-7.17 (m, 2H), 7.12 (d, J = 8.4 Hz, 1H), 7.04 (s, 1H), 6.68 (dd, J = 3.2 Hz, 1.6 Hz, 1H), 3.97 (s, 3H) |
| 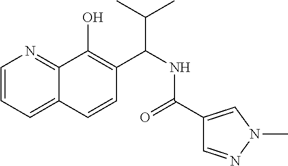 [SMILES: CC(C)C(NC(=O)c1cnn(C)c1)c1ccc2cccnc2c1O] | 5 | N-(1-(8-hydroxyquinolin-7-yl)-2-methylpropyl)-1- methyl-1H-pyrazole-4-carboxamide LC-MS: m/z 325 (M + H)⁺. 1H NMR (400 MHz, DMSO-d6) δ (ppm): 8.85 (d, J = 4.4 Hz, 1H), 8.31 (d, J = 9.6 Hz, 1H), 8.19 (s, 1H), 8.15 (d, J = 8.4 Hz, 1H), 7.88 (s, 1H), 7.66 (d, J = 8.4 Hz, 1H), 7.54 (dd, J = 8.4 Hz, 4.4 Hz, 1H), 7.40 (d, J = 8.4 Hz, 1H), 5.40 (t, J = 10.0 Hz, 1H), 3.85 (s, 3H), 2.18-2.06 (m, 1H), 1.02 (d, J = 6.8 Hz, 3H), 0.79 (d, J = 6.8 Hz, 3H) |
| 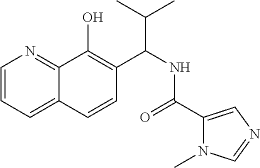 [SMILES: CC(C)C(NC(=O)c1cncn1C)c1ccc2cccnc2c1O] | 5 | N-(1-(8-hydroxyquinolin-7-yl)-2-methylpropyl)-1- methyl-1H-imidazole-5-carboxamide LC-MS: m/z 325 (M + H)⁺. 1H NMR (400 MHz, DMSO-d6) δ (ppm): 9.72 (br s, 1H), 8.85 (dd, J = 4.4 Hz, 1.2 Hz, 1H), 8.36 (dd, J = 9.6 Hz, 1H), 8.30 (dd, J = 8.4 Hz, 1.6 Hz, 1H), 7.71 (s, 2H), 7.66 (d, J = 8.4 Hz, 1H), 7.54 (dd, J = 8.4 Hz, 4.4 Hz, 1H), 7.41 (d, J = 8.4 Hz, 1H), 5.37 (t, J = 10.0 Hz, 1H), 3.75 (s, 3H), 2.24-2.12 (m, 1H), 1.04 (d, J = 6.8 Hz, 3H), 0.78 (d, J = 6.8 Hz, 3H) |
| 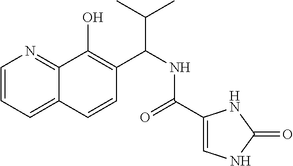 [SMILES: CC(C)C(NC(=O)c1c[nH]c(=O)[nH]1)c1ccc2cccnc2c1O] | 5 | N-(1-(8-hydroxyquinolin-7-yl)-2-methylpropyl)-2- oxo-2,3-dihydro-1H-imidazole-4-carboxamide LC-MS: m/z 327 (M + H)⁺. 1H NMR (400 MHz, DMSO-d6) δ (ppm): 10.28 (br s, 2H), 9.67 (br s, 1H), 8.86 (dd, J = 4.4 Hz, 1.2 Hz, 1H), 8.31 (dd, J = 8.4 Hz, 1.6 Hz, 1H), 7.89 (d, J = 9.2 Hz, 1H), 7.58-7.52 (m, 2H), 7.41 (d, J = 8.4 Hz, 1H), 7.12 (s, 1H), 5.34 (t, J = 9.6 Hz, 1H), 2.19-2.09 (m, 1H), 1.00 (d, J = 6.8 Hz, 3H), 0.80 (d, J = 6.8 Hz, 3H) |
| 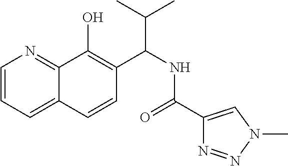 [SMILES: CC(C)C(NC(=O)c1cn(C)nn1)c1ccc2cccnc2c1O] | 5 | N-(1-(8-hydroxyquinolin-7-yl)-2-methylpropyl)-1- methyl-1H-1,2,3-triazole-4-carboxamide LC-MS: m/z 326 (M + H)⁺. 1H NMR (400 MHz, DMSO-d6) δ (ppm): 8.97 (d, J = 4.8 Hz, 1H), 8.52 (d, J = 8.4 Hz, 1H), 8.21 (d, J = 9.6 Hz, 1H), 8.03 (s, 1H), 7.65 (dd, J = 8.4 Hz, 4.4 Hz, 1H), 7.61 (dd, J = 8.4 Hz, 4.4 Hz, 1H), 7.46 (d, J = 8.4 Hz, 1H), 5.14 (t, J = 9.6 Hz, 1H), 4.06 (s, 3H), 2.52-2.43 (m, 1H), 1.10 (d, J = 6.8 Hz, 3H), 0.82 (d, J = 6.8 Hz, 3H) |
| 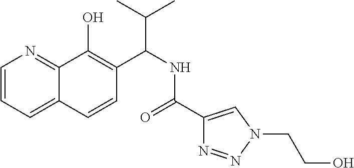 [SMILES: CC(C)C(NC(=O)c1cn(CCO)nn1)c1ccc2cccnc2c1O] | 5 | 1-(2-hydroxyethyl)-N-(1-(8-hydroxyquinolin-7-yl)- 2-methylpropyl)-1H-1,2,3-triazole-4-carboxamide LC-MS: m/z 356 (M + H)⁺. 1H NMR (400 MHz, DMSO-d6) δ (ppm): 8.86 (dd, J = 4.4 Hz, 1.6 Hz, 1H), 8.71 (d, J = 9.6 Hz, 1H), 8.47 (s, 1H), 8.30 (dd, J = 8.4 Hz, 1.6 Hz, 1H), 7.68 (d, J = 8.4 Hz, 1H), 7.54 (dd, J = 8.4 Hz, 4.4 Hz, 1H), 7.40 (d, J = 8.4 Hz, 1H), 5.29 (t, J = 9.6 Hz, 1H), 5.02 (t, J = 4.0 Hz, 1H), 4.44 (t, J = 4.8 Hz, 2H), 3.81-3.76 (m, 2H), 2.34-2.27 (m, 1H), 1.03 (d, J = 6.8 Hz, 3H), 0.77 (d, J = 6.4 Hz, 3H) |
| 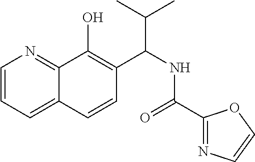 [SMILES: CC(C)C(NC(=O)c1ncco1)c1ccc2cccnc2c1O] | 5 | N-(1-(8-hydroxyquinolin-7-yl)-2- methylpropyl)oxazole-2-carboxamide LC-MS: m/z 312 (M + H)⁺. 1H NMR (400 MHz, DMSO-d6) δ (ppm): 9.13 (d, J = 9.2 Hz, 1H), 8.85 (dd, J = 4.4 Hz, 1.2 Hz, 1H), 8.32-8.28 (m, 2H), 7.70 (d, J = 8.8 Hz, 1H), 7.55 (dd, J = 8.4 Hz, 4.4 Hz, 1H), 7.45 (s, 1H), 7.41 (d, J = 8.8 Hz, 1H), 5.29 (t, J = 9.6 Hz, 1H), 2.34-2.26 (m, 1H), 1.04 (d, J = 6.8 Hz, 3H), 0.76 (d, J = 6.4 Hz, 3H) |
| 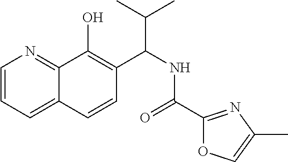 [SMILES: Cc1coc(C(=O)NC(c2ccc3cccnc3c2O)C(C)C)n1] | 5 | N-(1-(8-hydroxyquinolin-7-yl)-2-methylpropyl)-4- methyloxazole-2-carboxamide LC-MS: m/z 326 (M + H)⁺. 1H NMR (400 MHz, CDCl3) δ (ppm): 8.71 (dd, J = 4.4 Hz, 1.6 Hz, 1H), 8.29 (d, J = 9.2 Hz, 1H), 8.07 (dd, J = 8.4 Hz, 1.2 Hz, 1H), 7.45 (s, 1H), 7.42-7.34 (m, 2H), 7.27 (d, J = 8.4 Hz, 1H), 5.03 (t, J = 9.2 Hz, 1H), 2.46-2.35 (m, 1H), 2.15 (s, 3H), 1.05 (d, J = 6.8 Hz, 3H), 0.81 (d, J = 6.8 Hz, 3H) |
| 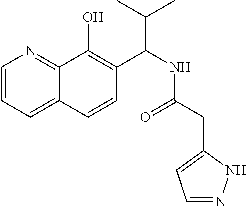 [SMILES: CC(C)C(NC(=O)Cc1ccn[nH]1)c1ccc2cccnc2c1O] | 5 | N-(1-(8-hydroxyquinolin-7-yl)-2-methylpropyl)-2- (1H-pyrazol-5-yl)acetamide LC-MS: m/z 325 (M + H)⁺. 1H NMR (400 MHz, DMSO-d6) δ (ppm): 12.53 (br s, 1H), 9.68 (br s, 1H), 8.84 (dd, J = 4.4 Hz, 1.2 Hz, 1H), 8.31-8.27 (m, 2H), 7.59-7.50 (m, 3H), 7.39 (d, J = 8.4 Hz, 1H), 6.07 (br s, 1H), 5.24 (t, J = 10.4 Hz, 1H), 3.50 (s, 2H), 2.12-2.02 (m, 1H), 0.91 (d, J = 6.8 Hz, 3H), 0.78 (d, J = 6.8 Hz, 3H) |
| 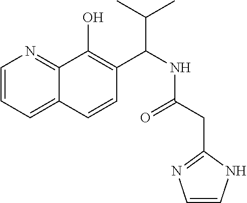 [SMILES: CC(C)C(NC(=O)Cc1ncc[nH]1)c1ccc2cccnc2c1O] | 5 | N-(1-(8-hydroxyquinolin-7-yl)-2-methylpropyl)-2- (1H-imidazol-2-yl)acetamide LC-MS: m/z 325 (M + H)⁺. 1H NMR (400 MHz, CD3OD) δ (ppm): 8.77 (dd, J = 4.0 Hz, 1.6 Hz, 1H), 8.25 (d, J = 8.4 Hz, 1H), 7.48 (dd, J = 8.4 Hz, 1.6 Hz, 1H), 7.45 (d, J = 8.4 Hz, 1H), 7.36 (d, J = 8.4 Hz, 1H), 6.94 (s, 2H), 5.16 (d, J = 10.4 Hz, 1H), 3.31 (s, 2H), 2.31-2.22 (m, 1H), 1.02 (d, J = 6.8 Hz, 3H), 0.79 (d, J = 6.8 Hz, 3H) |
| 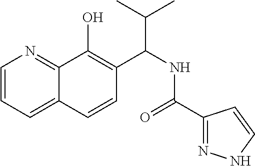 [SMILES: CC(C)C(NC(=O)c1cc[nH]n1)c1ccc2cccnc2c1O] | 5 | N-(1-(8-hydroxyquinolin-7-yl)-2-methylpropyl)- 1H-pyrazole-3-carboxamide LC-MS: m/z 311 (M + H)⁺. 1H NMR (400 MHz, DMSO-d6) δ (ppm): 13.24 (br s, 1H), 9.66 (br s, 1H), 8.66 (d, J = 4.4 Hz, 1H), 8.38 (d, J = 9.2 Hz, 1H), 8.30 (d, J = 8.4 Hz, 1H), 7.76 (s, 1H), 7.63 ( d, J = 8.4 Hz, 1H), 7.54 (dd, J = 8.4 Hz, 4.4 Hz, 1H), 7.39 (d, J = 8.4 Hz, 1H), 6.63 (br s, 1H), 5.24 (br s, 1H), 2.32-2.25 (m, 1H), 1.02 (d, J = 6.8 Hz, 3H), 0.77 (d, J = 6.8 Hz, 3H) |
| 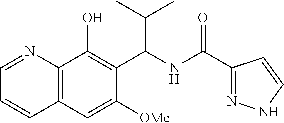 [SMILES: COc1cc2cccnc2c(O)c1C(NC(=O)c1cc[nH]n1)C(C)C] | 5 | N-(1-(8-hydroxy-6-methoxyquinolin-7-yl)-2- methylpropyl)-1H-pyrazole-3-carboxamide LC-MS: m/z 341 (M + H)⁺. 1H NMR (400 MHz, CD3OD) δ (ppm): 8.77 (d, J = 4.4 Hz, 1H), 8.65 (d, J = 8.8 Hz, 1H), 7.78 (dd, J = 8.4 Hz, 4.4 Hz, 1H), 7.69 (d, J = 2.0 Hz, 1H), 7.15 (s, 1H), 6.76 (d, J = 2.0 Hz, 1H), 5.47 (d, J = 10.4 Hz, 1H), 4.08 (s, 3H), 2.69-2.61 (m, 1H), |
| | | 1.15 (d, J = 6.8 Hz, 3H), 0.80 (d, J = 6.8 Hz, 3H) |
| 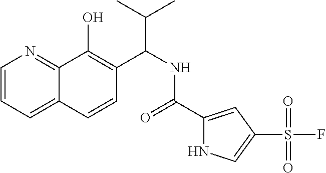 [SMILES: CC(C)C(NC(=O)c1cc(S(=O)(=O)F)c[nH]1)c1ccc2cccnc2c1O] | 5 | 5-((1-(8-hydroxyquinolin-7-yl)-2- methylpropyl)carbamoyl)-1H-pyrrole-3-sulfonyl fluoride LC-MS: m/z 392 (M + H)⁺. 1H NMR (400 MHz, DMSO-d6) δ (ppm): 12.99 (br s, 1H), 9.78 (br s, 1H), 8.86 (d, J = 4.4 Hz, 1H), 8.58 (d, J = 9.2 Hz, 1H), 8.30 (d, J = 8.4 Hz, 1H), 7.92 (s, 1H), 7.67-7.64 (m, 2H), 7.55 (dd, J = 8.4 Hz, 4.4 Hz, 1H), 7.42 (d, J = 8.4 Hz, 1H), 5.43 (t, J = 9.2 Hz, 1H), 2.25-2.15 (m, 1H), 1.04 (d, J = 6.8 Hz, 3H), 0.81 (d, J = 6.8 Hz, 3H) |
| 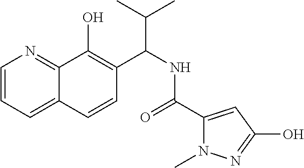 [SMILES: CC(C)C(NC(=O)c1cc(O)nn1C)c1ccc2cccnc2c1O] | 5 | 3-hydroxy-N-(1-(8-hydroxyquinolin-7-yl)-2- methylpropyl)-1-methyl-1H-pyrazole-5- carboxamide LC-MS: m/z 341 (M + H)⁺. 1H NMR (400 MHz, DMSO-d6) δ (ppm): 9.69 (br s, 1H), 8.86 (dd, J = 4.4 Hz, 1.6 Hz, 1H), 8.48 (d, J = 9.2 Hz, 1H), 8.33 (d, J = 8.4 Hz, 1.6 Hz, 1H), 7.66 (d, J = 8.4 Hz, 1H), 7.55 (dd, J = 8.4 Hz, 4.4 Hz, 1H), 7.41 (d, J = 8.4 Hz, 1H), 6.18 (s, 1H), 5.36 (t, J = 9.2 Hz, 1H), 3.76 (s, 3H), 2.23-2.11 (m, 1H), 1.04 (d, J = 6.8 Hz, 3H), 0.77 (d, J = 6.8 Hz, |
| | | 3H) |
| 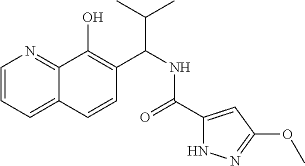 [SMILES: COc1cc(C(=O)NC(c2ccc3cccnc3c2O)C(C)C)[nH]n1] | 5 | N-(1-(8-hydroxyquinolin-7-yl)-2-methylpropyl)-3- methoxy-1H-pyrazole-5-carboxamide LC-MS: m/z 341 (M + H)⁺. 1H NMR (400 MHz, DMSO-d6) δ (ppm): 12.60 (br s, 1H), 9.55 (br s, 1H), 8.86 (d, J = 4.4 Hz, 1H), 8.46 (br s, 1H), 8.31 (d, J = 9.2 Hz, 1H), 7.65 (d, J = 8.4 Hz, 1H), 7.55 (dd, J = 8.4 Hz, 4.4 Hz, 1H), 7.42 (d, J = 8.4 Hz, 1H), 6.43 (br s, 1H), 5.39 (br s, 1H), 3.79 (s, 3H), 2.24-2.13 (m, 1H), 1.03 (d, J = 6.8 Hz, 3H), 0.79 (d, J = 6.8 Hz, 3H) |
| 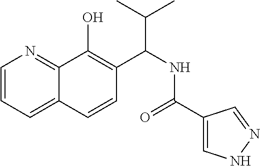 [SMILES: CC(C)C(NC(=O)c1cn[nH]c1)c1ccc2cccnc2c1O] | 5 | N-(1-(8-hydroxyquinolin-7-yl)-2-methylpropyl)- 1H-pyrazole-4-carboxamide LC-MS: m/z 311 (M + H)⁺. 1H NMR (400 MHz, CD3OD) δ (ppm): 8.80 (dd, J = 4.0 Hz, 1.2 Hz, 1H), 8.22 (dd, J = 8.4 Hz, 1.6 Hz, 1H), 8.09 (s, 2H), 7.53 (d, J = 8.4 Hz, 1H), 7.47 (dd, J = 8.4 Hz, 4.0 Hz, 1H), 7.38 (d, J = 8.4 Hz, 1H), 5.29 (d, J = 10.0 Hz, 1H), 2.42-2.35 (m, 1H), 1.14 (d, J = 6.8 Hz, 3H), 0.85 (d, J = 6.8 Hz, 3H) |
| 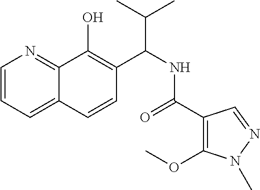 [SMILES: COc1c(C(=O)NC(c2ccc3cccnc3c2O)C(C)C)cnn1C] | 5 | N-(1-(8-hydroxyquinolin-7-yl)-2-methylpropyl)-5- methoxy-1-methyl-1H-pyrazole-4-carboxamide LC-MS: m/z 355 (M + H)⁺. 1H NMR (400 MHz, CD3OD) δ (ppm): 8.98 (dd, J = 4.8 Hz, 1.2 Hz, 1H), 8.88 (d, J = 8.4 Hz, 1H), 7.90 (dd, J = 8.4 Hz, 5.2 Hz, 1H), 7.82-7.71 (m, 3H), 5.09 (d, J = 10.4 Hz, 1H), 4.03 (s, 3H), 3.66 (s, 3H), 2.58-2.50 (m, 1H), 1.21 (d, J = 6.4 Hz, 3H), 0.84 (d, J = 6.4 Hz, 3H) |
| 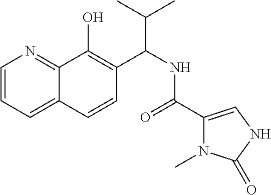 [SMILES: CC(C)C(NC(=O)c1c[nH]c(=O)n1C)c1ccc2cccnc2c1O] | 5 | N-(1-(8-hydroxyquinolin-7-yl)-2-methylpropyl)-3- methyl-2-oxo-2,3-dihydro-1H-imidazole-4- carboxamide LC-MS: m/z 341 (M + H)⁺. 1H NMR (400 MHz, CD3OD) δ (ppm): 9.00 (dd, J = 4.4 Hz, 1.2 Hz, 1H), 8.94 (d, J = 8.0 Hz, 1H), 7.94 (dd, J = 8.4 Hz, 4.8 Hz, 1H), 7.81 (d, J = 8.4 Hz, 1H), 7.78 (d, J = 8.4 Hz, 1H), 7.24 (s, 1H), 5.10 (t, J = 10.4 Hz, 1H), 3.48 (s, 3H), 2.49-2.40 (m, 1H), 1.20 (d, J = 6.8 Hz, 3H), 0.82 (d, J = 6.8 Hz, 3H) |
| 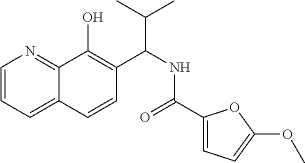 [SMILES: COc1ccc(C(=O)NC(c2ccc3cccnc3c2O)C(C)C)o1] | 5 | N-(1-(8-hydroxyquinolin-7-yl)-2-methylpropyl)-5- methoxyfuran-2-carboxamide LC-MS: m/z 341 (M + H)⁺. 1H NMR (400 MHz, DMSO-d6) δ (ppm): 9.86 (br s, 1H), 8.86 (d, J = 4.4 Hz, 1.2 Hz, 1H), 8.30 (dd, J = 8.4 Hz, 1.6 Hz, 1H), 8.20 (d, J = 9.2 Hz, 1H), 7.67 (d, J = 8.4 Hz, 1H), 7.56 (dd, J = 8.4 Hz, 4.4 Hz, 1H), 7.40 (d, J = 8.4 Hz, 1H), 7.06 (d, J = 4.0 Hz, 1H), 5.54 (d, J = 4.0 Hz, 1H), 5.29 (t, J = 9.2 Hz, 1H), 3.80 (s, 3H), 2.26-2.17 (m, 1H), 1.02 (d, J = 6.8 Hz, 3H), 0.76 (d, J = 6.8 Hz, 3H) |
| 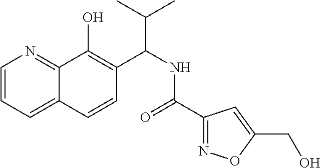 [SMILES: CC(C)C(NC(=O)c1cc(CO)on1)c1ccc2cccnc2c1O] | 5 | 5-(hydroxymethyl)-N-(1-(8-hydroxyquinolin-7-yl)- 2-methylpropyl)isoxazole-3-carboxamide LC-MS: m/z 342 (M + H)⁺. 1H NMR (400 MHz, DMSO-d6) δ (ppm): 9.88 (br s, 1H), 8.99 (d, J = 9.2 Hz 1H), 8.87 (dd, J = 4.4 Hz, 1H), 8.31 (d, J = 8.4 Hz, 1H), 7.69 (d, J = 8.4 Hz, 1H), 7.56 (dd, J = 8.4 Hz, 4.4 Hz, 1H), 7.41 (d, J = 8.4 Hz, 1H), 6.64 (s, 1H), 5.72 (t, J = 6.0 Hz, 1H), 5.36 (t, J = 9.2 Hz, 1H), 4.61 (d, J = 6.0 Hz, 2H), 2.24-2.20 (m, 1H), 1.05 (d, J = 6.8 Hz, 3H), 0.77 (d, J = 6.8 Hz, 3H) |
| 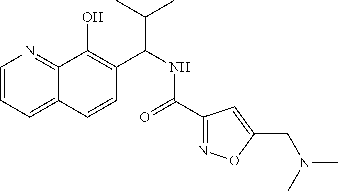 [SMILES: CC(C)C(NC(=O)c1cc(CN(C)C)on1)c1ccc2cccnc2c1O] | 5 | 5-((dimethylamino)methyl)-N-(1-(8- hydroxyquinolin-7-yl)-2-methylpropyl)isoxazole- 3-carboxamide LC-MS: m/z 369 (M + H)⁺. 1H NMR (400 MHz, DMSO-d6) δ (ppm): 9.86 (br s, 1H), 8.99 (d, J = 8.8 Hz 1H), 8.86 (dd, J = 4.4 Hz, 1.6 Hz, 1H), 8.31 (dd, J = 8.4 Hz, 1.2 Hz, 1H), 7.69 (d, J = 8.4 Hz, 1H), 7.56 (dd, J = 8.4 Hz, 4.4 Hz, 1H), 7.41 (d, J = 8.4 Hz, 1H), 6.67 (s, 1H), 5.36 (t, J = 9.2 Hz, 1H), 3.66 (s, 2H), 2.28-2.18 (m, 1H), 2.23 (s, 6H), 1.05 (d, J = 6.8 Hz, 3H), 0.77 (d, J = 6.8 Hz, 3H) |
| 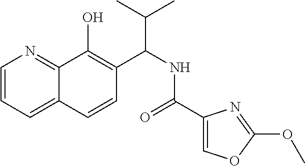 [SMILES: COc1nc(C(=O)NC(c2ccc3cccnc3c2O)C(C)C)co1] | 5 | N-(1-(8-hydroxyquinolin-7-yl)-2-methylpropyl)-2- methoxyoxazole-4-carboxamide LC-MS: m/z 342 (M + H)⁺. 1H NMR (400 MHz, DMSO-d6) δ (ppm): 8.87 (d, J = 4.4 Hz, 1.6 Hz 1H), 8.34-8.30 (m, 2H), 8.14 (s, 1H), 7.60 (d, J = 8.4 Hz, 1H), 7.56 (dd, J = 8.4 Hz, 4.4 Hz, 1H), 7.41 (d, J = 8.4 Hz, 1H), 5.17 (t, J = 9.2 Hz, 1H), 4.11 (s, 3H), 2.36-2.27 (m, 1H), 1.01 (d, J = 6.8 Hz, 3H), 0.77 (d, J = 6.8 Hz, 3H) |
| 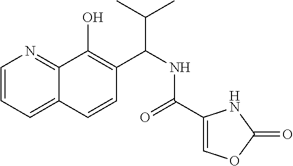 [SMILES: CC(C)C(NC(=O)c1coc(=O)[nH]1)c1ccc2cccnc2c1O] | 5 | N-(1-(8-hydroxyquinolin-7-yl)-2-methylpropyl)-2- oxo-2,3-dihydrooxazole-4-carboxamide LC-MS: m/z 328 (M + H)⁺. 1H NMR (400 MHz, DMSO-d6) δ (ppm): 8.87 (d, J = 4.4 Hz, 1.6 Hz 1H), 8.32 (d, J = 8.4 Hz, 1H), 8.30 (d, J = 8.8 Hz, 1H), 7.78 (s, 1H), 7.57-7.52 (m, 2H), 7.42 (d, J = 8.4 Hz, 1H), 5.33 (t, J = 9.2 Hz, 1H), 2.20-2.13 (m, 1H), 1.01 (d, J = 6.8 Hz, 3H), 0.80 (d, J = 6.8 Hz, 3H) |
| 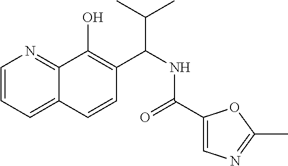 [SMILES: Cc1ncc(C(=O)NC(c2ccc3cccnc3c2O)C(C)C)o1] | 5 | N-(1-(8-hydroxyquinolin-7-yl)-2-methylpropyl)-2- methyloxazole-5-carboxamide LC-MS: m/z 326 (M + H)⁺. 1H NMR (400 MHz, CD3OD) δ (ppm): 8.99 (d, J = 4.4 Hz, 1.6 Hz 1H), 8.90 (dd, J = 8.4 Hz, 1.2 Hz, 1H), 7.93 (dd, J = 8.4 Hz, 4.8 Hz, 1H), 7.82 (d, J = 8.8 Hz, 1H), 7.75 (d, J = 8.8 Hz, 1H), 7.61 (s, 1H), 5.20 (d, J = 10.4 Hz, 1H), 2.53 (s, 3H), 2.55- 2.46 (m, 1H), 1.21 (d, J = 6.8 Hz, 3H), 0.84 (d, J = 6.8 Hz, 3H) |
| 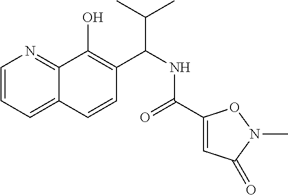 [SMILES: CC(C)C(NC(=O)c1cc(=O)n(C)o1)c1ccc2cccnc2c1O] | 5 | N-(1-(8-hydroxyquinolin-7-yl)-2-methylpropyl)-2- methyl-3-oxo-2,3-dihydroisoxazole-5-carboxamide LC-MS: m/z 342 (M + H)⁺. 1H NMR (400 MHz, CD3OD) δ (ppm): 8.98 (d, J = 4.4 Hz, 1.6 Hz 1H), 8.82 (d, J = 8.0 Hz, 1H), 7.88 (dd, J = 8.4 Hz, 4.4 Hz, 1H), 7.75 (d, J = 8.4 Hz, 1H), 7.70 (d, J = 8.4 Hz, 1H), 6.40 (s, 1H), 5.25 (d, J = 10.4 Hz, 1H), 3.61 (s, 3H), 2.46-2.39 (m, 1H), 1.19 (d, J = 6.8 Hz, 3H), 0.83 (d, J = 6.8 Hz, 3H) |
| 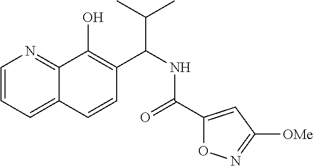 [SMILES: COc1cc(C(=O)NC(c2ccc3cccnc3c2O)C(C)C)on1] | 5 | N-(1-(8-hydroxyquinolin-7-yl)-2-methylpropyl)-3- methoxyisoxazole-5-carboxamide LC-MS: m/z 342 (M + H)⁺. 1H NMR (400 MHz, DMSO-d6) δ (ppm): 9.85 (br s, 1H), 9.10 (d, J = 8.4 Hz, 1H), 8.86 (d, J = 1.6 Hz, 1H), 8.31 (d, J = 8.8 Hz, 1H), 7.68 (d, J = 8.4 Hz, 1H), 7.56 (dd, J = 8.4 Hz, 4.4 Hz, 1H), 7.42 (d, J = 8.4 Hz, 1H), 6.85 (s, 1H), 5.36 (t, J = 8.4 Hz, 1H), 3.94 (s, 3H), 2.29-2.18 (m, 1H), 1.05 (d, J = 6.8 Hz, 3H), 0.77 (d, J = 6.8 Hz, 3H) |
| 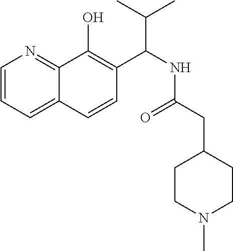 [SMILES: CC(C)C(NC(=O)CC1CCN(C)CC1)c1ccc2cccnc2c1O] | 5 | N-(1-(8-hydroxyquinolin-7-yl)-2-methylpropyl)-2- (1-methylpiperidin-4-yl)acetamide LC-MS: m/z 356 (M + H)⁺. 1H NMR (400 MHz, CD3OD) δ (ppm): 8.80 (dd, J = 4.4 Hz, 1.6 Hz, 1H), 8.21 (dd, J = 8.4 Hz, 1.6 Hz, 1H), 7.46 (dd, J = 8.0 Hz, 4.0 Hz, 1H), 7.43 (d, J = 8.4 Hz, 1H), 7.35 (d, J = 8.8 Hz, 1H), 5.15 (d, J = 9.2 Hz, 1H), 2.89-2.83 (m, 2H), 2.28 (s, 3H), 2.30- 2.26 (m, 1H), 2.18 (d, J = 6.4 Hz, 2H), 2.12-1.95 (m, 2H), 1.78-1.71 (m, 2H), 1.65-1.56 (m, 1H), 1.38-1.26 (m, 2H), 1.06 (d, J = 6.8 Hz, 3H), 0.83 (d, J = 6.8 Hz, 3H) |
| 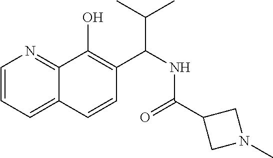 [SMILES: CC(C)C(NC(=O)C1CN(C)C1)c1ccc2cccnc2c1O] | 5 | N-(1-(8-hydroxyquinolin-7-yl)-2-methylpropyl)-1- methylazetidine-3-carboxamide LC-MS: m/z 314 (M + H)⁺. 1H NMR (400 MHz, CD3OD) δ (ppm): 8.80 (dd, J = 4.4 Hz, 1.6 Hz, 1H), 8.22 (dd, J = 8.4 Hz, 1.6 Hz, 1H), 7.47 (dd, J = 8.4 Hz, 4.4 Hz, 1H), 7.41 (d, J = 8.4 Hz, 1H), 7.35 (d, J = 8.4 Hz, 1H), 5.16 (d, J = 9.2 Hz, 1H), 3.70-3.56 (m, 2H), 3.47-3.40 (m, 2H), 3.38-3.22 (m, 1H), 2.35(s, 3H), 2.33-2.23 (m, 1H), 1.06 (d, J = 6.8 Hz, 3H), 0.80 (d, J = 6.8 Hz, 3H) |
| 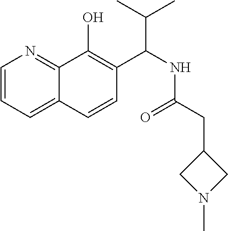 [SMILES: CC(C)C(NC(=O)CC1CN(C)C1)c1ccc2cccnc2c1O] | 5 | N-(1-(8-hydroxyquinolin-7-yl)-2-methylpropyl)-2- (1-methylazetidin-3-yl)acetamide LC-MS: m/z 328 (M + H)⁺. 1H NMR (400 MHz, DMSO-d6) δ (ppm): 9.67 (br s, 1H), 8.84 (dd, J = 8.4 Hz, 1.6 Hz, 1H), 8.29 (dd, J = 8.4 Hz, 1.6 Hz, 1H), 8.16 (d, J = 9.2 Hz, 1H), 7.53 (dd, J = 8.4 Hz, 4.4 Hz, 1H), 7.50 (d, J = 8.8 Hz, 1H), 7.39 (d, J = 8.4 Hz, 1H), 5.23 (t, J = 8.4 Hz, 1H), 3.25-3.14 (m, 2H), 2.78-2.68 (m, 2H), 2.59-2.46 (m, 1H), 2.45-2.30 (m, 2H), 2.13 (s, 3H), 2.09-1.93 (m, 1H), 0.91 (d, J = 6.4 Hz, 3H), 0.77 (d, J = 6.8 Hz, 3H) |
| 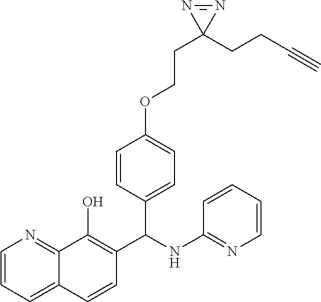 [SMILES: C#CCCC1(CCOc2ccc(C(Nc3ccccn3)c3ccc4cccnc4c3O)cc2)N=N1] | 2 | 7-((4-(2-(3-(but-3-yn-1-yl)-3H-diazirin-3- yl)ethoxy)phenyl)(pyridin-2- ylamino)methyl)quinolin-8-ol LC-MS: m/z 464 (M + H)⁺. 1H NMR (400 MHz, DMSO-d6) δ 9.89 (brs, 1H), 8.84 (dd, J = 4.2, 1.6 Hz, 1H), 8.28 (dd, J = 8.3, 1.6 Hz, 1H), 7.90 (dd, J = 5.2, 1.8 Hz, 1H), 7.62 (d, J = 8.5 Hz, 1H), 7.52 (dd, J = 8.3, 4.2 Hz, 1H), 7.42- 7.28 (m, 3H), 7.24 (d, J = 8.7 Hz, 2H), 6.86-6.80 (m, 2H), 6.76 (d, J = 8.3 Hz, 1H), 6.65 (d, J = 8.4 Hz, 1H), 6.45 (dd, J = 6.9, 5.2 Hz, 1H), 3.75 (t, J = 6.0 Hz, 2H), 2.82 (t, J = 2.7 Hz, 1H), 2.01 (td, J = 7.4, 2.7 Hz, 2H), 1.84 (t, J = 6.0 Hz, 2H), 1.63 (t, J = 7.4 Hz, 2H). |
| 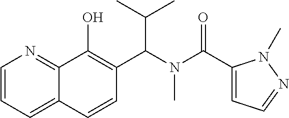 [SMILES: CC(C)C(c1ccc2cccnc2c1O)N(C)C(=O)c1ccnn1C] | 5 | N-(1-(8-hydroxyquinolin-7-yl)-2-methylpropyl)- N,1-dimethyl-1H-pyrazole-5-carboxamide LC-MS: m/z 339 (M + H)⁺. 1H NMR (400 MHz, CD3OD) mixture of rotamers (ratio ~3:2) δ (ppm): 8.85-8.79 (m, 1H), 8.30-8.23 (m, 1H), 7.72-7.27 (m, 4H), 6.80 (minor) and 6.40 (major) (two sets of s, 1H), 5.90 (major) and 5.40 (minor) (two sets of d, J = 12 Hz, 1H), 3.85 (major) |
| | | and 3.79 (minor) (two sets of s, 3H), 2.99 (minor) |
| | | and 2.85 (major) (two sets of s, 3H), 2.97-2.78 (m, |
| | | 1H), 1.18 (major) and 1.16 (minor) (two sets of d, J = |
| | | 6.8 Hz, 3H), 0.99 (major) and 0.79 (minor) (two |
| | | sets of d, J = 6.8 Hz, 3H) |
| 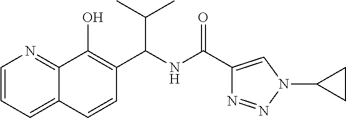 [SMILES: CC(C)C(NC(=O)c1cn(C2CC2)nn1)c1ccc2cccnc2c1O] | 5 | 1-cyclopropyl-N-(1-(8-hydroxyquinolin-7-yl)-2- methylpropyl)-1H-1,2,3-triazole-4-carboxamide LC-MS: m/z 352 (M + H)⁺. 1H NMR (400 MHz, CD3OD) δ (ppm): 8.84 (d, J = 2.4 Hz, 1H), 8.35 (s, 1H), 8.24 (d, J = 8.0 Hz, 1H), 7.52-7.45 (m, 2H), 7.35 (d, J = 8.4 Hz, 1H), 5.20 (d, J = 9.2 Hz, 1H), 4.00-3.91 (m, 1H), 2.52- 2.39 (m, 1H), 1.28-1.23 (m, 2H), 1.22-1.13 (m, |
| | | 2H), 1.11 (d, J = 6.8 Hz, 3H), 0.85 (d, J = 6.8 Hz, |
| | | 3H) |
| 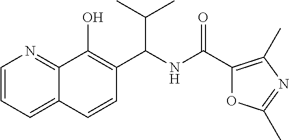 [SMILES: Cc1nc(C)c(C(=O)NC(c2ccc3cccnc3c2O)C(C)C)o1] | 5 | N-(1-(8-hydroxyquinolin-7-yl)-2-methylpropyl)- 2,4-dimethyloxazole-5-carboxamide LC-MS: m/z 340 (M + H)⁺. 1H NMR (400 MHz, CD3OD) δ (ppm): 8.83 (dd, J = 8.4 Hz, 1.6 Hz, 1H), 8.29 (d, J = 8.4 Hz, 1H), 7.55-7.49 (m, 2H), 7.40 (d, J = 8.4 Hz, 1H), 5.18 (d, J = 9.6 Hz, 1H), 2.50 (s, 3H), 2.47-2.37 (m, 1H), 2.36 (s, 3H), 1.13 (d, J = 6.8 Hz, 3H), 0.85 (d, J = 6.8 Hz, 3H) |
| 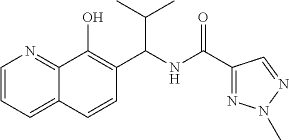 [SMILES: CC(C)C(NC(=O)c1cnn(C)n1)c1ccc2cccnc2c1O] | 5 | N-(1-(8-hydroxyquinolin-7-yl)-2-methylpropyl)-2- methyl-2H-1,2,3-triazole-4-carboxamide LC-MS: m/z 326 (M + H)⁺. 1H NMR (400 MHz, CD3OD) δ (ppm): 8.84 (dd, J = 8.4 Hz, 1.6 Hz, 1H), 8.31 (d, J = 8.4 Hz, 1H), 7.99 (s, 1H), 7.57-7.50 (m, 2H), 7.42 (d, J = 8.4 Hz, 1H), 5.20 (d, J = 10.0 Hz, 1H), 4.23 (s, 3H), 2.51-2.39 (m, 1H), 1.12 (d, J = 6.4 Hz, 3H), 0.86 (d, J = 6.8 Hz, 3H) |
| 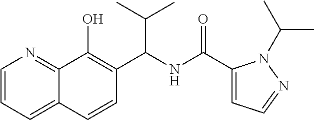 [SMILES: CC(C)C(NC(=O)c1ccnn1C(C)C)c1ccc2cccnc2c1O] | 5 | N-(1-(8-hydroxyquinolin-7-yl)-2-methylpropyl)-1- isopropyl-1H-pyrazole-5-carboxamide LC-MS: m/z 353 (M + H)⁺. 1H NMR (400 MHz, CD3OD) δ (ppm): 8.82 (dd, J = 8.4 Hz, 1.6 Hz, 1H), 8.27 (dd, J = 8.4 Hz, 1.2 Hz 1H), 7.55-7.48 (m, 3H), 7.40 (d, J = 8.4 Hz, 1H), 6.73 (d, J = 2.0 Hz, 1H), 5.33-5.21 (m, 2H), 2.44-2.29 (m, 1H), 1.42 (d, J = 6.4 Hz, 3H), 1.36 |
| | | (d, J = 6.8 Hz, 3H), 1.15 (d, J = 6.8 Hz, 3H), 0.86 |
| | | (d, J = 6.8 Hz, 3H) |
| 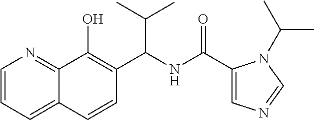 [SMILES: CC(C)C(NC(=O)c1cncn1C(C)C)c1ccc2cccnc2c1O] | 5 | N-(1-(8-hydroxyquinolin-7-yl)-2-methylpropyl)-1- isopropyl-1H-imidazole-5-carboxamide LC-MS: m/z 353 (M + H)⁺. 1H NMR (400 MHz, DMSO-d6) δ (ppm): 9.72 (br s, 1H), 8.85 (d, J = 2.8 Hz, 1H), 8.46 (d, J = 8.8 Hz, 1H), 8.29 (d, J = 7.6 Hz, 1H), 7.94 (s,, 1H), 7.66 (d, J = 8.4 Hz, 1H), 7.61 (s, 1H), 7.53 (dd, J = 8.4 Hz, 4.0 Hz, 1H), 7.41 (d, J = 8.4 Hz, 1H), 5.37 (t, J = |
| | | 9.6 Hz, 1H), 5.08 (hept, J = 6.8 Hz, 1H), 2.24- |
| | | 2.09 (m, 1H), 1.37 (d, J = 6.8 Hz, 3H), 1.30 (d, J = |
| | | 6.8 Hz, 3H), 1.04 (d, J = 6.4 Hz, 3H), 0.78 (d, J = |
| | | 6.8 Hz, 3H) |
| 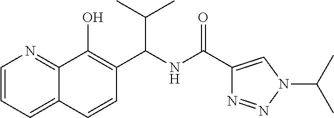 [SMILES: CC(C)C(NC(=O)c1cn(C(C)C)nn1)c1ccc2cccnc2c1O] | 5 | N-(1-(8-hydroxyquinolin-7-yl)-2-methylpropyl)-1- isopropyl-1H-1,2,3-triazole-4-carboxamide LC-MS: m/z 354 (M + H)⁺. 1H NMR (400 MHz, DMSO-d6) δ (ppm): 9.99 (br s, 1H), 8.86 (dd, J = 4.4 Hz, 1.6 Hz, 1H), 8.70 (d, J = 9.6 Hz, 1H), 8.62 (s, 1H), 8.32 (dd, J = 8.4 Hz, 1.2 Hz, 1H), 7.68 (d, J = 8.4 Hz, 1H), 7.55 (dd, J = 8.4 Hz, 4.0 Hz, 1H), 7.40 (d, J = 8.4 Hz, 1H), 5.29 |
| | | (t, J = 9.6 Hz, 1H), 4.86 (hept, J = 6.8 Hz, 1H), |
| | | 2.39-2.26 (m, 1H), 1.49 (d, J = 6.8 Hz, 3H), 1.48 |
| | | (d, J = 6.8 Hz, 3H), 1.03 (d, J = 6.4 Hz, 3H), 0.77 |
| | | (d, J = 6.8 Hz, 3H) |
| 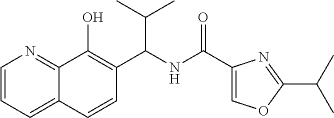 [SMILES: CC(C)c1nc(C(=O)NC(c2ccc3cccnc3c2O)C(C)C)co1] | 5 | N-(1-(8-hydroxyquinolin-7-yl)-2-methylpropyl)-2- isopropyloxazole-4-carboxamide LC-MS: m/z 354 (M + H)⁺. 1H NMR (400 MHz, CD3Cl) δ (ppm): 8.77 (dd, J = 4.4 Hz, 1.6 Hz, 1H), 8.21 (d, J = 9.6 Hz, 1H), 8.13 (dd, J = 8.0 Hz, 1.6 Hz, 1H), 8.04 (s, 1H), 7.43 (d, J = 8.4 Hz, 1H), 7.41 (dd, J = 8.4 Hz, 4.4 Hz, 1H), 7.30 (d, J = 8.4 Hz, 1H), 5.13 (t, J = 9.6 |
| | | Hz, 1H), 3.09 (hept, J = 6.8 Hz, 1H), 2.54-2.32 (m, |
| | | 1H), 1.35 (d, J = 6.8 Hz, 6H), 1.12 (d, J = 6.8 Hz, |
| | | 3H), 0.88 (d, J = 6.8 Hz, 3H) |
| 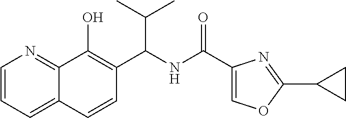 [SMILES: CC(C)C(NC(=O)c1coc(C2CC2)n1)c1ccc2cccnc2c1O] | 5 | 2-cyclopropyl-N-(1-(8-hydroxyquinolin-7-yl)-2- methylpropyl)oxazole-4-carboxamide LC-MS: m/z 352 (M + H)⁺. 1H NMR (400 MHz, CD3OD) δ (ppm): 8.83 (dd, J = 4.4 Hz, 1.2 Hz, 1H), 8.31 (d, J = 8.0 Hz, 1H), 8.15 (s, 1H), 7.53 (dd, J = 8.0 Hz, 4.0 Hz, 1H), 7.50 (d, J = 8.4 Hz, 1H), 7.41 (d, J = 8.4 Hz, 1H), 5.14 (t, J = 9.6 Hz, 1H), 2.51-2.40 (m, 1H), 2.18- |
| | | 2.08 (m, 1H), 1.13-1.06 (m, 7H), 0.86 (d, J = 6.8 |
| | | Hz, 3H) |
| 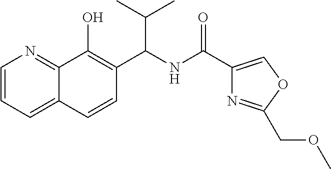 [SMILES: COCc1nc(C(=O)NC(c2ccc3cccnc3c2O)C(C)C)co1] | 5 | N-(1-(8-hydroxyquinolin-7-yl)-2-methylpropyl)-2- (methoxymethyl)oxazole-4-carboxamide LC-MS: m/z 356 (M + H)⁺. 1H NMR (400 MHz, CDCl3) δ (ppm): 8.77 (dd, J = 4.0 Hz, 1.6 Hz, 1H), 8.19 (d, J = 9.6 Hz, 1H), 8.15 (s, 1H), 8.13 (dd, J = 8.4 Hz, 1.6 Hz, 1H), 7.42 (d, J = 8.4 Hz, 1H), 7.41 (dd, J = 8.4 Hz, 4.4 Hz, 1H), 7.30 (d, J = 8.4 Hz, 1H), 5.13 (5, J = 9.6 Hz, 1H), 4.54 (s, 2H), 3.47 (s, 3H), 2.53-2.40 (m, 1H), 1.11 (d, J = 6.4 Hz, 3H), 0.88 (d, J = 6.4 Hz, 3H) |
| 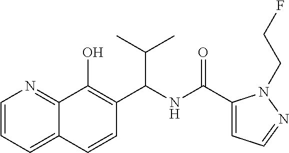 [SMILES: CC(C)C(NC(=O)c1ccnn1CCF)c1ccc2cccnc2c1O] | 5 | 1-(2-fluoroethyl)-N-(1-(8-hydroxyquinolin-7-yl)-2- methylpropyl)-1H-pyrazole-5-carboxamide LC-MS: m/z 357 (M + H)⁺. 1H NMR (400 MHz, DMSO-d6) δ (ppm): 9.76 (br s, 1H), 8.85 (dd, J = 4.0 Hz, 1.6 Hz, 1H), 8.70 (d, J = 9.2 Hz, 1H), 8.29 (dd, J = 8.4 Hz, 1.2 Hz, 1H), 7.67 (d, J = 8.4 Hz, 1H), 7.56-7.51 (m, 2H), 7.41 (d, J = 8.4 Hz, 1H), 6.98 (d, J = 2.0 Hz, 1H), 5.39 (5, J = 9.2 Hz, 1H), 4.84-4.52 (m, 4H), 2.24-2.11 (m, 1H), 1.04 (d, J = 6.8 Hz, 3H), 0.78 (d, J = 6.8 |
| | | Hz, 3H) |
| 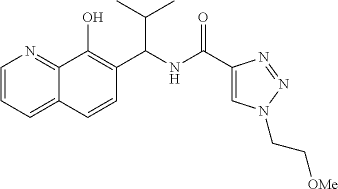 [SMILES: COCCn1cc(C(=O)NC(c2ccc3cccnc3c2O)C(C)C)nn1] | 5 | N-(1-(8-hydroxyquinolin-7-yl)-2-methylpropyl)-1- (2-methoxyethyl)-1H-1,2,3-triazole-4-carboxamide LC-MS: m/z 370 (M + H)⁺. 1H NMR (400 MHz, DMSO-d6) δ (ppm): 10.02 (br s, 1H), 8.87 (dd, J = 4.4 Hz, 1.6 Hz, 1H), 8.75 (d, J = 9.6 Hz, 1H), 8.48 (s, 1H), 8.34 (dd, J = 8.4 Hz, 1.6 Hz, 1H), 7.69 (d, J = 8.4 Hz, 1H), 7.56 (dd, J = 8.4 Hz, 4.4 Hz, 1H), 7.40 (d, J = 8.8 Hz, 1H), 5.29 (5, J = 9.6 Hz, 1H), 4.57 (t, J = 5.2 Hz, 2H), 3.73 (t, J = 5.2 Hz, 2H), 3.22 (s, 3H), 2.37- 2.25 (m, 1H), 1.04 (d, J = 6.8 Hz, 3H), 0.77 (d, J = 6.8 Hz, 3H) |
| 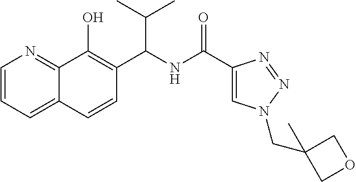 [SMILES: CC(C)C(NC(=O)c1cn(CC2(C)COC2)nn1)c1ccc2cccnc2c1O] | 5 | N-(1-(8-hydroxyquinolin-7-yl)-2-methylpropyl)-3- ((3-methyloxetan-3-yl)methyl)-3H-pyrazole-5- carboxamide LC-MS: m/z 395 (M + H)⁺. 1H NMR (400 MHz, DMSO-d6) δ (ppm): d 9.98 (br s, 1H), 8:86 (dd, J = 4.4 Hz, 1.6 Hz, 1H), 8.75 (d, J = 9.2 Hz, 1H), 8.61 (s, 1H), 8.32 (dd, J = 8.4 Hz, 1.6 Hz, 1H), 7.68 (d, J = 8.4 Hz, 1H), 7.55 (dd, J = 8.0 Hz, 4.0 Hz, 1H), 7.40 (d, J = 8.4 Hz, 1H), 5.29 (5, J = 9.6 Hz, 1H), 4.65 (s, 2H), 4.55 (t, J = 6.4 Hz, 2H), 4.23 (t, J = 6.4 Hz, 2H), 2.37-2.26 (m, 1H), 1.14 (s, 3H), 1.03 (d, J = 6.8 Hz, 3H), |
| | | 0.77 (d, J = 6.8 Hz, 3H) |
| 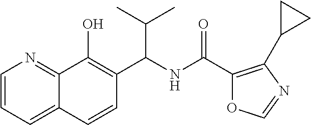 [SMILES: CC(C)C(NC(=O)c1ocnc1C1CC1)c1ccc2cccnc2c1O] | 5 | 4-cyclopropyl-N-(1-(8-hydroxyquinolin-7-yl)-2- methylpropyl)oxazole-5-carboxamide LC-MS: m/z 352 (M + H)⁺. 1H NMR (400 MHz, CD3OD) δ (ppm): 8:81-8.78 (m, 1H), 8.21 (d, J = 8.4 Hz, 1H), 8.05 (s, 1H), 7.50-7.44 (m, 2H), 7.34 (d, J = 8.0 Hz, 1H), 5.19 (d, J = 9.2 Hz, 1H), 2.65-2.56 (m, 1H), 2.46-2.38 (m, 1H), 1.12 (d, J = 6.8 Hz, 2H), 0.99-0.65 (m, |
| | | 4H), 0.86 (d, J = 6.8 Hz, 3H) |
| 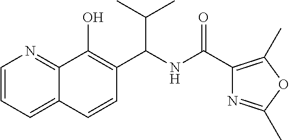 [SMILES: Cc1nc(C(=O)NC(c2ccc3cccnc3c2O)C(C)C)c(C)o1] | 5 | N-(1-(8-hydroxyquinolin-7-yl)-2-methylpropyl)- 2,5-dimethyloxazole-4-carboxamide LC-MS: m/z 340 (M + H)⁺. 1H NMR (400 MHz, CD3OD) δ (ppm): d 8.80 (dd, J = 4.4 Hz, 1.6 Hz, 1H), 8.23 (dd, J = 8.4 Hz, 1.6 Hz, 1H), 7.48 (dd, J = 8.0 Hz, 4.0 Hz, 1H), 7.45 (d, J = 8.0 Hz, 1H), 7.37 (d, J = 8.4 Hz, 1H), 5.12 (d, J = 9.2 Hz, 1H), 2.52 (s, 3H), 2.41 (s, 3H), 2.48-2.37 (m, 1H), 1.08 (d, J = 6.8 Hz, 3H), 0.87 (d, J = 6.8 |
| | | Hz, 3H) |

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A compound of Formula (I): 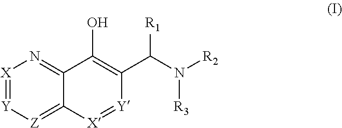
wherein
X, X′, Y, Y′, and Z are independently selected from N and C(R); provided that no more than two of X, X′, Y, Y′, and Z are N;
if Z and Y, or Y and X, or X′ and Y′, are C(R), then the two adjacent instances of R on any of them taken together may form a fused 3-8 membered ring;
R is independently selected from H, alkyl, alkenyl, alkynyl, cycloalkyl, heteroalkyl, cycloheteroalkyl, substituted or unsubstituted aryl, substituted or unsubstituted -alkylene-aryl, substituted or unsubstituted heteroaryl, substituted or unsubstituted -alkylene-heteroaryl, haloalkyl, halocycloalkyl, halocycloheteroalkyl, —O-alkyl, —O-haloalkyl, —O-cycloalkyl, —N-alkyl, —N-haloalkyl, —N-cycloalkyl, —S-alkyl, —S-haloalkyl, —S-cycloalkyl, —O-heteroalkyl, —O— cycloheteroalkyl, —N-heteroalkyl, —N-cycloheteroalkyl, —S-heteroalkyl, —S-cycloheteroalkyl, —O-aryl, —N-aryl, —S-aryl, —O-heteroaryl, —N-heteroaryl, —S-heteroaryl, substituted or unsubstituted —O-alkylene-aryl, substituted or unsubstituted —N-alkylene-aryl, substituted or unsubstituted —S— alkylene-aryl, substituted or unsubstituted —O-alkylene-heteroaryl, substituted or unsubstituted —N-alkylene-heteroaryl, substituted or unsubstituted —S-alkylene-heteroaryl, halide, —CN, —NO₂, —S(O)Rₐ, —S(O)₂Rₐ, —C(O)Rₐ, —C(O)₂Rₐ, —C(O)NRₐR_{b}, OH, and C(O)NR′C(NR′)NRₐR_{b};
R′ is H, or alkyl;
Rₐ and R_{b} are independently H, alkyl, alkenyl, alkynyl, substituted or unsubstituted aryl, cycloalkyl, heteroalkyl, haloalkyl, cycloheteroalkyl, halocycloalkyl, halocycloheteroalkyl, substituted or unsubstituted heteroaryl, substituted or unsubstituted -alkylene-aryl, substituted or unsubstituted -alkylene-heteroaryl, alkylene-OR′, alkylene-NR′, alkylene-SR′, or Rₐ and R_{b} taken together with the nitrogen atom to which they are attached may form a 3-8 membered ring;
R₁ is H, alkyl, alkenyl, alkynyl, haloalkyl, heteroalkyl, heterohaloalkyl, cycloalkyl, halocycloalkyl, heterocycloalkyl, heterohalocycloalkyl, unsubstituted or substituted aryl, unsubstituted or substituted alkenyl-aryl, unsubstituted or substituted heteroaryl, unsubstituted or substituted alkenyl-heteroaryl, alkylene-OR′, alkylene-NR′, or alkylene-SR′; and
R₂, and R₃ are independently selected from H, alkyl, alkenyl, alkynyl, unsubstituted or substituted heteroaryl, unsubstituted or substituted aryl, haloalkyl, heteroalkyl, heterohaloalkyl, cycloalkyl, halocycloalkyl, heterocycloalkyl, heterohalocycloalkyl, alkylene-cycloalkyl, alkylene-aryl, alkylene-heteroaryl, alkylene-OR′, alkylene-NR′, alkylene-SR′, —S(O)Rₐ, —S(O)₂Rₐ, —C(O)Rₐ, —C(O)₂Rₐ, —C(O)NRₐR_{b}, —C(NRₐ)NRₐR_{b}, —N(Rₐ)C(NRₐ)NRₐR_{b}, and —C(O)NR′C(NR′)NRₐR_{b}; or R₂ and R₃ taken together may form a 4-8 membered ring.

2. The compound of claim 1, wherein X is C(R).

3. The compound of claim 1, wherein Y is C(R).

4. The compound of claim 1, wherein Z is C(R).

5. The compound of claim 1, wherein X′ is C(R).

6. The compound of claim 1, wherein Y′ is C(R).

7. The compound of claim 1, wherein X, Y, W, and Z are C(R).

8. The compound of claim 1, wherein X, X′, Y, Y′, W, and Z are C(R).

9. The compound of any one of claims 1-8, wherein R is H.

10. The compound of any one of claims 1-8, wherein R is independently H or halide.

11. The compound of claim 10, wherein R is Cl, F, or Br.

12. The compound of any one of claims 1-11, wherein R₁ is alkyl.

13. The compound of claim 12, wherein alkyl is methyl, ethyl, i-propyl, n-propyl, n-butyl, butyl, or t-butyl.

14. The compound of any one of claims 1-11, wherein R₁ is alkenyl.

15. The compound of any one of claims 1-11, wherein R₁ is cycloalkyl.

16. The compound of claim 15, wherein cycloalkyl is cyclopropyl, cyclobutyl, cyclopentyl, or cyclohexyl.

17. The compound of any one of claims 1-11, wherein R₁ is unsubstituted aryl.

18. The compound of any one of claims 1-11, wherein R₁ is substituted aryl.

19. The compound of claim 17 or 18, wherein aryl is phenyl.

20. The compound of claim 18 or 19, wherein aryl or phenyl is substituted with one or more of —OMe, F, —CN, and —SO₂F.

21. The compound of any one of claims 1-11, wherein R₁ is alkynyl

22. The compound of any one of claims 1-21, wherein R₂ is alkyl.

23. The compound of any one of claims 1-21, wherein R₂ unsubstituted heteroaryl.

24. The compound of claim 23, wherein heteroaryl is pyridinyl.

25. The compound of any one of claims 1-21, wherein R₂ unsubstituted aryl.

26. The compound of claim 25, wherein aryl is phenyl.

27. The compound of any one of claims 1-21, wherein R₂ alkylene-cycloalkyl.

28. The compound of any one of claims 1-21, wherein R₂ alkylene-aryl.

29. The compound of any one of claims 1-21, wherein R₂ alkylene-O-alkyl.

30. The compound of any one of claims 1-21, wherein R₂ heterocycloalkyl.

31. The compound of any one of claims 1-30, wherein R₃ is H.

32. The compound of claim 1, wherein the compound is:

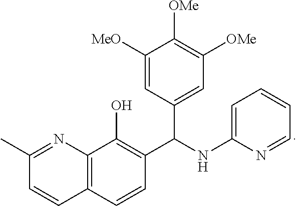 [SMILES: COc1cc(C(Nc2ccccn2)c2ccc3ccc(C)nc3c2O)cc(OC)c1OC]

33. The compound of claim 1, wherein the compound is selected from:

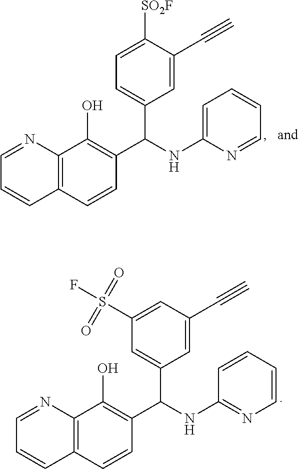 [SMILES: C#Cc1cc(C(Nc2ccccn2)c2ccc3cccnc3c2O)ccc1S(=O)(=O)F ; C#Cc1cc(C(Nc2ccccn2)c2ccc3cccnc3c2O)cc(S(=O)(=O)F)c1]

34. The compound of claim 1, wherein the compound is selected from:

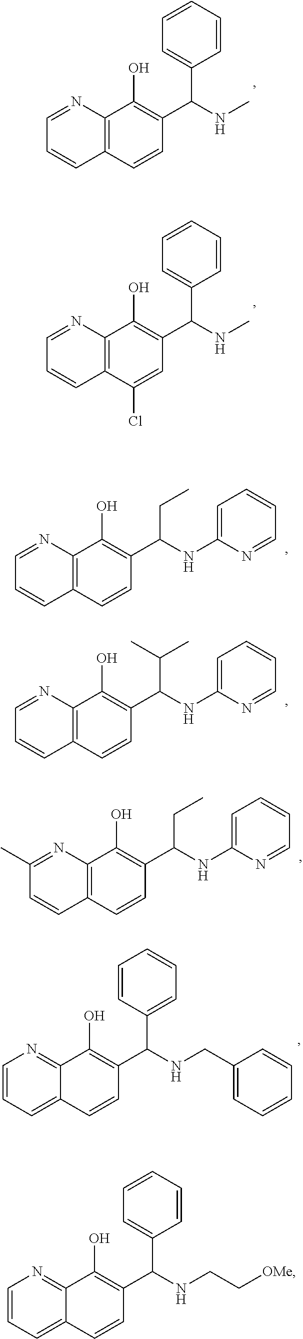

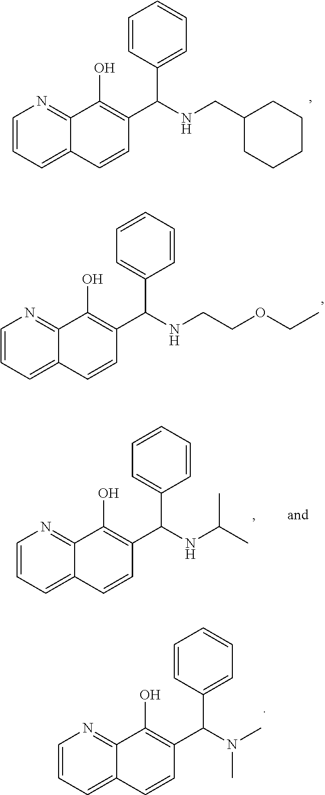 [SMILES: Oc1c(C(NCC2CCCCC2)c2ccccc2)ccc2cccnc12 ; CCOCCNC(c1ccccc1)c1ccc2cccnc2c1O ; CC(C)NC(c1ccccc1)c1ccc2cccnc2c1O ; CN(C)C(c1ccccc1)c1ccc2cccnc2c1O]

35. The compound of claim 1, wherein the compound is selected from:

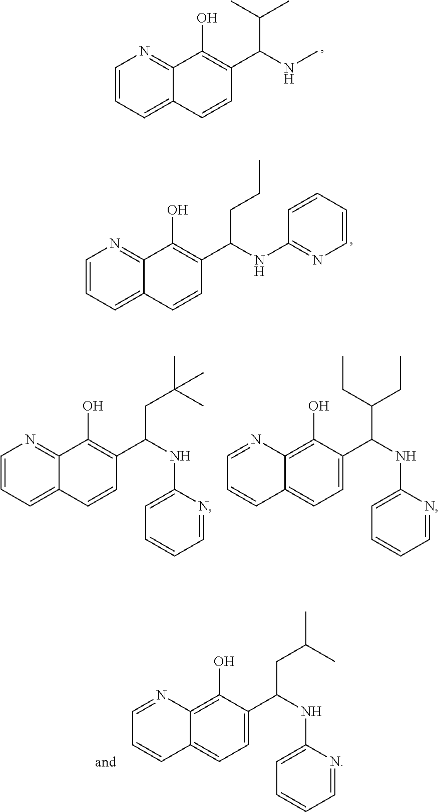

36. The compound of claim 1, wherein the compound is selected from:

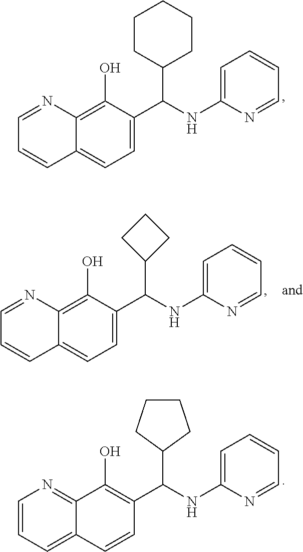 [SMILES: Oc1c(C(Nc2ccccn2)C2CCCCC2)ccc2cccnc12 ; Oc1c(C(Nc2ccccn2)C2CCC2)ccc2cccnc12 ; Oc1c(C(Nc2ccccn2)C2CCCC2)ccc2cccnc12]

37. The compound of claim 1, wherein the compound is:

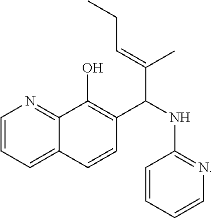 [SMILES: CC/C=C(\C)C(Nc1ccccn1)c1ccc2cccnc2c1O]

38. The compound of claim 1, wherein the compound is selected from the following table:

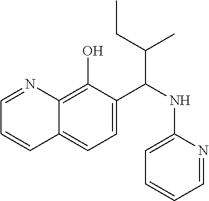 [SMILES: CCC(C)C(Nc1ccccn1)c1ccc2cccnc2c1O]

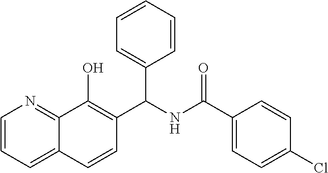 [SMILES: O=C(NC(c1ccccc1)c1ccc2cccnc2c1O)c1ccc(Cl)cc1]

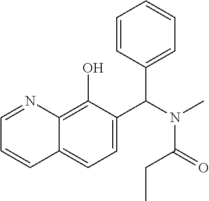 [SMILES: CCC(=O)N(C)C(c1ccccc1)c1ccc2cccnc2c1O]

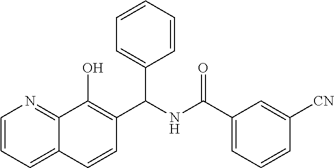 [SMILES: N#Cc1cccc(C(=O)NC(c2ccccc2)c2ccc3cccnc3c2O)c1]

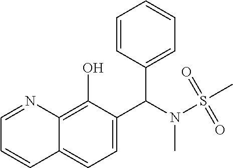 [SMILES: CN(C(c1ccccc1)c1ccc2cccnc2c1O)S(C)(=O)=O]

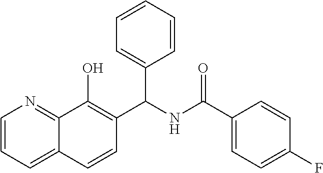 [SMILES: O=C(NC(c1ccccc1)c1ccc2cccnc2c1O)c1ccc(F)cc1]

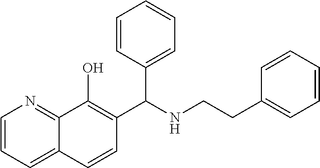 [SMILES: Oc1c(C(NCCc2ccccc2)c2ccccc2)ccc2cccnc12]

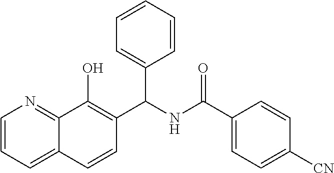 [SMILES: N#Cc1ccc(C(=O)NC(c2ccccc2)c2ccc3cccnc3c2O)cc1]

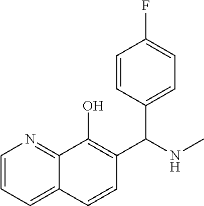 [SMILES: CNC(c1ccc(F)cc1)c1ccc2cccnc2c1O]

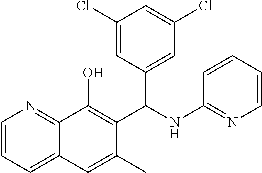 [SMILES: Cc1cc2cccnc2c(O)c1C(Nc1ccccn1)c1cc(Cl)cc(Cl)c1]

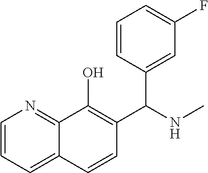 [SMILES: CNC(c1cccc(F)c1)c1ccc2cccnc2c1O]

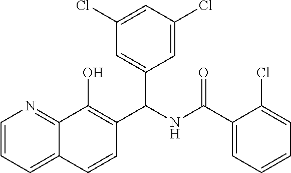 [SMILES: O=C(NC(c1cc(Cl)cc(Cl)c1)c1ccc2cccnc2c1O)c1ccccc1Cl]

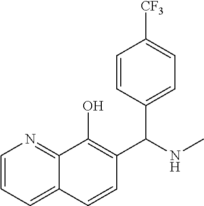 [SMILES: CNC(c1ccc(C(F)(F)F)cc1)c1ccc2cccnc2c1O]

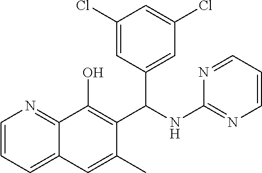 [SMILES: Cc1cc2cccnc2c(O)c1C(Nc1ncccn1)c1cc(Cl)cc(Cl)c1]

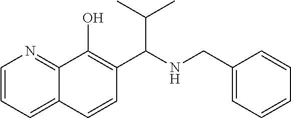 [SMILES: CC(C)C(NCc1ccccc1)c1ccc2cccnc2c1O]

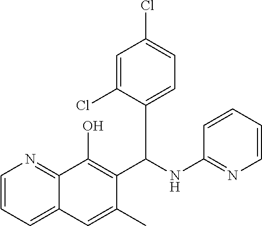 [SMILES: Cc1cc2cccnc2c(O)c1C(Nc1ccccn1)c1ccc(Cl)cc1Cl]

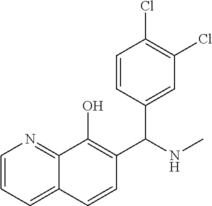 [SMILES: CNC(c1ccc(Cl)c(Cl)c1)c1ccc2cccnc2c1O]

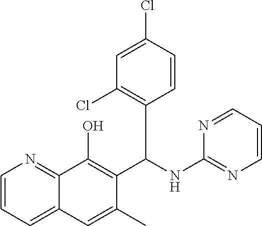 [SMILES: Cc1cc2cccnc2c(O)c1C(Nc1ncccn1)c1ccc(Cl)cc1Cl]

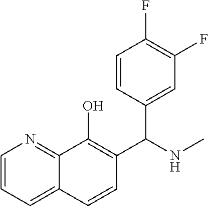 [SMILES: CNC(c1ccc(F)c(F)c1)c1ccc2cccnc2c1O]

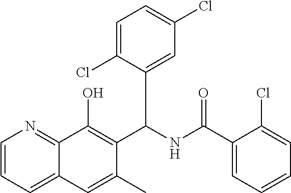 [SMILES: Cc1cc2cccnc2c(O)c1C(NC(=O)c1ccccc1Cl)c1cc(Cl)ccc1Cl]

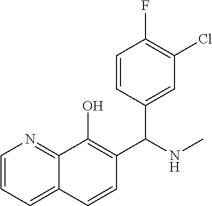 [SMILES: CNC(c1ccc(F)c(Cl)c1)c1ccc2cccnc2c1O]

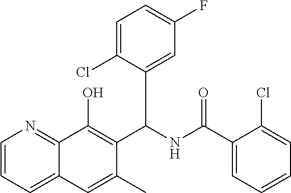 [SMILES: Cc1cc2cccnc2c(O)c1C(NC(=O)c1ccccc1Cl)c1cc(F)ccc1Cl]

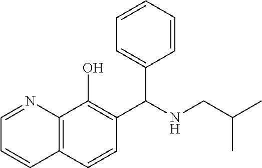 [SMILES: CC(C)CNC(c1ccccc1)c1ccc2cccnc2c1O]

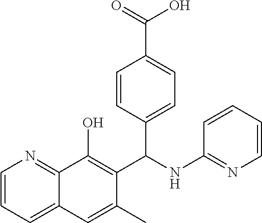 [SMILES: Cc1cc2cccnc2c(O)c1C(Nc1ccccn1)c1ccc(C(=O)O)cc1]

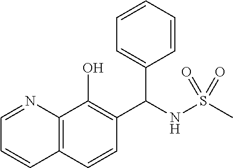 [SMILES: CS(=O)(=O)NC(c1ccccc1)c1ccc2cccnc2c1O]

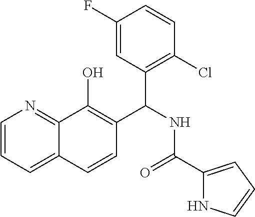 [SMILES: O=C(NC(c1cc(F)ccc1Cl)c1ccc2cccnc2c1O)c1ccc[nH]1]

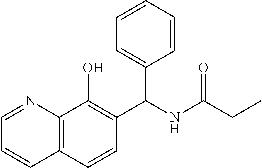 [SMILES: CCC(=O)NC(c1ccccc1)c1ccc2cccnc2c1O]

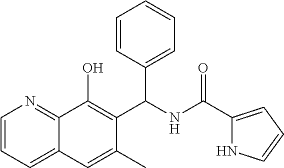 [SMILES: Cc1cc2cccnc2c(O)c1C(NC(=O)c1ccc[nH]1)c1ccccc1]

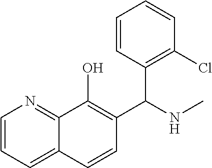 [SMILES: CNC(c1ccccc1Cl)c1ccc2cccnc2c1O]

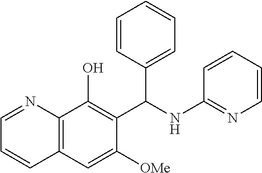 [SMILES: COc1cc2cccnc2c(O)c1C(Nc1ccccn1)c1ccccc1]

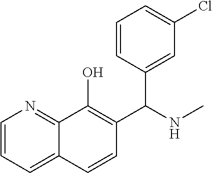 [SMILES: CNC(c1cccc(Cl)c1)c1ccc2cccnc2c1O]

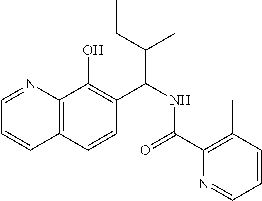 [SMILES: CCC(C)C(NC(=O)c1ncccc1C)c1ccc2cccnc2c1O]

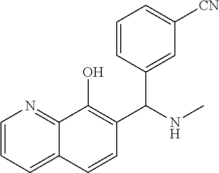 [SMILES: CNC(c1cccc(C#N)c1)c1ccc2cccnc2c1O]

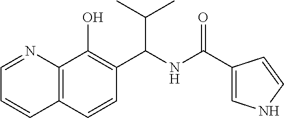 [SMILES: CC(C)C(NC(=O)c1cc[nH]c1)c1ccc2cccnc2c1O]

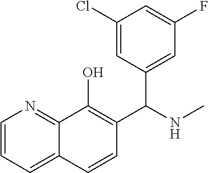 [SMILES: CNC(c1cc(F)cc(Cl)c1)c1ccc2cccnc2c1O]

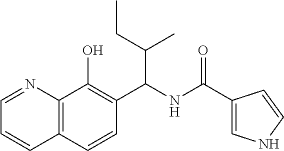 [SMILES: CCC(C)C(NC(=O)c1cc[nH]c1)c1ccc2cccnc2c1O]

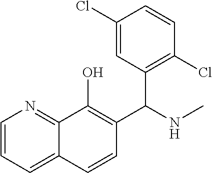 [SMILES: CNC(c1cc(Cl)ccc1Cl)c1ccc2cccnc2c1O]

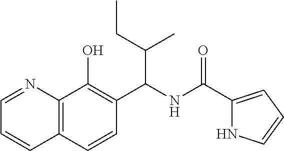 [SMILES: CCC(C)C(NC(=O)c1ccc[nH]1)c1ccc2cccnc2c1O]

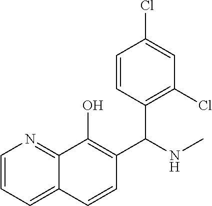 [SMILES: CNC(c1ccc(Cl)cc1Cl)c1ccc2cccnc2c1O]

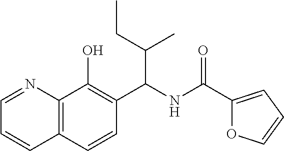 [SMILES: CCC(C)C(NC(=O)c1ccco1)c1ccc2cccnc2c1O]

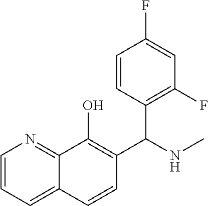 [SMILES: CNC(c1ccc(F)cc1F)c1ccc2cccnc2c1O]

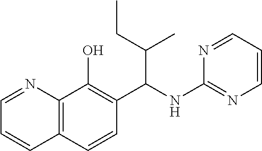 [SMILES: CCC(C)C(Nc1ncccn1)c1ccc2cccnc2c1O]

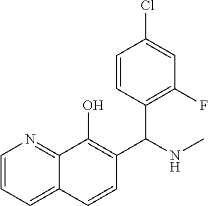 [SMILES: CNC(c1ccc(Cl)cc1F)c1ccc2cccnc2c1O]

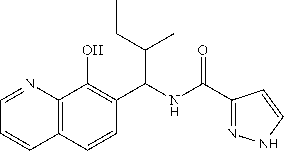 [SMILES: CCC(C)C(NC(=O)c1cc[nH]n1)c1ccc2cccnc2c1O]

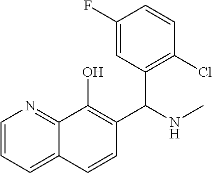 [SMILES: CNC(c1cc(F)ccc1Cl)c1ccc2cccnc2c1O]

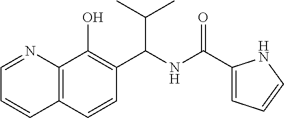 [SMILES: CC(C)C(NC(=O)c1ccc[nH]1)c1ccc2cccnc2c1O]

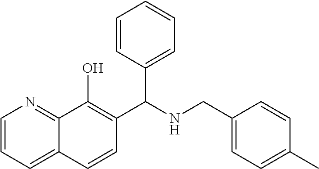 [SMILES: Cc1ccc(CNC(c2ccccc2)c2ccc3cccnc3c2O)cc1]

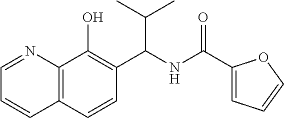 [SMILES: CC(C)C(NC(=O)c1ccco1)c1ccc2cccnc2c1O]

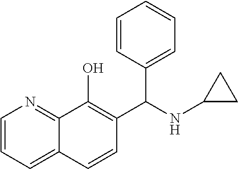 [SMILES: Oc1c(C(NC2CC2)c2ccccc2)ccc2cccnc12]

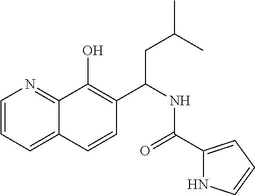 [SMILES: CC(C)CC(NC(=O)c1ccc[nH]1)c1ccc2cccnc2c1O]

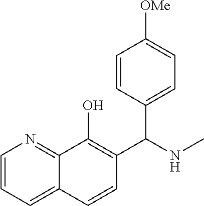 [SMILES: CNC(c1ccc(OC)cc1)c1ccc2cccnc2c1O]

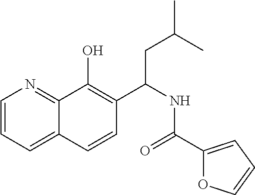 [SMILES: CC(C)CC(NC(=O)c1ccco1)c1ccc2cccnc2c1O]

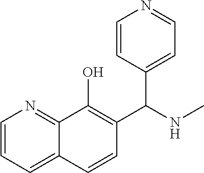 [SMILES: CNC(c1ccncc1)c1ccc2cccnc2c1O]

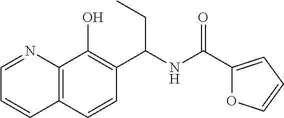 [SMILES: CCC(NC(=O)c1ccco1)c1ccc2cccnc2c1O]

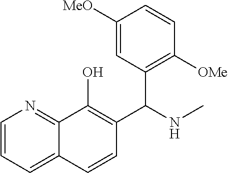 [SMILES: CNC(c1cc(OC)ccc1OC)c1ccc2cccnc2c1O]

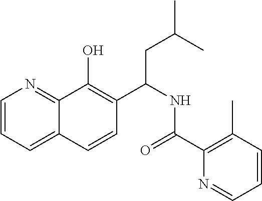 [SMILES: Cc1cccnc1C(=O)NC(CC(C)C)c1ccc2cccnc2c1O]

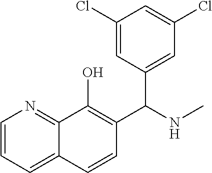 [SMILES: CNC(c1cc(Cl)cc(Cl)c1)c1ccc2cccnc2c1O]

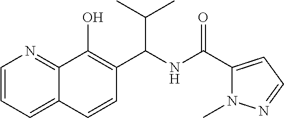 [SMILES: CC(C)C(NC(=O)c1ccnn1C)c1ccc2cccnc2c1O]

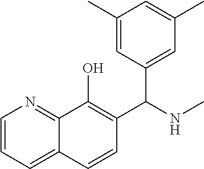 [SMILES: CNC(c1cc(C)cc(C)c1)c1ccc2cccnc2c1O]

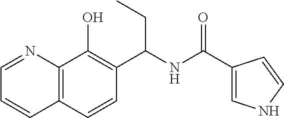 [SMILES: CCC(NC(=O)c1cc[nH]c1)c1ccc2cccnc2c1O]

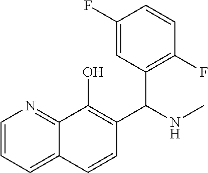 [SMILES: CNC(c1cc(F)ccc1F)c1ccc2cccnc2c1O]

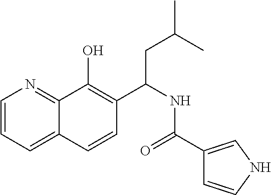 [SMILES: CC(C)CC(NC(=O)c1cc[nH]c1)c1ccc2cccnc2c1O]

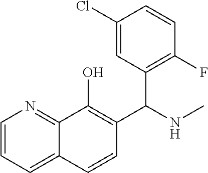 [SMILES: CNC(c1cc(Cl)ccc1F)c1ccc2cccnc2c1O]

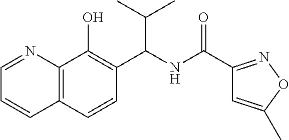 [SMILES: Cc1cc(C(=O)NC(c2ccc3cccnc3c2O)C(C)C)no1]

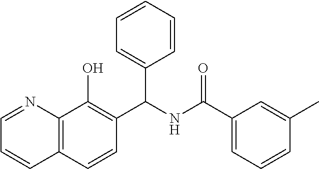 [SMILES: Cc1cccc(C(=O)NC(c2ccccc2)c2ccc3cccnc3c2O)c1]

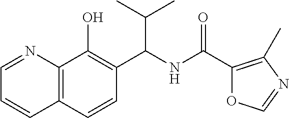 [SMILES: Cc1ncoc1C(=O)NC(c1ccc2cccnc2c1O)C(C)C]

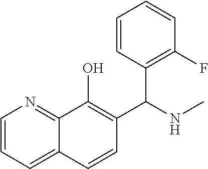 [SMILES: CNC(c1ccccc1F)c1ccc2cccnc2c1O]

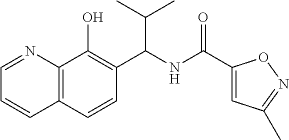 [SMILES: Cc1cc(C(=O)NC(c2ccc3cccnc3c2O)C(C)C)on1]

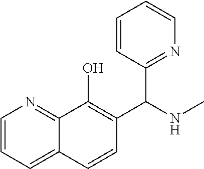 [SMILES: CNC(c1ccccn1)c1ccc2cccnc2c1O]

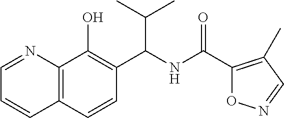 [SMILES: Cc1cnoc1C(=O)NC(c1ccc2cccnc2c1O)C(C)C]

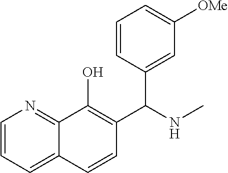 [SMILES: CNC(c1cccc(OC)c1)c1ccc2cccnc2c1O]

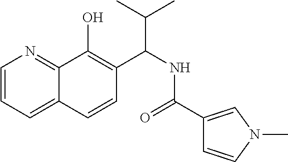 [SMILES: CC(C)C(NC(=O)c1ccn(C)c1)c1ccc2cccnc2c1O]

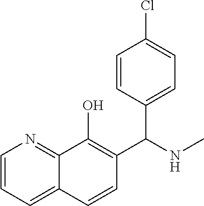 [SMILES: CNC(c1ccc(Cl)cc1)c1ccc2cccnc2c1O]

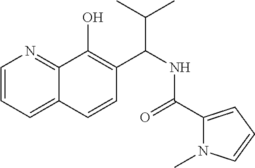 [SMILES: CC(C)C(NC(=O)c1cccn1C)c1ccc2cccnc2c1O]

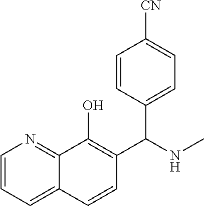 [SMILES: CNC(c1ccc(C#N)cc1)c1ccc2cccnc2c1O]

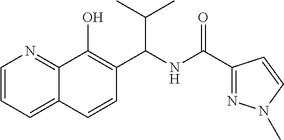 [SMILES: CC(C)C(NC(=O)c1ccn(C)n1)c1ccc2cccnc2c1O]

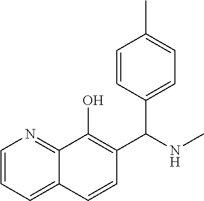 [SMILES: CNC(c1ccc(C)cc1)c1ccc2cccnc2c1O]

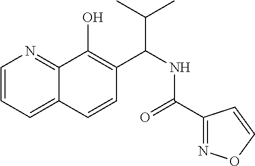 [SMILES: CC(C)C(NC(=O)c1ccon1)c1ccc2cccnc2c1O]

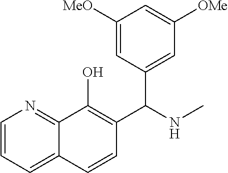 [SMILES: CNC(c1cc(OC)cc(OC)c1)c1ccc2cccnc2c1O]

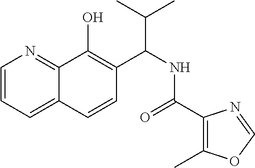 [SMILES: Cc1ocnc1C(=O)NC(c1ccc2cccnc2c1O)C(C)C]

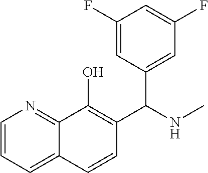 [SMILES: CNC(c1cc(F)cc(F)c1)c1ccc2cccnc2c1O]

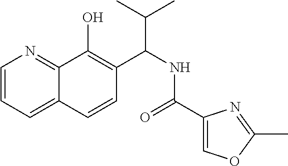 [SMILES: Cc1nc(C(=O)NC(c2ccc3cccnc3c2O)C(C)C)co1]

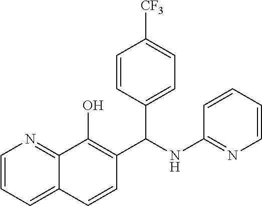 [SMILES: Oc1c(C(Nc2ccccn2)c2ccc(C(F)(F)F)cc2)ccc2cccnc12]

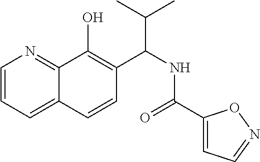 [SMILES: CC(C)C(NC(=O)c1ccno1)c1ccc2cccnc2c1O]

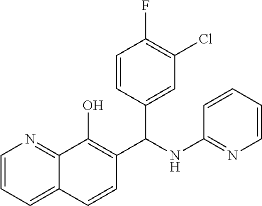 [SMILES: Oc1c(C(Nc2ccccn2)c2ccc(F)c(Cl)c2)ccc2cccnc12]

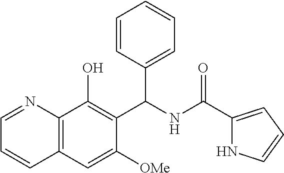 [SMILES: COc1cc2cccnc2c(O)c1C(NC(=O)c1ccc[nH]1)c1ccccc1]

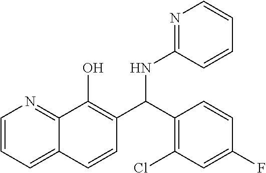 [SMILES: Oc1c(C(Nc2ccccn2)c2ccc(F)cc2Cl)ccc2cccnc12]

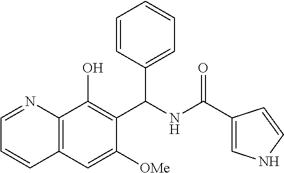 [SMILES: COc1cc2cccnc2c(O)c1C(NC(=O)c1cc[nH]c1)c1ccccc1]

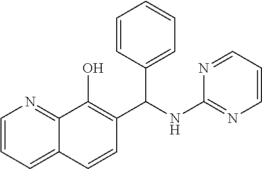 [SMILES: Oc1c(C(Nc2ncccn2)c2ccccc2)ccc2cccnc12]

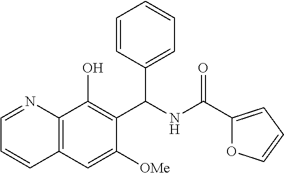 [SMILES: COc1cc2cccnc2c(O)c1C(NC(=O)c1ccco1)c1ccccc1]

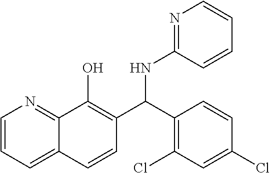 [SMILES: Oc1c(C(Nc2ccccn2)c2ccc(Cl)cc2Cl)ccc2cccnc12]

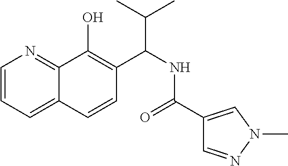 [SMILES: CC(C)C(NC(=O)c1cnn(C)c1)c1ccc2cccnc2c1O]

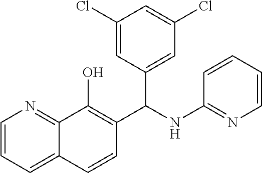 [SMILES: Oc1c(C(Nc2ccccn2)c2cc(Cl)cc(Cl)c2)ccc2cccnc12]

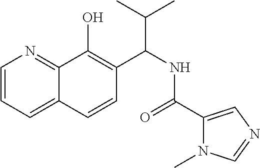 [SMILES: CC(C)C(NC(=O)c1cncn1C)c1ccc2cccnc2c1O]

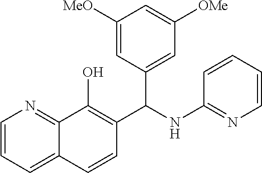 [SMILES: COc1cc(OC)cc(C(Nc2ccccn2)c2ccc3cccnc3c2O)c1]

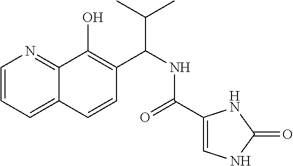 [SMILES: CC(C)C(NC(=O)c1c[nH]c(=O)[nH]1)c1ccc2cccnc2c1O]

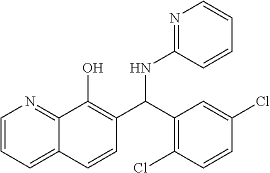 [SMILES: Oc1c(C(Nc2ccccn2)c2cc(Cl)ccc2Cl)ccc2cccnc12]

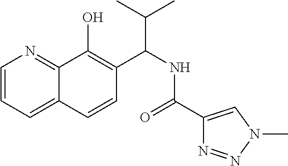 [SMILES: CC(C)C(NC(=O)c1cn(C)nn1)c1ccc2cccnc2c1O]

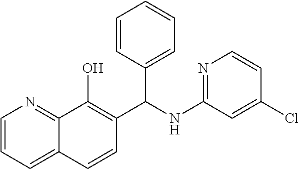 [SMILES: Oc1c(C(Nc2cc(Cl)ccn2)c2ccccc2)ccc2cccnc12]

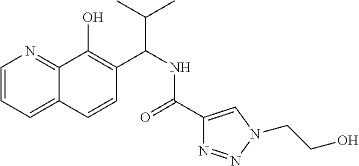 [SMILES: CC(C)C(NC(=O)c1cn(CCO)nn1)c1ccc2cccnc2c1O]

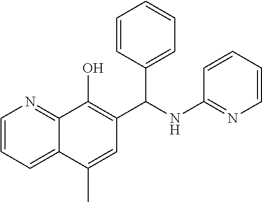 [SMILES: Cc1cc(C(Nc2ccccn2)c2ccccc2)c(O)c2ncccc12]

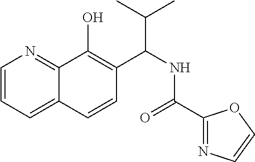 [SMILES: CC(C)C(NC(=O)c1ncco1)c1ccc2cccnc2c1O]

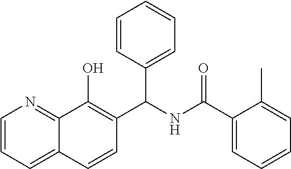 [SMILES: Cc1ccccc1C(=O)NC(c1ccccc1)c1ccc2cccnc2c1O]

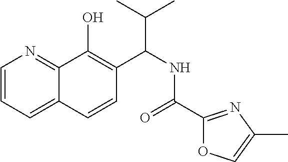 [SMILES: Cc1coc(C(=O)NC(c2ccc3cccnc3c2O)C(C)C)n1]

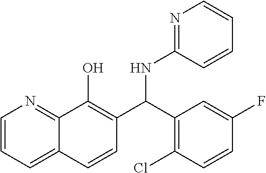 [SMILES: Oc1c(C(Nc2ccccn2)c2cc(F)ccc2Cl)ccc2cccnc12]

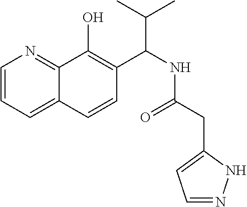 [SMILES: CC(C)C(NC(=O)Cc1ccn[nH]1)c1ccc2cccnc2c1O]

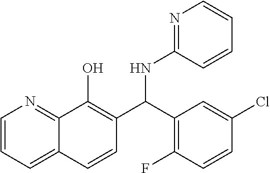 [SMILES: Oc1c(C(Nc2ccccn2)c2cc(Cl)ccc2F)ccc2cccnc12]

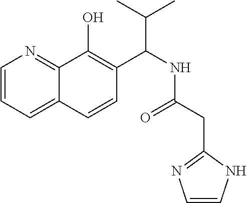 [SMILES: CC(C)C(NC(=O)Cc1ncc[nH]1)c1ccc2cccnc2c1O]

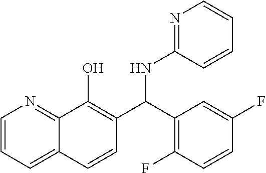 [SMILES: Oc1c(C(Nc2ccccn2)c2cc(F)ccc2F)ccc2cccnc12]

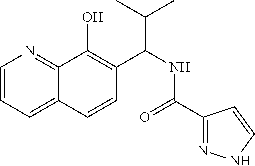 [SMILES: CC(C)C(NC(=O)c1cc[nH]n1)c1ccc2cccnc2c1O]

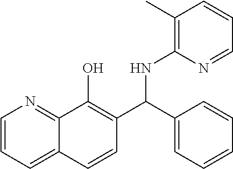 [SMILES: Cc1cccnc1NC(c1ccccc1)c1ccc2cccnc2c1O]

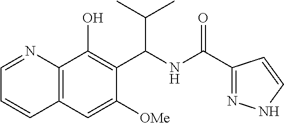 [SMILES: COc1cc2cccnc2c(O)c1C(NC(=O)c1cc[nH]n1)C(C)C]

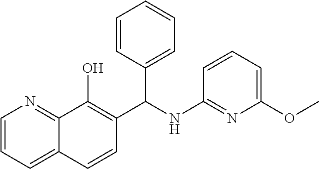 [SMILES: COc1cccc(NC(c2ccccc2)c2ccc3cccnc3c2O)n1]

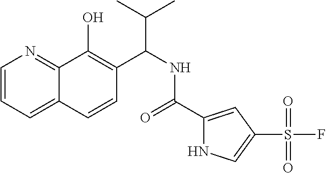 [SMILES: CC(C)C(NC(=O)c1cc(S(=O)(=O)F)c[nH]1)c1ccc2cccnc2c1O]

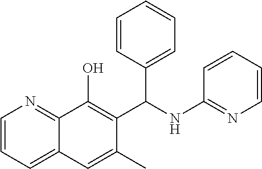 [SMILES: Cc1cc2cccnc2c(O)c1C(Nc1ccccn1)c1ccccc1]

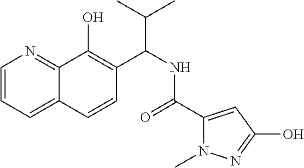 [SMILES: CC(C)C(NC(=O)c1cc(O)nn1C)c1ccc2cccnc2c1O]

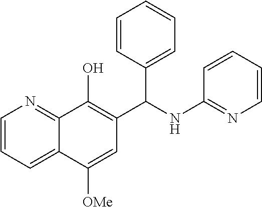 [SMILES: COc1cc(C(Nc2ccccn2)c2ccccc2)c(O)c2ncccc12]

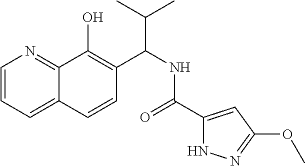 [SMILES: COc1cc(C(=O)NC(c2ccc3cccnc3c2O)C(C)C)[nH]n1]

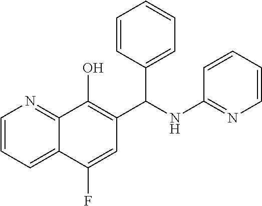 [SMILES: Oc1c(C(Nc2ccccn2)c2ccccc2)cc(F)c2cccnc12]

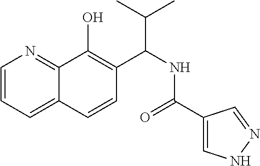 [SMILES: CC(C)C(NC(=O)c1cn[nH]c1)c1ccc2cccnc2c1O]

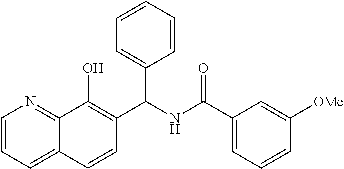 [SMILES: COc1cccc(C(=O)NC(c2ccccc2)c2ccc3cccnc3c2O)c1]

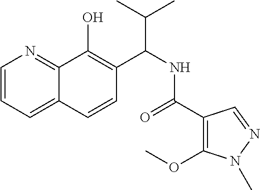 [SMILES: COc1c(C(=O)NC(c2ccc3cccnc3c2O)C(C)C)cnn1C]

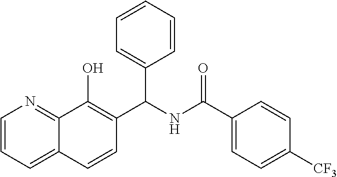 [SMILES: O=C(NC(c1ccccc1)c1ccc2cccnc2c1O)c1ccc(C(F)(F)F)cc1]

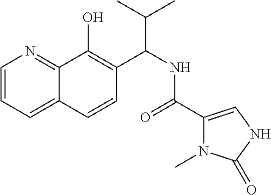 [SMILES: CC(C)C(NC(=O)c1c[nH]c(=O)n1C)c1ccc2cccnc2c1O]

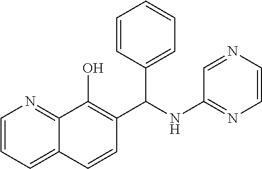 [SMILES: Oc1c(C(Nc2cnccn2)c2ccccc2)ccc2cccnc12]

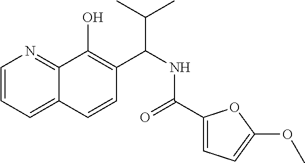 [SMILES: COc1ccc(C(=O)NC(c2ccc3cccnc3c2O)C(C)C)o1]

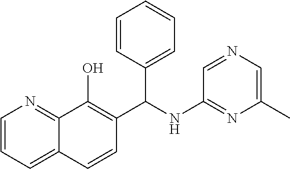 [SMILES: Cc1cncc(NC(c2ccccc2)c2ccc3cccnc3c2O)n1]

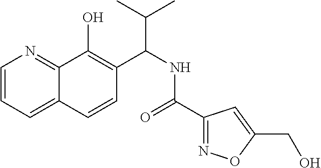 [SMILES: CC(C)C(NC(=O)c1cc(CO)on1)c1ccc2cccnc2c1O]

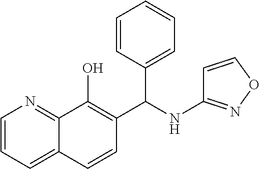 [SMILES: Oc1c(C(Nc2ccon2)c2ccccc2)ccc2cccnc12]

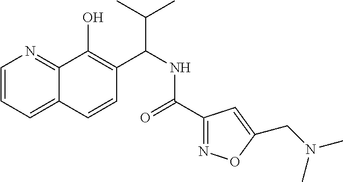 [SMILES: CC(C)C(NC(=O)c1cc(CN(C)C)on1)c1ccc2cccnc2c1O]

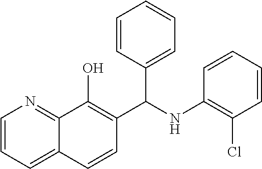 [SMILES: Oc1c(C(Nc2ccccc2Cl)c2ccccc2)ccc2cccnc12]

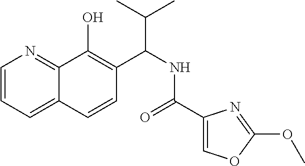 [SMILES: COc1nc(C(=O)NC(c2ccc3cccnc3c2O)C(C)C)co1]

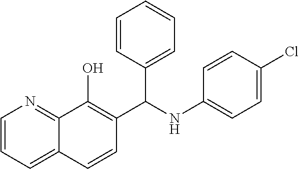 [SMILES: Oc1c(C(Nc2ccc(Cl)cc2)c2ccccc2)ccc2cccnc12]

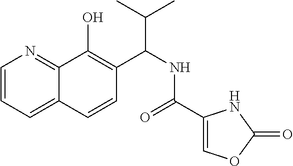 [SMILES: CC(C)C(NC(=O)c1coc(=O)[nH]1)c1ccc2cccnc2c1O]

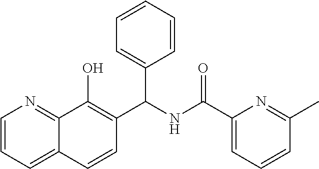 [SMILES: Cc1cccc(C(=O)NC(c2ccccc2)c2ccc3cccnc3c2O)n1]

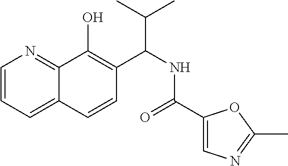 [SMILES: Cc1ncc(C(=O)NC(c2ccc3cccnc3c2O)C(C)C)o1]

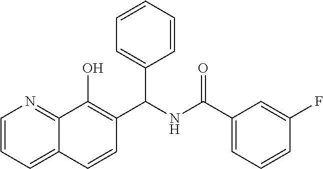 [SMILES: O=C(NC(c1ccccc1)c1ccc2cccnc2c1O)c1cccc(F)c1]

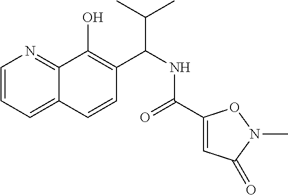 [SMILES: CC(C)C(NC(=O)c1cc(=O)n(C)o1)c1ccc2cccnc2c1O]

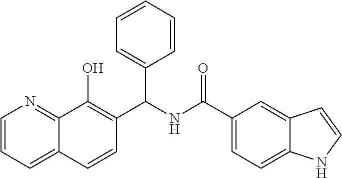 [SMILES: O=C(NC(c1ccccc1)c1ccc2cccnc2c1O)c1ccc2[nH]ccc2c1]

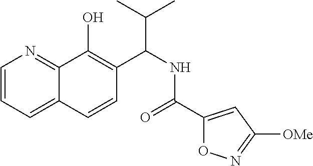 [SMILES: COc1cc(C(=O)NC(c2ccc3cccnc3c2O)C(C)C)on1]

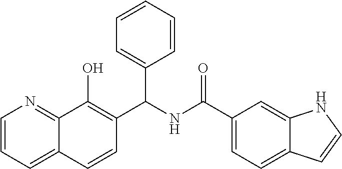 [SMILES: O=C(NC(c1ccccc1)c1ccc2cccnc2c1O)c1ccc2cc[nH]c2c1]

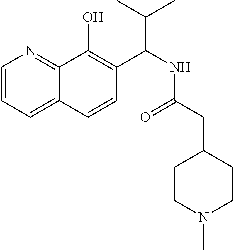 [SMILES: CC(C)C(NC(=O)CC1CCN(C)CC1)c1ccc2cccnc2c1O]

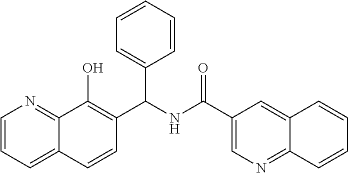 [SMILES: O=C(NC(c1ccccc1)c1ccc2cccnc2c1O)c1cnc2ccccc2c1]

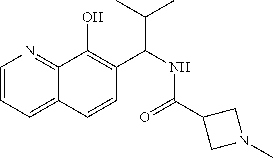 [SMILES: CC(C)C(NC(=O)C1CN(C)C1)c1ccc2cccnc2c1O]

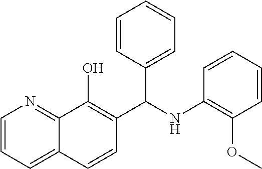 [SMILES: COc1ccccc1NC(c1ccccc1)c1ccc2cccnc2c1O]

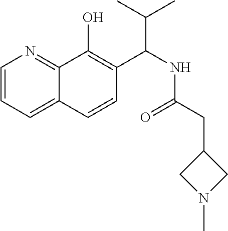 [SMILES: CC(C)C(NC(=O)CC1CN(C)C1)c1ccc2cccnc2c1O]

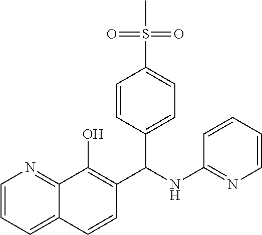 [SMILES: CS(=O)(=O)c1ccc(C(Nc2ccccn2)c2ccc3cccnc3c2O)cc1]

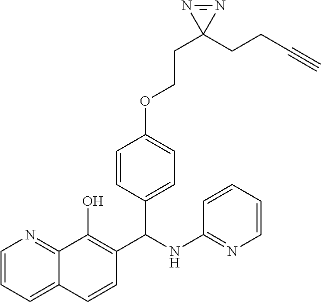 [SMILES: C#CCCC1(CCOc2ccc(C(Nc3ccccn3)c3ccc4cccnc4c3O)cc2)N=N1]

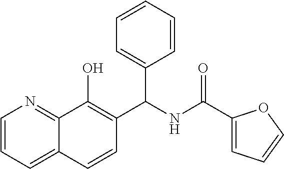 [SMILES: O=C(NC(c1ccccc1)c1ccc2cccnc2c1O)c1ccco1]

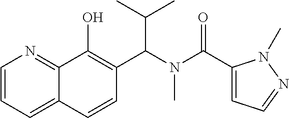 [SMILES: CC(C)C(c1ccc2cccnc2c1O)N(C)C(=O)c1ccnn1C]

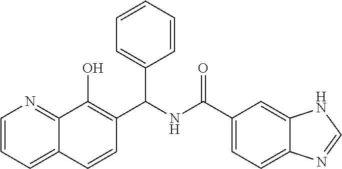 [SMILES: O=C(NC(c1ccccc1)c1ccc2cccnc2c1O)c1ccc2nc[nH]c2c1]

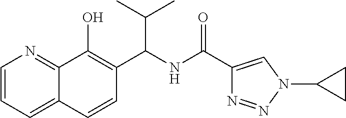 [SMILES: CC(C)C(NC(=O)c1cn(C2CC2)nn1)c1ccc2cccnc2c1O]

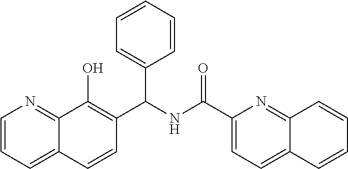 [SMILES: O=C(NC(c1ccccc1)c1ccc2cccnc2c1O)c1ccc2ccccc2n1]

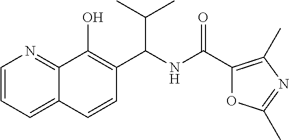 [SMILES: Cc1nc(C)c(C(=O)NC(c2ccc3cccnc3c2O)C(C)C)o1]

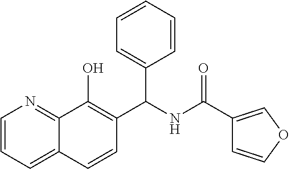 [SMILES: O=C(NC(c1ccccc1)c1ccc2cccnc2c1O)c1ccoc1]

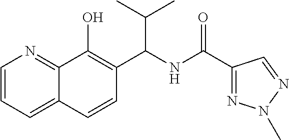 [SMILES: CC(C)C(NC(=O)c1cnn(C)n1)c1ccc2cccnc2c1O]

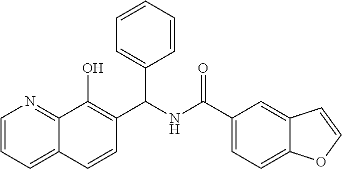 [SMILES: O=C(NC(c1ccccc1)c1ccc2cccnc2c1O)c1ccc2occc2c1]

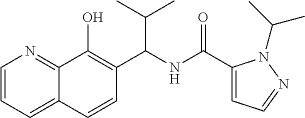 [SMILES: CC(C)C(NC(=O)c1ccnn1C(C)C)c1ccc2cccnc2c1O]

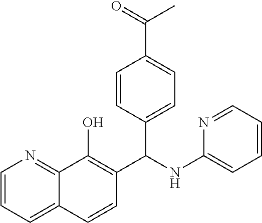 [SMILES: CC(=O)c1ccc(C(Nc2ccccn2)c2ccc3cccnc3c2O)cc1]

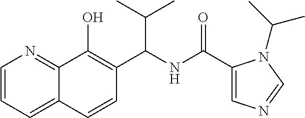 [SMILES: CC(C)C(NC(=O)c1cncn1C(C)C)c1ccc2cccnc2c1O]

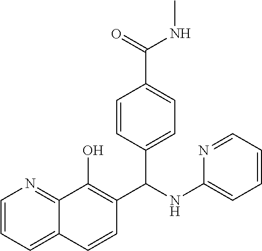 [SMILES: CNC(=O)c1ccc(C(Nc2ccccn2)c2ccc3cccnc3c2O)cc1]

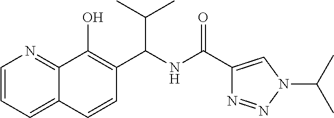 [SMILES: CC(C)C(NC(=O)c1cn(C(C)C)nn1)c1ccc2cccnc2c1O]

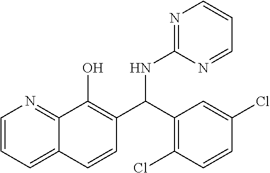 [SMILES: Oc1c(C(Nc2ncccn2)c2cc(Cl)ccc2Cl)ccc2cccnc12]

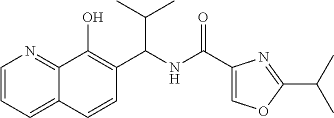 [SMILES: CC(C)c1nc(C(=O)NC(c2ccc3cccnc3c2O)C(C)C)co1]

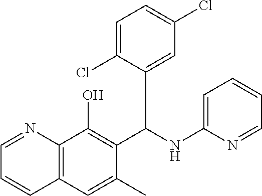 [SMILES: Cc1cc2cccnc2c(O)c1C(Nc1ccccn1)c1cc(Cl)ccc1Cl]

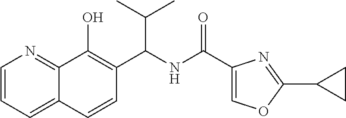 [SMILES: CC(C)C(NC(=O)c1coc(C2CC2)n1)c1ccc2cccnc2c1O]

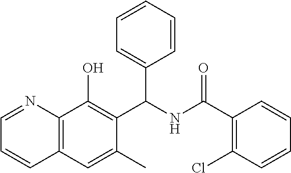 [SMILES: Cc1cc2cccnc2c(O)c1C(NC(=O)c1ccccc1Cl)c1ccccc1]

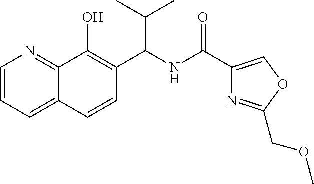 [SMILES: COCc1nc(C(=O)NC(c2ccc3cccnc3c2O)C(C)C)co1]

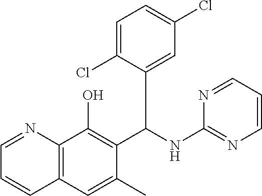 [SMILES: Cc1cc2cccnc2c(O)c1C(Nc1ncccn1)c1cc(Cl)ccc1Cl]

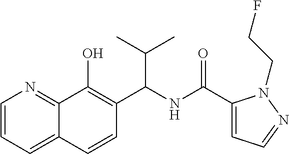 [SMILES: CC(C)C(NC(=O)c1ccnn1CCF)c1ccc2cccnc2c1O]

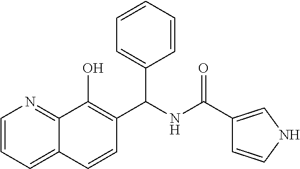 [SMILES: O=C(NC(c1ccccc1)c1ccc2cccnc2c1O)c1cc[nH]c1]

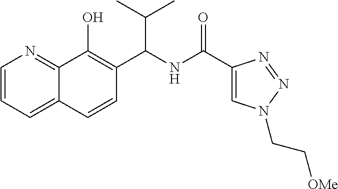 [SMILES: COCCn1cc(C(=O)NC(c2ccc3cccnc3c2O)C(C)C)nn1]

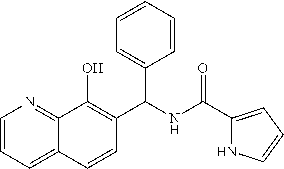 [SMILES: O=C(NC(c1ccccc1)c1ccc2cccnc2c1O)c1ccc[nH]1]

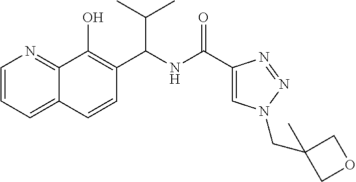 [SMILES: CC(C)C(NC(=O)c1cn(CC2(C)COC2)nn1)c1ccc2cccnc2c1O]

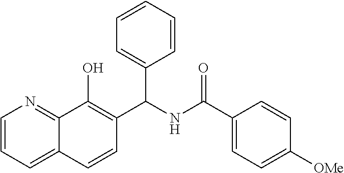 [SMILES: COc1ccc(C(=O)NC(c2ccccc2)c2ccc3cccnc3c2O)cc1]

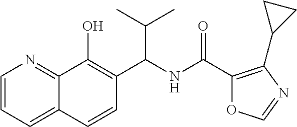 [SMILES: CC(C)C(NC(=O)c1ocnc1C1CC1)c1ccc2cccnc2c1O]

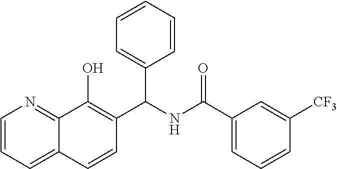 [SMILES: O=C(NC(c1ccccc1)c1ccc2cccnc2c1O)c1cccc(C(F)(F)F)c1]

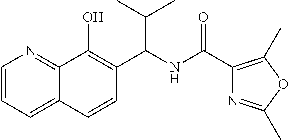 [SMILES: Cc1nc(C(=O)NC(c2ccc3cccnc3c2O)C(C)C)c(C)o1]

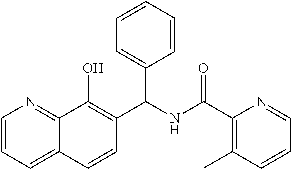 [SMILES: Cc1cccnc1C(=O)NC(c1ccccc1)c1ccc2cccnc2c1O]

39. A pharmaceutical composition, comprising a compound of any one of claims 1-38; and a pharmaceutical acceptable excipient.

40. A method of treating or preventing a disease or disorder associated with a SLC6A8 mutation, comprising administering to a subject in need thereof an effective amount of a compound of any one of claims 1-38.

41. The method of claim 40, wherein the disease or disorder is creatine transporter deficiency.

42. The method of claim 40, wherein the disease or disorder is motor dysfunction.

43. The method of claim 40, wherein the disease or disorder is intellectual disability.

44. The method of claim 40, wherein the disease or disorder is language delay or speech delay.

45. The method of claim 40, wherein the disease or disorder is hypotonia.

46. A method of improving function of a cellular creatine transporter, comprising administering to a subject in need thereof an effective amount of a compound of any one of claims 1-38.

47. The method of claim 46, wherein the creatine transporter is SLC6A8.

48. The method of claim 46 or 47, wherein the creatine transporter is a mutant creatine transporter.

49. A method of decreasing accumulation or the concentration of guanidinoacetic acid or a salt thereof in a cell, comprising administering to a mammal in need thereof a therapeutically effective amount of a compound of any one of claims 1-38.

50. The method of claim 49, wherein the compound decreases intracellular accumulation of guanidinoacetic acid or a salt thereof.

51. The method of claim 49, wherein the compound decreases the intracellular concentration of guanidinoacetic acid or a salt thereof.

52. The method of any one of claims 49-51, wherein the mutant creatine transporter is mutant SLC6A8.

53. The method of any one of claims 49-52, wherein the cell is a brain cell.

54. The method of any one of claims 49-53, wherein the mammal is a male.

55. The method of any one of claims 49-53, wherein the mammal is a female.

56. The method of any one of claims 49-55, wherein the mammal is a primate, equine, bovine, ovine, feline, or canine.

57. The method of any one of claims 49-55, wherein the mammal is a human.

58. A method of increasing transport of guanidinoacetic acid or a salt thereof across the blood-brain barrier, comprising administering to a mammal in need thereof a therapeutically effective amount of a compound of any one of claims 1-38.

59. The method of claim 58, wherein the mutant creatine transporter is mutant SLC6A8.

60. The method of claim 58 or 59, wherein the mammal is a male.

61. The method of claim 58 or 59, wherein the mammal is a female.

62. The method of any one of claims 58-61, wherein the mammal is a primate, equine, bovine, ovine, feline, or canine.

63. The method of any one of claims 58-61, wherein the mammal is a human.

64. A method of treating an inflammatory disease, comprising administering to a mammal in need thereof a therapeutically effective amount of a compound of any one of claims 1-38.

65. The method of claim 64, wherein the inflammatory disease is acute.

66. The method of claim 64, wherein the inflammatory disease is chronic.

67. The method of any one of claims 64-66, wherein the inflammatory disease is selected from inflammatory bowel diseases (for example, ulcerative colitis or Crohn's disease), multiple sclerosis, psoriasis, arthritis, rheumatoid arthritis, osteoarthritis, juvenile arthritis, psoriatic arthritis, reactive arthritis, ankylosing spondylitis, cryopyrin associated periodic syndromes, Muckle-Wells syndrome, familial cold auto-inflammatory syndrome, neonatal-onset multisystem inflammatory disease, TNF receptor associated periodic syndrome, acute and chronic pancreatitis, atherosclerosis, gout, ankylosing spondylitis, fibrotic disorders (for example, hepatic fibrosis or idiopathic pulmonary fibrosis), nephropathy, sarcoidosis, scleroderma, anaphylaxis, diabetes (for example, diabetes mellitus type 1 or diabetes mellitus type 2), diabetic retinopathy, Still's disease, vasculitis, sarcoidosis, pulmonary inflammation, acute respiratory distress syndrome, wet and dry age-related macular degeneration, autoimmune hemolytic syndromes, autoimmune and inflammatory hepatitis, autoimmune neuropathy, autoimmune ovarian failure, autoimmune orchitis, autoimmune thrombocytopenia, silicone implant associated autoimmune disease, Sjogren's syndrome, familial Mediterranean fever, systemic lupus erythematosus, vasculitis syndromes (for example, temporal, Takayasu's and giant cell arteritis, Behçet's disease or Wegener's granulomatosis), vitiligo, secondary hematologic manifestation of autoimmune diseases (for example, anemias), drug-induced autoimmunity, Hashimoto's thyroiditis, hypophysitis, idiopathic thrombocytic pupura, metal-induced autoimmunity, myasthenia gravis, pemphigus, autoimmune deafness (for example, Meniere's disease), Goodpasture's syndrome, Graves' disease, HW-related autoimmune syndromes, Gullain-Barre disease, Addison's disease, anti-phospholipid syndrome, asthma, atopic dermatitis, Celiac disease, Cushing's syndrome, dermatomyositis, idiopathic adrenal adrenal atrophy, idiopathic thrombocytopenia, Kawasaki syndrome, Lambert-Eaton Syndrome, pernicious anemia, pollinosis, polyarteritis nodosa, primary biliary cirrhosis, primary sclerosing cholangitis, Raynaud's, Reiter's Syndrome, relapsing polychondritis, Schmidt's syndrome, thyrotoxidosis, sepsis, septic shock, endotoxic shock, exotoxin-induced toxic shock, gram negative sepsis, toxic shock syndrome, glomerulonephritis, peritonitis, interstitial cystitis, hyperoxia-induced inflammations, chronic obstructive pulmonary disease (COPD), vasculitis, graft vs. host reaction (for example, graft vs. host disease), allograft rejections (for example, acute allograft rejection or chronic allograft rejection), early transplantation rejection (for example, acute allograft rejection), reperfusion injury, pain (for example, acute pain, chronic pain, neuropathic pain, or fibromyalgia), chronic infections, meningitis, encephalitis, myocarditis, gingivitis, post surgical trauma, tissue injury, traumatic brain injury, enterocolitis, sinusitis, uveitis, ocular inflammation, optic neuritis, gastric ulcers, esophagitis, peritonitis, periodontitis, dermatomyositis, gastritis, myositis, polymyalgia, pneumonia and bronchitis.

68. The method of any one of claims 64-67, wherein the mammal is a male.

69. The method of any one of claims 64-67, wherein the mammal is a female.

70. The method of any one of claims 64-69, wherein the mammal is a primate, equine, bovine, ovine, feline, or canine.

71. The method of any one of claims 64-69, wherein the mammal is a human.
